(19) United States
(12) Patent Application Publication
(10) Pub. No.: US 2022/0106604 A1
(43) Pub. Date: Apr. 7, 2022

(54) METHOD FOR INCREASING CANNABIS YIELD VIA GENE EDITING

(71) Applicant: BETTERSEEDS LTD., Givat Chen (IL)

(72) Inventors: Ido MARGALIT, Givat Chen (IL); Tal SHERMAN, Givat Chen (IL); Shira COREM, Givat Chen (IL)

(73) Assignee: BETTERSEEDS LTD., Givat Chen (IL)

(21) Appl. No.: 17/555,540

(22) Filed: Dec. 20, 2021

Related U.S. Application Data
(63) Continuation-in-part of application No. PCT/IL2020/050683, filed on Jun. 18, 2020.
(60) Provisional application No. 62/863,279, filed on Jun. 19, 2019.

(51) Int. Cl. C12N 15/82; C12N 15/11; C12N 9/22

(52) U.S. Cl. CPC C12N 15/8213; C12N 15/827; C12N 2310/20; C12N 9/22; C12N 15/11

(57) ABSTRACT

The present invention discloses a method for increasing flower yield in Cannabis plants via genome editing approach. More particularly, the method comprises steps of: (a) selecting a gene involved in the flowering pathways of said Cannabis species; (b) synthesizing or designing a gRNA expression cassette corresponding to a targeted cleavage locus along the Cannabis genome; (c) transforming said Cannabis plant cells to insert genetic material into them; (d) culturing said Cannabis plant cells; (e) selecting said Cannabis cells which express desired mutations in the editing target region, and (f) regenerating a plant from said transformed plant cell, plant cell nucleus, or plant tissue.

### CROSS REFERENCE TO RELATED APPLICATIONS

This is a Continuation in Part (CIP) of International Application No. PCT/IL2020/050683 filed Jun. 18, 2020; which in turn claims the benefit of U.S. Provisional Patent Application No. 62/863,279, filed Jun. 19, 2019. The contents of the foregoing patent applications are incorporated by reference herein in their entirety.

### FIELD OF INVENTION

The present invention generally relates to the field of improving traits in plants. More particularly, the present invention relates to improving flower yield in Cannabis plants using the CRISPR/Cas genome editing approach.

### BACKGROUND OF THE INVENTION

The Cannabis market is enjoying an unprecedented spike in activity following the wide spread legalization trend across the world. It is estimated that the American market alone would reach a value of at least $30B by 2025, with an exceptional growth rate of 30% per annum. This has led to an increase in demand not only for Cannabis products in general but in particular for products with very specific traits, be it medicinal or recreational use. That demand at times meets a lacking supply, for numerous varied reasons. To allow profitability, growers must leave the environmentally controlled indoor grow facility and go out to the greenhouse or field. Under greenhouse and field conditions, plant performance, for example in terms of growth, development, biomass accumulation and yield, depends on a plant's tolerance and acclimation ability to numerous environmental conditions, changes and stresses. Thus, this transition from the indoor to the outdoor poses several obstacles to growers, and a central such hurdle is achieving consistent high yield.

Numerous avenues have been put forward by inventors and scientists around the world in an attempt to improve Cannabis yield, including photoacoustic energy (US20180127327A1), light (intensity, wavelength, directionality; US20160184237A1, CA2958257C) or transgenic plants with specific traits transformed (U.S. Pat. No. 8,344,205B2).

However, the Cannabis cultivation community has only recently began adopting hard science and the gradual shift from traditional cultivation methods to modern, science-based techniques is still in its infancy. The most acute scientific deficiency in that regard is the lack of fully developed and robust genetics of Cannabis sativa, a shortcoming which hinders the availability and use of genetically enhanced seeds. Without a rapid adoption of genetic tools it is unlikely that Cannabis growers would be able to both meet demand as well as turn a profit, since commercial competition has significantly cut revenues per grower while traditional cultivation measures fail to increase yield in order to compensate for said losses. Further still, the unstable nature of the Cannabis product generated by traditional methods prevents users from enjoying a stable and consistent product, one that would fit particular needs of different consumers. However, big agro companies have yet to jump on the Cannabis wagon due to its still tremulous legal standing.

Since Cannabis cultivation has been illegal for many decades, and only recently has been partially legalized, it still predominantly relies on traditional horticultural techniques, methods, and traditions. These growing practices severely lack scientific rigor and are not suitable for the transition into large-scale Cannabis production. The most flagrant lacuna characterizing this lax scientific approach is the absence of genetic data and tools. Further still, scientists and inventors have so far focused their gaze on improving the production of cannabinoids (WO2018035450A1) rather than ameliorating the physiological parameters of the Cannabis sativa plant as a whole. As a caveat one must acknowledge the fact that attempts have been made in the transgenic front within the context of improving crop yield in general. However, considering the fact that Cannabis users are wearier than others about the GMO status of their product, the insertion of foreign DNA into the Cannabis plant in that fashion may deter a considerable portion of the potential market from such transgenic products. Furthermore, while the emphasis given to cannabinoids is predictable and understandable, neglecting the whole plant physiology is a major hindrance to the industry's ability to meet the growing market demand.

In light of the above, it is the aim of the present invention to provide a novel method of effectively and consistently increasing yield of a transgene-free Cannabis plant. The method is based on gene editing of the Cannabis plant genome at a specific nucleic acid sequence, which results in a set of desired traits which ameliorate the flowering process.

The challenge here is to efficiently induce precise and predictable targeted point mutations pivotal to the flowering process in the cannabis plant using the CRISPR/Cas9 system.

A significant added value of gene editing is that it does not qualify as genetic modification so the resultant transgene-free plant will therefore be not considered a GMO plant/product, at the least in the USA. While the exact and operational definition of genetically modified is hotly debated and contested, it is generally agreed upon and accepted that genetic modification refers to plants and animals that have been altered in a way that wouldn't have arisen naturally through evolution. The clearest and most obvious example is a transgenic organism whose genome now incorporates a gene from another species inserted to bestow a novel trait to that organism, such as pest resistance. The situation is different with CRISPR, as it is not necessarily integrated into the plant genome, and is used as a gene editing tool which allows to directly mutate the organism's genetic code. There is therefore a long felt unmet need to provide Cannabis strains with increased yields.

### SUMMARY OF THE INVENTION

It is therefore one object of the present invention to disclose a method for increasing yield in Cannabis plants selected from the group consisting of C. sativa, C. indica, and C. ruderalis, comprising steps of;
a) selecting a gene involved in the flowering pathways of said Cannabis species;
b) synthesizing or designing a gRNA (guide RNA) expression cassette corresponding to a targeted cleavage locus along the Cannabis genome;
c) transforming said Cannabis plant cells to insert genetic material into them;
d) culturing said Cannabis plant cells;
e) selecting said Cannabis cells which express desired mutations in the editing target region, and
f) regenerating a plant from said transformed plant cell, plant cell nucleus, or plant tissue.

It is a further object of the present invention to disclose the method as defined above, wherein the gene involved in the flowering pathways of said Cannabis species is selected from the group consisting of CsSFT1, CsSFT2, CsSFT3, CsSPGB, CsMultiflora, CsJumonji, CsBif1 and CsBif2; and detailed in the file titled “3309_1_3_SEQ_LISTING”. It is a further object of the present invention to disclose the method as defined above, wherein the gRNAs and their corresponding protospacer adjacent motif (PAMs) are selected from a group consisting of CsSFT1, CsSFT2, CsSFT3, CsSPGB, CsMultiflora, CsJumonji, CsBif1 and CsBif2 and detailed in the file titled “3309_1_3_SEQ_LISTING”. It is a further object of the present invention to disclose the method as defined above, wherein the target domain sequence is selected from the group comprising of: 1) a nucleic acid sequence encoding the polypeptide of CsSFT1 (2) a nucleic acid sequence comprising the sequence of CsSFT2, (3) a nucleic acid sequence encoding the polypeptide of CsSFT3,

(4) a nucleic acid sequence encoding the polypeptide of CsSPGB (5) a nucleic acid sequence encoding the polypeptide of CsMultiflora (6) a nucleic acid sequence encoding the polypeptide of CsJumonji (7) a nucleic acid sequence encoding the polypeptide of CsBif1 (8) a nucleic acid sequence encoding the polypeptide of CsBif2, (9) a nucleic acid sequence having at least 80% sequence identity to at least 200 contiguous nucleotides of the nucleic acid sequence of CsSFT1, (10) a nucleic acid sequence having at least 80% sequence identity to at least 200 contiguous nucleotides of the nucleic acid sequence of CsSFT2, (11) a nucleic acid sequence having at least 80% sequence identity to at least 200 contiguous nucleotides of the nucleic acid sequence of CsSFT3, (12) a nucleic acid sequence having at least 80% sequence identity to at least 200 contiguous nucleotides of the nucleic acid sequence of CsSPGB, (13) a nucleic acid sequence having at least 80% sequence identity to at least 200 contiguous nucleotides of the nucleic acid sequence of CsMultiflora, (14) a nucleic acid sequence having at least 80% sequence identity to at least 200 contiguous nucleotides of the nucleic acid sequence of CsJumonji, (15) a nucleic acid sequence having at least 80% sequence identity to at least 200 contiguous nucleotides of the nucleic acid sequence of CsBif1 (16) a nucleic acid sequence having at least 80% sequence identity to at least 200 contiguous nucleotides of the nucleic acid sequence of CsBif2.

It is a further object of the present invention to disclose the method as defined above, wherein the transformation is carried out using Agrobacterium to deliver an expression cassette comprised of a) a selection marker, b) a nucleotide sequence encoding one or more gRNA molecules comprising a DNA sequence which is complementary with a target domain sequence selected from the group pf genes comprised of CsSFT1, CsSFT2, CsSFT3, CsSPGB, CsMultiflora, CsJumonji, CsBif1 and CsBif2, and c) a nucleotide sequence encoding a Cas molecule from, but not limited to Streptococcus pyogenes or Staphylococcus aureus.

It is a further object of the present invention to disclose the method as defined above, wherein the method comprises administering a nucleic acid composition that comprises: a) a first nucleotide sequence encoding the gRNA molecule and b) a second nucleotide sequence encoding the Cas molecule.

It is a further object of the present invention to disclose the method as defined above, wherein the CRISPR/Cas system is delivered to the cell by a plant virus. It is a further object of the present invention to disclose the method as defined above, wherein the Cas protein is selected from a group comprising but not limited to Cpf1, Cas9, Cas12, Cas13, Cas14, CasX or CasY.

It is a further object of the present invention to disclose the method as defined above, wherein increasing Cannabis yield comprising steps of:
(a) introducing into a Cannabis plant or a cell thereof (i) at least one RNA-guided endonuclease comprising at least one nuclear localization signal or nucleic acid encoding at least one RNA-guided endonuclease comprising at least one nuclear localization signal, (ii) at least one guide RNA or DNA encoding at least one guide RNA, and, optionally, (iii) at least one donor polynucleotide; and
(b) culturing the Cannabis plant or cell thereof such that each guide RNA directs an RNA-guided endonuclease to a targeted site in the chromosomal sequence where the RNA-guided endonuclease introduces a double-stranded break in the targeted site, and the double-stranded break is repaired by a DNA repair process such that the chromosomal sequence is modified, wherein the targeted site is located in the CsSFT1, CsSFT2, CsSFT3, CsSPGB, CsMultiflora, CsJumonji, CsBif1 and CsBif2 genes and the chromosomal modification interrupts or interferes with transcription and/or translation of the CsSFT1, CsSFT2, CsSFT3, CsSPGB, CsMultiflora, CsJumonji, CsBif1 and CsBif2genes.

It is a further object of the present invention to disclose the method as defined above, wherein the RNA-guided endonuclease is derived from a clustered regularly interspersed short palindromic repeats (CRISPR)/CRISPR-associated (Cas) system. It is a further object of the present invention to disclose the method as defined above, wherein the introduction of CsSFT1, CsSFT2, CsSFT3, CsSPGB, CsMultiflora, CsJumonji, CsBif1 and CsBif2 does not insert exogenous genetic material and produces a non-naturally occurring Cannabis plant or cell thereof.

It is a further object of the present invention to disclose the method as defined above, wherein increasing Cannabis yield comprises;
(a) identifying at least one locus within a DNA sequence in a Cannabis plant or a cell thereof for CsSFT1, CsSFT2, CsSFT3, CsSPGB, CsMultiflora, CsJumonji, CsBif1 and CsBif2;
(b) identifying at least one custom endonuclease recognition sequence within the at least one locus of CsSFT1, CsSFT2, CsSFT3, CsSPGB, CsMultiflora, CsJumonji, CsBif1 or CsBif2;
(c) introducing into the Cannabis plant or a cell thereof at least a first custom endonuclease, wherein the Cannabis plant or a cell thereof comprises the recognition sequence for the custom endonuclease in or proximal to the loci of CsSFT1, CsSFT2, CsSFT3, CsSPGB, CsMultiflora, CsJumonji, CsBif1 or CsBif2, and the custom endonuclease is expressed transiently or stably;
(d) assaying the Cannabis plant or a cell thereof for a custom endonuclease-mediated modification in the DNA making up or flanking the loci of CsSFT1, CsSFT2, CsSFT3, CsSPGB, CsMultiflora, CsJumonji, CsBif1 or CsBif2
(e) identifying the Cannabis plant, a cell thereof, or a progeny cell thereof as comprising a modification in the loci of CsSFT1, CsSFT2, CsSFT3, CsSPGB, CsMultiflora, CsJumonji, CsBif1 or CsBif2.

It is a further object of the present invention to disclose the method as defined above, wherein increasing said Cannabis yield is selected from a group consisting of: increasing the number of flowers, increasing the size of the flowers, increasing the weight of the flowers, increasing the number of buds, increasing the size of the buds, increasing the weight of the buds and any combination thereof.

It is a further object of the present invention to disclose a method for increasing yield in Cannabis plants selected from a group consisting of C. sativa, C. indica, and C. ruderalis, comprising steps of;
a) selecting a gene involved in the flowering pathways of said Cannabis species;
b) extracting cells of said Cannabis plants;
c) editing said genes involved in the flowering pathways of said cells;
d) culturing said cells;
e) selecting said cells expressing desired mutations in the editing target region, and
f) regenerating a Cannabis plant from said cell, plant cell nucleus, or plant tissue.

It is a further object of the present invention to disclose the method as defined above, wherein the editing is executed by means selected from a group consisting of: CRISPR/Cas, cleaving the genome of said cell using zinc finger nucleases, cleaving the genome of said cell using meganucleases (homing endonucleases), cleaving the genome of said cell using transcription activator-like effector nucleases (TALEN), and any combination thereof.

It is therefore another object of the present invention to disclose a Cannabis plant produced by the method described above;

It is therefore another object of the present invention to disclose a Cannabis seed of the plant of described above.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

FIG. 1 is a depiction of the transformation process of various Cannabis tissues using the GUS reporter gene;

FIG. 2 is a depiction of transformed leaf tissue screened by PCR for the presence of the Cas9 two weeks post transformation; and

FIG. 3 is a depiction of In vivo specific DNA cleavage by Cas9+gRNA (Ribonucleoprotein protein complex, RNP).

### BRIEF DESCRIPTION OF THE DESCRIBED SEQUENCES

The nucleic and/or amino acid sequences provided herewith are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file named 3309_1_3_SEQ_LISTING.txt, created Dec. 19, 2021, about 299 KB, which is incorporated by reference herein.

### DETAILED DESCRIPTION OF THE INVENTION

The following description is provided, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide a method for increasing flower yield in Cannabis plants.

Introduction to Terms and Explanations Used in the Disclosure of the Present Invention:

The present invention disclosed herein provides a method for producing a plant with increased yield as compared to a corresponding wild type plant comprising increasing or generating one or more activities in a plant or a part thereof. The present invention provides plant cells with enhanced or improved traits of a gene-edited plant, plants comprising such cells, progeny, seed and pollen derived from such plants, and methods of making and methods of using such plant cell(s) or plant(s), progeny, seed(s) or pollen. Particularly, said improved trait(s) are manifested in an increased yield, preferably by improving one or more yield-related trait(s) number of flowers per plant, number of flowering buds per plant, flower weight, total flower yield per m².

Heterosis and Crop Yield

Heterosis (aka hybrid vigor or outbreeding enhancement) defines the enhanced function (or vigor) of a biological trait in a hybrid offspring. An offspring is heterotic if its traits are enhanced as a result of mixing (Mendelian or not) the genetic contributions of its parents. In crop breeding, this kind of outbreeding has come to generally mean a higher-yielding and a more robust plant under cultivation conditions (but not necessarily in the wild), Two non-mutually exclusive yet competing hypotheses have been proposed to account for this tendency of outbred strains to exceed both inbred parents in fitness. According to the dominance hypothesis, the enhanced vigor stems from the suppression of undesirable recessive alleles from one parent by dominant alleles from the other. Dominance assumes complementation, i.e. that crossing two strains of a plant, carrying different homozygous recessive mutations that produce the same mutant phenotype, will produce offspring with the wild-type phenotype. This will occur only if the mutations are in different genes such that strain's genome complements the mutated allele of one strain with a wild type allele of the other (since the mutations are recessive).

According to the overdominance hypothesis, certain combinations of alleles that can be obtained by crossing two inbred strains are advantageous in the heterozygote. Thus, a heterozygote provides an advantage to the survival of deleterious alleles in homozygotes and the high fitness of heterozygous genotypes favors the persistence of an allelic polymorphism in the population. The overdominance model states that intralocus allelic interactions at one or more heterozygous genes lead to increased vigor. Theoretically, overdominance requires only a single heterozygous gene to achieve heterosis.

Under dominance, few genes should be under-expressed in the heterozygous offspring compared to the parents. Furthermore, for any given gene, the expression should be comparable to the one observed in the fitter of the two parents. However, under overdominance, there should be an over-expression of certain genes in the heterozygous offspring compared to the homozygous parents.

Krieger et al. (2010) were first to document an example of overdominance at a locus for yield and suggest that single heterozygous mutations may indeed improve crop productivity. The authors report a robust heterozygosity, under various environmental conditions, for the tomato SFT (single flower truss) gene (the genetic originator of the flowering hormone florigen), increased yield by ˜60%. Florigen is a systemic signal for the transition to flowering in plants. Florigen is produced in the leaves, and acts in the shoot apical meristem of buds and growing tips. It is graft-transmissible, and even functions between species. The florigen cascade pathway is initiated by the production of a mRNA coding transcription factor CONSTANS (CO). CO mRNA is produced approximately 12 hours after dawn and then translated into CO protein. CO protein is stable only in light and promotes transcription of another gene called Flowering Locus T (FT). Thus, FT can be produced only on long days. FT is then transported via the phloem to the shoot apical meristem. There, FT interacts with a transcription factor (FD protein) to activate floral identity genes and induce flowering. The authors concluded that several traits integrate pleiotropically to drive heterosis in a multiplicative manner, and that these effects derive from a suppression of growth termination mediated by the SP (self-pruning) gene, an antagonist of SFT.

Self-pruning (SP) genes are Florigen paralog and flowering repressors that control the regularity of the vegetative-reproductive switch during sympodial growth along the compound shoot of tomato and thus conditions the ‘determinate’ (sp/sp) and ‘indeterminate’ (SP) growth habits of the plant. In wild-type ‘indeterminate’ plants, inflorescences are separated by three vegetative nodes. In ‘determinate’ plants homozygous for the recessive allele of the Self-pruning (SP) gene, by two consecutive inflorescences. SP is a development regulator homologous to the Flowering locus T (FT) gene in Arabidopsis. SP is a gene family in tomato composed of at least six genes. The G-box (CACGTG) is a ubiquitous, cis-acting DNA regulatory element found in plant genomes. G-box factors (GBFs) bind to G-boxes in a context-specific manner, mediating a wide variety of gene expression patterns. SPGB (Self-pruning G-box) has been shown to interact with the tomato SP protein and the SFT protein.

Jumonji-C (JmjC) proteins play important roles in plant growth and development, particularly in regulating circadian clock and period length. The first plant JmjC genes characterized were involved in the flowering cascade, either as floral activators or repressors.

Bifurcate flower truss (bif) is a mutant tomato gene which leads to a significant increase in the number of branches per truss and flower number. Bif shows a significant interaction with exposure to low temperature during truss development.

Gene Editing

Mutation breeding refers to a host of techniques designed to rapidly and effectively induce desired or remove unwanted traits via artificial mutations in a target organism. Gene editing is such a mutation breeding tool which offers significant advantages over genetic modification. Genetic modification is a molecular technology involving inserting a DNA sequence of interest, coding for a desirable trait, into an organism's genome. Gene editing is a mutation breeding tool which allows precise modification of the genome. It works when molecular scissors (a protein complex from the Cas family) are precisely directed toward an exact genome locus using a guide RNA, and then incise the genome at that site.

One advantage to using the CRISPR/Cas system over genetic modification is that Cas family proteins are easily programmed to make a DNA double strand break (DSB) in any desirable locus. The initial cut is followed by repairing chromosomal DSBs. There are two major cellular repair pathways in that respect: Non-homologous end joining (NHEJ) and Homology directed repair (HDR). This invention concerns itself with NHEJ which is active throughout the cell cycle and has a higher capacity for repair, as there is no requirement for a repair template (sister chromatid or homologue) or extensive DNA synthesis. NHEJ also finishes repair of most types of breaks in tens of minutes—an order of magnitude faster than HDR. NHEJ-mediated repair of DSBs is useful if the intent is to make a null allele (knockout) in a gene of interest, as it is prone to generating indel errors. Indel errors generated in the course of repair by NHEJ are typically small (1-10 bp) but extremely heterogeneous. There is consequently about a two-thirds chance of causing a frameshift mutation. Of some importance, the deletion can be less heterogeneous when constrained by sequence identities in flanking sequence (microhomologies).

Additionally, there is no foreign DNA left over in the plant after selection for plants which contain the desired editing event and do not carry the CRISPR/Cas machinery. This significant advantage has allowed gene editing to be viewed by many (though not all) legal systems around the world as GMO-free.

Significant advances have been made recently in an attempt to more efficiently target and cleave genomic DNA by site specific nucleases [e.g. zinc finger nucleases (ZFNs), meganucleases, transcription activator-like effector nucleases (TALENS)]. More recently, RNA-guided endonucleases (RGENs) have been introduced, and they are directed to their target sites by a complementary RNA molecule. These systems have a DNA-binding domain that localizes the nuclease to a target site. The site is then cut by the nuclease. These systems are used to induce targeted mutagenesis, induce targeted deletions of cellular DNA sequences, and facilitate targeted recombination of an exogenous donor DNA polynucleotide within a predetermined genomic locus. Most notable and successful of RGENs is Clustered Regularly Interspaced Short Palindromic Repeats/CRISPR-associated nuclease (CRISPR/Cas) with an engineered crRNA/tracr RNA. CRISPR/Cas9 are cognates that find each other on the target DNA.

The CRISPR-Cas9 system has rapidly become a tool of choice in gene editing because it is faster, cheaper, more accurate, and more efficient than other available RGENs. This system was adapted from a naturally occurring genome editing system in bacteria designed to produce viral resistance such that bacteria capture snippets of DNA from invading viruses and use them to create DNA segments known as CRISPR arrays. The CRISPR arrays allow the bacteria to “remember” the viruses (or closely related ones). If the viruses attack again, the bacteria produce RNA segments from the CRISPR arrays to target the viruses' DNA. The bacteria then use Cas9 or a similar enzyme to cut the DNA, which disables the virus. In lab conditions, scientists create a small piece of RNA with a short “guide” sequence (gRNA) that binds to a specific target sequence of DNA in a genome. The RNA also binds to the Cas9 enzyme. As in bacteria, the modified RNA is used to recognize the DNA sequence, and the Cas9 enzyme cuts the DNA at the targeted location. Although Cas9 is the enzyme that is used most often, other enzymes (for example Cpf1) can also be used. Once the DNA is cut, the cell's own DNA repair machinery add or delete pieces of genetic material resulting in mutation.

Ribonucleoprotein Protein Complex (RNP)

Ribonucleoprotein protein complex is formed when a Cas protein is incubated with gRNA molecules and then transformed into cells in order to induce editing events in the cell. RNP's can be delivered using biolistics.

Biolistics

Biolistics is a method for the delivery of nucleic acid and or proteins to cells by high-speed particle bombardment. The technique uses a pressurized gun (gene gun) to forcibly propel a payload comprised of an elemental particle of a heavy metal coated with plasmid DNA to transform plant cellular organelles. After the DNA-carrying vector has been delivered, the DNA is used as a template for transcription and sometimes it integrates into a plant chromosome (“stable” transformation). If the vector also delivered a selectable marker, then stably transformed cells can be selected and cultured. Transformed plants can become totipotent and even display novel and heritable phenotypes.

The skeletal biolistic vector design includes not only the desired gene to be inserted into the cell, but also promoter and terminator sequences as well as a reporter gene used to enable the ensuing detection and removal cells which failed to incorporate the exogenous DNA. In addition to DNA, the use of a Cas9 protein and a gRNA molecule could be used for biolistic delivery. The advantage of using a protein and a

RNA molecule is that the complex initiates editing upon reaching the cell nucleus: when using DNA for editing the DNA first has to be transcribed in the nucleus but when using RNA for editing, RNA is translated already in the cytoplasm. This forces the Cas protein to shuttle back to the nucleus, find the relevant guides and only then can editing be achieved.

As used herein, the term “CRISPR” refers to an acronym that means Clustered Regularly Interspaced Short Palindromic Repeats of DNA sequences. CRISPR is a series of repeated DNA sequences with unique DNA sequences in between the repeats. RNA transcribed from the unique strands of DNA serves as guides for directing cleaving. CRISPR is used as a gene editing tool. In one embodiment, CRISPR is used in conjunction with (but not limited to) Cpf1, Cas9, Cas12, Cas13, Cas14, CasX or CasY.

As used herein, the term “transformation” refers to the deliberate insertion of genetic material into plant cells. In one embodiment transformation is executed using, but not limited to, bacteria and/or viruses. In another embodiment, transformation is executed via biolistics using, but not limited to, DNA or RNPs.

As used herein, the term “Cas” refers to CRISPR associated proteins that act as enzymes cutting the genome at specific sequences. Cas9 refers to a specific group of proteins known in the art. RNA molecules direct various classes of Cas enzymes to cut a certain sequence found in the genome. In one embodiment, the CRISPR/Cas9 system cleaves one or two chromosomal strands at known DNA sequence. In one embodiment, one of the two chromosomal strands is mutated. In one embodiment, two of the two chromosomal strands are mutated.

As used herein, the term “chromosomal strand” refers to a sequence of DNA within the chromosome. When the CRISPR/Cas9 system cleaves the chromosomal strands, the strands are cut leaving the possibility of one or two strands being mutated, either the template strand or coding strand.

As used herein, the term “PAM” (protospacer adjacent motif) refers to a targeting component of the transformation expression cassette which is a very short (2-6 base pair) DNA sequence immediately following the DNA sequence targeted by the Cas9 nuclease in the CRISPR system.

Within the context of this disclosure, other examples of endonuclease enzymes include, but are not limited to, Cpf1, Cas9, Cas12, Cas13, Cas14, CasX or CasY.

The invention is characterized by a plurality of embodiments in which gRNAs direct the CRISPR/Cas system to cleave chromosomal strands coding for various genes (CsBif1, CsBif2, CsJumonji, CsMultiflora, CsSFT1, CsSFT2, CsSFT3 and CsSPGB). The full genomic sequences of these various genes are all documented in the seq.listing file, listed as SEQ ID NOs: SEQ ID NO:1, SEQ ID NO:171, SEQ ID NO:390, SEQ ID NO:726, SEQ ID NO:936, SEQ ID NO:1015, SEQ ID NO:1106 and SEQ ID NO:1335.

In another embodiment of the present invention, the coding sequences (CDS) of the above genes are all documented in the seq.listing file, listed as SEQ ID NOs: SEQ ID NO:2, SEQ ID NO:172, SEQ ID NO:391, SEQ ID NO:727, SEQ ID NO:937, SEQ ID NO:1016, SEQ ID NO:1107 and SEQ ID NO:1336.

In yet another embodiment of the present invention, the amino acids (AA) sequences of the proteins translated from the above genes are all documented in the seq.listing file, listed as SEQ ID NOs: SEQ ID NO:3, SEQ ID NO:173, SEQ ID NO:392, SEQ ID NO:728, SEQ ID NO:938, SEQ ID NO:1017, SEQ ID NO:1108 and SEQ ID NO:1337.

The invention is further characterized by a plurality of embodiments in which gRNAs of a given sequence are paired with a specific complementary PAMs. These gRNAs are all documented in full in Tables 1-8, and in the seq.listing file listed as SEQ ID Nos: SEQ ID NOs:4-170 (for CsBif1), SEQ ID NOs:174-389 (for CsBif2), SEQ ID NOs:393-725 (for CsJumonji), SEQ ID NOs: 729-935 (for CsMultiflora), SEQ ID NOs: 939-1014 (for CsSFT1), SEQ ID NOs: 1018-1105 (for CsSFT2), SEQ ID NOs: 1109-1334 (for CsSFT3) and SEQ ID NOs: 1338-1500 (for Cs SPGB).

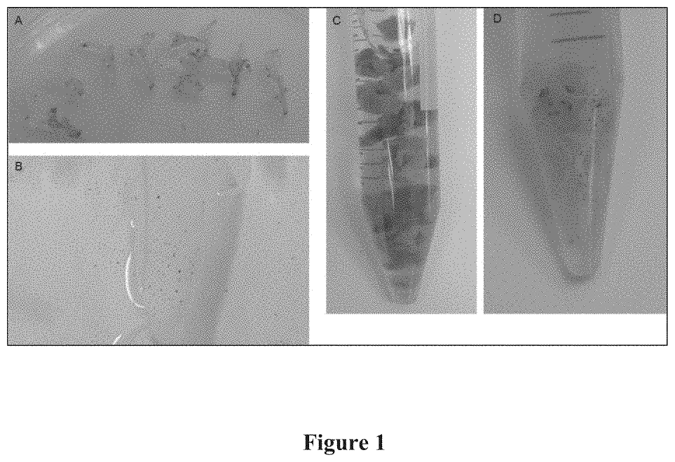

Example 1: A generalized scheme of the process for generating genome edited plants Reference is now made to FIGS. 1-3 disclosing the process of generating genome edited Cannabis plants. Various Cannabis sativa tissues ([A] Auxiliary buds; [B] Mature leaf; [C] Calli; [D] Cotyledons, as depicted in FIG. 1) were transformed using the GUS (β-glucuronidase) reporter gene. In order to achieve a successful transformation, the following protocol was used:
1. Design and synthesize gRNA's corresponding to a sequence targeted for editing. Editing event should be designed flanking a unique restriction site sequence to allow easier screening of successful editing.
2. Transformation using Agrobacterium or biolistics. For Agrobacterium and bioloistics using a DNA plasmid, construct a vector containing a selection marker, Cas9 gene and relevant gRNAs. For biolistics using Ribonucleoprotein (RNP) complexes, create RNP complexes by mixing the Cas9 protein with relevant gRNAs.
3. Regeneration in tissue culture. When transforming DNA, use antibiotics for selection of positive transformants.
4. Selection of positive transformants. Once regenerated plants appear in tissue culture, sample leaf, extract DNA and preform PCR using primers flanking the editing region. Digest PCR products with enzymes recognizing the restriction site near original gRNA sequence. If editing event occurred, the restriction site will be disrupted and PCR product will not be cleaved. No editing event will result in a cleaved PCR product.

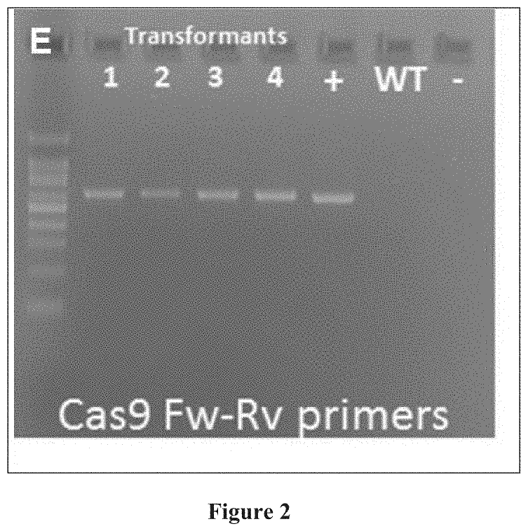

FIG. 2 depicts the transformed leaf tissue screened for the presence of the Cas9 gene two weeks post transformation. PCR products of the Cas9 gene that were amplified from four transformed plants two weeks post transformation.

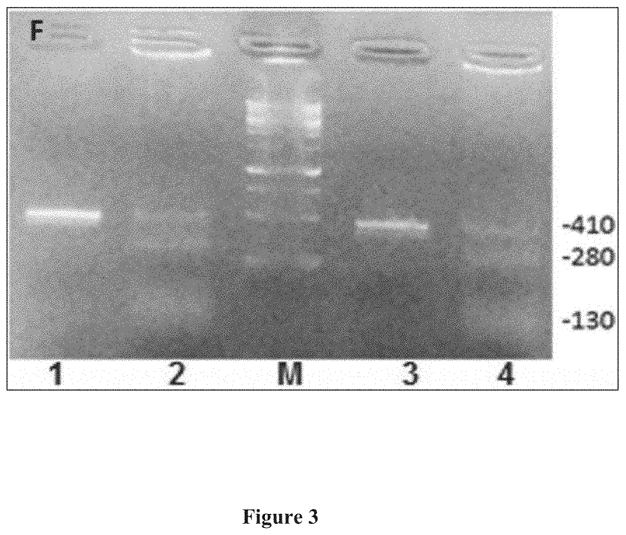

FIG. 3 depicts In vivo specific DNA cleavage by Cas9+gRNA (RNP). Fig represents a gel showing successful digestion of the resulted PCR amplicon containing a specific gRNA sequence, by a ribonucleoprotein (RNP) complex containing Cas9. The analysis included the following steps:
1) Amplicon was isolated from two exemplified Cannabis strains by primers flanking the sequence of the gene of interest targeted by the predesigned sgRNA.
2) RNP complex was incubated with the isolated amplicon.
3) The reaction mix was then loaded on agarose gel to evaluate Cas9 cleavage activity at the target site.

Legend to FIG. 3: (1) Sample 1 PCR (no DNA digest) product; (2) Sample 1 PCR product

+RNP (digested DNA); (3) Sample 2 PCR (no DNA digest) product; (4): Sample 2 PCR product+RNP (digested DNA); (M) marker.

### Example 2: gRNA Sequences for Cannabis sativa Genes Disclosed in the Current Application

Reference is now made to the following tables presenting non-binding examples of gRNA sequences of the Cannabis sativa genes disclosed in this application, and their respective position, strand and PAM (protospacer adjacent motif).

TABLE 1
gRNA (guide RNA) sequences and complementing PAMs
(protospacer adjacent motif) of CsBif1 (referred
to as SEQ ID NOs: 4-170 in the seq.listing
file).

| Seq# | Position | Strand | Sequence | PAM |
|---|---|---|---|---|
| 4 | 14 | -1 | CACATTACATAATAAAATTA | AGG |
| 5 | 89 | 1 | ATGTCTTATATAAAGACTTC | AGG |
| 6 | 161 | -1 | TTTTTGTGATAAAACTCTTC | TGG |
| 7 | 202 | -1 | TATATATATTTTTCAATTGA | GGG |
| 8 | 203 | -1 | TTATATATATTTTTCAATTG | AGG |
| 9 | 269 | 1 | TTAGAAAATAAAAAAATTTA | AGG |
| 10 | 380 | -1 | ATTTTATGTATTTTTATGTA | TGG |
| 11 | 451 | 1 | GATATTACATCTACAAATAG | TGG |
| 12 | 477 | 1 | GATCAGATCAACGATTAGTA | AGG |
| 13 | 499 | 1 | GCGTTGACTATCCTTATCAC | AGG |
| 14 | 499 | -1 | TTTTTCTTTGACCTGTGATA | AGG |
| 15 | 526 | -1 | GAAGAAGAAAGAAGAATTAT | AGG |
| 16 | 577 | -1 | TAGTGTTTTGATTCATTAGG | TGG |
| 17 | 580 | -1 | TAGTAGTGTTTTGATTCATT | AGG |
| 18 | 599 | 1 | ATCAAAACACTACTACAACA | AGG |
| 19 | 684 | 1 | TAAAAAGAGACTCGCATGAG | TGG |
| 20 | 718 | -1 | TTTGCTTATTATAAGGAGGA | GGG |
| 21 | 719 | -1 | CTTTGCTTATTATAAGGAGG | AGG |
| 22 | 722 | -1 | TTCCTTTGCTTATTATAAGG | AGG |
| 23 | 725 | -1 | CATTTCCTTTGCTTATTATA | AGG |
| 24 | 731 | 1 | CTCCTCCTTATAATAAGCAA | AGG |
| 25 | 737 | 1 | CTTATAATAAGCAAAGGAAA | TGG |
| 26 | 777 | 1 | TATTATTATTAAGCATACTG | AGG |
| 27 | 799 | 1 | GATTGAGTGCTATAGCCTCC | TGG |
| 28 | 803 | -1 | GAAAGATATGATCTACCAGG | AGG |
| 29 | 806 | -1 | AATGAAAGATATGATCTACC | AGG |
| 30 | 834 | 1 | TTCATTTGTTCTTCTGTCAA | AGG |
| 31 | 861 | 1 | TGTTCGAATTCGAAAGAGAA | AGG |
| 32 | 876 | 1 | GAGAAAGGATTTGAGCACAC | TGG |
| 33 | 895 | 1 | CTGGTTCATCAGCAACATCA | TGG |
| 34 | 911 | 1 | ATCATGGAGTCTTGTCAAAT | AGG |
| 35 | 912 | 1 | TCATGGAGTCTTGTCAAATA | GGG |
| 36 | 963 | -1 | TCAGTGCCTAACTAACTTGG | GGG |
| 37 | 964 | -1 | ATCAGTGCCTAACTAACTTG | GGG |
| 38 | 965 | -1 | TATCAGTGCCTAACTAACTT | GGG |
| 39 | 966 | -1 | ATATCAGTGCCTAACTAACT | TGG |
| 40 | 968 | 1 | AAAGTTCCCCCAAGTTAGTT | AGG |
| 41 | 989 | 1 | GGCACTGATATCTGAATCAA | AGG |
| 42 | 1005 | 1 | TCAAAGGAGAATGCAAAGAC | AGG |
| 43 | 1072 | -1 | GATCCAAATAGAAGAATTAC | TGG |
| 44 | 1080 | 1 | TTACCAGTAATTCTTCTATT | TGG |
| 45 | 1108 | 1 | ATGTCAACATTTTCTCAATG | AGG |
| 46 | 1115 | 1 | CATTTTCTCAATGAGGTCTA | TGG |
| 47 | 1122 | 1 | TCAATGAGGTCTATGGCCAG | TGG |
| 48 | 1127 | -1 | TTTCCCACATGTTCATCCAC | TGG |
| 49 | 1134 | 1 | ATGGCCAGTGGATGAACATG | TGG |
| 50 | 1135 | 1 | TGGCCAGTGGATGAACATGT | GGG |
| 51 | 1151 | -1 | CCCTCGCCAACAGTTAGGCA | GGG |
| 52 | 1152 | -1 | TCCCTCGCCAACAGTTAGGC | AGG |
| 53 | 1156 | 1 | GGAAAGCCCTGCCTAACTGT | TGG |
| 54 | 1156 | -1 | TCGTTCCCTCGCCAACAGTT | AGG |
| 55 | 1161 | 1 | GCCCTGCCTAACTGTTGGCG | AGG |
| 56 | 1162 | 1 | CCCTGCCTAACTGTTGGCGA | GGG |
| 57 | 1170 | 1 | AACTGTTGGCGAGGGAACGA | AGG |
| 58 | 1171 | 1 | ACTGTTGGCGAGGGAACGAA | GGG |
| 59 | 1188 | -1 | AAGATGCTAAAAGATACATA | CGG |
| 60 | 1225 | -1 | ACACCAACAGAGTCAGATCT | CGG |
| 61 | 1233 | 1 | AAACCGAGATCTGACTCTGT | TGG |
| 62 | 1249 | -1 | TTCTGTGTTTCTTAGCTGCT | AGG |
| 63 | 1318 | 1 | ATAACACGCAAGAACTTAGA | TGG |
| 64 | 1334 | 1 | TAGATGGTAAAAATAAACAA | AGG |
| 65 | 1352 | 1 | AAAGGAAAGCTGTATCTAAT | TGG |
| 66 | 1353 | 1 | AAGGAAAGCTGTATCTAATT | GGG |
| 67 | 1373 | -1 | TAAAAGAACTTTTGGAACTG | TGG |
| 68 | 1381 | -1 | TTTAATGCTAAAAGAACTTT | TGG |
| 69 | 1406 | 1 | AGCATTAAATGAAGAAAAAT | TGG |
| 70 | 1415 | 1 | TGAAGAAAAATTGGCAAAGA | TGG |
| 71 | 1421 | 1 | AAAATTGGCAAAGATGGAAA | AGG |
| 72 | 1441 | 1 | AGGTCTCAATAGTTGAAATT | TGG |
| 73 | 1510 | -1 | TGCGCTTATTGACAGAGGTA | AGG |
| 74 | 1515 | -1 | TCAGATGCGCTTATTGACAG | AGG |
| 75 | 1546 | 1 | TGATGAACATGATCTTTGCC | TGG |
| 76 | 1553 | -1 | AATAGGAAGCCTTTGTTTCC | AGG |
| 77 | 1555 | 1 | TGATCTTTGCCTGGAAACAA | AGG |
| 78 | 1570 | -1 | TCTTTATGGAGCTTATGAAT | AGG |
| 79 | 1584 | -1 | GTCTGTTGGTTGCATCTTTA | TGG |
| 80 | 1598 | -1 | TCAATAGATGTGTGGTCTGT | TGG |
| 81 | 1606 | -1 | ATACTGCTTCAATAGATGTG | TGG |
| 82 | 1645 | 1 | GAAGAGTTCAACAACAACTC | AGG |
| 83 | 1659 | -1 | TGTTGTCACCAGATGGTACA | GGG |
| 84 | 1660 | -1 | ATGTTGTCACCAGATGGTAC | AGG |
| 85 | 1662 | 1 | CTCAGGAGCCCTGTACCATC | TGG |
| 86 | 1666 | -1 | CTGAGTATGTTGTCACCAGA | TGG |
| 87 | 1698 | 1 | AGTCATATACTCATTTTCCG | CGG |
| 88 | 1701 | 1 | CATATACTCATTTTCCGCGG | TGG |
| 89 | 1702 | 1 | ATATACTCATTTTCCGCGGT | GGG |
| 90 | 1704 | -1 | TGGTCTTGCTCGTCCCACCG | CGG |
| 91 | 1724 | -1 | GATCTTAAAATATGTGATTT | TGG |
| 92 | 1757 | 1 | CACAATTCGCATTAAGCAAA | AGG |
| 93 | 1764 | 1 | CGCATTAAGCAAAAGGTTGC | TGG |
| 94 | 1765 | 1 | GCATTAAGCAAAAGGTTGCT | GGG |
| 95 | 1785 | -1 | TTCTGCTAATGTTATACACA | GGG |
| 96 | 1786 | -1 | ATTCTGCTAATGTTATACAC | AGG |
| 97 | 1822 | -1 | TCTTGTACCAGATTCTTCGA | GGG |
| 98 | 1823 | -1 | TTCTTGTACCAGATTCTTCG | AGG |
| 99 | 1826 | 1 | ACTTCAACCCTCGAAGAATC | TGG |
| 100 | 1890 | -1 | AACTTAATTCTAGCTTTTCA | AGG |
| 101 | 1903 | 1 | TTGAAAAGCTAGAATTAAGT | TGG |
| 102 | 1915 | -1 | GTCTTTGTTTATTGACTTCA | TGG |
| 103 | 1949 | -1 | TTTATCAGAGGAGCATTGTC | AGG |
| 104 | 1961 | -1 | TTCAAATCAAGGTTTATCAG | AGG |
| 105 | 1972 | -1 | CAAATTATTCGTTCAAATCA | AGG |
| 106 | 1984 | 1 | CTTGATTTGAACGAATAATT | TGG |
| 107 | 2009 | -1 | CTACATTGCTACTGAGCTCA | TGG |
| 108 | 2046 | 1 | TCAGTAAATTCTCGCCGCAA | AGG |
| 109 | 2049 | 1 | GTAAATTCTCGCCGCAAAGG | TGG |
| 110 | 2049 | -1 | ATGTGATTCCACCACCTTTG | CGG |
| 111 | 2052 | 1 | AATTCTCGCCGCAAAGGTGG | TGG |
| 112 | 2072 | -1 | ACTACAGATTGTAGCTATAA | GGG |
| 113 | 2073 | -1 | TACTACAGATTGTAGCTATA | AGG |
| 114 | 2111 | -1 | TTTTGTTGATTATATATAAA | TGG |
| 115 | 2139 | -1 | TATAGGGTACATAAAAGTCA | AGG |
| 116 | 2155 | -1 | TATCTATTCTATACAATATA | GGG |
| 117 | 2156 | -1 | ATATCTATTCTATACAATAT | AGG |
| 118 | 2180 | -1 | ATGTTTTCTTTTTCATTAAT | TGG |
| 119 | 2246 | -1 | GTACAAAATGAATTTATAAA | TGG |
| 120 | 2275 | 1 | TGTACAGAGTCAATGTAAAT | TGG |
| 121 | 2293 | -1 | ATTGATGATTTGGGTAAATT | AGG |
| 122 | 2302 | -1 | GTATTTTTAATTGATGATTT | GGG |
| 123 | 2303 | -1 | AGTATTTTTAATTGATGATT | TGG |
| 124 | 2347 | -1 | TGTGCCTAATTTTCTGTTTT | TGG |
| 125 | 2354 | 1 | ATCACCAAAAACAGAAAATT | AGG |
| 126 | 2440 | -1 | GATTAAGCTTCTTCGTCATT | TGG |
| 127 | 2464 | -1 | GGATGCCAAACGCACGCTTC | GGG |
| 128 | 2465 | -1 | TGGATGCCAAACGCACGCTT | CGG |
| 129 | 2470 | 1 | AATCTCCCGAAGCGTGCGTT | TGG |
| 130 | 2485 | -1 | TAACGCCTTTGATAATCATA | TGG |
| 131 | 2491 | 1 | GGCATCCATATGATTATCAA | AGG |
| 132 | 2527 | -1 | GAATTCGGAGACGAATGAGA | TGG |
| 133 | 2542 | -1 | TTACAGCTCGGTTTTGAATT | CGG |
| 134 | 2554 | -1 | TTTGTTTTTGAATTACAGCT | CGG |
| 135 | 2592 | -1 | GATTTTGATTCGCTACTTTT | CGG |
| 136 | 2639 | -1 | GAGGAGCTTATGGTATCGTT | TGG |
| 137 | 2649 | -1 | CCTATTGGTAGAGGAGCTTA | TGG |
| 138 | 2658 | -1 | CCGATTATGCCTATTGGTAG | AGG |
| 139 | 2660 | 1 | CCATAAGCTCCTCTACCAAT | AGG |
| 140 | 2664 | -1 | CGACCTCCGATTATGCCTAT | TGG |
| 141 | 2669 | 1 | CCTCTACCAATAGGCATAAT | CGG |
| 142 | 2672 | 1 | CTACCAATAGGCATAATCGG | AGG |
| 143 | 2683 | 1 | CATAATCGGAGGTCGATACT | TGG |
| 144 | 2715 | -1 | TACATTCAGTACAACATATT | TGG |
| 145 | 2739 | 1 | ACTGAATGTACTGACCTCCA | TGG |
| 146 | 2740 | 1 | CTGAATGTACTGACCTCCAT | GGG |
| 147 | 2742 | -1 | CCCGCGATTCCTTCCCATGG | AGG |
| 148 | 2744 | 1 | ATGTACTGACCTCCATGGGA | AGG |
| 149 | 2745 | -1 | TTTCCCGCGATTCCTTCCCA | TGG |
| 150 | 2752 | 1 | ACCTCCATGGGAAGGAATCG | CGG |
| 151 | 2753 | 1 | CCTCCATGGGAAGGAATCGC | GGG |
| 152 | 2770 | -1 | CGGAGGAGGACCTCAGTACA | CGG |
| 153 | 2771 | 1 | GCGGGAAAATCCGTGTACTG | AGG |
| 154 | 2782 | 1 | CGTGTACTGAGGTCCTCCTC | CGG |
| 155 | 2784 | -1 | GCCGCTCCCGGAGCCGGAGG | AGG |
| 156 | 2787 | -1 | GCCGCCGCTCCCGGAGCCGG | AGG |
| 157 | 2788 | 1 | CTGAGGTCCTCCTCCGGCTC | CGG |
| 158 | 2789 | 1 | TGAGGTCCTCCTCCGGCTCC | GGG |
| 159 | 2790 | -1 | GGTGCCGCCGCTCCCGGAGC | CGG |
| 160 | 2794 | 1 | TCCTCCTCCGGCTCCGGGAG | CGG |
| 161 | 2796 | -1 | CTTCCCGGTGCCGCCGCTCC | CGG |
| 162 | 2797 | 1 | TCCTCCGGCTCCGGGAGCGG | CGG |
| 163 | 2803 | 1 | GGCTCCGGGAGCGGCGGCAC | CGG |
| 164 | 2804 | 1 | GCTCCGGGAGCGGCGGCACC | GGG |
| 165 | 2811 | -1 | GCCACTCATAACAATCTTCC | CGG |
| 166 | 2821 | 1 | ACCGGGAAGATTGTTATGAG | TGG |
| 167 | 2839 | -1 | AGAGAAGAGAAACTTCAATA | TGG |
| 168 | 2967 | -1 | TGGTTTAAAAGTCTTGTCTT | TGG |
| 169 | 2987 | -1 | ATTTTTGTTTGATTGAAATT | TGG |
| 170 | 3056 | 1 | TTATTATTATTATTATTATG | AGG |

TABLE 2
gRNA (guide RNA) sequences and complementing PAMs
(protospacer adjacent motif) of CsBif2 (referred
to as SEQ ID NOs: 174-389 in the seq.listing
file).

| Seq# | Position | Strand | Sequence | PAM |
|---|---|---|---|---|
| 174 | 8 | -1 | AAATTAAATGAAAAAGTTTT | AGG |
| 175 | 113 | 1 | AATATTATCGAATATTTTTT | TGG |
| 176 | 221 | 1 | ATATTGATAAAAAGAATATA | TGG |
| 177 | 238 | 1 | ATATGGAAACGATCCTTGAA | AGG |
| 178 | 239 | 1 | TATGGAAACGATCCTTGAAA | GGG |
| 179 | 240 | -1 | TTTTATATTACACCCTTTCA | AGG |
| 180 | 311 | -1 | TGTGTGGATTTTTCTTGAAT | TGG |
| 181 | 327 | -1 | TTGTAAATTGTAATGATGTG | TGG |
| 182 | 353 | -1 | GGTGCTCATATTTTGACTCT | TGG |
| 183 | 374 | -1 | GGGTGTAATTATTAATTTGT | GGG |
| 184 | 375 | -1 | TGGGTGTAATTATTAATTTG | TGG |
| 185 | 394 | -1 | GATTGGTTTAAAAGATATTT | GGG |
| 186 | 395 | -1 | TGATTGGTTTAAAAGATATT | TGG |
| 187 | 411 | -1 | CTAATTTAAGTAAATGTGAT | TGG |
| 188 | 475 | 1 | AAAATTAATTAATTAATTAA | TGG |
| 189 | 476 | 1 | AAATTAATTAATTAATTAAT | GGG |
| 190 | 525 | -1 | TTTTATTGTTGTTGTTATTT | TGG |
| 191 | 575 | -1 | CCAAAGGAGTGTAATAAATT | TGG |
| 192 | 586 | 1 | CCAAATTTATTACACTCCTT | TGG |
| 193 | 591 | -1 | TTCCTTTGAAGAAGAACCAA | AGG |
| 194 | 600 | 1 | CTCCTTTGGTTCTTCTTCAA | AGG |
| 195 | 623 | -1 | TGTTATGTAGTTTTATTTTG | AGG |
| 196 | 681 | 1 | AGATATAGTCTCATAATTAT | AGG |
| 197 | 716 | -1 | GCAACCATGGTTATCTTGTA | AGG |
| 198 | 723 | 1 | TACACCTTACAAGATAACCA | TGG |
| 199 | 729 | -1 | TTTGCATTGCATAGCAACCA | TGG |
| 200 | 764 | -1 | TTGGTTTTTAACAACTACTT | TGG |
| 201 | 783 | -1 | TGATTCTGCATTCAAAGATT | TGG |
| 202 | 797 | 1 | AATCTTTGAATGCAGAATCA | TGG |
| 203 | 877 | 1 | TATAGTAGATAGATAGTACT | AGG |
| 204 | 878 | 1 | ATAGTAGATAGATAGTACTA | GGG |
| 205 | 887 | 1 | AGATAGTACTAGGGTACTGC | TGG |
| 206 | 901 | -1 | TGACTTAAATTGAGAGCTTT | TGG |
| 207 | 926 | 1 | TTTAAGTCAAGAAAAAGAAA | AGG |
| 208 | 954 | 1 | TTTTTTTTTTAATGAAAGAG | AGG |
| 209 | 1001 | 1 | CTATTGACAGAAGCAGCTTC | AGG |
| 210 | 1005 | 1 | TGACAGAAGCAGCTTCAGGA | TGG |
| 211 | 1034 | -1 | CAATGATAAGGGAAATGATG | TGG |
| 212 | 1045 | -1 | TTTGATGGAACCAATGATAA | GGG |
| 213 | 1046 | 1 | CACATCATTTCCCTTATCAT | TGG |
| 214 | 1046 | -1 | ATTTGATGGAACCAATGATA | AGG |
| 215 | 1060 | -1 | TGATATAGATGAGAATTTGA | TGG |
| 216 | 1074 | 1 | TCAAATTCTCATCTATATCA | AGG |
| 217 | 1082 | 1 | TCATCTATATCAAGGCTGAT | TGG |
| 218 | 1089 | 1 | TATCAAGGCTGATTGGTACC | TGG |
| 219 | 1090 | 1 | ATCAAGGCTGATTGGTACCT | GGG |
| 220 | 1094 | 1 | AGGCTGATTGGTACCTGGGC | AGG |
| 221 | 1096 | -1 | GAGACGAAACCCTCCTGCCC | AGG |
| 222 | 1097 | 1 | CTGATTGGTACCTGGGCAGG | AGG |
| 223 | 1098 | 1 | TGATTGGTACCTGGGCAGGA | GGG |
| 224 | 1131 | -1 | CTTCAACACCCTTACATGTC | AGG |
| 225 | 1133 | 1 | TCGTATAGTCCTGACATGTA | AGG |
| 226 | 1134 | 1 | CGTATAGTCCTGACATGTAA | GGG |
| 227 | 1172 | 1 | GTAACAGTGATCCTCTTTGT | TGG |
| 228 | 1172 | -1 | TTGTGTTTGATCCAACAAAG | AGG |
| 229 | 1214 | 1 | TCTAGCAAATCTATTGCTAA | TGG |
| 230 | 1224 | 1 | CTATTGCTAATGGATCAGCA | TGG |
| 231 | 1225 | 1 | TATTGCTAATGGATCAGCAT | GGG |
| 232 | 1226 | 1 | ATTGCTAATGGATCAGCATG | GGG |
| 233 | 1237 | 1 | ATCAGCATGGGGATATAGCC | TGG |
| 234 | 1244 | -1 | CTAGGGGGACACATTTTTCC | AGG |
| 235 | 1259 | -1 | AATCCCTGCCTTATTCTAGG | GGG |
| 236 | 1260 | -1 | AAATCCCTGCCTTATTCTAG | GGG |
| 237 | 1261 | -1 | AAAATCCCTGCCTTATTCTA | GGG |
| 238 | 1262 | 1 | AAATGTGTCCCCCTAGAATA | AGG |
| 239 | 1262 | -1 | TAAAATCCCTGCCTTATTCT | AGG |
| 240 | 1266 | 1 | GTGTCCCCCTAGAATAAGGC | AGG |
| 241 | 1267 | 1 | TGTCCCCCTAGAATAAGGCA | GGG |
| 242 | 1285 | 1 | CAGGGATTTTATGAACTTTC | TGG |
| 243 | 1292 | 1 | TTTATGAACTTTCTGGCCTT | TGG |
| 244 | 1297 | -1 | CGAGTTTATTGATAATCCAA | AGG |
| 245 | 1326 | 1 | ACTCGATATCTGTTTCACGC | TGG |
| 246 | 1341 | -1 | AAACTAATCATCAATGTTCT | TGG |
| 247 | 1359 | 1 | CATTGATGATTAGTTTAAGC | TGG |
| 248 | 1365 | 1 | TGATTAGTTTAAGCTGGTTG | AGG |
| 249 | 1378 | 1 | CTGGTTGAGGCATTCAGTTC | CGG |
| 250 | 1379 | 1 | TGGTTGAGGCATTCAGTTCC | GGG |
| 251 | 1386 | -1 | GGTAGAAAACCAATCTTTCC | CGG |
| 252 | 1388 | 1 | CATTCAGTTCCGGGAAAGAT | TGG |
| 253 | 1401 | 1 | GAAAGATTGGTTTTCTACCA | AGG |
| 254 | 1407 | -1 | TGCATATTTGCTGAAATCCT | TGG |
| 255 | 1431 | -1 | TCCATTGATGTCTGGTCTGT | AGG |
| 256 | 1439 | -1 | ATGGAACATCCATTGATGTC | TGG |
| 257 | 1441 | 1 | TCCTACAGACCAGACATCAA | TGG |
| 258 | 1458 | -1 | CTTCTCTGTTGTGACAACTA | TGG |
| 259 | 1477 | 1 | GTCACAACAGAGAAGTAGCT | CGG |
| 260 | 1478 | 1 | TCACAACAGAGAAGTAGCTC | GGG |
| 261 | 1499 | -1 | CAGAATATGTTGTGACTCGC | TGG |
| 262 | 1532 | -1 | CGAGAACCAGTAGAGGAAAT | GGG |
| 263 | 1533 | -1 | GCGAGAACCAGTAGAGGAAA | TGG |
| 264 | 1537 | 1 | GAATTGCCCATTTCCTCTAC | TGG |
| 265 | 1539 | -1 | GGTCTAGCGAGAACCAGTAG | AGG |
| 266 | 1560 | -1 | GACCTAAAGATATGCGATTT | TGG |
| 267 | 1569 | 1 | GACCAAAATCGCATATCTTT | AGG |
| 268 | 1590 | 1 | GGTCACAATTAGCATTGACA | AGG |
| 269 | 1593 | 1 | CACAATTAGCATTGACAAGG | AGG |
| 270 | 1600 | 1 | AGCATTGACAAGGAGGTTCC | CGG |
| 271 | 1601 | 1 | GCATTGACAAGGAGGTTCCC | GGG |
| 272 | 1607 | -1 | TTCACCGAGACTTGAAGCCC | GGG |
| 273 | 1608 | -1 | CTTCACCGAGACTTGAAGCC | CGG |
| 274 | 1614 | 1 | GGTTCCCGGGCTTCAAGTCT | CGG |
| 275 | 1658 | -1 | CTGTTGTGCAGTTGCTTCGC | GGG |
| 276 | 1659 | -1 | ACTGTTGTGCAGTTGCTTCG | CGG |
| 277 | 1690 | 1 | AGTGAAACATTCATAAGTAA | TGG |
| 278 | 1704 | -1 | CAGTGGATTGATGCAATTTT | CGG |
| 279 | 1721 | -1 | CTTTTAAGTACTGTTTTCAG | TGG |
| 280 | 1750 | 1 | AAAAGAACTGTAAAACACAC | AGG |
| 281 | 1796 | -1 | CTGCAAATACTTTTTATTTC | AGG |
| 282 | 1824 | 1 | TTGCAGTGATCACTAGAAAG | CGG |
| 283 | 1832 | 1 | ATCACTAGAAAGCGGTTGTG | AGG |
| 284 | 1849 | 1 | GTGAGGACTTAATAATTTGA | TGG |
| 285 | 1852 | 1 | AGGACTTAATAATTTGATGG | AGG |
| 286 | 1871 | -1 | TTATCTGGTTTATGAGCTCA | TGG |
| 287 | 1886 | -1 | AACTTTCAAAGATGTTTATC | TGG |
| 288 | 1911 | 1 | TTGAAAGTTGCTCTATGAAT | AGG |
| 289 | 1934 | -1 | TGAGAATGTGATTGCTTTAA | AGG |
| 290 | 1968 | -1 | TGAGGGAGTTGAAGCTTCTT | AGG |
| 291 | 1985 | -1 | AGATGCCCTGAGGACTCTGA | GGG |
| 292 | 1986 | -1 | TAGATGCCCTGAGGACTCTG | AGG |
| 293 | 1990 | 1 | TCAACTCCCTCAGAGTCCTC | AGG |
| 294 | 1991 | 1 | CAACTCCCTCAGAGTCCTCA | GGG |
| 295 | 1995 | -1 | AGAACCGCGTAGATGCCCTG | AGG |
| 296 | 2002 | 1 | GAGTCCTCAGGGCATCTACG | CGG |
| 297 | 2063 | -1 | GGTGTGCTCGTCGATCAATA | GGG |
| 298 | 2064 | -1 | TGGTGTGCTCGTCGATCAAT | AGG |
| 299 | 2084 | 1 | CGACGAGCACACCACGCCAT | AGG |
| 300 | 2084 | -1 | AGGGAGAGGTGCCTATGGCG | TGG |
| 301 | 2089 | -1 | CCAATAGGGAGAGGTGCCTA | TGG |
| 302 | 2098 | -1 | CCTATCAAACCAATAGGGAG | AGG |
| 303 | 2100 | 1 | CCATAGGCACCTCTCCCTAT | TGG |
| 304 | 2103 | -1 | ATGTTCCTATCAAACCAATA | GGG |
| 305 | 2104 | -1 | TATGTTCCTATCAAACCAAT | AGG |
| 306 | 2109 | 1 | CCTCTCCCTATTGGTTTGAT | AGG |
| 307 | 2120 | 1 | TGGTTTGATAGGAACATACT | TGG |
| 308 | 2154 | -1 | GAAAGCATTACTACTCAATG | TGG |
| 309 | 2176 | -1 | CCTAATGGAGTTAGGCAACA | AGG |
| 310 | 2184 | -1 | TTGATCCCCCTAATGGAGTT | AGG |
| 311 | 2187 | 1 | CCTTGTTGCCTAACTCCATT | AGG |
| 312 | 2188 | 1 | CTTGTTGCCTAACTCCATTA | GGG |
| 313 | 2189 | 1 | TTGTTGCCTAACTCCATTAG | GGG |
| 314 | 2190 | 1 | TGTTGCCTAACTCCATTAGG | GGG |
| 315 | 2191 | -1 | ACTTTGGTTGATCCCCCTAA | TGG |
| 316 | 2207 | -1 | TGTAGGGAAAATGGCAACTT | TGG |
| 317 | 2216 | -1 | TCGGTGATTTGTAGGGAAAA | TGG |
| 318 | 2223 | -1 | ATTGATTTCGGTGATTTGTA | GGG |
| 319 | 2224 | -1 | GATTGATTTCGGTGATTTGT | AGG |
| 320 | 2235 | -1 | AAATGGGTGTTGATTGATTT | CGG |
| 321 | 2251 | -1 | CTTGTGATTTAATCTTAAAT | GGG |
| 322 | 2252 | -1 | TCTTGTGATTTAATCTTAAA | TGG |
| 323 | 2346 | -1 | TTTTAACTCATTGTATAACT | GGG |
| 324 | 2347 | -1 | GTTTTAACTCATTGTATAAC | TGG |
| 325 | 2376 | 1 | AAAACTAAGAAAAAGTTAAG | AGG |
| 326 | 2436 | -1 | AGAAGTAGAATTGGCAAGTA | AGG |
| 327 | 2445 | -1 | TCATAACCCAGAAGTAGAAT | TGG |
| 328 | 2449 | 1 | TTACTTGCCAATTCTACTTC | TGG |
| 329 | 2450 | 1 | TACTTGCCAATTCTACTTCT | GGG |
| 330 | 2472 | 1 | GTTATGATCTTCTCCCTATA | AGG |
| 331 | 2474 | -1 | GTGTAGAGAATAACCTTATA | GGG |
| 332 | 2475 | -1 | AGTGTAGAGAATAACCTTAT | AGG |
| 333 | 2505 | -1 | TTTACTATTTAAAAAAAGAG | GGG |
| 334 | 2506 | -1 | TTTTACTATTTAAAAAAAGA | GGG |
| 335 | 2507 | -1 | ATTTTACTATTTAAAAAAAG | AGG |
| 336 | 2531 | -1 | AGGGAAAAAGGCATAAAAGT | GGG |
| 337 | 2532 | -1 | AAGGGAAAAAGGCATAAAAG | TGG |
| 338 | 2543 | -1 | GAGAGAAGTCTAAGGGAAAA | AGG |
| 339 | 2550 | -1 | AAGGAGAGAGAGAAGTCTAA | GGG |
| 340 | 2551 | -1 | AAAGGAGAGAGAGAAGTCTA | AGG |
| 341 | 2569 | -1 | AAGTGAACAAAGTGAGGAAA | AGG |
| 342 | 2575 | -1 | GGTCATAAGTGAACAAAGTG | AGG |
| 343 | 2596 | -1 | GGATTAAGTATATGAGAGGA | TGG |
| 344 | 2600 | -1 | AAGAGGATTAAGTATATGAG | AGG |
| 345 | 2617 | -1 | TGAGAGGTTTTTTTAGGAAG | AGG |
| 346 | 2623 | -1 | TGAATTTGAGAGGTTTTTTT | AGG |
| 347 | 2633 | -1 | GTGTGTGAACTGAATTTGAG | AGG |
| 348 | 2673 | -1 | AAAAGATTGTTGTTTTGTTG | AGG |
| 349 | 2696 | -1 | AAGGAGTTGTAAGAATCTAG | AGG |
| 350 | 2715 | -1 | GAGAATAAGGAGAAGAATTA | AGG |
| 351 | 2728 | -1 | TTGGCTTAATTTTGAGAATA | AGG |
| 352 | 2747 | -1 | CAGAGTAAAAGTTTGATTCT | TGG |
| 353 | 2772 | -1 | TTTTGAAGGAATCTTTTACT | TGG |
| 354 | 2786 | -1 | TTGCTTGGTTTTGTTTTTGA | AGG |
| 355 | 2801 | -1 | TTGGAAAGATGGGTTTTGCT | TGG |
| 356 | 2811 | -1 | AGCTTTATTTTTGGAAAGAT | GGG |
| 357 | 2812 | -1 | GAGCTTTATTTTTGGAAAGA | TGG |
| 358 | 2820 | -1 | AAAGTACTGAGCTTTATTTT | TGG |
| 359 | 2845 | -1 | TTTCTTTTCTTTTAATTGGA | GGG |
| 360 | 2846 | -1 | TTTTCTTTTCTTTTAATTGG | AGG |
| 361 | 2849 | -1 | ATTTTTTCTTTTCTTTTAAT | TGG |
| 362 | 2886 | 1 | TTTTTGCAGAAACCCCAAAA | AGG |
| 363 | 2887 | 1 | TTTTGCAGAAACCCCAAAAA | GGG |
| 364 | 2887 | -1 | TCTTTCTTTTTCCCTTTTTG | GGG |
| 365 | 2888 | -1 | CTCTTTCTTTTTCCCTTTTT | GGG |
| 366 | 2889 | -1 | TCTCTTTCTTTTTCCCTTTT | TGG |
| 367 | 2922 | -1 | ATGTTGGTTGTCAATTATTG | AGG |
| 368 | 2938 | -1 | ATTTTTCTGATTATTCATGT | TGG |
| 369 | 2965 | 1 | GAAAAATCTGAGATAATTGA | AGG |
| 370 | 2993 | -1 | CACAGACCTGTTTCAGATAA | AGG |
| 371 | 2998 | 1 | CAATATCCTTTATCTGAAAC | AGG |
| 372 | 3050 | -1 | TTCTGTTCAAGACGATTTGA | AGG |
| 373 | 3072 | 1 | TCTTGAACAGAAAAAAACTT | AGG |
| 374 | 3089 | -1 | AAGTTTGTATCTTTAATGGT | GGG |
| 375 | 3090 | -1 | AAAGTTTGTATCTTTAATGG | TGG |
| 376 | 3093 | -1 | CTTAAAGTTTGTATCTTTAA | TGG |
| 377 | 3120 | -1 | TTTTTCTCATTTTAAGTATT | GGG |
| 378 | 3121 | -1 | ATTTTTCTCATTTTAAGTAT | TGG |
| 379 | 3134 | 1 | AATACTTAAAATGAGAAAAA | TGG |
| 380 | 3140 | 1 | TAAAATGAGAAAAATGGAAA | CGG |
| 381 | 3141 | 1 | AAAATGAGAAAAATGGAAAC | GGG |
| 382 | 3146 | 1 | GAGAAAAATGGAAACGGGTT | TGG |
| 383 | 3147 | 1 | AGAAAAATGGAAACGGGTTT | GGG |
| 384 | 3148 | 1 | GAAAAATGGAAACGGGTTTG | GGG |
| 385 | 3155 | 1 | GGAAACGGGTTTGGGGAGAA | TGG |
| 386 | 3156 | 1 | GAAACGGGTTTGGGGAGAAT | GGG |
| 387 | 3157 | 1 | AAACGGGTTTGGGGAGAATG | GGG |
| 388 | 3161 | 1 | GGGTTTGGGGAGAATGGGGC | AGG |
| 389 | 3183 | -1 | GGTAGTGTCATGGGTGGGTT | TGG |

TABLE 3
gRNA (guide RNA) sequences and complementing PAMs
(protospacer adjacent motif) of CsJumonji
(referred to as SEQ ID NOs: 393-725 in the
seq.listing file).

| Seq# | Position | Strand | Sequence | PAM |
|---|---|---|---|---|
| 393 | 1321 | 1 | GAAAATGAAGTTAGCAAGTC | AGG |
| 394 | 1340 | 1 | CAGGCTCTAGTTTGATATTG | TGG |
| 395 | 1354 | 1 | ATATTGTGGCTCCAGAGAGC | AGG |
| 396 | 1354 | -1 | TTTTTTATTGACCTGCTCTC | TGG |
| 397 | 1369 | 1 | AGAGCAGGTCAATAAAAAAA | TGG |
| 398 | 1392 | 1 | TGTGTAATCTAATAATGAAA | TGG |
| 399 | 1400 | 1 | CTAATAATGAAATGGTGAAA | TGG |
| 400 | 1426 | -1 | GGTTAGATTGAAAGAAGAAC | AGG |
| 401 | 1447 | -1 | TAGGGTCAGAGACAGGTCAC | AGG |
| 402 | 1454 | -1 | TCATTTCTAGGGTCAGAGAC | AGG |
| 403 | 1465 | -1 | TCAGAGGCCCTTCATTTCTA | GGG |
| 404 | 1466 | -1 | GTCAGAGGCCCTTCATTTCT | AGG |
| 405 | 1468 | 1 | GTCTCTGACCCTAGAAATGA | AGG |
| 406 | 1469 | 1 | TCTCTGACCCTAGAAATGAA | GGG |
| 407 | 1481 | -1 | GCAAAACGCCTTAAAGTCAG | AGG |
| 408 | 1484 | 1 | ATGAAGGGCCTCTGACTTTA | AGG |
| 409 | 1505 | -1 | GAACCACACCCAGTAGATAT | TGG |
| 410 | 1507 | 1 | CGTTTTGCTCCAATATCTAC | TGG |
| 411 | 1508 | 1 | GTTTTGCTCCAATATCTACT | GGG |
| 412 | 1513 | 1 | GCTCCAATATCTACTGGGTG | TGG |
| 413 | 1527 | -1 | AGAGTTGGGTAAGAGCACGA | GGG |
| 414 | 1528 | -1 | TAGAGTTGGGTAAGAGCACG | AGG |
| 415 | 1541 | -1 | GATAGGTTTCAGTTAGAGTT | GGG |
| 416 | 1542 | -1 | GGATAGGTTTCAGTTAGAGT | TGG |
| 417 | 1558 | -1 | GAGAACAACCCAAAAAGGAT | AGG |
| 418 | 1560 | 1 | TAACTGAAACCTATCCTTTT | TGG |
| 419 | 1561 | 1 | AACTGAAACCTATCCTTTTT | GGG |
| 420 | 1563 | -1 | ACTGAGAGAACAACCCAAAA | AGG |
| 421 | 1598 | -1 | TTCAAGAATGTGGTTAATGA | GGG |
| 422 | 1599 | -1 | ATTCAAGAATGTGGTTAATG | AGG |
| 423 | 1608 | -1 | CTCTATAAAATTCAAGAATG | TGG |
| 424 | 1624 | 1 | TTCTTGAATTTTATAGAGAT | CGG |
| 425 | 1629 | 1 | GAATTTTATAGAGATCGGAA | TGG |
| 426 | 1643 | -1 | AGAAAGATTTGTCAAGGAAG | GGG |
| 427 | 1644 | -1 | CAGAAAGATTTGTCAAGGAA | GGG |
| 428 | 1645 | -1 | ACAGAAAGATTTGTCAAGGA | AGG |
| 429 | 1649 | -1 | AACGACAGAAAGATTTGTCA | AGG |
| 430 | 1701 | 1 | CTATGATTAGTGAGCGTAGT | TGG |
| 431 | 1730 | -1 | TCAGGTTGCACCAGAATTGT | GGG |
| 432 | 1731 | 1 | AGAATTTGATCCCACAATTC | TGG |
| 433 | 1731 | -1 | GTCAGGTTGCACCAGAATTG | TGG |
| 434 | 1748 | -1 | GACAGAAGATCTGGGTCGTC | AGG |
| 435 | 1756 | -1 | TTCAGCCTGACAGAAGATCT | GGG |
| 436 | 1757 | -1 | TTTCAGCCTGACAGAAGATC | TGG |
| 437 | 1762 | 1 | GACGACCCAGATCTTCTGTC | AGG |
| 438 | 1798 | -1 | CTGAAATTTTTAATGGACAG | GGG |
| 439 | 1799 | -1 | GCTGAAATTTTTAATGGACA | GGG |
| 440 | 1800 | -1 | TGCTGAAATTTTTAATGGAC | AGG |
| 441 | 1805 | -1 | CTGGTTGCTGAAATTTTTAA | TGG |
| 442 | 1824 | -1 | CAATTGATGATTAACTTTTC | TGG |
| 443 | 1840 | 1 | AAAGTTAATCATCAATTGTT | AGG |
| 444 | 1852 | 1 | CAATTGTTAGGACACAATAC | TGG |
| 445 | 1866 | 1 | CAATACTGGTTTATGAAACT | AGG |
| 446 | 1898 | 1 | TTACGTGTAAACTAAGTACC | TGG |
| 447 | 1901 | 1 | CGTGTAAACTAAGTACCTGG | TGG |
| 448 | 1905 | -1 | ATCGAAGCATTCTGACCACC | AGG |
| 449 | 1956 | -1 | ACGTCGTTCAACACAGAAGA | TGG |
| 450 | 1983 | -1 | TTCTGATTCAGAGATATTCA | GGG |
| 451 | 1984 | -1 | GTTCTGATTCAGAGATATTC | AGG |
| 452 | 2020 | 1 | TCACTTTCTTCATCTAGAGT | AGG |
| 453 | 2032 | -1 | ATTCTCATGAAAAGCTGGTT | AGG |
| 454 | 2037 | -1 | AGATTATTCTCATGAAAAGC | TGG |
| 455 | 2080 | 1 | GTCAAGTGTTCATCACATGA | CGG |
| 456 | 2081 | 1 | TCAAGTGTTCATCACATGAC | GGG |
| 457 | 2082 | 1 | CAAGTGTTCATCACATGACG | GGG |
| 458 | 2119 | -1 | AGTTTTCAGAAGACTTGTCA | CGG |
| 459 | 2158 | -1 | GGAACACTAGCTTTGATGTG | AGG |
| 460 | 2179 | -1 | TTTATTTTCTCCAGGTACAT | GGG |
| 461 | 2180 | 1 | AGCTAGTGTTCCCATGTACC | TGG |
| 462 | 2180 | -1 | ATTTATTTTCTCCAGGTACA | TGG |
| 463 | 2187 | -1 | TGCATCTATTTATTTTCTCC | AGG |
| 464 | 2244 | 1 | GCACTCTAAATAATTCTGAA | AGG |
| 465 | 2279 | 1 | ATACAGAAGTAGCCAACTAA | AGG |
| 466 | 2280 | -1 | AGATACAGATCTCCTTTAGT | TGG |
| 467 | 2302 | 1 | AGATCTGTATCTTACATTGC | TGG |
| 468 | 2303 | 1 | GATCTGTATCTTACATTGCT | GGG |
| 469 | 2314 | 1 | TACATTGCTGGGAGTGATTG | CGG |
| 470 | 2324 | 1 | GGAGTGATTGCGGCTTCCGT | AGG |
| 471 | 2325 | 1 | GAGTGATTGCGGCTTCCGTA | GGG |
| 472 | 2329 | -1 | TTCTCTTCCTGTAGACCCTA | CGG |
| 473 | 2333 | 1 | GCGGCTTCCGTAGGGTCTAC | AGG |
| 474 | 2350 | 1 | TACAGGAAGAGAATGTAGAG | TGG |
| 475 | 2356 | 1 | AAGAGAATGTAGAGTGGATG | TGG |
| 476 | 2362 | 1 | ATGTAGAGTGGATGTGGTAC | AGG |
| 477 | 2385 | -1 | AGTTTAAATTCTGAAATGTT | AGG |
| 478 | 2414 | -1 | AGTGCACAGGTGTCAAGTAG | TGG |
| 479 | 2427 | -1 | TCATGTTTCGTGTAGTGCAC | AGG |
| 480 | 2482 | 1 | TGAGTAGTATCACTAAGTTT | TGG |
| 481 | 2483 | 1 | GAGTAGTATCACTAAGTTTT | GGG |
| 482 | 2503 | 1 | GGGATCGAGTCAAATTTGAT | CGG |
| 483 | 2512 | 1 | TCAAATTTGATCGGACAGTA | AGG |
| 484 | 2532 | -1 | GTCACATAAAGTTAAGCAGG | AGG |
| 485 | 2535 | -1 | CATGTCACATAAAGTTAAGC | AGG |
| 486 | 2557 | 1 | TTATGTGACATGTTTGCTAA | TGG |
| 487 | 2589 | -1 | GATTAATCAGCAATCTAATA | GGG |
| 488 | 2590 | -1 | AGATTAATCAGCAATCTAAT | AGG |
| 489 | 2611 | 1 | TGCTGATTAATCTTCTTTTC | TGG |
| 490 | 2631 | -1 | AGCTAGAAAGTTTGAAATGG | AGG |
| 491 | 2634 | -1 | TGCAGCTAGAAAGTTTGAAA | TGG |
| 492 | 2661 | -1 | AAGGGAGGATATATCAGAAA | TGG |
| 493 | 2676 | -1 | CTGTACGCTCTGTTTAAGGG | AGG |
| 494 | 2679 | -1 | ACACTGTACGCTCTGTTTAA | GGG |
| 495 | 2680 | -1 | GACACTGTACGCTCTGTTTA | AGG |
| 496 | 2700 | 1 | GAGCGTACAGTGTCTTCCAC | AGG |
| 497 | 2705 | -1 | ACGTCTTTAAATTTTTCCTG | TGG |
| 498 | 2776 | 1 | TAAGAGTGTGTAAGAATCCA | AGG |
| 499 | 2782 | -1 | AATGCTATATGTTTCTGCCT | TGG |
| 500 | 2795 | 1 | AAGGCAGAAACATATAGCAT | TGG |
| 501 | 2811 | 1 | GCATTGGTAAAATCCCCCTG | TGG |
| 502 | 2812 | 1 | CATTGGTAAAATCCCCCTGT | GGG |
| 503 | 2813 | -1 | ACTTAACAGCAGCCCACAGG | GGG |
| 504 | 2814 | -1 | TACTTAACAGCAGCCCACAG | GGG |
| 505 | 2815 | -1 | CTACTTAACAGCAGCCCACA | GGG |
| 506 | 2816 | -1 | CCTACTTAACAGCAGCCCAC | AGG |
| 507 | 2827 | 1 | CCTGTGGGCTGCTGTTAAGT | AGG |
| 508 | 2832 | 1 | GGGCTGCTGTTAAGTAGGAA | TGG |
| 509 | 2847 | -1 | CCAATGGGACTTTTGATACT | GGG |
| 510 | 2848 | -1 | CCCAATGGGACTTTTGATAC | TGG |
| 511 | 2858 | 1 | CCCAGTATCAAAAGTCCCAT | TGG |
| 512 | 2859 | 1 | CCAGTATCAAAAGTCCCATT | GGG |
| 513 | 2862 | -1 | AATTATGACCTCTACCCAAT | GGG |
| 514 | 2863 | -1 | GAATTATGACCTCTACCCAA | TGG |
| 515 | 2865 | 1 | TCAAAAGTCCCATTGGGTAG | AGG |
| 516 | 2899 | -1 | GTATTCATCACCATTTTAAT | GGG |
| 517 | 2900 | 1 | AAGTTTATATCCCATTAAAA | TGG |
| 518 | 2900 | -1 | TGTATTCATCACCATTTTAA | TGG |
| 519 | 2988 | -1 | CAGCCTAGTGTTTGCGTGTT | TGG |
| 520 | 2996 | 1 | AATCCAAACACGCAAACACT | AGG |
| 521 | 3000 | 1 | CAAACACGCAAACACTAGGC | TGG |
| 522 | 3023 | 1 | ATAGAAGCATACCATGACGA | AGG |
| 523 | 3023 | -1 | CATCCTGTCTGCCTTCGTCA | TGG |
| 524 | 3031 | 1 | ATACCATGACGAAGGCAGAC | AGG |
| 525 | 3095 | 1 | CACGTTTACATAAGCTGCAC | AGG |
| 526 | 3113 | -1 | AGTGTGTCTGCAAATCGGCA | TGG |
| 527 | 3118 | -1 | GTACTAGTGTGTCTGCAAAT | CGG |
| 528 | 3145 | -1 | GCTGGTTGCTAATGAAATCA | GGG |
| 529 | 3146 | -1 | CGCTGGTTGCTAATGAAATC | AGG |
| 530 | 3163 | -1 | GTTTCTTGCATCGAGCTCGC | TGG |
| 531 | 3176 | 1 | AGCGAGCTCGATGCAAGAAA | CGG |
| 532 | 3204 | 1 | CTTCACAAAAGAAGTTTTAA | TGG |
| 533 | 3216 | 1 | AGTTTTAATGGAACAATGAG | AGG |
| 534 | 3228 | -1 | GGATCATTCTTCTGCTGACT | TGG |
| 535 | 3249 | -1 | GAGTTTAGAGCTTGAAGATT | TGG |
| 536 | 3304 | 1 | TTGCAAAGTAGTTCTTCATG | AGG |
| 537 | 3313 | 1 | AGTTCTTCATGAGGAAGCAA | AGG |
| 538 | 3328 | -1 | AACGCTATGCACTTCTTAGT | AGG |
| 539 | 3341 | 1 | TACTAAGAAGTGCATAGCGT | TGG |
| 540 | 3360 | 1 | TTGGCTAGCAACAGCACCCA | AGG |
| 541 | 3361 | 1 | TGGCTAGCAACAGCACCCAA | GGG |
| 542 | 3365 | -1 | ATTGGTGACTGGTTTCCCTT | GGG |
| 543 | 3366 | -1 | AATTGGTGACTGGTTTCCCT | TGG |
| 544 | 3376 | -1 | TGAACTTTGCAATTGGTGAC | TGG |
| 545 | 3383 | -1 | GAAGCAGTGAACTTTGCAAT | TGG |
| 546 | 3407 | -1 | GGAACTGTAGGCTTCAATTG | TGG |
| 547 | 3419 | -1 | ACTTTTCCTTTTGGAACTGT | AGG |
| 548 | 3424 | 1 | TTGAAGCCTACAGTTCCAAA | AGG |
| 549 | 3428 | -1 | AGAAATTGTACTTTTCCTTT | TGG |
| 550 | 3529 | -1 | TATCATGCTGGATTTAATCA | TGG |
| 551 | 3541 | -1 | TTCCCTAGAGCATATCATGC | TGG |
| 552 | 3549 | 1 | AATCCAGCATGATATGCTCT | AGG |
| 553 | 3550 | 1 | ATCCAGCATGATATGCTCTA | GGG |
| 554 | 3573 | 1 | AACGTAACTATGAACTCTCC | AGG |
| 555 | 3580 | -1 | TACAAGGCAGTGCAAAAACC | TGG |
| 556 | 3596 | -1 | TCATGACGTCCCTGTGTACA | AGG |
| 557 | 3597 | 1 | TTTTGCACTGCCTTGTACAC | AGG |
| 558 | 3598 | 1 | TTTGCACTGCCTTGTACACA | GGG |
| 559 | 3620 | -1 | ATTTCCTCCTAATATTCTAT | TGG |
| 560 | 3624 | 1 | TCATGATCCAATAGAATATT | AGG |
| 561 | 3627 | 1 | TGATCCAATAGAATATTAGG | AGG |
| 562 | 3654 | -1 | GGGCTTTTGATGTTTTATTG | GGG |
| 563 | 3655 | -1 | GGGGCTTTTGATGTTTTATT | GGG |
| 564 | 3656 | -1 | TGGGGCTTTTGATGTTTTAT | TGG |
| 565 | 3674 | -1 | TTCTGCTGATGGGGAGGATG | GGG |
| 566 | 3675 | -1 | TTTCTGCTGATGGGGAGGAT | GGG |
| 567 | 3676 | -1 | CTTTCTGCTGATGGGGAGGA | TGG |
| 568 | 3680 | -1 | CATCCTTTCTGCTGATGGGG | AGG |
| 569 | 3683 | -1 | TGACATCCTTTCTGCTGATG | GGG |
| 570 | 3684 | -1 | ATGACATCCTTTCTGCTGAT | GGG |
| 571 | 3685 | -1 | CATGACATCCTTTCTGCTGA | TGG |
| 572 | 3688 | 1 | CATCCTCCCCATCAGCAGAA | AGG |
| 573 | 3722 | -1 | GCAGTTTGAAAAGGTTGTTA | GGG |
| 574 | 3723 | -1 | TGCAGTTTGAAAAGGTTGTT | AGG |
| 575 | 3731 | -1 | TGCTGCTTTGCAGTTTGAAA | AGG |
| 576 | 3750 | 1 | AACTGCAAAGCAGCATGACC | TGG |
| 577 | 3757 | -1 | AAAACTTGGTATGGCATTCC | AGG |
| 578 | 3766 | -1 | GGTGCTTCAAAAACTTGGTA | TGG |
| 579 | 3771 | -1 | ACTGCGGTGCTTCAAAAACT | TGG |
| 580 | 3787 | -1 | AGTATTAATTATCATCACTG | CGG |
| 581 | 3828 | 1 | GAGTGAGAAAATTAATAGAG | TGG |
| 582 | 3829 | 1 | AGTGAGAAAATTAATAGAGT | GGG |
| 583 | 3830 | 1 | GTGAGAAAATTAATAGAGTG | GGG |
| 584 | 3870 | 1 | AAATTAGCATGAAAGTTTTA | TGG |
| 585 | 3890 | -1 | AGGTATATGTTATTCTTGAG | AGG |
| 586 | 3910 | -1 | TCGAGGATCATTATCTATAC | AGG |
| 587 | 3927 | -1 | CATGTTTGCTTGGCATGTCG | AGG |
| 588 | 3937 | -1 | TGCTATTTAGCATGTTTGCT | TGG |
| 589 | 3962 | -1 | ACTGATCCTATGCTCTATAT | TGG |
| 590 | 3967 | 1 | AGCATTCCAATATAGAGCAT | AGG |
| 591 | 3989 | -1 | AATGTTTATTTTTTTTCACA | GGG |
| 592 | 3990 | -1 | TAATGTTTATTTTTTTTCAC | AGG |
| 593 | 4068 | 1 | AGATAAGTTAGTATTGTTAC | AGG |
| 594 | 4119 | -1 | TTTTGTTTCAGAGACTGTCA | AGG |
| 595 | 4227 | -1 | CTAGATCTAAGGGTGTTTTT | TGG |
| 596 | 4237 | -1 | ACAAAATTGTCTAGATCTAA | GGG |
| 597 | 4238 | -1 | CACAAAATTGTCTAGATCTA | AGG |
| 598 | 4279 | 1 | ATTATAATTTACCAAAAACT | AGG |
| 599 | 4279 | -1 | GCACTATTTTTCCTAGTTTT | TGG |
| 600 | 4301 | -1 | AGTTCAGGCTCTTATGTTAT | AGG |
| 601 | 4316 | -1 | GGTCTTCCCCTATTTAGTTC | AGG |
| 602 | 4319 | 1 | CATAAGAGCCTGAACTAAAT | AGG |
| 603 | 4320 | 1 | ATAAGAGCCTGAACTAAATA | GGG |
| 604 | 4321 | 1 | TAAGAGCCTGAACTAAATAG | GGG |
| 605 | 4337 | -1 | TGTAGCTATGTTTTTGTTTT | TGG |
| 606 | 4386 | -1 | AAGGACAGAAAGAAGCTGTA | TGG |
| 607 | 4405 | -1 | TACGTTTTCTGTTCAATGCA | AGG |
| 608 | 4596 | 1 | TCATTATAATGATTACATTA | CGG |
| 609 | 4652 | -1 | GAAGGATAATAATTTTTTTG | AGG |
| 610 | 4670 | -1 | TATTTATATTGGTTAGTTGA | AGG |
| 611 | 4681 | -1 | ATGTATGTACGTATTTATAT | TGG |
| 612 | 4706 | -1 | TTCTGTCCGTAATCTTTACT | TGG |
| 613 | 4711 | 1 | ACATATCCAAGTAAAGATTA | CGG |
| 614 | 4751 | 1 | GATCACAAACAAGAATAAAA | AGG |
| 615 | 4783 | -1 | GAATAGCAAATGGAACTTGA | AGG |
| 616 | 4793 | -1 | ATCAGCTTGGGAATAGCAAA | TGG |
| 617 | 4805 | -1 | CTTCTCCCAGTGATCAGCTT | GGG |
| 618 | 4806 | -1 | GCTTCTCCCAGTGATCAGCT | TGG |
| 619 | 4810 | 1 | GCTATTCCCAAGCTGATCAC | TGG |
| 620 | 4811 | 1 | CTATTCCCAAGCTGATCACT | GGG |
| 621 | 4842 | -1 | GAGTATGCTTGTGATGTTGA | TGG |
| 622 | 4864 | 1 | ACAAGCATACTCCACCGTTT | CGG |
| 623 | 4864 | -1 | TTGTGGAAAGACCGAAACGG | TGG |
| 624 | 4867 | -1 | TGCTTGTGGAAAGACCGAAA | CGG |
| 625 | 4881 | -1 | TTTTGGCATGAGATTGCTTG | TGG |
| 626 | 4898 | -1 | CATATCTGGAAAAGGAATTT | TGG |
| 627 | 4906 | -1 | TCCTGCCTCATATCTGGAAA | AGG |
| 628 | 4912 | 1 | AAATTCCTTTTCCAGATATG | AGG |
| 629 | 4912 | -1 | TAGTCTTCCTGCCTCATATC | TGG |
| 630 | 4916 | 1 | TCCTTTTCCAGATATGAGGC | AGG |
| 631 | 4935 | -1 | TCTAGACGATATTATAGCTC | TGG |
| 632 | 4963 | -1 | TTTTGAGAAAATGGCAAACA | AGG |
| 633 | 4972 | -1 | ATTCCGTGATTTTGAGAAAA | TGG |
| 634 | 4980 | 1 | TTGCCATTTTCTCAAAATCA | CGG |
| 635 | 5014 | -1 | TCTCATTTTTTGAATATGGA | TGG |
| 636 | 5018 | -1 | TTTTTCTCATTTTTTGAATA | TGG |
| 637 | 5068 | -1 | AAGTGTGTATACTAAATAGA | AGG |
| 638 | 5092 | -1 | TTACATTTTTCATGAGCGGA | AGG |
| 639 | 5096 | -1 | AAAGTTACATTTTTCATGAG | CGG |
| 640 | 5130 | -1 | ACCTCTCCGTCTAGCTGAGT | GGG |
| 641 | 5131 | -1 | AACCTCTCCGTCTAGCTGAG | TGG |
| 642 | 5135 | 1 | CAGTGTCCCACTCAGCTAGA | CGG |
| 643 | 5140 | 1 | TCCCACTCAGCTAGACGGAG | AGG |
| 644 | 5150 | 1 | CTAGACGGAGAGGTTGCACT | CGG |
| 645 | 5151 | 1 | TAGACGGAGAGGTTGCACTC | GGG |
| 646 | 5170 | 1 | CGGGTTGTGAACTTAAATCC | CGG |
| 647 | 5177 | -1 | GTATTGATGAAGGAGCAACC | GGG |
| 648 | 5178 | -1 | TGTATTGATGAAGGAGCAAC | CGG |
| 649 | 5187 | -1 | AGCTGGGGTTGTATTGATGA | AGG |
| 650 | 5200 | 1 | TTCATCAATACAACCCCAGC | TGG |
| 651 | 5202 | -1 | GACTGCTTCTGTTCCAGCTG | GGG |
| 652 | 5203 | -1 | TGACTGCTTCTGTTCCAGCT | GGG |
| 653 | 5204 | -1 | GTGACTGCTTCTGTTCCAGC | TGG |
| 654 | 5218 | 1 | GCTGGAACAGAAGCAGTCAC | AGG |
| 655 | 5249 | -1 | TTTTTTTGCTTGATAATTTC | AGG |
| 656 | 5314 | 1 | GAATAGACAGCTGCAACTTA | AGG |
| 657 | 5326 | 1 | GCAACTTAAGGTTTATGTAT | TGG |
| 658 | 5340 | -1 | TCCCACTTTGTTATTATTAT | TGG |
| 659 | 5349 | 1 | AACCAATAATAATAACAAAG | TGG |
| 660 | 5350 | 1 | ACCAATAATAATAACAAAGT | GGG |
| 661 | 5395 | 1 | TGTGTGTGCGAAAAATAAAA | CGG |
| 662 | 5396 | 1 | GTGTGTGCGAAAAATAAAAC | GGG |
| 663 | 5436 | 1 | AAATAAAAATTAAAAGAAAA | AGG |
| 664 | 5471 | 1 | ACTATTTTTCTTTTTAAATC | AGG |
| 665 | 5482 | 1 | TTTTAAATCAGGATAGAGAA | AGG |
| 666 | 5500 | -1 | TTGTAGCTTGTTACGAAGCT | TGG |
| 667 | 5550 | -1 | AGTGTTCATATCTGTACTTC | AGG |
| 668 | 5574 | -1 | ATTTCTAAAGTTAGGCTAAG | AGG |
| 669 | 5582 | -1 | AGAGCTACATTTCTAAAGTT | AGG |
| 670 | 5618 | 1 | TTATAAAGATAAAGAAATTA | TGG |
| 671 | 5667 | -1 | AGAAATTAGGCTTCATCATA | TGG |
| 672 | 5680 | -1 | AACCTTTATTACAAGAAATT | AGG |
| 673 | 5689 | 1 | AGCCTAATTTCTTGTAATAA | AGG |
| 674 | 5708 | 1 | AAGGTTAAATTAATAATATA | AGG |
| 675 | 5713 | 1 | TAAATTAATAATATAAGGTG | TGG |
| 676 | 5725 | -1 | TGAGTATTTTATGATGAATG | TGG |
| 677 | 5753 | -1 | TATTTATAATATAGCAGTAG | TGG |
| 678 | 5817 | -1 | AGGCACCATCACATATCAGT | TGG |
| 679 | 5823 | 1 | AATGACCAACTGATATGTGA | TGG |
| 680 | 5832 | 1 | CTGATATGTGATGGTGCCTC | AGG |
| 681 | 5837 | -1 | TAGTTTTCCACATTGTCCTG | AGG |
| 682 | 5841 | 1 | GATGGTGCCTCAGGACAATG | TGG |
| 683 | 5850 | 1 | TCAGGACAATGTGGAAAACT | AGG |
| 684 | 5902 | 1 | TAGATAGCATTTGCTTTTTC | TGG |
| 685 | 6041 | -1 | GCTCCAGAAGCTTCTGGATA | TGG |
| 686 | 6047 | -1 | AAGATCGCTCCAGAAGCTTC | TGG |
| 687 | 6049 | 1 | ATACCATATCCAGAAGCTTC | TGG |
| 688 | 6076 | 1 | ATCTTCTGCAAATAAATCAG | AGG |
| 689 | 6077 | 1 | TCTTCTGCAAATAAATCAGA | GGG |
| 690 | 6090 | -1 | TCCAACAAAAGAGGAGTTTG | AGG |
| 691 | 6099 | -1 | TATATATTATCCAACAAAAG | AGG |
| 692 | 6100 | 1 | TCCTCAAACTCCTCTTTTGT | TGG |
| 693 | 6112 | 1 | TCTTTTGTTGGATAATATAT | AGG |
| 694 | 6121 | 1 | GGATAATATATAGGACACTC | AGG |
| 695 | 6145 | -1 | TTGACACAAGTGATCTGGAA | TGG |
| 696 | 6150 | -1 | TAAGTTTGACACAAGTGATC | TGG |
| 697 | 6174 | -1 | TGTTATTTCGAAGCGGAAGG | TGG |
| 698 | 6177 | -1 | GGATGTTATTTCGAAGCGGA | AGG |
| 699 | 6181 | -1 | GGAAGGATGTTATTTCGAAG | CGG |
| 700 | 6198 | -1 | AAATGGTCCTTTAAGTGGGA | AGG |
| 701 | 6202 | 1 | ATAACATCCTTCCCACTTAA | AGG |
| 702 | 6202 | -1 | GATCAAATGGTCCTTTAAGT | GGG |
| 703 | 6203 | -1 | GGATCAAATGGTCCTTTAAG | TGG |
| 704 | 6215 | -1 | GATGTTTTTTCTGGATCAAA | TGG |
| 705 | 6224 | -1 | GGCAATGCAGATGTTTTTTC | TGG |
| 706 | 6245 | -1 | TCTTGCGGTGTTAGATTACA | TGG |
| 707 | 6260 | -1 | TTAAGATCTTCAGCATCTTG | CGG |
| 708 | 6290 | -1 | AGTACAATGGCTCGAAGTGG | AGG |
| 709 | 6293 | -1 | AGCAGTACAATGGCTCGAAG | TGG |
| 710 | 6303 | -1 | AGAAACTATCAGCAGTACAA | TGG |
| 711 | 6327 | -1 | CAAAAGGCTTCAGCGTATGA | AGG |
| 712 | 6343 | -1 | TGGAGTTTCTGAAGCGCAAA | AGG |
| 713 | 6363 | -1 | AAAGGAGGTCAGAAATGGGT | TGG |
| 714 | 6367 | -1 | TATCAAAGGAGGTCAGAAAT | GGG |
| 715 | 6368 | -1 | TTATCAAAGGAGGTCAGAAA | TGG |
| 716 | 6378 | -1 | AAGGGTTTGTTTATCAAAGG | AGG |
| 717 | 6381 | -1 | GGGAAGGGTTTGTTTATCAA | AGG |
| 718 | 6396 | -1 | TGTTTGACAGGTGGAGGGAA | GGG |
| 719 | 6397 | -1 | TTGTTTGACAGGTGGAGGGA | AGG |
| 720 | 6401 | -1 | AAACTTGTTTGACAGGTGGA | GGG |
| 721 | 6402 | -1 | TAAACTTGTTTGACAGGTGG | AGG |
| 722 | 6405 | -1 | ATGTAAACTTGTTTGACAGG | TGG |
| 723 | 6408 | -1 | CCCATGTAAACTTGTTTGAC | AGG |
| 724 | 6418 | 1 | ACCTGTCAAACAAGTTTACA | TGG |
| 725 | 6419 | 1 | CCTGTCAAACAAGTTTACAT | GGG |

TABLE 4
gRNA (guide RNA) sequences and complementing PAMs
(protospacer adjacent motif) of CsMultiflora (referred to a
SEQ ID NOs: 729-935 in the seq.listing file).

| Seq# | Position | Strand | Sequence | PAM |
|---|---|---|---|---|
| 729 | 370 | 1 | ATAAGAAGTGATGTTGTGTC | TGG |
| 730 | 377 | 1 | GTGATGTTGTGTCTGGCTAG | AGG |
| 731 | 391 | -1 | GCTCTTGTTTTTGGTAGTAT | TGG |
| 732 | 400 | -1 | CGTTTTGTTGCTCTTGTTTT | TGG |
| 733 | 412 | 1 | CAAAAACAAGAGCAACAAAA | CGG |
| 734 | 437 | 1 | TTGCATCATTGTTTCTCACT | TGG |
| 735 | 441 | 1 | ATCATTGTTTCTCACTTGGC | TGG |
| 736 | 502 | 1 | AGAACTCAGTTCTCCAAAGC | TGG |
| 737 | 504 | -1 | TTGCTTATATGAACCAGCTT | TGG |
| 738 | 524 | 1 | GTTCATATAAGCAATCATTA | TGG |
| 739 | 544 | 1 | TGGCTTCATCAAACCGACAC | TGG |
| 740 | 546 | -1 | TGAACATACTAGGCCAGTGT | CGG |
| 741 | 556 | -1 | GGCTTGGACTTGAACATACT | AGG |
| 742 | 572 | -1 | GTGGTGGCTATTGCAAGGCT | TGG |
| 743 | 577 | -1 | CATTGGTGGTGGCTATTGCA | AGG |
| 744 | 588 | -1 | TGTCATGCTGCCATTGGTGG | TGG |
| 745 | 589 | 1 | CTTGCAATAGCCACCACCAA | TGG |
| 746 | 591 | -1 | TGATGTCATGCTGCCATTGG | TGG |
| 747 | 594 | -1 | GGTTGATGTCATGCTGCCAT | TGG |
| 748 | 615 | -1 | ATCCACTAGTCATGAGCGAT | GGG |
| 749 | 616 | -1 | TATCCACTAGTCATGAGCGA | TGG |
| 750 | 624 | 1 | AACCCATCGCTCATGACTAG | TGG |
| 751 | 639 | -1 | CTGAAGCGTACGGAGGTCTG | TGG |
| 752 | 646 | -1 | TATATACCTGAAGCGTACGG | AGG |
| 753 | 649 | -1 | ATATATATACCTGAAGCGTA | CGG |
| 754 | 651 | 1 | CACAGACCTCCGTACGCTTC | AGG |
| 755 | 716 | -1 | TTGACAACTTTTCAAAAAAT | AGG |
| 756 | 763 | -1 | GGAATATTGCATGTACTTCT | TGG |
| 757 | 784 | -1 | AATTATAAAATGCAACACAT | GGG |
| 758 | 785 | -1 | TAATTATAAAATGCAACACA | TGG |
| 759 | 811 | -1 | CCACCAACTGTAGTATAGAA | AGG |
| 760 | 819 | 1 | AAACCTTTCTATACTACAGT | TGG |
| 761 | 822 | 1 | CCTTTCTATACTACAGTTGG | TGG |
| 762 | 823 | 1 | CTTTCTATACTACAGTTGGT | GGG |
| 763 | 835 | 1 | CAGTTGGTGGGTGTGATGAG | AGG |
| 764 | 842 | 1 | TGGGTGTGATGAGAGGAGTC | CGG |
| 765 | 850 | -1 | TTCCATCTGGGCTTCGGCTC | CGG |
| 766 | 856 | -1 | TTCGGGTTCCATCTGGGCTT | CGG |
| 767 | 859 | 1 | GTCCGGAGCCGAAGCCCAGA | TGG |
| 768 | 862 | -1 | TCCGGTTTCGGGTTCCATCT | GGG |
| 769 | 863 | -1 | CTCCGGTTTCGGGTTCCATC | TGG |
| 770 | 872 | 1 | GCCCAGATGGAACCCGAAAC | CGG |
| 771 | 873 | -1 | TTCGAATCTGCTCCGGTTTC | GGG |
| 772 | 874 | -1 | ATTCGAATCTGCTCCGGTTT | CGG |
| 773 | 880 | -1 | TCAAGGATTCGAATCTGCTC | CGG |
| 774 | 897 | -1 | CAGAGTTAAAGATCGCTTCA | AGG |
| 775 | 909 | 1 | CTTGAAGCGATCTTTAACTC | TGG |
| 776 | 914 | 1 | AGCGATCTTTAACTCTGGCA | TGG |
| 777 | 931 | -1 | CTTCTAATCTCGTCTCTCGG | GGG |
| 778 | 932 | -1 | CCTTCTAATCTCGTCTCTCG | GGG |
| 779 | 933 | -1 | TCCTTCTAATCTCGTCTCTC | GGG |
| 780 | 934 | -1 | ATCCTTCTAATCTCGTCTCT | CGG |
| 781 | 943 | 1 | CCCCGAGAGACGAGATTAGA | AGG |
| 782 | 962 | -1 | TTGTCCATACTCTTGCAATT | GGG |
| 783 | 963 | -1 | CTTGTCCATACTCTTGCAAT | TGG |
| 784 | 969 | 1 | AGAGCCCAATTGCAAGAGTA | TGG |
| 785 | 978 | 1 | TTGCAAGAGTATGGACAAGT | TGG |
| 786 | 995 | -1 | TTGAAACCAATAAAACACAT | TGG |
| 787 | 1000 | 1 | GTGATGCCAATGTGTTTTAT | TGG |
| 788 | 1021 | 1 | GGTTTCAAAACAGAAAATCT | AGG |
| 789 | 1047 | -1 | GTTTGGAGTTTTGGAGGTGG | CGG |
| 790 | 1050 | -1 | GTTGTTTGGAGTTTTGGAGG | TGG |
| 791 | 1053 | -1 | TTTGTTGTTTGGAGTTTTGG | AGG |
| 792 | 1056 | -1 | GGGTTTGTTGTTTGGAGTTT | TGG |
| 793 | 1064 | -1 | ATTTTGAAGGGTTTGTTGTT | TGG |
| 794 | 1076 | -1 | AGTTGGAGTTGGATTTTGAA | GGG |
| 795 | 1077 | -1 | GAGTTGGAGTTGGATTTTGA | AGG |
| 796 | 1087 | -1 | GAGGAAGGAGGAGTTGGAGT | TGG |
| 797 | 1093 | -1 | GTGGCTGAGGAAGGAGGAGT | TGG |
| 798 | 1099 | -1 | GGAGCGGTGGCTGAGGAAGG | AGG |
| 799 | 1102 | -1 | GAAGGAGCGGTGGCTGAGGA | AGG |
| 800 | 1106 | -1 | AGAGGAAGGAGCGGTGGCTG | AGG |
| 801 | 1112 | -1 | AGACGAAGAGGAAGGAGCGG | TGG |
| 802 | 1115 | -1 | AGAAGACGAAGAGGAAGGAG | CGG |
| 803 | 1120 | -1 | GACGAAGAAGACGAAGAGGA | AGG |
| 804 | 1124 | -1 | AGACGACGAAGAAGACGAAG | AGG |
| 805 | 1159 | -1 | AAGCCTCTCGATCGGGATTT | GGG |
| 806 | 1160 | -1 | GAAGCCTCTCGATCGGGATT | TGG |
| 807 | 1166 | -1 | TGATGAGAAGCCTCTCGATC | GGG |
| 808 | 1167 | 1 | TCTCCCAAATCCCGATCGAG | AGG |
| 809 | 1167 | -1 | ATGATGAGAAGCCTCTCGAT | CGG |
| 810 | 1184 | 1 | GAGAGGCTTCTCATCATCAT | TGG |
| 811 | 1185 | 1 | AGAGGCTTCTCATCATCATT | GGG |
| 812 | 1206 | -1 | GATGATTAGTAGTACTGGCT | AGG |
| 813 | 1211 | -1 | ATGATGATGATTAGTAGTAC | TGG |
| 814 | 1242 | -1 | AAGAAGTTGGACTAGTTTGA | TGG |
| 815 | 1255 | -1 | TTATTATTGACCGAAGAAGT | TGG |
| 816 | 1256 | 1 | TCAAACTAGTCCAACTTCTT | CGG |
| 817 | 1283 | -1 | AATGTTGTTGGTTTGAAAGA | TGG |
| 818 | 1295 | -1 | AGTATTATTATTAATGTTGT | TGG |
| 819 | 1333 | -1 | AAGAAATAGTGTTGATCGGT | TGG |
| 820 | 1337 | -1 | CGGGAAGAAATAGTGTTGAT | CGG |
| 821 | 1349 | 1 | CGATCAACACTATTTCTTCC | CGG |
| 822 | 1356 | -1 | GGTGATGATTAGGCACGGCC | GGG |
| 823 | 1357 | -1 | GGGTGATGATTAGGCACGGC | CGG |
| 824 | 1361 | -1 | ACTGGGGTGATGATTAGGCA | CGG |
| 825 | 1366 | -1 | GTAGAACTGGGGTGATGATT | AGG |
| 826 | 1377 | -1 | CAGGAACAGGAGTAGAACTG | GGG |
| 827 | 1378 | -1 | GCAGGAACAGGAGTAGAACT | GGG |
| 828 | 1379 | -1 | AGCAGGAACAGGAGTAGAAC | TGG |
| 829 | 1390 | -1 | TGACTGGTATTAGCAGGAAC | AGG |
| 830 | 1396 | -1 | AACCCTTGACTGGTATTAGC | AGG |
| 831 | 1404 | 1 | GTTCCTGCTAATACCAGTCA | AGG |
| 832 | 1405 | 1 | TTCCTGCTAATACCAGTCAA | GGG |
| 833 | 1406 | -1 | AGGGAAGCAAAACCCTTGAC | TGG |
| 834 | 1425 | -1 | AGAGCTCATGATTTTGAGGA | GGG |
| 835 | 1426 | -1 | GAGAGCTCATGATTTTGAGG | AGG |
| 836 | 1429 | -1 | TGAGAGAGCTCATGATTTTG | AGG |
| 837 | 1443 | 1 | CAAAATCATGAGCTCTCTCA | TGG |
| 838 | 1444 | 1 | AAAATCATGAGCTCTCTCAT | GGG |
| 839 | 1445 | 1 | AAATCATGAGCTCTCTCATG | GGG |
| 840 | 1465 | -1 | TTCTGTATCTCCATGTGATG | AGG |
| 841 | 1466 | 1 | GGTAGTACTACCTCATCACA | TGG |
| 842 | 1482 | 1 | CACATGGAGATACAGAATAT | AGG |
| 843 | 1483 | 1 | ACATGGAGATACAGAATATA | GGG |
| 844 | 1495 | -1 | CTAAGTAAAAGGCTTGTACA | AGG |
| 845 | 1506 | -1 | TCATGATCTCACTAAGTAAA | AGG |
| 846 | 1527 | 1 | AGTGAGATCATGAACCAAAA | CGG |
| 847 | 1530 | -1 | CTTTCTTTAAGTCACCGTTT | TGG |
| 848 | 1553 | 1 | CTTAAAGAAAGACCATCACG | AGG |
| 849 | 1554 | -1 | GATAATTATTCACCTCGTGA | TGG |
| 850 | 1598 | 1 | GATGATGAAGATGAGCTCTA | CGG |
| 851 | 1651 | 1 | CTTGTCTGATGACGCCGACT | AGG |
| 852 | 1654 | -1 | GTAGTGGCCGGAGTCCTAGT | CGG |
| 853 | 1658 | 1 | GATGACGCCGACTAGGACTC | CGG |
| 854 | 1666 | -1 | ACGTTAGACGGAGTAGTGGC | CGG |
| 855 | 1670 | -1 | GACGACGTTAGACGGAGTAG | TGG |
| 856 | 1678 | -1 | GATGAAACGACGACGTTAGA | CGG |
| 857 | 1702 | -1 | GCAGTAGTAGCGTGGTGGTG | AGG |
| 858 | 1707 | -1 | TCGTTGCAGTAGTAGCGTGG | TGG |
| 859 | 1710 | -1 | TAGTCGTTGCAGTAGTAGCG | TGG |
| 860 | 1744 | -1 | ATTTGATTGAGGGGGACAGA | TGG |
| 861 | 1752 | -1 | TACCTTGGATTTGATTGAGG | GGG |
| 862 | 1753 | -1 | TTACCTTGGATTTGATTGAG | GGG |
| 863 | 1754 | -1 | TTTACCTTGGATTTGATTGA | GGG |
| 864 | 1755 | -1 | TTTTACCTTGGATTTGATTG | AGG |
| 865 | 1761 | 1 | GTCCCCCTCAATCAAATCCA | AGG |
| 866 | 1767 | -1 | CATTTTATTTTATTTTACCT | TGG |
| 867 | 1789 | 1 | TAAAATAAAATGTTACCACA | TGG |
| 868 | 1793 | -1 | TTTTCAGTTACAGTTCCATG | TGG |
| 869 | 1820 | 1 | CTGAAAATCGAAATTTTGTT | TGG |
| 870 | 1881 | 1 | GTTATATAAAAATTTTAACA | CGG |
| 871 | 1882 | 1 | TTATATAAAAATTTTAACAC | GGG |
| 872 | 1887 | 1 | TAAAAATTTTAACACGGGAG | AGG |
| 873 | 1934 | 1 | ATTTACAAATTATTATACAT | TGG |
| 874 | 1946 | 1 | TTATACATTGGTAAAAGATC | AGG |
| 875 | 1963 | 1 | ATCAGGACAATATTGTCTAA | TGG |
| 876 | 1964 | 1 | TCAGGACAATATTGTCTAAT | GGG |
| 877 | 1977 | -1 | TCCATCTACACTCAGATCGC | TGG |
| 878 | 1987 | 1 | TCCAGCGATCTGAGTGTAGA | TGG |
| 879 | 1999 | 1 | AGTGTAGATGGACGTGTGCA | TGG |
| 880 | 2005 | 1 | GATGGACGTGTGCATGGATT | TGG |
| 881 | 2008 | 1 | GGACGTGTGCATGGATTTGG | TGG |
| 882 | 2014 | 1 | GTGCATGGATTTGGTGGTGT | TGG |
| 883 | 2022 | 1 | ATTTGGTGGTGTTGGTCCTT | AGG |
| 884 | 2027 | -1 | AATATGAAACAGTCGACCTA | AGG |
| 885 | 2052 | 1 | TTTCATATTGATCTTTGTAT | TGG |
| 886 | 2053 | 1 | TTCATATTGATCTTTGTATT | GGG |
| 887 | 2118 | 1 | TTATTTATGACGAAAGTAAT | AGG |
| 888 | 2119 | 1 | TATTTATGACGAAAGTAATA | GGG |
| 889 | 2128 | 1 | CGAAAGTAATAGGGTACTAC | TGG |
| 890 | 2129 | 1 | GAAAGTAATAGGGTACTACT | GGG |
| 891 | 2154 | 1 | TATTTAATTAATAAATTATT | AGG |
| 892 | 2189 | -1 | AAAAAGCAAAGTTATAAAGG | AGG |
| 893 | 2192 | -1 | CAGAAAAAGCAAAGTTATAA | AGG |
| 894 | 2231 | -1 | TCTCCACGATCGATCGAATA | AGG |
| 895 | 2239 | 1 | ATACCTTATTCGATCGATCG | TGG |
| 896 | 2277 | 1 | TTACATATATTTATGAGAAC | AGG |
| 897 | 2286 | 1 | TTTATGAGAACAGGTGTTGT | AGG |
| 898 | 2300 | 1 | TGTTGTAGGAGAAGATGAAC | AGG |
| 899 | 2301 | 1 | GTTGTAGGAGAAGATGAACA | GGG |
| 900 | 2306 | 1 | AGGAGAAGATGAACAGGGAG | TGG |
| 901 | 2309 | 1 | AGAAGATGAACAGGGAGTGG | TGG |
| 902 | 2310 | 1 | GAAGATGAACAGGGAGTGGT | GGG |
| 903 | 2316 | 1 | GAACAGGGAGTGGTGGGTCC | AGG |
| 904 | 2319 | 1 | CAGGGAGTGGTGGGTCCAGG | TGG |
| 905 | 2323 | -1 | TTTGCCACACCATCTCCACC | TGG |
| 906 | 2325 | 1 | GTGGTGGGTCCAGGTGGAGA | TGG |
| 907 | 2330 | 1 | GGGTCCAGGTGGAGATGGTG | TGG |
| 908 | 2345 | 1 | TGGTGTGGCAAATAAAACGA | TGG |
| 909 | 2363 | 1 | GATGGTGTTCATAAACGACG | TGG |
| 910 | 2372 | 1 | CATAAACGACGTGGCCTTCG | AGG |
| 911 | 2375 | 1 | AAACGACGTGGCCTTCGAGG | TGG |
| 912 | 2375 | -1 | TGGCCCAGCGGCCACCTCGA | AGG |
| 913 | 2382 | 1 | GTGGCCTTCGAGGTGGCCGC | TGG |
| 914 | 2383 | 1 | TGGCCTTCGAGGTGGCCGCT | GGG |
| 915 | 2387 | -1 | GCGCACGTTGAATGGCCCAG | CGG |
| 916 | 2395 | -1 | AAAGCCTCGCGCACGTTGAA | TGG |
| 917 | 2402 | 1 | TGGGCCATTCAACGTGCGCG | AGG |
| 918 | 2409 | 1 | TTCAACGTGCGCGAGGCTTT | TGG |
| 919 | 2439 | 1 | GCTGTACTTATTCACTCCAA | TGG |
| 920 | 2444 | -1 | GGTGGGAACAAGTTGACCAT | TGG |
| 921 | 2461 | -1 | GTGACGCCCCAGTCGTCGGT | GGG |
| 922 | 2462 | -1 | AGTGACGCCCCAGTCGTCGG | TGG |
| 923 | 2464 | 1 | AACTTGTTCCCACCGACGAC | TGG |
| 924 | 2465 | 1 | ACTTGTTCCCACCGACGACT | GGG |
| 925 | 2465 | -1 | AGGAGTGACGCCCCAGTCGT | CGG |
| 926 | 2466 | 1 | CTTGTTCCCACCGACGACTG | GGG |
| 927 | 2485 | -1 | GCGCCGTGCTGGAGGGAGTG | AGG |
| 928 | 2492 | -1 | GTAAAAAGCGCCGTGCTGGA | GGG |
| 929 | 2493 | 1 | ACTCCTCACTCCCTCCAGCA | CGG |
| 930 | 2493 | -1 | AGTAAAAAGCGCCGTGCTGG | AGG |
| 931 | 2496 | -1 | GATAGTAAAAAGCGCCGTGC | TGG |
| 932 | 2515 | 1 | GCGCTTTTTACTATCTTATC | TGG |
| 933 | 2537 | -1 | ACGTAAATAATGGTGTGAAA | AGG |
| 934 | 2547 | -1 | AAAAAGATTTACGTAAATAA | TGG |
| 935 | 2579 | 1 | TTATTTGATTATAATATTTG | TGG |

TABLE 5
gRNA (guide RNA) sequences and complementing PAMs (protospacer
adjacent motif) of CsSFT1 (referred to as SEQ ID NOs: 939-1014
in the seq.listing file).

| Seq# | Position | Strand | Sequence | PAM |
|---|---|---|---|---|
| 939 | 1697 | -1 | TGTAGAGAGAGATCAGAAGA | GGG |
| 940 | 1698 | -1 | ATGTAGAGAGAGATCAGAAG | AGG |
| 941 | 1742 | 1 | ATAGTTGTTATATATATAAA | TGG |
| 942 | 1750 | 1 | TATATATATAAATGGCTAAT | AGG |
| 943 | 1751 | 1 | ATATATATAAATGGCTAATA | GGG |
| 944 | 1763 | 1 | GGCTAATAGGGATCCTCTTG | TGG |
| 945 | 1765 | -1 | ATCACTCTCCCAACCACAAG | AGG |
| 946 | 1767 | 1 | AATAGGGATCCTCTTGTGGT | TGG |
| 947 | 1768 | 1 | ATAGGGATCCTCTTGTGGTT | GGG |
| 948 | 1790 | 1 | GAGAGTGATAAGTGATGTGT | TGG |
| 949 | 1803 | -1 | GAGACACAGTTTTTGTGAAA | GGG |
| 950 | 1804 | -1 | AGAGACACAGTTTTTGTGAA | AGG |
| 951 | 1833 | 1 | TCTCTTAAAGTATCATATAG | TGG |
| 952 | 1834 | 1 | CTCTTAAAGTATCATATAGT | GGG |
| 953 | 1840 | 1 | AAGTATCATATAGTGGGAAT | AGG |
| 954 | 1841 | 1 | AGTATCATATAGTGGGAATA | GGG |
| 955 | 1854 | 1 | GGGAATAGGGCTATTAACAA | TGG |
| 956 | 1872 | -1 | TAGCAACTTGAGAAGGTCTA | AGG |
| 957 | 1879 | -1 | GGTGAGTTAGCAACTTGAGA | AGG |
| 958 | 1894 | 1 | CTCAAGTTGCTAACTCACCT | AGG |
| 959 | 1895 | 1 | TCAAGTTGCTAACTCACCTA | GGG |
| 960 | 1900 | -1 | TCTCCACCAATCTCAACCCT | AGG |
| 961 | 1905 | 1 | AACTCACCTAGGGTTGAGAT | TGG |
| 962 | 1908 | 1 | TCACCTAGGGTTGAGATTGG | TGG |
| 963 | 1921 | 1 | AGATTGGTGGAGATGATCTC | AGG |
| 964 | 1937 | 1 | TCTCAGGACTTTCTACACTT | TGG |
| 965 | 2020 | 1 | ATTAATACTGTTAATGTTGC | AGG |
| 966 | 2026 | 1 | ACTGTTAATGTTGCAGGTTA | TGG |
| 967 | 2043 | -1 | TCGCTAGGGTTAGGAGCATC | AGG |
| 968 | 2052 | -1 | AGATTAGGTTCGCTAGGGTT | AGG |
| 969 | 2057 | -1 | CTTTAAGATTAGGTTCGCTA | GGG |
| 970 | 2058 | -1 | TCTTTAAGATTAGGTTCGCT | AGG |
| 971 | 2067 | -1 | TGCAAATATTCTTTAAGATT | AGG |
| 972 | 2082 | 1 | ATCTTAAAGAATATTTGCAT | TGG |
| 973 | 2154 | 1 | AACTTTTAGATATATTACTT | AGG |
| 974 | 2164 | 1 | TATATTACTTAGGAATCACA | AGG |
| 975 | 2178 | -1 | TAGTGGCACACACTTATTGA | TGG |
| 976 | 2195 | -1 | TAAAAATTAATAATTATTAG | TGG |
| 977 | 2255 | 1 | TTAAAGTGTATAATTTTGTC | AGG |
| 978 | 2259 | 1 | AGTGTATAATTTTGTCAGGT | TGG |
| 979 | 2282 | -1 | AAGCCTGTTCCCGTAGTTGC | GGG |
| 980 | 2283 | 1 | GACTGATATTCCCGCAACTA | CGG |
| 981 | 2283 | -1 | AAAGCCTGTTCCCGTAGTTG | CGG |
| 982 | 2284 | 1 | ACTGATATTCCCGCAACTAC | GGG |
| 983 | 2290 | 1 | ATTCCCGCAACTACGGGAAC | AGG |
| 984 | 2296 | 1 | GCAACTACGGGAACAGGCTT | TGG |
| 985 | 2386 | -1 | TTAACTTACCATTAACTTAT | TGG |
| 986 | 2389 | 1 | AAATAACACCAATAAGTTAA | TGG |
| 987 | 2492 | 1 | TGTGTGTGATGATTTAATGA | TGG |
| 988 | 2493 | 1 | GTGTGTGATGATTTAATGAT | GGG |
| 989 | 2511 | 1 | ATGGGCGTACGCATATATGT | AGG |
| 990 | 2547 | -1 | GAATTCCCACCGTCGGTCTT | GGG |
| 991 | 2548 | -1 | TGAATTCCCACCGTCGGTCT | TGG |
| 992 | 2549 | 1 | CTACGAGAGCCCAAGACCGA | CGG |
| 993 | 2552 | 1 | CGAGAGCCCAAGACCGACGG | TGG |
| 994 | 2553 | 1 | GAGAGCCCAAGACCGACGGT | GGG |
| 995 | 2554 | -1 | AAGCGATGAATTCCCACCGT | CGG |
| 996 | 2592 | 1 | TTTGTTCTGTTTCGACAGTT | AGG |
| 997 | 2596 | 1 | TTCTGTTTCGACAGTTAGGA | AGG |
| 998 | 2616 | 1 | AGGCAGACAGTGTATGCACC | CGG |
| 999 | 2617 | 1 | GGCAGACAGTGTATGCACCC | GGG |
| 1000 | 2620 | 1 | AGACAGTGTATGCACCCGGG | TGG |
| 1001 | 2623 | -1 | TTGAAGTTATGTCGCCACCC | GGG |
| 1002 | 2624 | -1 | GTTGAAGTTATGTCGCCACC | CGG |
| 1003 | 2667 | 1 | TTTGCTGAAATCTACAACCT | TGG |
| 1004 | 2673 | -1 | CGGCAGCAACAGGCAATCCA | AGG |
| 1005 | 2683 | -1 | TTGAAGTAAACGGCAGCAAC | AGG |
| 1006 | 2693 | -1 | CTTTTGGCAGTTGAAGTAAA | CGG |
| 1007 | 2705 | 1 | CGTTTACTTCAACTGCCAAA | AGG |
| 1008 | 2709 | -1 | CACCACAGCCAAGTTCCTTT | TGG |
| 1009 | 2712 | 1 | TTCAACTGCCAAAAGGAACT | TGG |
| 1010 | 2718 | 1 | TGCCAAAAGGAACTTGGCTG | TGG |
| 1011 | 2721 | 1 | CAAAAGGAACTTGGCTGTGG | TGG |
| 1012 | 2725 | 1 | AGGAACTTGGCTGTGGTGGA | AGG |
| 1013 | 2728 | 1 | AACTTGGCTGTGGTGGAAGG | AGG |
| 1014 | 2798 | -1 | GACATATATAGATAGATAGA | TGG |

TABLE 6
gRNA (guide RNA) sequences and complementing PAMs (protospacer
adjacent motif) of CsSFT2 (referred to as SEQ ID NOs: 1018-1105
in the seq.listing file).

| Seq# | Position | Strand | Sequence | PAM |
|---|---|---|---|---|
| 1018 | 910 | 1 | CAATATAATAATATGACATA | TGG |
| 1019 | 926 | 1 | CATATGGATAGATAGATAGA | TGG |
| 1020 | 986 | -1 | AACTTGGCTGTGGTGGAAGG | AGG |
| 1021 | 989 | -1 | AGGAACTTGGCTGTGGTGGA | AGG |
| 1022 | 993 | -1 | CAAAAGGAACTTGGCTGTGG | TGG |
| 1023 | 996 | -1 | TGCCAAAAGGAACTTGGCTG | TGG |
| 1024 | 1002 | -1 | TTCAACTGCCAAAAGGAACT | TGG |
| 1025 | 1005 | 1 | CACCACAGCCAAGTTCCTTT | TGG |
| 1026 | 1009 | -1 | CGTTTACTTCAACTGCCAAA | AGG |
| 1027 | 1031 | 1 | TTGAAGTAAACGACAGCAAC | AGG |
| 1028 | 1041 | 1 | CGACAGCAACAGGCAATCCA | AGG |
| 1029 | 1047 | -1 | TTTGCTGAGATCTACAACCT | TGG |
| 1030 | 1091 | 1 | TTGAAGTTATGTCGCCACCC | AGG |
| 1031 | 1094 | -1 | AGACAGTGTATGCACCTGGG | TGG |
| 1032 | 1097 | -1 | GGCAGACAGTGTATGCACCT | GGG |
| 1033 | 1098 | -1 | AGGCAGACAGTGTATGCACC | TGG |
| 1034 | 1118 | -1 | TTCTGTTTCGACAGTTAGGA | AGG |
| 1035 | 1122 | -1 | TTTGTTCTGTTTCGACAGTT | AGG |
| 1036 | 1160 | 1 | TAGCGATGTATTCCCACCGT | CGG |
| 1037 | 1161 | -1 | GAGAGCCCTAGACCGACGGT | GGG |
| 1038 | 1162 | -1 | CGAGAGCCCTAGACCGACGG | TGG |
| 1039 | 1165 | -1 | CTACGAGAGCCCTAGACCGA | CGG |
| 1040 | 1166 | 1 | TGTATTCCCACCGTCGGTCT | AGG |
| 1041 | 1167 | 1 | GTATTCCCACCGTCGGTCTA | GGG |
| 1042 | 1203 | -1 | GTGTATATACGCATATATGT | AGG |
| 1043 | 1346 | 1 | TTAACTTATCGTTAACTTAT | TGG |
| 1044 | 1431 | 1 | AGTGTATTAATATGTACCAA | AGG |
| 1045 | 1436 | -1 | GCAACTACGGGAACAGCCTT | TGG |
| 1046 | 1448 | -1 | ACTGATATTCCCGCAACTAC | GGG |
| 1047 | 1449 | 1 | AAAGGCTGTTCCCGTAGTTG | CGG |
| 1048 | 1449 | -1 | GACTGATATTCCCGCAACTA | CGG |
| 1049 | 1450 | 1 | AAGGCTGTTCCCGTAGTTGC | GGG |
| 1050 | 1473 | -1 | AGTGTATAACTTTTTCAGGT | TGG |
| 1051 | 1477 | -1 | TTTAAGTGTATAACTTTTTC | AGG |
| 1052 | 1532 | 1 | ATAAAATTAATAATTATTAG | TGG |
| 1053 | 1548 | 1 | TTAGTGGCACACACTATTGA | TGG |
| 1054 | 1562 | -1 | AATATTACTAACGAATGACA | AGG |
| 1055 | 1612 | -1 | TGTACTTAGTAATATCATTT | CGG |
| 1056 | 1637 | -1 | ATCTTAAAGAATATTTGCAT | TGG |
| 1057 | 1652 | 1 | TGCAAATATTCTTTAAGATT | AGG |
| 1058 | 1661 | 1 | TCTTTAAGATTAGGTTCGCT | AGG |
| 1059 | 1662 | 1 | CTTTAAGATTAGGTTCGCTA | GGG |
| 1060 | 1667 | 1 | AGATTAGGTTCGCTAGGGTT | AGG |
| 1061 | 1692 | -1 | ACTGTTAATGTTGCAGGTTA | TGG |
| 1062 | 1698 | -1 | ATTAATACTGTTAATGTTGC | AGG |
| 1063 | 1818 | 1 | CGTATACTTAATTTTATGCT | AGG |
| 1064 | 1959 | 1 | TATTTGTGCAATTATAAGTT | AGG |
| 1065 | 1971 | -1 | GTCAAAGACCCACAACAATA | AGG |
| 1066 | 1973 | 1 | TAAGTTAGGCCTTATTGTTG | TGG |
| 1067 | 1974 | 1 | AAGTTAGGCCTTATTGTTGT | GGG |
| 1068 | 2030 | 1 | TTAACAGTGTTAATTTTAAA | CGG |
| 1069 | 2175 | -1 | TTGGTTTATGAAAAATTAGT | GGG |
| 1070 | 2176 | -1 | CTTGGTTTATGAAAAATTAG | TGG |
| 1071 | 2194 | -1 | TGCCTATATAACTAAATTCT | TGG |
| 1072 | 2203 | 1 | AACCAAGAATTTAGTTATAT | AGG |
| 1073 | 2253 | -1 | TCTCAGGACTTTCTACACTT | TGG |
| 1074 | 2269 | -1 | AGATTGGTGGAGATGATCTC | AGG |
| 1075 | 2282 | -1 | TCACCTAGGGTTGAGATTGG | TGG |
| 1076 | 2285 | -1 | AACTCACCTAGGGTTGAGAT | TGG |
| 1077 | 2290 | 1 | TCTCCACCAATCTCAACCCT | AGG |
| 1078 | 2295 | -1 | TCAAGTTGCTAACTCACCTA | GGG |
| 1079 | 2296 | -1 | CTCAAGTTGCTAACTCACCT | AGG |
| 1080 | 2311 | 1 | GGTGAGTTAGCAACTTGAGA | AGG |
| 1081 | 2318 | 1 | TAGCAACTTGAGAAGGTCTA | AGG |
| 1082 | 2336 | -1 | GGGATTAGGGCTATTAACAA | TGG |
| 1083 | 2349 | -1 | AGTATCATATAGTGGGATTA | GGG |
| 1084 | 2350 | -1 | AAGTATCATATAGTGGGATT | AGG |
| 1085 | 2356 | -1 | CTCTTAAAGTATCATATAGT | GGG |
| 1086 | 2357 | -1 | TCTCTTAAAGTATCATATAG | TGG |
| 1087 | 2386 | 1 | AGAGAGACAGTTTTTGTGAA | AGG |
| 1088 | 2387 | 1 | GAGAGACAGTTTTTGTGAAA | GGG |
| 1089 | 2400 | -1 | GAGAGTGATAAGTGATGTGT | TGG |
| 1090 | 2422 | -1 | ATAGGGATCCTCTTGTGGTT | GGG |
| 1091 | 2423 | -1 | AATAGGGATCCTCTTGTGGT | TGG |
| 1092 | 2425 | 1 | ATCACTCTCCCAACCACAAG | AGG |
| 1093 | 2427 | -1 | GGCTAATAGGGATCCTCTTG | TGG |
| 1094 | 2439 | -1 | ATATATATAAATGGCTAATA | GGG |
| 1095 | 2440 | -1 | TATATATATAAATGGCTAAT | AGG |
| 1096 | 2448 | -1 | ATAGTTGTTATATATATAAA | TGG |
| 1097 | 2499 | 1 | AGAGAGATAGAGAGAAGAA | AGG |
| | | | G | |
| 1098 | 2500 | 1 | GAGAGATAGAGAGAAGAAG | GGG |
| | | | A | |
| 1099 | 2516 | 1 | AAGAGGGTTTGATGAGTTTT | TGG |
| 1100 | 2529 | 1 | GAGTTTTTGGTTGTATAATT | TGG |
| 1101 | 2532 | 1 | TTTTTGGTTGTATAATTTGG | TGG |
| 1102 | 2553 | 1 | GGCTGACATTCAACAATTTA | TGG |
| 1103 | 2609 | 1 | TTAGCTTTTGTAACATCAAA | AGG |
| 1104 | 2627 | 1 | AAAGGTTCTAATATATATTG | TGG |
| 1105 | 2684 | 1 | TTTATATATTCTTGTAACAA | TGG |

TABLE 7
gRNA (guide RNA) sequences and complementing PAMs (protospacer
adjacent motif) of CsSFT3 (referred to as SEQ ID NOs: 1109-1334
in the seq.listing file).

| Seq# | Position | strand | Sequence | PAM |
|---|---|---|---|---|
| 1109 | 364 | -1 | TTACTCTACCAACCACAAGA | GGG |
| 1110 | 365 | -1 | ATTACTCTACCAACCACAAG | AGG |
| 1111 | 367 | 1 | GATAGGGACCCTCTTGTGGT | TGG |
| 1112 | 379 | 1 | CTTGTGGTTGGTAGAGTAAT | AGG |
| 1113 | 390 | 1 | TAGAGTAATAGGAGATGTTT | TGG |
| 1114 | 404 | 1 | ATGTTTTGGATCCTTTTACA | AGG |
| 1115 | 404 | -1 | AGAGAGACTGACCTTGTAAA | AGG |
| 1116 | 430 | 1 | GTCTCTCTTAGAGTGAGTTA | TGG |
| 1117 | 441 | 1 | AGTGAGTTATGGTAATAGAG | AGG |
| 1118 | 451 | 1 | GGTAATAGAGAGGTCAACAA | TGG |
| 1119 | 476 | -1 | GGTTGGTTAACAATTTGGGA | AGG |
| 1120 | 480 | -1 | ACGAGGTTGGTTAACAATTT | GGG |
| 1121 | 481 | -1 | CACGAGGTTGGTTAACAATT | TGG |
| 1122 | 493 | -1 | CACCAATATCAACACGAGGT | TGG |
| 1123 | 497 | -1 | TCACCACCAATATCAACACG | AGG |
| 1124 | 502 | 1 | AACCAACCTCGTGTTGATAT | TGG |
| 1125 | 505 | 1 | CAACCTCGTGTTGATATTGG | TGG |
| 1126 | 518 | 1 | ATATTGGTGGTGATGACCTA | AGG |
| 1127 | 523 | -1 | CCAAAGTGTAGAAGGTCCTT | AGG |
| 1128 | 531 | -1 | TTAATTTACCAAAGTGTAGA | AGG |
| 1129 | 534 | 1 | CCTAAGGACCTTCTACACTT | TGG |
| 1130 | 569 | -1 | TGAAATCATTATGAATATTG | AGG |
| 1131 | 648 | 1 | CATATATTGAAAATTATTAC | AGG |
| 1132 | 654 | 1 | TTGAAAATTATTACAGGTCA | TGG |
| 1133 | 657 | 1 | AAAATTATTACAGGTCATGG | TGG |
| 1134 | 663 | 1 | ATTACAGGTCATGGTGGATC | CGG |
| 1135 | 671 | -1 | TTGCTAGGGCTAGGAGCATC | CGG |
| 1136 | 680 | -1 | AGATTGGGGTTGCTAGGGCT | AGG |
| 1137 | 685 | -1 | CCCTTAGATTGGGGTTGCTA | GGG |
| 1138 | 686 | -1 | TCCCTTAGATTGGGGTTGCT | AGG |
| 1139 | 694 | -1 | GCAAATACTCCCTTAGATTG | GGG |
| 1140 | 695 | 1 | GCCCTAGCAACCCCAATCTA | AGG |
| 1141 | 695 | -1 | TGCAAATACTCCCTTAGATT | GGG |
| 1142 | 696 | 1 | CCCTAGCAACCCCAATCTAA | GGG |
| 1143 | 696 | -1 | ATGCAAATACTCCCTTAGAT | TGG |
| 1144 | 710 | 1 | ATCTAAGGGAGTATTTGCAT | TGG |
| 1145 | 768 | 1 | ATATTATTATTAAATAGATG | AGG |
| 1146 | 769 | 1 | TATTATTATTAAATAGATGA | GGG |
| 1147 | 901 | 1 | TTTAATTTTGTATAAAACTT | TGG |
| 1148 | 1020 | -1 | TTCATGCACACAACACATGT | TGG |
| 1149 | 1081 | -1 | CAAAAAGTAAAGACATATTT | TGG |
| 1150 | 1093 | 1 | CAAAATATGTCTTTACTTTT | TGG |
| 1151 | 1128 | 1 | CATTTTATAAAGATGTTAGT | TGG |
| 1152 | 1129 | 1 | ATTTTATAAAGATGTTAGTT | GGG |
| 1153 | 1317 | 1 | TTTTAGTGTCAGTTTTGAAT | TGG |
| 1154 | 1335 | -1 | ACAAAATTCTGTAATTATTA | GGG |
| 1155 | 1336 | -1 | TACAAAATTCTGTAATTATT | AGG |
| 1156 | 1368 | -1 | ACAAATTAAAACAAGCTTTA | GGG |
| 1157 | 1369 | -1 | AACAAATTAAAACAAGCTTT | AGG |
| 1158 | 1392 | 1 | TTTAATTTGTTAAAGTGACT | AGG |
| 1159 | 1440 | -1 | AACATGTAAAAAGAATTTAA | GGG |
| 1160 | 1441 | -1 | AAACATGTAAAAAGAATTTA | AGG |
| 1161 | 1470 | -1 | GCTAGCATATATGGAATTTG | TGG |
| 1162 | 1479 | -1 | ATTTATATAGCTAGCATATA | TGG |
| 1163 | 1500 | 1 | TAGCTATATAAATATAAATA | TGG |
| 1164 | 1505 | 1 | ATATAAATATAAATATGGAA | AGG |
| 1165 | 1513 | 1 | ATAAATATGGAAAGGATATA | TGG |
| 1166 | 1514 | 1 | TAAATATGGAAAGGATATAT | GGG |
| 1167 | 1563 | 1 | AAAGCTGATGAGAAAGAATG | TGG |
| 1168 | 1568 | 1 | TGATGAGAAAGAATGTGGTT | TGG |
| 1169 | 1569 | 1 | GATGAGAAAGAATGTGGTTT | GGG |
| 1170 | 1570 | 1 | ATGAGAAAGAATGTGGTTTG | GGG |
| 1171 | 1591 | 1 | GGATGAATTTTGAATGATGA | AGG |
| 1172 | 1592 | 1 | GATGAATTTTGAATGATGAA | GGG |
| 1173 | 1598 | 1 | TTTTGAATGATGAAGGGATG | AGG |
| 1174 | 1610 | 1 | AAGGGATGAGGCTGTGTGTG | TGG |
| 1175 | 1633 | -1 | GGGACATGCTATAGCTAGCA | GGG |
| 1176 | 1634 | -1 | GGGGACATGCTATAGCTAGC | AGG |
| 1177 | 1653 | -1 | TTTTAATGGTGGGACAAAAG | GGG |
| 1178 | 1654 | -1 | ATTTTAATGGTGGGACAAAA | GGG |
| 1179 | 1655 | -1 | CATTTTAATGGTGGGACAAA | AGG |
| 1180 | 1663 | -1 | GAGGTGGCCATTTTAATGGT | GGG |
| 1181 | 1664 | -1 | TGAGGTGGCCATTTTAATGG | TGG |
| 1182 | 1667 | 1 | CTTTTGTCCCACCATTAAAA | TGG |
| 1183 | 1667 | -1 | GTGTGAGGTGGCCATTTTAA | TGG |
| 1184 | 1679 | -1 | AAAACCTTCTTAGTGTGAGG | TGG |
| 1185 | 1682 | -1 | GTGAAAACCTTCTTAGTGTG | AGG |
| 1186 | 1686 | 1 | ATGGCCACCTCACACTAAGA | AGG |
| 1187 | 1755 | 1 | ATATATACATACACATGTAT | AGG |
| 1188 | 1756 | 1 | TATATACATACACATGTATA | GGG |
| 1189 | 1824 | -1 | CTATGTTCGAATTCAAATTC | GGG |
| 1190 | 1825 | -1 | TCTATGTTCGAATTCAAATT | CGG |
| 1191 | 1847 | 1 | TTCGAACATAGACTCAGATT | TGG |
| 1192 | 1860 | -1 | AGGGTTCAGGGTCGAATTTA | GGG |
| 1193 | 1861 | -1 | CAGGGTTCAGGGTCGAATTT | AGG |
| 1194 | 1872 | -1 | AGTTCGTGTTTCAGGGTTCA | GGG |
| 1195 | 1873 | -1 | AAGTTCGTGTTTCAGGGTTC | AGG |
| 1196 | 1879 | -1 | GAGTCTAAGTTCGTGTTTCA | GGG |
| 1197 | 1880 | -1 | TGAGTCTAAGTTCGTGTTTC | AGG |
| 1198 | 1898 | 1 | CACGAACTTAGACTCAGACC | TGG |
| 1199 | 1904 | 1 | CTTAGACTCAGACCTGGACC | TGG |
| 1200 | 1905 | -1 | TTGGGTCAAGGTCCAGGTCC | AGG |
| 1201 | 1911 | -1 | CGGGGTTTGGGTCAAGGTCC | AGG |
| 1202 | 1917 | -1 | TCGGGTCGGGGTTTGGGTCA | AGG |
| 1203 | 1923 | -1 | CGGGGTTCGGGTCGGGGTTT | GGG |
| 1204 | 1924 | -1 | TCGGGGTTCGGGTCGGGGTT | TGG |
| 1205 | 1929 | -1 | TCGAGTCGGGGTTCGGGTCG | GGG |
| 1206 | 1930 | -1 | TTCGAGTCGGGGTTCGGGTC | GGG |
| 1207 | 1931 | -1 | GTTCGAGTCGGGGTTCGGGT | CGG |
| 1208 | 1935 | -1 | CGGGGTTCGAGTCGGGGTTC | GGG |
| 1209 | 1936 | -1 | TCGGGGTTCGAGTCGGGGTT | CGG |
| 1210 | 1941 | -1 | TAGTTTCGGGGTTCGAGTCG | GGG |
| 1211 | 1942 | -1 | CTAGTTTCGGGGTTCGAGTC | GGG |
| 1212 | 1943 | -1 | TCTAGTTTCGGGGTTCGAGT | CGG |
| 1213 | 1953 | -1 | CCAGGTCCAGTCTAGTTTCG | GGG |
| 1214 | 1954 | -1 | TCCAGGTCCAGTCTAGTTTC | GGG |
| 1215 | 1955 | -1 | GTCCAGGTCCAGTCTAGTTT | CGG |
| 1216 | 1958 | 1 | CTCGAACCCCGAAACTAGAC | TGG |
| 1217 | 1964 | 1 | CCCCGAAACTAGACTGGACC | TGG |
| 1218 | 1971 | 1 | ACTAGACTGGACCTGGACTC | TGG |
| 1219 | 1971 | -1 | TAGGTCCTAGGCCAGAGTCC | AGG |
| 1220 | 1977 | 1 | CTGGACCTGGACTCTGGCCT | AGG |
| 1221 | 1983 | -1 | CTAGACCCGAGCTAGGTCCT | AGG |
| 1222 | 1988 | 1 | CTCTGGCCTAGGACCTAGCT | CGG |
| 1223 | 1989 | 1 | TCTGGCCTAGGACCTAGCTC | GGG |
| 1224 | 1990 | -1 | CTGAACTCTAGACCCGAGCT | AGG |
| 1225 | 2002 | 1 | CTAGCTCGGGTCTAGAGTTC | AGG |
| 1226 | 2012 | 1 | TCTAGAGTTCAGGTCCAGTC | CGG |
| 1227 | 2013 | 1 | CTAGAGTTCAGGTCCAGTCC | GGG |
| 1228 | 2014 | 1 | TAGAGTTCAGGTCCAGTCCG | GGG |
| 1229 | 2015 | -1 | CCTGACCTCGGACCCCGGAC | TGG |
| 1230 | 2020 | -1 | CAGATCCTGACCTCGGACCC | CGG |
| 1231 | 2021 | 1 | CAGGTCCAGTCCGGGGTCCG | AGG |
| 1232 | 2026 | 1 | CCAGTCCGGGGTCCGAGGTC | AGG |
| 1233 | 2027 | -1 | ACGAACCCAGATCCTGACCT | CGG |
| 1234 | 2032 | 1 | CGGGGTCCGAGGTCAGGATC | TGG |
| 1235 | 2033 | 1 | GGGGTCCGAGGTCAGGATCT | GGG |
| 1236 | 2045 | 1 | CAGGATCTGGGTTCGTGTTC | TGG |
| 1237 | 2046 | 1 | AGGATCTGGGTTCGTGTTCT | GGG |
| 1238 | 2047 | 1 | GGATCTGGGTTCGTGTTCTG | GGG |
| 1239 | 2053 | 1 | GGGTTCGTGTTCTGGGGTTC | AGG |
| 1240 | 2057 | 1 | TCGTGTTCTGGGGTTCAGGT | TGG |
| 1241 | 2058 | 1 | CGTGTTCTGGGGTTCAGGTT | GGG |
| 1242 | 2062 | 1 | TTCTGGGGTTCAGGTTGGGT | TGG |
| 1243 | 2063 | 1 | TCTGGGGTTCAGGTTGGGTT | GGG |
| 1244 | 2068 | 1 | GGTTCAGGTTGGGTTGGGTC | TGG |
| 1245 | 2075 | 1 | GTTGGGTTGGGTCTGGAGTC | TGG |
| 1246 | 2076 | 1 | TTGGGTTGGGTCTGGAGTCT | GGG |
| 1247 | 2082 | 1 | TGGGTCTGGAGTCTGGGTCT | AGG |
| 1248 | 2083 | 1 | GGGTCTGGAGTCTGGGTCTA | GGG |
| 1249 | 2095 | 1 | TGGGTCTAGGGTCCAGATTC | AGG |
| 1250 | 2096 | -1 | CCTGAACCCGATCCTGAATC | TGG |
| 1251 | 2100 | 1 | CTAGGGTCCAGATTCAGGAT | CGG |
| 1252 | 2101 | 1 | TAGGGTCCAGATTCAGGATC | GGG |
| 1253 | 2107 | 1 | CCAGATTCAGGATCGGGTTC | AGG |
| 1254 | 2113 | 1 | TCAGGATCGGGTTCAGGTTA | AGG |
| 1255 | 2131 | 1 | TAAGGTTTGAGTCTGAGTCC | AGG |
| 1256 | 2137 | 1 | TTGAGTCTGAGTCCAGGTAT | AGG |
| 1257 | 2138 | -1 | TCCCGACCAGAACCTATACC | TGG |
| 1258 | 2143 | 1 | CTGAGTCCAGGTATAGGTTC | TGG |
| 1259 | 2147 | 1 | GTCCAGGTATAGGTTCTGGT | CGG |
| 1260 | 2148 | 1 | TCCAGGTATAGGTTCTGGTC | GGG |
| 1261 | 2176 | 1 | AGTTCGAGAGTTTGAATTCA | AGG |
| 1262 | 2186 | 1 | TTTGAATTCAAGGTCCAATT | TGG |
| 1263 | 2189 | -1 | GAACTCATCCAACTCCAAAT | TGG |
| 1264 | 2192 | 1 | TTCAAGGTCCAATTTGGAGT | TGG |
| 1265 | 2209 | 1 | AGTTGGATGAGTTCATGTCA | TGG |
| 1266 | 2275 | -1 | TTTAAAATTTTAATAGTGTT | TGG |
| 1267 | 2391 | 1 | ATTCATAATTTTTAAATTAG | AGG |
| 1268 | 2392 | 1 | TTCATAATTTTTAAATTAGA | GGG |
| 1269 | 2408 | -1 | TTATTTTTATCTTACTTATA | GGG |
| 1270 | 2409 | -1 | ATTATTTTTATCTTACTTAT | AGG |
| 1271 | 2520 | -1 | TTTACTGTACCGAATATTCA | CGG |
| 1272 | 2522 | 1 | TTGTAGTTACCGTGAATATT | CGG |
| 1273 | 2537 | 1 | ATATTCGGTACAGTAAATTA | AGG |
| 1274 | 2541 | 1 | TCGGTACAGTAAATTAAGGA | TGG |
| 1275 | 2622 | -1 | ATATATAAAAATATAAATTG | TGG |
| 1276 | 2653 | 1 | TATATTATTAATCTAGATAA | TGG |
| 1277 | 2705 | -1 | ATAATTATACTATATATTAT | AGG |
| 1278 | 2735 | 1 | TTATAATAATTATACATGTT | TGG |
| 1279 | 2750 | 1 | ATGTTTGGCAATTTCAATTT | AGG |
| 1280 | 2754 | 1 | TTGGCAATTTCAATTTAGGT | TGG |
| 1281 | 2770 | 1 | AGGTTGGTGACTGATATTCC | TGG |
| 1282 | 2777 | -1 | AAGCTTGGCCCGGTAGTTCC | AGG |
| 1283 | 2779 | 1 | ACTGATATTCCTGGAACTAC | CGG |
| 1284 | 2780 | 1 | CTGATATTCCTGGAACTACC | GGG |
| 1285 | 2787 | -1 | CGGCTCACCGAAGCTTGGCC | CGG |
| 1286 | 2791 | 1 | GGAACTACCGGGCCAAGCTT | CGG |
| 1287 | 2792 | -1 | ATGAACGGCTCACCGAAGCT | TGG |
| 1288 | 2807 | -1 | GTATTATTATGAAGTATGAA | CGG |
| 1289 | 2878 | 1 | ACGCTGTAAACAAAATAGTG | CGG |
| 1290 | 2980 | 1 | ATTAATTGTTTATTATGTGT | AGG |
| 1291 | 2988 | 1 | TTTATTATGTGTAGGACAAG | AGG |
| 1292 | 2991 | 1 | ATTATGTGTAGGACAAGAGG | TGG |
| 1293 | 3011 | 1 | TGGTGTGCTACGAGAACCCG | CGG |
| 1294 | 3016 | -1 | GAATCCCCACCGTCGGCCGC | GGG |
| 1295 | 3017 | -1 | TGAATCCCCACCGTCGGCCG | CGG |
| 1296 | 3018 | 1 | CTACGAGAACCCGCGGCCGA | CGG |
| 1297 | 3021 | 1 | CGAGAACCCGCGGCCGACGG | TGG |
| 1298 | 3022 | 1 | GAGAACCCGCGGCCGACGGT | GGG |
| 1299 | 3023 | 1 | AGAACCCGCGGCCGACGGTG | GGG |
| 1300 | 3023 | -1 | TACCGATGAATCCCCACCGT | CGG |
| 1301 | 3032 | 1 | GGCCGACGGTGGGGATTCAT | CGG |
| 1302 | 3053 | 1 | GGTATGTATTTGTGTTGTTC | CGG |
| 1303 | 3060 | 1 | ATTTGTGTTGTTCCGGCAAT | TGG |
| 1304 | 3061 | 1 | TTTGTGTTGTTCCGGCAATT | GGG |
| 1305 | 3061 | -1 | CCGTTTGCCTTCCCAATTGC | CGG |
| 1306 | 3065 | 1 | TGTTGTTCCGGCAATTGGGA | AGG |
| 1307 | 3072 | 1 | CCGGCAATTGGGAAGGCAAA | CGG |
| 1308 | 3084 | 1 | AAGGCAAACGGTGTTCGCGC | CGG |
| 1309 | 3085 | 1 | AGGCAAACGGTGTTCGCGCC | GGG |
| 1310 | 3086 | 1 | GGCAAACGGTGTTCGCGCCG | GGG |
| 1311 | 3089 | 1 | AAACGGTGTTCGCGCCGGGG | TGG |
| 1312 | 3092 | -1 | TTGAAGTTCTGACGCCACCC | CGG |
| 1313 | 3117 | -1 | ATAAAGCTCAGCAAAGTCTT | TGG |
| 1314 | 3136 | 1 | TTTGCTGAGCTTTATAACCT | TGG |
| 1315 | 3142 | -1 | GGGCAGCAACAGGCAAACCA | AGG |
| 1316 | 3152 | -1 | TTGTAATAAAGGGCAGCAAC | AGG |
| 1317 | 3162 | -1 | CCTTTGGCAGTTGTAATAAA | GGG |
| 1318 | 3163 | -1 | CCCTTTGGCAGTTGTAATAA | AGG |
| 1319 | 3173 | 1 | CCCTTTATTACAACTGCCAA | AGG |
| 1320 | 3174 | 1 | CCTTTATTACAACTGCCAAA | GGG |
| 1321 | 3178 | -1 | CCCCAGATCCTGTCTCCCTT | TGG |
| 1322 | 3181 | 1 | TACAACTGCCAAAGGGAGAC | AGG |
| 1323 | 3187 | 1 | TGCCAAAGGGAGACAGGATC | TGG |
| 1324 | 3188 | 1 | GCCAAAGGGAGACAGGATCT | GGG |
| 1325 | 3189 | 1 | CCAAAGGGAGACAGGATCTG | GGG |
| 1326 | 3190 | 1 | CAAAGGGAGACAGGATCTGG | GGG |
| 1327 | 3194 | 1 | GGGAGACAGGATCTGGGGGA | AGG |
| 1328 | 3197 | 1 | AGACAGGATCTGGGGGAAGG | AGG |
| 1329 | 3210 | -1 | ATATATATATGTCTATGTGG | AGG |
| 1330 | 3213 | -1 | TATATATATATATGTCTATG | TGG |
| 1331 | 3296 | 1 | GATTCTTAATGATGATATCA | TGG |
| 1332 | 3322 | -1 | AAAGCTTATTATATTAATAA | TGG |
| 1333 | 3379 | 1 | AAGATGAAGAAGAAGAAAAC | AGG |
| 1334 | 3465 | 1 | ATTATTGTACTTAATTCAGC | TGG |

TABLE 8
gRNA (guide RNA) sequences and complementing PAMs (protospacer
adjacent motif) of CsSPGB (referred to as SEQ ID NOs: 1338-1500
in the seq.listing file).

| Seq# | Position | Strand | Sequence | PAM |
|---|---|---|---|---|
| 1338 | 93 | -1 | TTATATGTATAAATATCATA | TGG |
| 1339 | 108 | 1 | ATGATATTTATACATATAAT | TGG |
| 1340 | 120 | 1 | CATATAATTGGCCACACCCA | CGG |
| 1341 | 120 | -1 | CTACTGTAAATCCGTGGGTG | TGG |
| 1342 | 125 | -1 | TGTGGCTACTGTAAATCCGT | GGG |
| 1343 | 126 | -1 | CTGTGGCTACTGTAAATCCG | TGG |
| 1344 | 143 | -1 | GTATGCAAAAACTAATTCTG | TGG |
| 1345 | 239 | 1 | AAACACATACAAACAAACAA | AGG |
| 1346 | 242 | 1 | CACATACAAACAAACAAAGG | AGG |
| 1347 | 254 | -1 | AGGCTGGAACTTAACTTGGT | GGG |
| 1348 | 255 | -1 | GAGGCTGGAACTTAACTTGG | TGG |
| 1349 | 258 | -1 | TAAGAGGCTGGAACTTAACT | TGG |
| 1350 | 270 | -1 | CTTGTTATTTCCTAAGAGGC | TGG |
| 1351 | 271 | 1 | AAGTTAAGTTCCAGCCTCTT | AGG |
| 1352 | 274 | -1 | TTTCCTTGTTATTTCCTAAG | AGG |
| 1353 | 282 | 1 | CAGCCTCTTAGGAAATAACA | AGG |
| 1354 | 345 | -1 | AACATGTAAGTGGTATTTTA | AGG |
| 1355 | 355 | -1 | TTTATATCGAAACATGTAAG | TGG |
| 1356 | 379 | -1 | TGCTTTTGTTGGGGAAAGGA | AGG |
| 1357 | 383 | -1 | TGTATGCTTTTGTTGGGGAA | AGG |
| 1358 | 388 | -1 | ATATATGTATGCTTTTGTTG | GGG |
| 1359 | 389 | -1 | TATATATGTATGCTTTTGTT | GGG |
| 1360 | 390 | -1 | ATATATATGTATGCTTTTGT | TGG |
| 1361 | 443 | -1 | TTGTTATGACTAATGAAGAG | GGG |
| 1362 | 444 | -1 | GTTGTTATGACTAATGAAGA | GGG |
| 1363 | 445 | -1 | AGTTGTTATGACTAATGAAG | AGG |
| 1364 | 477 | -1 | CACTACCTTAGCTTGAGAGA | GGG |
| 1365 | 478 | -1 | TCACTACCTTAGCTTGAGAG | AGG |
| 1366 | 483 | 1 | AGAGACCCTCTCTCAAGCTA | AGG |
| 1367 | 505 | 1 | GTAGTGAGATATATAGTGTT | AGG |
| 1368 | 515 | 1 | ATATAGTGTTAGGAAAGTAA | AGG |
| 1369 | 550 | 1 | TATATATACTCATATTAAAA | TGG |
| 1370 | 585 | 1 | TCGTTGAAGACTACGCTTTT | TGG |
| 1371 | 586 | 1 | CGTTGAAGACTACGCTTTTT | GGG |
| 1372 | 621 | -1 | TGTTTTTAAGATTAATGAAG | AGG |
| 1373 | 664 | -1 | TTTTGCTTTCTTCTATTTTG | GGG |
| 1374 | 665 | -1 | ATTTTGCTTTCTTCTATTTT | GGG |
| 1375 | 666 | -1 | TATTTTGCTTTCTTCTATTT | TGG |
| 1376 | 727 | 1 | TAGTTAATACAAACTTTACC | TGG |
| 1377 | 734 | -1 | ACTTAGAAGAAGGCAAAACC | AGG |
| 1378 | 744 | -1 | AAAATGAAAGACTTAGAAGA | AGG |
| 1379 | 794 | -1 | ACAGGCATATACAAATGAGC | TGG |
| 1380 | 812 | 1 | ATTTGTATATGCCTGTAAAT | AGG |
| 1381 | 812 | -1 | TTTTTTTGTCACCTATTTAC | AGG |
| 1382 | 841 | -1 | TTCTTAGAGGGTATCTATCT | GGG |
| 1383 | 842 | -1 | ATTCTTAGAGGGTATCTATC | TGG |
| 1384 | 853 | -1 | TTTGTATTAATATTCTTAGA | GGG |
| 1385 | 854 | -1 | CTTTGTATTAATATTCTTAG | AGG |
| 1386 | 897 | -1 | GTTTAATTTGCAAAAGTACA | TGG |
| 1387 | 980 | 1 | ATAAGTTAATAACCCATTTG | AGG |
| 1388 | 981 | -1 | TTTACGTGCTTTCCTCAAAT | GGG |
| 1389 | 982 | -1 | ATTTACGTGCTTTCCTCAAA | TGG |
| 1390 | 1015 | -1 | TCGATCAAGAGCTAGAAAAC | AGG |
| 1391 | 1064 | -1 | CGGGGGACAGACGAAACAAG | AGG |
| 1392 | 1081 | -1 | TGAAAATGGGTCAGCTTCGG | GGG |
| 1393 | 1082 | -1 | CTGAAAATGGGTCAGCTTCG | GGG |
| 1394 | 1083 | -1 | ACTGAAAATGGGTCAGCTTC | GGG |
| 1395 | 1084 | -1 | AACTGAAAATGGGTCAGCTT | CGG |
| 1396 | 1094 | -1 | AAAGGTTCCAAACTGAAAAT | GGG |
| 1397 | 1095 | -1 | AAAAGGTTCCAAACTGAAAA | TGG |
| 1398 | 1098 | 1 | AAGCTGACCCATTTTCAGTT | TGG |
| 1399 | 1112 | -1 | TAAACACTTTTGGGAAGAAA | AGG |
| 1400 | 1121 | -1 | CTTGTTTGTTAAACACTTTT | GGG |
| 1401 | 1122 | -1 | TCTTGTTTGTTAAACACTTT | TGG |
| 1402 | 1138 | 1 | AGTGTTTAACAAACAAGAAG | AGG |
| 1403 | 1157 | 1 | GAGGAATCTAAAGTCTCAAA | TGG |
| 1404 | 1158 | 1 | AGGAATCTAAAGTCTCAAAT | GGG |
| 1405 | 1180 | 1 | GCTTGTGAAAGATGAAACAT | TGG |
| 1406 | 1188 | 1 | AAGATGAAACATTGGAGATG | TGG |
| 1407 | 1196 | 1 | ACATTGGAGATGTGGTTGTT | TGG |
| 1408 | 1204 | 1 | GATGTGGTTGTTTGGATGCA | AGG |
| 1409 | 1208 | 1 | TGGTTGTTTGGATGCAAGGA | TGG |
| 1410 | 1213 | 1 | GTTTGGATGCAAGGATGGAT | CGG |
| 1411 | 1218 | 1 | GATGCAAGGATGGATCGGTG | AGG |
| 1412 | 1233 | -1 | CCTGAGTTTCCATTTCTCGA | TGG |
| 1413 | 1235 | 1 | GTGAGGCTTCCATCGAGAAA | TGG |
| 1414 | 1244 | 1 | CCATCGAGAAATGGAAACTC | AGG |
| 1415 | 1245 | 1 | CATCGAGAAATGGAAACTCA | GGG |
| 1416 | 1257 | -1 | ACAGCATTGAGCTTGAATTC | TGG |
| 1417 | 1274 | 1 | TTCAAGCTCAATGCTGTAAG | AGG |
| 1418 | 1280 | 1 | CTCAATGCTGTAAGAGGCTG | AGG |
| 1419 | 1286 | 1 | GCTGTAAGAGGCTGAGGCAG | AGG |
| 1420 | 1301 | -1 | CTCCGGACAGTACTCTGTTT | GGG |
| 1421 | 1302 | -1 | TCTCCGGACAGTACTCTGTT | TGG |
| 1422 | 1310 | 1 | GACCCAAACAGAGTACTGTC | CGG |
| 1423 | 1316 | 1 | AACAGAGTACTGTCCGGAGA | CGG |
| 1424 | 1318 | -1 | CCCTCCTCCAGCTCCGTCTC | CGG |
| 1425 | 1322 | 1 | GTACTGTCCGGAGACGGAGC | TGG |
| 1426 | 1325 | 1 | CTGTCCGGAGACGGAGCTGG | AGG |
| 1427 | 1328 | 1 | TCCGGAGACGGAGCTGGAGG | AGG |
| 1428 | 1329 | 1 | CCGGAGACGGAGCTGGAGGA | GGG |
| 1429 | 1336 | 1 | CGGAGCTGGAGGAGGGACCA | TGG |
| 1430 | 1339 | 1 | AGCTGGAGGAGGGACCATGG | TGG |
| 1431 | 1342 | -1 | TGATCGACCTCCGACCACCA | TGG |
| 1432 | 1343 | 1 | GGAGGAGGGACCATGGTGGT | CGG |
| 1433 | 1346 | 1 | GGAGGGACCATGGTGGTCGG | AGG |
| 1434 | 1363 | 1 | CGGAGGTCGATCAGTGCAAA | AGG |
| 1435 | 1364 | 1 | GGAGGTCGATCAGTGCAAAA | GGG |
| 1436 | 1383 | 1 | AGGGTCTTGCTAAAAAGTCT | TGG |
| 1437 | 1398 | 1 | AGTCTTGGTAGATCATGTTT | CGG |
| 1438 | 1405 | 1 | GTAGATCATGTTTCGGTAGT | TGG |
| 1439 | 1410 | 1 | TCATGTTTCGGTAGTTGGTG | TGG |
| 1440 | 1436 | 1 | TTATTGATGTTGTTGAGTTG | TGG |
| 1441 | 1439 | 1 | TTGATGTTGTTGAGTTGTGG | TGG |
| 1442 | 1442 | 1 | ATGTTGTTGAGTTGTGGTGG | TGG |
| 1443 | 1445 | 1 | TTGTTGAGTTGTGGTGGTGG | TGG |
| 1444 | 1448 | 1 | TTGAGTTGTGGTGGTGGTGG | TGG |
| 1445 | 1514 | -1 | CAAAAGCCATGGAAGAAGTT | TGG |
| 1446 | 1519 | 1 | ATCTTTCCAAACTTCTTCCA | TGG |
| 1447 | 1525 | -1 | TCCTTCTACAACAAAAGCCA | TGG |
| 1448 | 1535 | 1 | TCCATGGCTTTTGTTGTAGA | AGG |
| 1449 | 1597 | 1 | AAAAGACTTCTTTGAAGAAG | AGG |
| 1450 | 1620 | -1 | AGCTTAAGCTTACACAACAC | AGG |
| 1451 | 1657 | 1 | ATTTCTTCTTCTTCTTCTTC | TGG |
| 1452 | 1713 | -1 | ATACTAATGTCGTCGTCATC | AGG |
| 1453 | 1735 | 1 | CGACATTAGTATAAGACTGA | TGG |
| 1454 | 1736 | 1 | GACATTAGTATAAGACTGAT | GGG |
| 1455 | 1822 | 1 | AGAGATGAGAAGAGTAGAGC | TGG |
| 1456 | 1850 | 1 | GTAGCTACAGCTATGTATGA | AGG |
| 1457 | 1888 | 1 | AGAGAGAGAAGAAGAAGAA | TGG |
| | | | C | |
| 1458 | 1914 | 1 | ATATTGTATTGAGCCACGCG | CGG |
| 1459 | 1916 | -1 | AAACAGTACTCTTCCGCGCG | TGG |
| 1460 | 1934 | 1 | CGGAAGAGTACTGTTTTTAT | TGG |
| 1461 | 1935 | 1 | GGAAGAGTACTGTTTTTATT | GGG |
| 1462 | 1938 | 1 | AGAGTACTGTTTTTATTGGG | AGG |
| 1463 | 1959 | -1 | CCTATAAGTCTCTTTAATTT | GGG |
| 1464 | 1960 | -1 | CCCTATAAGTCTCTTTAATT | TGG |
| 1465 | 1970 | 1 | CCCAAATTAAAGAGACTTAT | AGG |
| 1466 | 1971 | 1 | CCAAATTAAAGAGACTTATA | GGG |
| 1467 | 1976 | 1 | TTAAAGAGACTTATAGGGCC | TGG |
| 1468 | 1983 | -1 | AAGTATACAGTTTACAGGCC | AGG |
| 1469 | 1988 | -1 | GACTGAAGTATACAGTTTAC | AGG |
| 1470 | 2010 | -1 | TGTGGAACAGCAGATGAGAT | TGG |
| 1471 | 2028 | -1 | TTTAGATTCTCATAATGTTG | TGG |
| 1472 | 2042 | 1 | CAACATTATGAGAATCTAAA | AGG |
| 1473 | 2062 | -1 | CACTGTTAAAAAGGAGTGGT | GGG |
| 1474 | 2063 | -1 | GCACTGTTAAAAAGGAGTGG | TGG |
| 1475 | 2066 | -1 | GTGGCACTGTTAAAAAGGAG | TGG |
| 1476 | 2071 | -1 | GGTTAGTGGCACTGTTAAAA | AGG |
| 1477 | 2085 | -1 | ATATATAGGGGGATGGTTAG | TGG |
| 1478 | 2092 | -1 | CATGTACATATATAGGGGGA | TGG |
| 1479 | 2096 | -1 | CAAGCATGTACATATATAGG | GGG |
| 1480 | 2097 | -1 | ACAAGCATGTACATATATAG | GGG |
| 1481 | 2098 | -1 | TACAAGCATGTACATATATA | GGG |
| 1482 | 2099 | -1 | CTACAAGCATGTACATATAT | AGG |
| 1483 | 2163 | -1 | AAGACATAATGCTAAGTAAA | TGG |
| 1484 | 2192 | -1 | TAGTCAATTAATCTTATTCA | TGG |
| 1485 | 2225 | 1 | AATTTGCAACTACTCTCTCA | AGG |
| 1486 | 2228 | 1 | TTGCAACTACTCTCTCAAGG | TGG |
| 1487 | 2229 | 1 | TGCAACTACTCTCTCAAGGT | GGG |
| 1488 | 2245 | 1 | AGGTGGGTTGTGTATTAATT | AGG |
| 1489 | 2250 | 1 | GGTTGTGTATTAATTAGGCC | TGG |
| 1490 | 2257 | -1 | CAATATTCCACATTCACACC | AGG |
| 1491 | 2261 | 1 | AATTAGGCCTGGTGTGAATG | TGG |
| 1492 | 2269 | 1 | CTGGTGTGAATGTGGAATAT | TGG |
| 1493 | 2283 | 1 | GAATATTGGCATATATAGTA | TGG |
| 1494 | 2376 | 1 | CTTTGTTTTGAGATTTTAAA | AGG |
| 1495 | 2377 | 1 | TTTGTTTTGAGATTTTAAAA | GGG |
| 1496 | 2392 | -1 | AACTTTGCCTGTTTCATTCA | TGG |
| 1497 | 2396 | 1 | AGGGTGTCCATGAATGAAAC | AGG |
| 1498 | 2416 | 1 | AGGCAAAGTTTGTCTTTGAC | AGG |
| 1499 | 2421 | 1 | AAGTTTGTCTTTGACAGGAT | TGG |
| 1500 | 2433 | 1 | GACAGGATTGGTCTGAATTT | TGG |

### Example 3: Modification of Multiflora Gene in Cannabis Plants

The aim of the example was to introduce a mutation in the Multiflora gene, thereby inhibiting the expression of the multiflora protein. This may potentially increase the yield of flower in cannabis plants, by causing the plant to produce more flowers.

The Multiflora gene was mutated via CRISPR/Cas-9 system. Cannabis plants were transformed using Agrobacterium tumefaciens containing a binary plasmid harboring the Cas-9 gene and a gRNA expression cassette. Plant tissue samples were collected 10-14 days post transformation and DNA was extracted. The DNA was then used to perform PCR using specific primers (Fw 5-GGCGATTCCTGTTGCGGGTT-3 Rv 5-ATGAGAGGAGTCCGGAGCCG-3) flanking relevant guide sequences after which the PCR product was analyzed by Next Generation Sequencing (NGS) to identify editing events.

Editing of the Multiflora gene was carried out by use of the gRNA set forth as SEQ ID NO 750 (also referenced in Table 4, position 624). SEQ ID NO 750 was specifically chosen for use, since it is located at the beginning of the gene, and a mutation therein will lead to a stop codon, inhibiting the protein's expression. Base pairs were inserted by random, non-homologous insertion. Amplicon sequencing was executed at 100,000 reads per sample and analyzed to identify editing events. 95% of amplicons had the original Wild Type (WT) sequence while 1.8% had an “A” insertion at the 4^{th} position upstream to the PAM, 1.1% had an “C” insertion and 0.7% had an “T” insertion at the same position, set forth as SEQ ID Nos. 1501, 1502, and 1503, respectively.

1) A method for increasing yield in Cannabis plants selected from a group consisting of C. sativa, C. indica, and C. ruderalis, comprising steps of;
a) selecting a gene involved in the flowering pathways of said Cannabis species;
b) synthesizing or designing a gRNA expression cassette corresponding to a targeted cleavage locus along the Cannabis genome or a complex of gRNA and a protein (Ribonucleoprotein protein complex);
c) transforming said Cannabis plant cells to insert said gRNA expression cassette or said ribonucleoprotein protein complex into them;
d) culturing said Cannabis plant cells;
e) selecting said Cannabis cells which express desired mutations in the editing target region, and
f) regenerating a plant from said plant cell, plant cell nucleus, or plant tissue.

2) The method of claim 1, wherein the gene involved in the flowering pathways of said Cannabis species is selected from SEQ ID NO:1, SEQ ID NO:171, SEQ ID NO:390, SEQ ID NO:726, SEQ ID NO:936, SEQ ID NO:1015, SEQ ID NO:1106 or SEQ ID NO:1335.

3) The gRNAs of claim 1 and their corresponding PAMs are selected from a group consisting of SEQ ID NOs:4-170, SEQ ID NOs:174-389, SEQ ID NOs:393-725, SEQ ID NOs: 729-935, SEQ ID NOs: 939-1014, SEQ ID NOs: 1018-1105, SEQ ID NOs: 1109-1334 and SEQ ID NOs: 1338-1500.

4) The method of claim 2, wherein the target domain sequence is selected from the group comprising of: (1) a nucleic acid sequence encoding the polypeptide of SEQ ID NO:1, (2) a nucleic acid sequence comprising the sequence of SEQ ID NO:171, (3) a nucleic acid sequence encoding the polypeptide of SEQ ID NO:390, (4) a nucleic acid sequence encoding the polypeptide of SEQ ID NO:726, (5) a nucleic acid sequence encoding the polypeptide of SEQ ID NO:936, (6) a nucleic acid sequence encoding the polypeptide of SEQ ID NO:1015, (7) a nucleic acid sequence encoding the polypeptide of SEQ ID NO:1106, (8) a nucleic acid sequence encoding the polypeptide of SEQ ID NO:1335, (9) a nucleic acid sequence having at least 80% sequence identity to at least 200 contiguous nucleotides of the nucleic acid sequence of SEQ ID NO:1, (10) a nucleic acid sequence having at least 80% sequence identity to at least 200 contiguous nucleotides of the nucleic acid sequence of SEQ ID NO:171, (11) a nucleic acid sequence having at least 80% sequence identity to at least 200 contiguous nucleotides of the nucleic acid sequence of SEQ ID NO:390, (12) a nucleic acid sequence having at least 80% sequence identity to at least 200 contiguous nucleotides of the nucleic acid sequence of SEQ ID NO:726, (13) a nucleic acid sequence having at least 80% sequence identity to at least 200 contiguous nucleotides of the nucleic acid sequence of SEQ ID NO:936, (14) a nucleic acid sequence having at least 80% sequence identity to at least 200 contiguous nucleotides of the nucleic acid sequence of SEQ ID NO:1015, (15) a nucleic acid sequence having at least 80% sequence identity to at least 200 contiguous nucleotides of the nucleic acid sequence of SEQ ID NO:1106, (16) a nucleic acid sequence having at least 80% sequence identity to at least 200 contiguous nucleotides of the nucleic acid sequence of SEQ ID NO:1335.

5) The method of claim 1, wherein said transforming is executed by means selected from a group consisting of: the Agrobacterium-mediated transformation method, particle bombardment (biolistics), injection, viral transformation, in planta transformation, electroporation, lipofection, sonication, silicon carbide fiber mediated gene transfer, laser microbeam (UV) induced gene-transfer, co-cultivation with the explants tissue and any combination thereof.

6) The method of claim 5, wherein the transformation is carried out using Agrobacterium to deliver an expression cassette comprised of: (a) a selection marker; (b) a nucleotide sequence encoding one or more gRNA molecules comprising a DNA sequence which is complementary with a target domain sequence selected from the group pf genes consisting of SEQ ID NO:1, SEQ ID NO:171, SEQ ID NO:390, SEQ ID NO:726, SEQ ID NO:936, SEQ ID NO:1015, SEQ ID NO:1106 and SEQ ID NO:1335, and (c) a nucleotide sequence encoding a Cas molecule from, but not limited to Streptococcus pyogenes or Staphylococcus aureus.

7) The method of claim 6, wherein said method comprises administering a nucleic acid composition that comprises: (a) a first nucleotide sequence encoding the gRNA molecule; and (b) a second nucleotide sequence encoding the Cas protein.

8) The method of claim 5, wherein the CRISPR/Cas system is delivered to the cell by a plant virus.

9) The method of claim 7, wherein the Cas protein is selected from a group comprising but not limited to Cpf1, Cas9, Cas12, Cas13, Cas14, CasX or CasY.

10) The method of claim 1, wherein increasing Cannabis yield comprising steps of
(a) introducing into a Cannabis plant or a cell thereof (i) at least one RNA-guided endonuclease comprising at least one nuclear localization signal or nucleic acid encoding at least one RNA-guided endonuclease comprising at least one nuclear localization signal, (ii) at least one guide RNA or DNA encoding at least one guide RNA, and, optionally, (iii) at least one donor polynucleotide; and
(b) culturing the Cannabis plant or cell thereof such that each guide RNA directs an RNA-guided endonuclease to a targeted site in the chromosomal sequence where the RNA-guided endonuclease introduces a double-stranded break in the targeted site, and the double-stranded break is repaired by a DNA repair process such that the chromosomal sequence is modified, wherein the targeted site is located in genes selected from SEQ ID NO:1, SEQ ID NO:171, SEQ ID NO:390, SEQ ID NO:726, SEQ ID NO:936, SEQ ID NO:1015, SEQ ID NO:1106 and SEQ ID NO:1335 and the chromosomal modification interrupts or interferes with transcription and/or translation of the genes selected from SEQ ID NO:1, SEQ ID NO:171, SEQ ID NO:390, SEQ ID NO:726, SEQ ID NO:936, SEQ ID NO:1015, SEQ ID NO:1106 and SEQ ID NO:1335.

11) The method of claim 10, wherein the RNA-guided endonuclease is derived from a clustered regularly interspersed short palindromic repeats (CRISPR)/CRISPR-associated (Cas) system.

12) The method of claim 10, wherein the introduction of SEQ ID NO:1, SEQ ID NO:171, SEQ ID NO:390, SEQ ID NO:726, SEQ ID NO:936, SEQ ID NO:1015, SEQ ID NO:1106 and SEQ ID NO:1335 does not insert exogenous genetic material and produces a non-naturally occurring Cannabis plant or cell thereof.

13) The method of claim 1, wherein increasing Cannabis yield comprises;
(a) identifying at least one locus within a DNA sequence in a Cannabis plant or a cell thereof for SEQ ID NO:1, SEQ ID NO:171, SEQ ID NO:390, SEQ ID NO:726, SEQ ID NO:936, SEQ ID NO:1015, SEQ ID NO:1106 and SEQ ID NO:1335;
(b) identifying at least one custom endonuclease recognition sequence within the at least one locus of SEQ ID NO:1, SEQ ID NO:171, SEQ ID NO:390, SEQ ID NO:726, SEQ ID NO:936, SEQ ID NO:1015, SEQ ID NO:1106 and SEQ ID NO:1335;
(c) introducing into the Cannabis plant or a cell thereof at least a first custom endonuclease, wherein the Cannabis plant or a cell thereof comprises the recognition sequence for the custom endonuclease in or proximal to the loci of SEQ ID NO:1, SEQ ID NO:171, SEQ ID NO:390, SEQ ID NO:727, SEQ ID NO:937, SEQ ID NO:1016, SEQ ID NO:1107 and SEQ ID NO:1336, and the custom endonuclease is expressed transiently or stably;
(d) assaying the Cannabis plant or a cell thereof for a custom endonuclease-mediated modification in the DNA making up or flanking the loci of SEQ ID NO:1, SEQ ID NO:171, SEQ ID NO:390, SEQ ID NO:727, SEQ ID NO:937, SEQ ID NO:1016, SEQ ID NO:1107 and SEQ ID NO:1336, (e) identifying the Cannabis plant, a cell thereof, or a progeny cell thereof as comprising a modification in the loci of SEQ ID NO:1, SEQ ID NO:171, SEQ ID NO:390, SEQ ID NO:727, SEQ ID NO:937, SEQ ID NO:1016, SEQ ID NO:1107 and SEQ ID NO:1336.

14) The method of claim 13, wherein increasing said Cannabis yield is selected from a group consisting of: increasing the number of flowers, increasing the size of the flowers, increasing the weight of the flowers, increasing the number of buds, increasing the size of the buds, increasing the weight of the buds and any combination thereof.

15) A method for increasing yield in Cannabis plants selected from a group consisting of C. sativa, C. indica, and C. ruderalis, comprising steps of;
a) selecting a gene involved in the flowering pathways of said Cannabis species;
b) obtaining cells of said Cannabis plants;
c) editing said genes involved in the flowering pathways of said cells;
d) culturing said cells;
e) selecting said cells expressing desired mutations in the editing target region, and
f) regenerating a Cannabis plant from said cell, plant cell nucleus, or plant tissue.

16) The method of claim 15, wherein said editing is executed by means selected from a group consisting of: CRISPR/Cas, cleaving the genome of said cell using zinc finger nucleases, cleaving the genome of said cell using meganucleases (homing endonucleases), cleaving the genome of said cell using transcription activator-like effector nucleases (TALEN), and any combination thereof.

17) The method of claim 15, wherein increasing said Cannabis yield is selected from a group consisting of: increasing the number of flowers, increasing the size of the flowers, increasing the weight of the flowers, increasing the number of buds, increasing the size of the buds, increasing the weight of the buds and any combination thereof.

18) The method of claim 3, wherein the gRNA corresponds to a Multiflora gene.

19) The method of claim 1, wherein the mutation occurs in a Multiflora gene, and the mutation is set forth as SEQ ID NOs. 1501-1503.

20) A Cannabis plant produced according to claim 1.

21) A seed of the Cannabis plant of claim 20.

22) A mutated Cannabis plant comprising a mutation in gene Multiflora wherein the mutation of the Multiflora gene is set forth as SEQ ID NOs. 1501, 1502, or 1503.

23) A seed of the mutated cannabis plant of claim 22.


SEQUENCE LISTING (1503 sequences)

SEQ ID NO: 1 - DNA, 3084 bp, Cannabis sativa
tggtgatccttaattttattatgtaatgtgcttcatgaaataatatataagtatatatttaattttagttcatgtcttatataaagacttcaggagttaattaacagattaaagtcattaataatataataataataataataataatatacaaccagaagagttttatcacaaaaatattacttaaaaataataccctcaattgaaaaatatatataaaatgtttaactactataaaaaaaacttaaatgttagaaaataaaaaaatttaaggacttaattcaaataaacaaataaataaaagaagaagaagcaattttctttcttcattttatcatatgcacaacaacaatttttcagtacatcaatcaatccatacataaaaatacataaaatacaataaaattttaaaaaacaaaaaatacaactaaaagatattacatctacaaatagtggttcgatcagatcaacgattagtaaggcgttgactatccttatcacaggtcaaagaaaaaatgacctataattcttctttcttcttcttcttctttgacaatcataaagagatagccacctaatgaatcaaaacactactacaacaaggatacagatacacatttgtattgtatcatatttcttaatatttaacaaaattaaattaataaataaaaagagactcgcatgagtggcaaattaagcatattatatagaccctcctccttataataagcaaaggaaatggcattcttcatatattattattattattaagcatactgaggattgagtgctatagcctcctggtagatcatatctttcatttgttcttctgtcaaaggttgttgttcgaattcgaaagagaaaggatttgagcacactggttcatcagcaacatcatggagtcttgtcaaatagggatgagctaaagcttcttcaactgttaatacacaaaagttcccccaagttagttaggcactgatatctgaatcaaaggagaatgcaaagacaggattttcattttttgtattgctattcgaaatgtcaagtattgagtatgaaaatttaccagtaattcttctatttggatcaaatgtcaacattttctcaatgaggtctatggccagtggatgaacatgtgggaaagccctgcctaactgttggcgagggaacgaagggagttgccgtatgtatcttttagcatcttcatttcgaatgaaaccgagatctgactctgttggtgtgcctagcagctaagaaacacagaataatgcataagtaattacttcattaaacgtataatataacacgcaagaacttagatggtaaaaataaacaaaggaaagctgtatctaattgggaaatcattccacagttccaaaagttcttttagcattaaatgaagaaaaattggcaaagatggaaaaggtctcaatagttgaaatttggtattagaatctacagtgagacttcacttctgaaagaaattgttttaagtatatttgtccttacctctgtcaataagcgcatctgatgaacatgatctttgcctggaaacaaaggcttcctattcataagctccataaagatgcaaccaacagaccacacatctattgaagcagtataatctgaagagttcaacaacaactcaggagccctgtaccatctggtgacaacatactcagtcatatactcattttccgcggtgggacgagcaagaccaaaatcacatattttaagatcacaattcgcattaagcaaaaggttgctgggttttaaatccctgtgtataacattagcagaatgtatgtacttcaaccctcgaagaatctggtacaagaagtactgcataacaattaagaaaagaaaaaaatgtcagcatcaagccttgaaaagctagaattaagttggccatgaagtcaataaacaaagactagtttttttacctgacaatgctcctctgataaaccttgatttgaacgaataatttggtgaagatcagtgtccatgagctcagtagcaatgtagacatcagtaaattctcgccgcaaaggtggtggaatcacatcccttatagctacaatctgtagtagcataaacattcataccatttatatataatcaacaaaacaaaaccttgacttttatgtaccctatattgtatagaatagatattccaattaatgaaaaagaaaacataacacagctttgtatatgacttcttttatttatgtcaaaatttccatttataaattcattttgtacagagtcaatgtaaattggtaaagacctaatttacccaaatcatcaattaaaaatactgagaagagacaaattgaatcaccaaaaacagaaaattaggcacacaaaacaaaagattacaatttttatatataaatattcaacagataataatgttactgacattttcgtgatccaaatgacgaagaagcttaatctcccgaagcgtgcgtttggcatccatatgattatcaaaggcgttagcaattttcttcatagcaaccatctcattcgtctccgaattcaaaaccgagctgtaattcaaaaacaaaaacaatatcatcgatccgaaaagtagcgaatcaaaatcgaaagtcaacgattgaaatacttaccaaacgataccataagctcctctaccaataggcataatcggaggtcgatacttggttgtaatctcgaacaagttgccaaatatgttgtactgaatgtactgacctccatgggaaggaatcgcgggaaaatccgtgtactgaggtcctcctccggctccgggagcggcggcaccgggaagattgttatgagtggcatcaaccatattgaagtttctcttctcttctctttctttttctctctctagtttctctttctctctctagaaagtcttcagagacttgagattgaaacactgaaatcgaaatcgaatgagtattgaagattttccaaagacaagacttttaaaccaaatttcaatcaaacaaaaatatttcttattattattattattattattattattattattattattattattatgaggaatatatttttttaatttttttt

SEQ ID NO: 2 - DNA, 1161 bp, Artificial Sequence
  CsBif2 CDS
atggttgatgccactcataacaatcttcccggtgccgccgctcccggagccggaggaggacctcagtacacggattttcccgcgattccttcccatggaggtcagtacattcagtacaacatatttggcaacttgttcgagattacaaccaagtatcgacctccgattatgcctattggtagaggagcttatggtatcgttgttttgaattcggagacgaatgagatggttgctatgaagaaaattgctaacgcctttgataatcatatggatgccaaacgcacgcttcgggagattaagcttcttcgtcatttggatcacgaaaatcagattgtagctataagggatgtgattccaccacctttgcggcgagaatttactgatgtctacattgctactgagctcatggacactgatcttcaccaaattattcgttcaaatcaaggtttatcagaggagcattgtcagcagtacttcttgtaccagattcttcgagggttgaagtacatacattctgctaatgttatacacagggatttaaaacccagcaaccttttgcttaatgcgaattgtgatcttaaaatatgtgattttggtcttgctcgtcccaccgcggaaaatgagtatatgactgagtatgttgtcaccagatggtacagggctcctgagttgttgttgaactcttcagattatactgcttcaatagatgtgtggtctgttggttgcatctttatggagcttatgaataggaagcctttgtttccaggcaaagatcatgttcatcagatgcgcttattgacagaggtactgctaggcacaccaacagagtcagatctcggtttcattcgaaatgaagatgctaaaagatacatacggcaactcccttcgttccctcgccaacagttaggcagggctttcccacatgttcatccactggccatagacctcattgagaaaatgttgacatttgatccaaatagaagaattacagttgaagaagctttagctcatccctatttgacaagactccatgatgttgctgatgaaccagtgtgctcaaatcctttctctttcgaattcgaacaacaacctttgacagaagaacaaatgaaagatatgatctaccaggaggctatagcactcaatcctcagtatgcttaa

SEQ ID NO: 3 - protein, 387 aa, Artificial Sequence
  CsBif2 AA
MVDATHNNLPGAAAPGAGGGPQYTDFPAIPSHGGQYIQYNIFGNLFEITTKYRPPIMPIGRGAYGIVWVLNSETNEMVAMKKIANAFDNHMDAKRTLREIKLLRHLDHENQIVAIRDVIPPPLRREFTDVYIATELMDTDLHQIIRSNQGLSEEHCQQYFLYQILRGLKYIHSANVIHRDLKPSNLLLNANCDLKICDFGLARPTAENEYMTEYVVTRWYRAPELLLNSSDYTASIDVWSVGCIFMELMNRKPLFPGKDHVHQMRLLTEVLLGTPTESDLGFIRNEDAKRYIRQLPSFPRQQLGRAFPHVHPLAIDLIEKMLTFDPNRRITVEEALAHPYLTRLHDVADEPVCSNPFSFEFEQQPLTEEQMKDMIYQEAIALNPQYA

SEQ ID NO: 4 - DNA, 20 bp, Cannabis sativa
cacattacataataaaatta

SEQ ID NO: 5 - DNA, 20 bp, Cannabis sativa
atgtcttatataaagacttc

SEQ ID NO: 6 - DNA, 20 bp, Cannabis sativa
tttttgtgataaaactcttc

SEQ ID NO: 7 - DNA, 20 bp, Cannabis sativa
tatatatatttttcaattga

SEQ ID NO: 8 - DNA, 20 bp, Cannabis sativa
ttatatatatttttcaattg

SEQ ID NO: 9 - DNA, 20 bp, Cannabis sativa
ttagaaaataaaaaaattta

SEQ ID NO: 10 - DNA, 20 bp, Cannabis sativa
attttatgtatttttatgta

SEQ ID NO: 11 - DNA, 20 bp, Cannabis sativa
gatattacatctacaaatag

SEQ ID NO: 12 - DNA, 20 bp, Cannabis sativa
gatcagatcaacgattagta

SEQ ID NO: 13 - DNA, 20 bp, Cannabis sativa
gcgttgactatccttatcac

SEQ ID NO: 14 - DNA, 20 bp, Cannabis sativa
tttttctttgacctgtgata

SEQ ID NO: 15 - DNA, 20 bp, Cannabis sativa
gaagaagaaagaagaattat

SEQ ID NO: 16 - DNA, 20 bp, Cannabis sativa
tagtgttttgattcattagg

SEQ ID NO: 17 - DNA, 20 bp, Cannabis sativa
tagtagtgttttgattcatt

SEQ ID NO: 18 - DNA, 20 bp, Cannabis sativa
atcaaaacactactacaaca

SEQ ID NO: 19 - DNA, 20 bp, Cannabis sativa
taaaaagagactcgcatgag

SEQ ID NO: 20 - DNA, 20 bp, Cannabis sativa
tttgcttattataaggagga

SEQ ID NO: 21 - DNA, 20 bp, Cannabis sativa
ctttgcttattataaggagg

SEQ ID NO: 22 - DNA, 20 bp, Cannabis sativa
ttcctttgcttattataagg

SEQ ID NO: 23 - DNA, 20 bp, Cannabis sativa
catttcctttgcttattata

SEQ ID NO: 24 - DNA, 20 bp, Cannabis sativa
ctcctccttataataagcaa

SEQ ID NO: 25 - DNA, 20 bp, Cannabis sativa
cttataataagcaaaggaaa

SEQ ID NO: 26 - DNA, 20 bp, Cannabis sativa
tattattattaagcatactg

SEQ ID NO: 27 - DNA, 20 bp, Cannabis sativa
gattgagtgctatagcctcc

SEQ ID NO: 28 - DNA, 20 bp, Cannabis sativa
gaaagatatgatctaccagg

SEQ ID NO: 29 - DNA, 20 bp, Cannabis sativa
aatgaaagatatgatctacc

SEQ ID NO: 30 - DNA, 20 bp, Cannabis sativa
ttcatttgttcttctgtcaa

SEQ ID NO: 31 - DNA, 20 bp, Cannabis sativa
tgttcgaattcgaaagagaa

SEQ ID NO: 32 - DNA, 20 bp, Cannabis sativa
gagaaaggatttgagcacac

SEQ ID NO: 33 - DNA, 20 bp, Cannabis sativa
ctggttcatcagcaacatca

SEQ ID NO: 34 - DNA, 20 bp, Cannabis sativa
atcatggagtcttgtcaaat

SEQ ID NO: 35 - DNA, 20 bp, Cannabis sativa
tcatggagtcttgtcaaata

SEQ ID NO: 36 - DNA, 20 bp, Cannabis sativa
tcagtgcctaactaacttgg

SEQ ID NO: 37 - DNA, 20 bp, Cannabis sativa
atcagtgcctaactaacttg

SEQ ID NO: 38 - DNA, 20 bp, Cannabis sativa
tatcagtgcctaactaactt

SEQ ID NO: 39 - DNA, 20 bp, Cannabis sativa
atatcagtgcctaactaact

SEQ ID NO: 40 - DNA, 20 bp, Cannabis sativa
aaagttcccccaagttagtt

SEQ ID NO: 41 - DNA, 20 bp, Cannabis sativa
ggcactgatatctgaatcaa

SEQ ID NO: 42 - DNA, 20 bp, Cannabis sativa
tcaaaggagaatgcaaagac

SEQ ID NO: 43 - DNA, 20 bp, Cannabis sativa
gatccaaatagaagaattac

SEQ ID NO: 44 - DNA, 20 bp, Cannabis sativa
ttaccagtaattcttctatt

SEQ ID NO: 45 - DNA, 20 bp, Cannabis sativa
atgtcaacattttctcaatg

SEQ ID NO: 46 - DNA, 20 bp, Cannabis sativa
cattttctcaatgaggtcta

SEQ ID NO: 47 - DNA, 20 bp, Cannabis sativa
tcaatgaggtctatggccag

SEQ ID NO: 48 - DNA, 20 bp, Cannabis sativa
tttcccacatgttcatccac

SEQ ID NO: 49 - DNA, 20 bp, Cannabis sativa
atggccagtggatgaacatg

SEQ ID NO: 50 - DNA, 20 bp, Cannabis sativa
tggccagtggatgaacatgt

SEQ ID NO: 51 - DNA, 20 bp, Cannabis sativa
ccctcgccaacagttaggca

SEQ ID NO: 52 - DNA, 20 bp, Cannabis sativa
tccctcgccaacagttaggc

SEQ ID NO: 53 - DNA, 20 bp, Cannabis sativa
ggaaagccctgcctaactgt

SEQ ID NO: 54 - DNA, 20 bp, Cannabis sativa
tcgttccctcgccaacagtt

SEQ ID NO: 55 - DNA, 20 bp, Cannabis sativa
gccctgcctaactgttggcg

SEQ ID NO: 56 - DNA, 20 bp, Cannabis sativa
ccctgcctaactgttggcga

SEQ ID NO: 57 - DNA, 20 bp, Cannabis sativa
aactgttggcgagggaacga

SEQ ID NO: 58 - DNA, 20 bp, Cannabis sativa
actgttggcgagggaacgaa

SEQ ID NO: 59 - DNA, 20 bp, Cannabis sativa
aagatgctaaaagatacata

SEQ ID NO: 60 - DNA, 20 bp, Cannabis sativa
acaccaacagagtcagatct

SEQ ID NO: 61 - DNA, 20 bp, Cannabis sativa
aaaccgagatctgactctgt

SEQ ID NO: 62 - DNA, 20 bp, Cannabis sativa
ttctgtgtttcttagctgct

SEQ ID NO: 63 - DNA, 20 bp, Cannabis sativa
ataacacgcaagaacttaga

SEQ ID NO: 64 - DNA, 20 bp, Cannabis sativa
tagatggtaaaaataaacaa

SEQ ID NO: 65 - DNA, 20 bp, Cannabis sativa
aaaggaaagctgtatctaat

SEQ ID NO: 66 - DNA, 20 bp, Cannabis sativa
aaggaaagctgtatctaatt

SEQ ID NO: 67 - DNA, 20 bp, Cannabis sativa
taaaagaacttttggaactg

SEQ ID NO: 68 - DNA, 20 bp, Cannabis sativa
tttaatgctaaaagaacttt

SEQ ID NO: 69 - DNA, 20 bp, Cannabis sativa
agcattaaatgaagaaaaat

SEQ ID NO: 70 - DNA, 20 bp, Cannabis sativa
tgaagaaaaattggcaaaga

SEQ ID NO: 71 - DNA, 20 bp, Cannabis sativa
aaaattggcaaagatggaaa

SEQ ID NO: 72 - DNA, 20 bp, Cannabis sativa
aggtctcaatagttgaaatt

SEQ ID NO: 73 - DNA, 20 bp, Cannabis sativa
tgcgcttattgacagaggta

SEQ ID NO: 74 - DNA, 20 bp, Cannabis sativa
tcagatgcgcttattgacag

SEQ ID NO: 75 - DNA, 20 bp, Cannabis sativa
tgatgaacatgatctttgcc

SEQ ID NO: 76 - DNA, 20 bp, Cannabis sativa
aataggaagcctttgtttcc

SEQ ID NO: 77 - DNA, 20 bp, Cannabis sativa
tgatctttgcctggaaacaa

SEQ ID NO: 78 - DNA, 20 bp, Cannabis sativa
tctttatggagcttatgaat

SEQ ID NO: 79 - DNA, 20 bp, Cannabis sativa
gtctgttggttgcatcttta

SEQ ID NO: 80 - DNA, 20 bp, Cannabis sativa
tcaatagatgtgtggtctgt

SEQ ID NO: 81 - DNA, 20 bp, Cannabis sativa
atactgcttcaatagatgtg

SEQ ID NO: 82 - DNA, 20 bp, Cannabis sativa
gaagagttcaacaacaactc

SEQ ID NO: 83 - DNA, 20 bp, Cannabis sativa
tgttgtcaccagatggtaca

SEQ ID NO: 84 - DNA, 20 bp, Cannabis sativa
atgttgtcaccagatggtac

SEQ ID NO: 85 - DNA, 20 bp, Cannabis sativa
ctcaggagccctgtaccatc

SEQ ID NO: 86 - DNA, 20 bp, Cannabis sativa
ctgagtatgttgtcaccaga

SEQ ID NO: 87 - DNA, 20 bp, Cannabis sativa
agtcatatactcattttccg

SEQ ID NO: 88 - DNA, 20 bp, Cannabis sativa
catatactcattttccgcgg

SEQ ID NO: 89 - DNA, 20 bp, Cannabis sativa
atatactcattttccgcggt

SEQ ID NO: 90 - DNA, 20 bp, Cannabis sativa
tggtcttgctcgtcccaccg

SEQ ID NO: 91 - DNA, 20 bp, Cannabis sativa
gatcttaaaatatgtgattt

SEQ ID NO: 92 - DNA, 20 bp, Cannabis sativa
cacaattcgcattaagcaaa

SEQ ID NO: 93 - DNA, 20 bp, Cannabis sativa
cgcattaagcaaaaggttgc

SEQ ID NO: 94 - DNA, 20 bp, Cannabis sativa
gcattaagcaaaaggttgct

SEQ ID NO: 95 - DNA, 20 bp, Cannabis sativa
ttctgctaatgttatacaca

SEQ ID NO: 96 - DNA, 20 bp, Cannabis sativa
attctgctaatgttatacac

SEQ ID NO: 97 - DNA, 20 bp, Cannabis sativa
tcttgtaccagattcttcga

SEQ ID NO: 98 - DNA, 20 bp, Cannabis sativa
ttcttgtaccagattcttcg

SEQ ID NO: 99 - DNA, 20 bp, Cannabis sativa
acttcaaccctcgaagaatc

SEQ ID NO: 100 - DNA, 20 bp, Cannabis sativa
aacttaattctagcttttca

SEQ ID NO: 101 - DNA, 20 bp, Cannabis sativa
ttgaaaagctagaattaagt

SEQ ID NO: 102 - DNA, 20 bp, Cannabis sativa
gtctttgtttattgacttca

SEQ ID NO: 103 - DNA, 20 bp, Cannabis sativa
tttatcagaggagcattgtc

SEQ ID NO: 104 - DNA, 20 bp, Cannabis sativa
ttcaaatcaaggtttatcag

SEQ ID NO: 105 - DNA, 20 bp, Cannabis sativa
caaattattcgttcaaatca

SEQ ID NO: 106 - DNA, 20 bp, Cannabis sativa
cttgatttgaacgaataatt

SEQ ID NO: 107 - DNA, 20 bp, Cannabis sativa
ctacattgctactgagctca

SEQ ID NO: 108 - DNA, 20 bp, Cannabis sativa
tcagtaaattctcgccgcaa

SEQ ID NO: 109 - DNA, 20 bp, Cannabis sativa
gtaaattctcgccgcaaagg

SEQ ID NO: 110 - DNA, 20 bp, Cannabis sativa
atgtgattccaccacctttg

SEQ ID NO: 111 - DNA, 20 bp, Cannabis sativa
aattctcgccgcaaaggtgg

SEQ ID NO: 112 - DNA, 20 bp, Cannabis sativa
actacagattgtagctataa

SEQ ID NO: 113 - DNA, 20 bp, Cannabis sativa
tactacagattgtagctata

SEQ ID NO: 114 - DNA, 20 bp, Cannabis sativa
ttttgttgattatatataaa

SEQ ID NO: 115 - DNA, 20 bp, Cannabis sativa
tatagggtacataaaagtca

SEQ ID NO: 116 - DNA, 20 bp, Cannabis sativa
tatctattctatacaatata

SEQ ID NO: 117 - DNA, 20 bp, Cannabis sativa
atatctattctatacaatat

SEQ ID NO: 118 - DNA, 20 bp, Cannabis sativa
atgttttctttttcattaat

SEQ ID NO: 119 - DNA, 20 bp, Cannabis sativa
gtacaaaatgaatttataaa

SEQ ID NO: 120 - DNA, 20 bp, Cannabis sativa
tgtacagagtcaatgtaaat

SEQ ID NO: 121 - DNA, 20 bp, Cannabis sativa
attgatgatttgggtaaatt

SEQ ID NO: 122 - DNA, 20 bp, Cannabis sativa
gtatttttaattgatgattt

SEQ ID NO: 123 - DNA, 20 bp, Cannabis sativa
agtatttttaattgatgatt

SEQ ID NO: 124 - DNA, 20 bp, Cannabis sativa
tgtgcctaattttctgtttt

SEQ ID NO: 125 - DNA, 20 bp, Cannabis sativa
atcaccaaaaacagaaaatt

SEQ ID NO: 126 - DNA, 20 bp, Cannabis sativa
gattaagcttcttcgtcatt

SEQ ID NO: 127 - DNA, 20 bp, Cannabis sativa
ggatgccaaacgcacgcttc

SEQ ID NO: 128 - DNA, 20 bp, Cannabis sativa
tggatgccaaacgcacgctt

SEQ ID NO: 129 - DNA, 20 bp, Cannabis sativa
aatctcccgaagcgtgcgtt

SEQ ID NO: 130 - DNA, 20 bp, Cannabis sativa
taacgcctttgataatcata

SEQ ID NO: 131 - DNA, 20 bp, Cannabis sativa
ggcatccatatgattatcaa

SEQ ID NO: 132 - DNA, 20 bp, Cannabis sativa
gaattcggagacgaatgaga

SEQ ID NO: 133 - DNA, 20 bp, Cannabis sativa
ttacagctcggttttgaatt

SEQ ID NO: 134 - DNA, 20 bp, Cannabis sativa
tttgtttttgaattacagct

SEQ ID NO: 135 - DNA, 20 bp, Cannabis sativa
gattttgattcgctactttt

SEQ ID NO: 136 - DNA, 20 bp, Cannabis sativa
gaggagcttatggtatcgtt

SEQ ID NO: 137 - DNA, 20 bp, Cannabis sativa
cctattggtagaggagctta

SEQ ID NO: 138 - DNA, 20 bp, Cannabis sativa
ccgattatgcctattggtag

SEQ ID NO: 139 - DNA, 20 bp, Cannabis sativa
ccataagctcctctaccaat

SEQ ID NO: 140 - DNA, 20 bp, Cannabis sativa
cgacctccgattatgcctat

SEQ ID NO: 141 - DNA, 20 bp, Cannabis sativa
cctctaccaataggcataat

SEQ ID NO: 142 - DNA, 20 bp, Cannabis sativa
ctaccaataggcataatcgg

SEQ ID NO: 143 - DNA, 20 bp, Cannabis sativa
cataatcggaggtcgatact

SEQ ID NO: 144 - DNA, 20 bp, Cannabis sativa
tacattcagtacaacatatt

SEQ ID NO: 145 - DNA, 20 bp, Cannabis sativa
actgaatgtactgacctcca

SEQ ID NO: 146 - DNA, 20 bp, Cannabis sativa
ctgaatgtactgacctccat

SEQ ID NO: 147 - DNA, 20 bp, Cannabis sativa
cccgcgattccttcccatgg

SEQ ID NO: 148 - DNA, 20 bp, Cannabis sativa
atgtactgacctccatggga

SEQ ID NO: 149 - DNA, 20 bp, Cannabis sativa
tttcccgcgattccttccca

SEQ ID NO: 150 - DNA, 20 bp, Cannabis sativa
acctccatgggaaggaatcg

SEQ ID NO: 151 - DNA, 20 bp, Cannabis sativa
cctccatgggaaggaatcgc

SEQ ID NO: 152 - DNA, 20 bp, Cannabis sativa
cggaggaggacctcagtaca

SEQ ID NO: 153 - DNA, 20 bp, Cannabis sativa
gcgggaaaatccgtgtactg

SEQ ID NO: 154 - DNA, 20 bp, Cannabis sativa
cgtgtactgaggtcctcctc

SEQ ID NO: 155 - DNA, 20 bp, Cannabis sativa
gccgctcccggagccggagg

SEQ ID NO: 156 - DNA, 20 bp, Cannabis sativa
gccgccgctcccggagccgg

SEQ ID NO: 157 - DNA, 20 bp, Cannabis sativa
ctgaggtcctcctccggctc

SEQ ID NO: 158 - DNA, 20 bp, Cannabis sativa
tgaggtcctcctccggctcc

SEQ ID NO: 159 - DNA, 20 bp, Cannabis sativa
ggtgccgccgctcccggagc

SEQ ID NO: 160 - DNA, 20 bp, Cannabis sativa
tcctcctccggctccgggag

SEQ ID NO: 161 - DNA, 20 bp, Cannabis sativa
cttcccggtgccgccgctcc

SEQ ID NO: 162 - DNA, 20 bp, Cannabis sativa
tcctccggctccgggagcgg

SEQ ID NO: 163 - DNA, 20 bp, Cannabis sativa
ggctccgggagcggcggcac

SEQ ID NO: 164 - DNA, 20 bp, Cannabis sativa
gctccgggagcggcggcacc

SEQ ID NO: 165 - DNA, 20 bp, Cannabis sativa
gccactcataacaatcttcc

SEQ ID NO: 166 - DNA, 20 bp, Cannabis sativa
accgggaagattgttatgag

SEQ ID NO: 167 - DNA, 20 bp, Cannabis sativa
agagaagagaaacttcaata

SEQ ID NO: 168 - DNA, 20 bp, Cannabis sativa
tggtttaaaagtcttgtctt

SEQ ID NO: 169 - DNA, 20 bp, Cannabis sativa
atttttgtttgattgaaatt

SEQ ID NO: 170 - DNA, 20 bp, Cannabis sativa
ttattattattattattatg

SEQ ID NO: 171 - DNA, 3213 bp, Cannabis sativa
acctaaaactttttcatttaatttttttaatgataaagtgcgttataatttttaagaactgtaaatttttattatttattttttgaatattttataatattatcgaatatttttttggatattataaattatttaaaatttataaacaattacaatatatataaatcaatcgtaaacaaatattagactaaaaatgtcttgtgatattgataaaaagaatatatggaaacgatccttgaaagggtgtaatataaaatttactcaatatcataaaagcatgttttacaatatcacttttcactatccaattcaagaaaaatccacacatcattacaatttacaaataccaagagtcaaaatatgagcacccacaaattaataattacacccaaatatcttttaaaccaatcacatttacttaaattagaactcactcattgtcaagtcactatgtataaaaattaattaattaattaatgggtgtaatttgttgacagatactcatatcatttatcgtaccaaaataacaacaacaataaaaacaaaacaaacaaaagaacaaaaaaaaccaaatttattacactcctttggttcttcttcaaaggaaataataaagcctcaaaataaaactacataacagagtagaataacaaagtcatatatagatatagtctcataattataggtagctagcataattatatttacaccttacaagataaccatggttgctatgcaatgcaaagatctcaaactaccaaagtagttgttaaaaaccaaatctttgaatgcagaatcatggtgttaaaaattcaaatgctgaacaagtagcatcagtagtattcttcatagaaaataatatagtagatagatagtactagggtactgctggttccaaaagctctcaatttaagtcaagaaaaagaaaaggtttttttttttttttaatgaaagagaggcattttttgactcatatattgatactattgacagaagcagcttcaggatggtaatgaagcatttcactccacatcatttcccttatcattggttccatcaaattctcatctatatcaaggctgattggtacctgggcaggagggtttcgtctcgaatcgtatagtcctgacatgtaagggtgttgaagtgcttctgtaacagtgatcctctttgttggatcaaacacaagcattctctctagcaaatctattgctaatggatcagcatggggatatagcctggaaaaatgtgtccccctagaataaggcagggattttatgaactttctggcctttggattatcaataaactcgatatctgtttcacgctggctgccaagaacattgatgattagtttaagctggttgaggcattcagttccgggaaagattggttttctaccaaggatttcagcaaatatgcatcctacagaccagacatcaatggatgttccatagttgtcacaacagagaagtagctcgggtgcacgataccagcgagtcacaacatattctgtcatgaattgcccatttcctctactggttctcgctagaccaaaatcgcatatctttaggtcacaattagcattgacaaggaggttcccgggcttcaagtctcggtgaagaatgtttgcagagtgaagatacttgagcccgcgaagcaactgcacaacagtgaaacattcataagtaatggcaccgaaaattgcatcaatccactgaaaacagtacttaaaagaactgtaaaacacacaggcatatatagtataaacttgatattcaatcttgtacctgaaataaaaagtatttgcagtgatcactagaaagcggttgtgaggacttaataatttgatggaggtcagtatccatgagctcataaaccagataaacatctttgaaagttgctctatgaataggcatcataacatcctttaaagcaatcacattctcatgtcgaatatgcctaagaagcttcaactccctcagagtcctcagggcatctacgcggttctcaaacacgttgttgatcttcttaattgcaactttctcgttcgtttccctattgatcgacgagcacaccacgccataggcacctctccctattggtttgataggaacatacttggtgtcaatctcaaacaaagtttgccacattgagtagtaatgctttccttgttgcctaactccattagggggatcaaccaaagttgccattttccctacaaatcaccgaaatcaatcaacacccatttaagattaaatcacaagaaaaataattacaacaacataagttttacaacatacacttgttcaaagcttattaaactatgagcaacacaacccagttatacaatgagttaaaactaagaaaaagttaagaggaatgtacagagtatataaaagatgtgctgttaagactttagaatcaatccttacttgccaattctacttctgggttatgatcttctccctataaggttattctctacactttcttctcccctctttttttaaatagtaaaattcccacttttatgcctttttcccttagacttctctctctccttttcctcactttgttcacttatgaccatcctctcatatacttaatcctcttcctaaaaaaacctctcaaattcagttcacacacaaagctaatttattgaacctcaacaaaacaacaatcttttcctctagattcttacaactccttaattcttctccttattctcaaaattaagccaagaatcaaacttttactctgttccaagtaaaagattccttcaaaaacaaaaccaagcaaaacccatctttccaaaaataaagctcagtactttcaccctccaattaaaagaaaagaaaaaataaatttttgcagaaaccccaaaaagggaaaaagaaagagaaagagactaacctcaataattgacaaccaacatgaataatcagaaaaatctgagataattgaaggcaaatttcatcaatatcctttatctgaaacaggtctgtgcaatcagatcacgaaagaaaaaagcttaatgaatccttcaaatcgtcttgaacagaaaaaaacttaggcgatacccaccattaaagatacaaactttaagaaaacccaatacttaaaatgagaaaaatggaaacgggtttggggagaatggggcaggagatcatgagccaaacccacccatgacactacccacacggcttcgta

SEQ ID NO: 172 - DNA, 1113 bp, Artificial Sequence
  CsBif2 CDS
atggcaactttggttgatccccctaatggagttaggcaacaaggaaagcattactactcaatgtggcaaactttgtttgagattgacaccaagtatgttcctatcaaaccaatagggagaggtgcctatggcgtggtgtgctcgtcgatcaatagggaaacgaacgagaaagttgcaattaagaagatcaacaacgtgtttgagaaccgcgtagatgccctgaggactctgagggagttgaagcttcttaggcatattcgacatgagaatgtgattgctttaaaggatgttatgatgcctattcatagagcaactttcaaagatgtttatctggtttatgagctcatggatactgacctccatcaaattattaagtcctcacaaccgctttctagtgatcactgcaaatactttttacagttgcttcgcgggctcaagtatcttcactctgcaaacattcttcaccgagacttgaagcccgggaacctccttgtcaatgctaattgtgacctaaagatatgcgattttggtctagcgagaaccagtagaggaaatgggcaattcatgacagaatatgttgtgactcgctggtatcgtgcacccgagctacttctctgttgtgacaactatggaacatccattgatgtctggtctgtaggatgcatatttgctgaaatccttggtagaaaaccaatctttcccggaactgaatgcctcaaccagcttaaactaatcatcaatgttcttggcagccagcgtgaaacagatatcgagtttattgataatccaaaggccagaaagttcataaaatccctgccttattctagggggacacatttttccaggctatatccccatgctgatccattagcaatagatttgctagagagaatgcttgtgtttgatccaacaaagaggatcactgttacagaagcacttcaacacccttacatgtcaggactatacgattcgagacgaaaccctcctgcccaggtaccaatcagccttgatatagatgagaatttgatggaaccaatgataagggaaatgatgtggagtgaaatgcttcattaccatcctgaagctgcttctgtcaatagtatcaatatatga

SEQ ID NO: 173 - protein, 370 aa, Artificial Sequence
  CsBif2 AA
MATLVDPPNGVRQQGKHYYSMWQTLFEIDTKYVPIKPIGRGAYGVVCSSINRETNEKVAIKKINNVFENRVDALRTLRELKLLRHIRHENVIALKDVMMPIHRATFKDVYLVYELMDTDLHQIIKSSQPLSSDHCKYFLQLLRGLKYLHSANILHRDLKPGNLLVNANCDLKICDFGLARTSRGNGQFMTEYVVTRWYRAPELLLCCDNYGTSIDVWSVGCIFAEILGRKPIFPGTECLNQLKLIINVLGSQRETDIEFIDNPKARKFIKSLPYSRGTHFSRLYPHADPLAIDLLERMLVFDPTKRITVTEALQHPYMSGLYDSRRNPPAQVPISLDIDENLMEPMIREMMWSEMLHYHPEAASVNSINI

SEQ ID NO: 174 - DNA, 20 bp, Cannabis sativa
aaattaaatgaaaaagtttt

SEQ ID NO: 175 - DNA, 20 bp, Cannabis sativa
aatattatcgaatatttttt

SEQ ID NO: 176 - DNA, 20 bp, Cannabis sativa
atattgataaaaagaatata

SEQ ID NO: 177 - DNA, 20 bp, Cannabis sativa
atatggaaacgatccttgaa

SEQ ID NO: 178 - DNA, 20 bp, Cannabis sativa
tatggaaacgatccttgaaa

SEQ ID NO: 179 - DNA, 20 bp, Cannabis sativa
ttttatattacaccctttca

SEQ ID NO: 180 - DNA, 20 bp, Cannabis sativa
tgtgtggatttttcttgaat

SEQ ID NO: 181 - DNA, 20 bp, Cannabis sativa
ttgtaaattgtaatgatgtg

SEQ ID NO: 182 - DNA, 20 bp, Cannabis sativa
ggtgctcatattttgactct

SEQ ID NO: 183 - DNA, 20 bp, Cannabis sativa
gggtgtaattattaatttgt

SEQ ID NO: 184 - DNA, 20 bp, Cannabis sativa
tgggtgtaattattaatttg

SEQ ID NO: 185 - DNA, 20 bp, Cannabis sativa
gattggtttaaaagatattt

SEQ ID NO: 186 - DNA, 20 bp, Cannabis sativa
tgattggtttaaaagatatt

SEQ ID NO: 187 - DNA, 20 bp, Cannabis sativa
ctaatttaagtaaatgtgat

SEQ ID NO: 188 - DNA, 20 bp, Cannabis sativa
aaaattaattaattaattaa

SEQ ID NO: 189 - DNA, 20 bp, Cannabis sativa
aaattaattaattaattaat

SEQ ID NO: 190 - DNA, 20 bp, Cannabis sativa
ttttattgttgttgttattt

SEQ ID NO: 191 - DNA, 20 bp, Cannabis sativa
ccaaaggagtgtaataaatt

SEQ ID NO: 192 - DNA, 20 bp, Cannabis sativa
ccaaatttattacactcctt

SEQ ID NO: 193 - DNA, 20 bp, Cannabis sativa
ttcctttgaagaagaaccaa

SEQ ID NO: 194 - DNA, 20 bp, Cannabis sativa
ctcctttggttcttcttcaa

SEQ ID NO: 195 - DNA, 20 bp, Cannabis sativa
tgttatgtagttttattttg

SEQ ID NO: 196 - DNA, 20 bp, Cannabis sativa
agatatagtctcataattat

SEQ ID NO: 197 - DNA, 20 bp, Cannabis sativa
gcaaccatggttatcttgta

SEQ ID NO: 198 - DNA, 20 bp, Cannabis sativa
tacaccttacaagataacca

SEQ ID NO: 199 - DNA, 20 bp, Cannabis sativa
tttgcattgcatagcaacca

SEQ ID NO: 200 - DNA, 20 bp, Cannabis sativa
ttggtttttaacaactactt

SEQ ID NO: 201 - DNA, 20 bp, Cannabis sativa
tgattctgcattcaaagatt

SEQ ID NO: 202 - DNA, 20 bp, Cannabis sativa
aatctttgaatgcagaatca

SEQ ID NO: 203 - DNA, 20 bp, Cannabis sativa
tatagtagatagatagtact

SEQ ID NO: 204 - DNA, 20 bp, Cannabis sativa
atagtagatagatagtacta

SEQ ID NO: 205 - DNA, 20 bp, Cannabis sativa
agatagtactagggtactgc

SEQ ID NO: 206 - DNA, 20 bp, Cannabis sativa
tgacttaaattgagagcttt

SEQ ID NO: 207 - DNA, 20 bp, Cannabis sativa
tttaagtcaagaaaaagaaa

SEQ ID NO: 208 - DNA, 20 bp, Cannabis sativa
ttttttttttaatgaaagag

SEQ ID NO: 209 - DNA, 20 bp, Cannabis sativa
ctattgacagaagcagcttc

SEQ ID NO: 210 - DNA, 20 bp, Cannabis sativa
tgacagaagcagcttcagga

SEQ ID NO: 211 - DNA, 20 bp, Cannabis sativa
caatgataagggaaatgatg

SEQ ID NO: 212 - DNA, 20 bp, Cannabis sativa
tttgatggaaccaatgataa

SEQ ID NO: 213 - DNA, 20 bp, Cannabis sativa
cacatcatttcccttatcat

SEQ ID NO: 214 - DNA, 20 bp, Cannabis sativa
atttgatggaaccaatgata

SEQ ID NO: 215 - DNA, 20 bp, Cannabis sativa
tgatatagatgagaatttga

SEQ ID NO: 216 - DNA, 20 bp, Cannabis sativa
tcaaattctcatctatatca

SEQ ID NO: 217 - DNA, 20 bp, Cannabis sativa
tcatctatatcaaggctgat

SEQ ID NO: 218 - DNA, 20 bp, Cannabis sativa
tatcaaggctgattggtacc

SEQ ID NO: 219 - DNA, 20 bp, Cannabis sativa
atcaaggctgattggtacct

SEQ ID NO: 220 - DNA, 20 bp, Cannabis sativa
aggctgattggtacctgggc

SEQ ID NO: 221 - DNA, 20 bp, Cannabis sativa
gagacgaaaccctcctgccc

SEQ ID NO: 222 - DNA, 20 bp, Cannabis sativa
ctgattggtacctgggcagg

SEQ ID NO: 223 - DNA, 20 bp, Cannabis sativa
tgattggtacctgggcagga

SEQ ID NO: 224 - DNA, 20 bp, Cannabis sativa
cttcaacacccttacatgtc

SEQ ID NO: 225 - DNA, 20 bp, Cannabis sativa
tcgtatagtcctgacatgta

SEQ ID NO: 226 - DNA, 20 bp, Cannabis sativa
cgtatagtcctgacatgtaa

SEQ ID NO: 227 - DNA, 20 bp, Cannabis sativa
gtaacagtgatcctctttgt

SEQ ID NO: 228 - DNA, 20 bp, Cannabis sativa
ttgtgtttgatccaacaaag

SEQ ID NO: 229 - DNA, 20 bp, Cannabis sativa
tctagcaaatctattgctaa

SEQ ID NO: 230 - DNA, 20 bp, Cannabis sativa
ctattgctaatggatcagca

SEQ ID NO: 231 - DNA, 20 bp, Cannabis sativa
tattgctaatggatcagcat

SEQ ID NO: 232 - DNA, 20 bp, Cannabis sativa
attgctaatggatcagcatg

SEQ ID NO: 233 - DNA, 20 bp, Cannabis sativa
atcagcatggggatatagcc

SEQ ID NO: 234 - DNA, 20 bp, Cannabis sativa
ctagggggacacatttttcc

SEQ ID NO: 235 - DNA, 20 bp, Cannabis sativa
aatccctgccttattctagg

SEQ ID NO: 236 - DNA, 20 bp, Cannabis sativa
aaatccctgccttattctag

SEQ ID NO: 237 - DNA, 20 bp, Cannabis sativa
aaaatccctgccttattcta

SEQ ID NO: 238 - DNA, 20 bp, Cannabis sativa
aaatgtgtccccctagaata

SEQ ID NO: 239 - DNA, 20 bp, Cannabis sativa
taaaatccctgccttattct

SEQ ID NO: 240 - DNA, 20 bp, Cannabis sativa
gtgtccccctagaataaggc

SEQ ID NO: 241 - DNA, 20 bp, Cannabis sativa
tgtccccctagaataaggca

SEQ ID NO: 242 - DNA, 20 bp, Cannabis sativa
cagggattttatgaactttc

SEQ ID NO: 243 - DNA, 20 bp, Cannabis sativa
tttatgaactttctggcctt

SEQ ID NO: 244 - DNA, 20 bp, Cannabis sativa
cgagtttattgataatccaa

SEQ ID NO: 245 - DNA, 20 bp, Cannabis sativa
actcgatatctgtttcacgc

SEQ ID NO: 246 - DNA, 20 bp, Cannabis sativa
aaactaatcatcaatgttct

SEQ ID NO: 247 - DNA, 20 bp, Cannabis sativa
cattgatgattagtttaagc

SEQ ID NO: 248 - DNA, 20 bp, Cannabis sativa
tgattagtttaagctggttg

SEQ ID NO: 249 - DNA, 20 bp, Cannabis sativa
ctggttgaggcattcagttc

SEQ ID NO: 250 - DNA, 20 bp, Cannabis sativa
tggttgaggcattcagttcc

SEQ ID NO: 251 - DNA, 20 bp, Cannabis sativa
ggtagaaaaccaatctttcc

SEQ ID NO: 252 - DNA, 20 bp, Cannabis sativa
cattcagttccgggaaagat

SEQ ID NO: 253 - DNA, 20 bp, Cannabis sativa
gaaagattggttttctacca

SEQ ID NO: 254 - DNA, 20 bp, Cannabis sativa
tgcatatttgctgaaatcct

SEQ ID NO: 255 - DNA, 20 bp, Cannabis sativa
tccattgatgtctggtctgt

SEQ ID NO: 256 - DNA, 20 bp, Cannabis sativa
atggaacatccattgatgtc

SEQ ID NO: 257 - DNA, 20 bp, Cannabis sativa
tcctacagaccagacatcaa

SEQ ID NO: 258 - DNA, 20 bp, Cannabis sativa
cttctctgttgtgacaacta

SEQ ID NO: 259 - DNA, 20 bp, Cannabis sativa
gtcacaacagagaagtagct

SEQ ID NO: 260 - DNA, 20 bp, Cannabis sativa
tcacaacagagaagtagctc

SEQ ID NO: 261 - DNA, 20 bp, Cannabis sativa
cagaatatgttgtgactcgc

SEQ ID NO: 262 - DNA, 20 bp, Cannabis sativa
cgagaaccagtagaggaaat

SEQ ID NO: 263 - DNA, 20 bp, Cannabis sativa
gcgagaaccagtagaggaaa

SEQ ID NO: 264 - DNA, 20 bp, Cannabis sativa
gaattgcccatttcctctac

SEQ ID NO: 265 - DNA, 20 bp, Cannabis sativa
ggtctagcgagaaccagtag

SEQ ID NO: 266 - DNA, 20 bp, Cannabis sativa
gacctaaagatatgcgattt

SEQ ID NO: 267 - DNA, 20 bp, Cannabis sativa
gaccaaaatcgcatatcttt

SEQ ID NO: 268 - DNA, 20 bp, Cannabis sativa
ggtcacaattagcattgaca

SEQ ID NO: 269 - DNA, 20 bp, Cannabis sativa
cacaattagcattgacaagg

SEQ ID NO: 270 - DNA, 20 bp, Cannabis sativa
agcattgacaaggaggttcc

SEQ ID NO: 271 - DNA, 20 bp, Cannabis sativa
gcattgacaaggaggttccc

SEQ ID NO: 272 - DNA, 20 bp, Cannabis sativa
ttcaccgagacttgaagccc

SEQ ID NO: 273 - DNA, 20 bp, Cannabis sativa
cttcaccgagacttgaagcc

SEQ ID NO: 274 - DNA, 20 bp, Cannabis sativa
ggttcccgggcttcaagtct

SEQ ID NO: 275 - DNA, 20 bp, Cannabis sativa
ctgttgtgcagttgcttcgc

SEQ ID NO: 276 - DNA, 20 bp, Cannabis sativa
actgttgtgcagttgcttcg

SEQ ID NO: 277 - DNA, 20 bp, Cannabis sativa
agtgaaacattcataagtaa

SEQ ID NO: 278 - DNA, 20 bp, Cannabis sativa
cagtggattgatgcaatttt

SEQ ID NO: 279 - DNA, 20 bp, Cannabis sativa
cttttaagtactgttttcag

SEQ ID NO: 280 - DNA, 20 bp, Cannabis sativa
aaaagaactgtaaaacacac

SEQ ID NO: 281 - DNA, 20 bp, Cannabis sativa
ctgcaaatactttttatttc

SEQ ID NO: 282 - DNA, 20 bp, Cannabis sativa
ttgcagtgatcactagaaag

SEQ ID NO: 283 - DNA, 20 bp, Cannabis sativa
atcactagaaagcggttgtg

SEQ ID NO: 284 - DNA, 20 bp, Cannabis sativa
gtgaggacttaataatttga

SEQ ID NO: 285 - DNA, 20 bp, Cannabis sativa
aggacttaataatttgatgg

SEQ ID NO: 286 - DNA, 20 bp, Cannabis sativa
ttatctggtttatgagctca

SEQ ID NO: 287 - DNA, 20 bp, Cannabis sativa
aactttcaaagatgtttatc

SEQ ID NO: 288 - DNA, 20 bp, Cannabis sativa
ttgaaagttgctctatgaat

SEQ ID NO: 289 - DNA, 20 bp, Cannabis sativa
tgagaatgtgattgctttaa

SEQ ID NO: 290 - DNA, 20 bp, Cannabis sativa
tgagggagttgaagcttctt

SEQ ID NO: 291 - DNA, 20 bp, Cannabis sativa
agatgccctgaggactctga

SEQ ID NO: 292 - DNA, 20 bp, Cannabis sativa
tagatgccctgaggactctg

SEQ ID NO: 293 - DNA, 20 bp, Cannabis sativa
tcaactccctcagagtcctc

SEQ ID NO: 294 - DNA, 20 bp, Cannabis sativa
caactccctcagagtcctca

SEQ ID NO: 295 - DNA, 20 bp, Cannabis sativa
agaaccgcgtagatgccctg

SEQ ID NO: 296 - DNA, 20 bp, Cannabis sativa
gagtcctcagggcatctacg

SEQ ID NO: 297 - DNA, 20 bp, Cannabis sativa
ggtgtgctcgtcgatcaata

SEQ ID NO: 298 - DNA, 20 bp, Cannabis sativa
tggtgtgctcgtcgatcaat

SEQ ID NO: 299 - DNA, 20 bp, Cannabis sativa
cgacgagcacaccacgccat

SEQ ID NO: 300 - DNA, 20 bp, Cannabis sativa
agggagaggtgcctatggcg

SEQ ID NO: 301 - DNA, 20 bp, Cannabis sativa
ccaatagggagaggtgccta

SEQ ID NO: 302 - DNA, 20 bp, Cannabis sativa
cctatcaaaccaatagggag

SEQ ID NO: 303 - DNA, 20 bp, Cannabis sativa
ccataggcacctctccctat

SEQ ID NO: 304 - DNA, 20 bp, Cannabis sativa
atgttcctatcaaaccaata

SEQ ID NO: 305 - DNA, 20 bp, Cannabis sativa
tatgttcctatcaaaccaat

SEQ ID NO: 306 - DNA, 20 bp, Cannabis sativa
cctctccctattggtttgat

SEQ ID NO: 307 - DNA, 20 bp, Cannabis sativa
tggtttgataggaacatact

SEQ ID NO: 308 - DNA, 20 bp, Cannabis sativa
gaaagcattactactcaatg

SEQ ID NO: 309 - DNA, 20 bp, Cannabis sativa
cctaatggagttaggcaaca

SEQ ID NO: 310 - DNA, 20 bp, Cannabis sativa
ttgatccccctaatggagtt

SEQ ID NO: 311 - DNA, 20 bp, Cannabis sativa
ccttgttgcctaactccatt

SEQ ID NO: 312 - DNA, 20 bp, Cannabis sativa
cttgttgcctaactccatta

SEQ ID NO: 313 - DNA, 20 bp, Cannabis sativa
ttgttgcctaactccattag

SEQ ID NO: 314 - DNA, 20 bp, Cannabis sativa
tgttgcctaactccattagg

SEQ ID NO: 315 - DNA, 20 bp, Cannabis sativa
actttggttgatccccctaa

SEQ ID NO: 316 - DNA, 20 bp, Cannabis sativa
tgtagggaaaatggcaactt

SEQ ID NO: 317 - DNA, 20 bp, Cannabis sativa
tcggtgatttgtagggaaaa

SEQ ID NO: 318 - DNA, 20 bp, Cannabis sativa
attgatttcggtgatttgta

SEQ ID NO: 319 - DNA, 20 bp, Cannabis sativa
gattgatttcggtgatttgt

SEQ ID NO: 320 - DNA, 20 bp, Cannabis sativa
aaatgggtgttgattgattt

SEQ ID NO: 321 - DNA, 20 bp, Cannabis sativa
cttgtgatttaatcttaaat

SEQ ID NO: 322 - DNA, 20 bp, Cannabis sativa
tcttgtgatttaatcttaaa

SEQ ID NO: 323 - DNA, 20 bp, Cannabis sativa
ttttaactcattgtataact

SEQ ID NO: 324 - DNA, 20 bp, Cannabis sativa
gttttaactcattgtataac

SEQ ID NO: 325 - DNA, 20 bp, Cannabis sativa
aaaactaagaaaaagttaag

SEQ ID NO: 326 - DNA, 20 bp, Cannabis sativa
agaagtagaattggcaagta

SEQ ID NO: 327 - DNA, 20 bp, Cannabis sativa
tcataacccagaagtagaat

SEQ ID NO: 328 - DNA, 20 bp, Cannabis sativa
ttacttgccaattctacttc

SEQ ID NO: 329 - DNA, 20 bp, Cannabis sativa
tacttgccaattctacttct

SEQ ID NO: 330 - DNA, 20 bp, Cannabis sativa
gttatgatcttctccctata

SEQ ID NO: 331 - DNA, 20 bp, Cannabis sativa
gtgtagagaataaccttata

SEQ ID NO: 332 - DNA, 20 bp, Cannabis sativa
agtgtagagaataaccttat

SEQ ID NO: 333 - DNA, 20 bp, Cannabis sativa
tttactatttaaaaaaagag

SEQ ID NO: 334 - DNA, 20 bp, Cannabis sativa
ttttactatttaaaaaaaga

SEQ ID NO: 335 - DNA, 20 bp, Cannabis sativa
attttactatttaaaaaaag

SEQ ID NO: 336 - DNA, 20 bp, Cannabis sativa
agggaaaaaggcataaaagt

SEQ ID NO: 337 - DNA, 20 bp, Cannabis sativa
aagggaaaaaggcataaaag

SEQ ID NO: 338 - DNA, 20 bp, Cannabis sativa
gagagaagtctaagggaaaa

SEQ ID NO: 339 - DNA, 20 bp, Cannabis sativa
aaggagagagagaagtctaa

SEQ ID NO: 340 - DNA, 20 bp, Cannabis sativa
aaaggagagagagaagtcta

SEQ ID NO: 341 - DNA, 20 bp, Cannabis sativa
aagtgaacaaagtgaggaaa

SEQ ID NO: 342 - DNA, 20 bp, Cannabis sativa
ggtcataagtgaacaaagtg

SEQ ID NO: 343 - DNA, 20 bp, Cannabis sativa
ggattaagtatatgagagga

SEQ ID NO: 344 - DNA, 20 bp, Cannabis sativa
aagaggattaagtatatgag

SEQ ID NO: 345 - DNA, 20 bp, Cannabis sativa
tgagaggtttttttaggaag

SEQ ID NO: 346 - DNA, 20 bp, Cannabis sativa
tgaatttgagaggttttttt

SEQ ID NO: 347 - DNA, 20 bp, Cannabis sativa
gtgtgtgaactgaatttgag

SEQ ID NO: 348 - DNA, 20 bp, Cannabis sativa
aaaagattgttgttttgttg

SEQ ID NO: 349 - DNA, 20 bp, Cannabis sativa
aaggagttgtaagaatctag

SEQ ID NO: 350 - DNA, 20 bp, Cannabis sativa
gagaataaggagaagaatta

SEQ ID NO: 351 - DNA, 20 bp, Cannabis sativa
ttggcttaattttgagaata

SEQ ID NO: 352 - DNA, 20 bp, Cannabis sativa
cagagtaaaagtttgattct

SEQ ID NO: 353 - DNA, 20 bp, Cannabis sativa
ttttgaaggaatcttttact

SEQ ID NO: 354 - DNA, 20 bp, Cannabis sativa
ttgcttggttttgtttttga

SEQ ID NO: 355 - DNA, 20 bp, Cannabis sativa
ttggaaagatgggttttgct

SEQ ID NO: 356 - DNA, 20 bp, Cannabis sativa
agctttatttttggaaagat

SEQ ID NO: 357 - DNA, 20 bp, Cannabis sativa
gagctttatttttggaaaga

SEQ ID NO: 358 - DNA, 20 bp, Cannabis sativa
aaagtactgagctttatttt

SEQ ID NO: 359 - DNA, 20 bp, Cannabis sativa
tttcttttcttttaattgga

SEQ ID NO: 360 - DNA, 20 bp, Cannabis sativa
ttttcttttcttttaattgg

SEQ ID NO: 361 - DNA, 20 bp, Cannabis sativa
attttttcttttcttttaat

SEQ ID NO: 362 - DNA, 20 bp, Cannabis sativa
tttttgcagaaaccccaaaa

SEQ ID NO: 363 - DNA, 20 bp, Cannabis sativa
ttttgcagaaaccccaaaaa

SEQ ID NO: 364 - DNA, 20 bp, Cannabis sativa
tctttctttttccctttttg

SEQ ID NO: 365 - DNA, 20 bp, Cannabis sativa
ctctttctttttcccttttt

SEQ ID NO: 366 - DNA, 20 bp, Cannabis sativa
tctctttctttttccctttt

SEQ ID NO: 367 - DNA, 20 bp, Cannabis sativa
atgttggttgtcaattattg

SEQ ID NO: 368 - DNA, 20 bp, Cannabis sativa
atttttctgattattcatgt

SEQ ID NO: 369 - DNA, 20 bp, Cannabis sativa
gaaaaatctgagataattga

SEQ ID NO: 370 - DNA, 20 bp, Cannabis sativa
cacagacctgtttcagataa

SEQ ID NO: 371 - DNA, 20 bp, Cannabis sativa
caatatcctttatctgaaac

SEQ ID NO: 372 - DNA, 20 bp, Cannabis sativa
ttctgttcaagacgatttga

SEQ ID NO: 373 - DNA, 20 bp, Cannabis sativa
tcttgaacagaaaaaaactt

SEQ ID NO: 374 - DNA, 20 bp, Cannabis sativa
aagtttgtatctttaatggt

SEQ ID NO: 375 - DNA, 20 bp, Cannabis sativa
aaagtttgtatctttaatgg

SEQ ID NO: 376 - DNA, 20 bp, Cannabis sativa
cttaaagtttgtatctttaa

SEQ ID NO: 377 - DNA, 20 bp, Cannabis sativa
tttttctcattttaagtatt

SEQ ID NO: 378 - DNA, 20 bp, Cannabis sativa
atttttctcattttaagtat

SEQ ID NO: 379 - DNA, 20 bp, Cannabis sativa
aatacttaaaatgagaaaaa

SEQ ID NO: 380 - DNA, 20 bp, Cannabis sativa
taaaatgagaaaaatggaaa

SEQ ID NO: 381 - DNA, 20 bp, Cannabis sativa
aaaatgagaaaaatggaaac

SEQ ID NO: 382 - DNA, 20 bp, Cannabis sativa
gagaaaaatggaaacgggtt

SEQ ID NO: 383 - DNA, 20 bp, Cannabis sativa
agaaaaatggaaacgggttt

SEQ ID NO: 384 - DNA, 20 bp, Cannabis sativa
gaaaaatggaaacgggtttg

SEQ ID NO: 385 - DNA, 20 bp, Cannabis sativa
ggaaacgggtttggggagaa

SEQ ID NO: 386 - DNA, 20 bp, Cannabis sativa
gaaacgggtttggggagaat

SEQ ID NO: 387 - DNA, 20 bp, Cannabis sativa
aaacgggtttggggagaatg

SEQ ID NO: 388 - DNA, 20 bp, Cannabis sativa
gggtttggggagaatggggc

SEQ ID NO: 389 - DNA, 20 bp, Cannabis sativa
ggtagtgtcatgggtgggtt

SEQ ID NO: 390 - DNA, 7405 bp, Cannabis sativa
aataatatactatgcaacaaaacagaaatatactgtacaacaaatctacatactaacacctataaaaaataaaaataaaaatcgacataactttaaaataaaaaaagattttaacaaaactaatatagattatactataatttttaagtaattggtttttttaaaaaatatataaaaaacaataatgtattaaaagactgtaaataataaataatgtaatagcgtcatttctttacaaattttgaaataaaaacatttacttatataattttataatttaagttcatttactataattttttaaaattaacttgggtgtatatttttcaaacaaatagaagagactctaatcactagaaagaaggaagcaggagccgttcaaagcctcaccttataaggctaggctaatttgaaaaataaattacgtgtagttataaaaattaatattttcataattaaaatagggtgaactaagtattttccctttttttattgaatgaacttactctttgtgtgtatttacccttttttgggccccattataaagtcggtaatttgaaattggaccaactagaatctaattcaaaccaaatttaaatacggttcaaacatgataactttttactaagttctggaccaaatcgacccaaattatgaggaaatgtgcttagtacaataaaaacggcaaaatatatatatattaaatataattcaacgtatccgaatttagtattcaactctttctttctttttctttctttcttctttctttttcttttatttgtattcaaccgatttttttttctttttttgaaatggtattcaactgaattagcaatacttttgccgcatgaatgaaaatgtagaaatttagaagtgaaaagcgaagtaatgagagagagagggaacattctatgcatttattgtagtagtcaaagggtacaaggaccacactgctacaaaaagaataaataacctagaagcagcgttaaacaaaagaaatcttaaaagatgagatgaatgaaaaagagaattacccaacccacagaaacaaatttacatcactactcaacccaatctctctttctactgcaggaaggttaccctgccaaagaagtaaaaatactccggcccgagaaccttaagaatcatcaaccgtgcgtattgccccaatttgaggcctctgcacaaagaatataaattttgttgtgtttttcaaaaaaagaaaacctaaggttaatctgtcactgctcccagaatcatcaatcagaatatcttttcgtgttgttaggaccccattagagaaaatgaagttagcaagtcaggctctagtttgatattgtggctccagagagcaggtcaataaaaaaatggtgtgtaatctaataatgaaatggtgaaatggtttttgctttttttcctgttcttctttcaatctaacctgtgacctgtctctgaccctagaaatgaagggcctctgactttaaggcgttttgctccaatatctactgggtgtggttccctcgtgctcttacccaactctaactgaaacctatcctttttgggttgttctctcagtctgttactactaccctcattaaccacattcttgaattttatagagatcggaatggtaccccttccttgacaaatctttctgtcgttgaaagagtgtcaatagctctatgattagtgagcgtagttggttagtttagaatttgatcccacaattctggtgcaacctgacgacccagatcttctgtcaggctgaaatttcttaagtcgtctgaacccctgtccattaaaaatttcagcaaccagaaaagttaatcatcaattgttaggacacaatactggtttatgaaactaggatctaaagtttacgtgtaaactaagtacctggtggtcagaatgcttcgataatatgttatcattcatgttcttcttttccatcttctgtgttgaacgacgtttaaccctgaatatctctgaatcagaactatcactttcttcatctagagtaggcctaaccagcttttcatgagaataatcttgatttacagtcaagtgttcatcacatgacggggaagacagagtagattcaagtgcactccgtgacaagtcttctgaaaactttctagagtcaagactcctcacatcaaagctagtgttcccatgtacctggagaaaataaatagatgcaacaaattagaatttcaaacacatgcactctaaataattctgaaaggatgcatatcagaatacagaagtagccaactaaaggagatctgtatcttacattgctgggagtgattgcggcttccgtagggtctacaggaagagaatgtagagtggatgtggtacaggacatagatgttcctaacatttcagaatttaaactcatgttccactacttgacacctgtgcactacacgaaacatgatcttgctcttttgattgatactgagtagtatcactaagttttgggatcgagtcaaatttgatcggacagtaaggaatgtatacctcctgcttaactttatgtgacatgtttgctaatggatctgaatacaagtcatgaaccctattagattgctgattaatcttcttttctggtactctatcctccatttcaaactttctagctgcatcttccatttctgatatatcctcccttaaacagagcgtacagtgtcttccacaggaaaaatttaaagacgtcatatctgcaacaagaaaagcaacaaacagatgaaaataagagtgtgtaagaatccaaggcagaaacatatagcattggtaaaatccccctgtgggctgctgttaagtaggaatggcaacccagtatcaaaagtcccattgggtagaggtcataattcaaaaagtttatatcccattaaaatggtgatgaatacacttctctctaatcaagtagagacagatcttttcaagataaataagcaattgaaatctcatataatccaaacacgcaaacactaggctggatagaagcataccatgacgaaggcagacaggatgcatgtaacagctacagttgacaaaagcaacataacagtcacgtttacataagctgcacaggatagttccatgccgatttgcagacacactagtacatatccctgatttcattagcaaccagcgagctcgatgcaagaaacggatcaacttcacaaaagaagttttaatggaacaatgagaggccaagtcagcagaagaatgatccaaatcttcaagctctaaactcgaatagagaagcattgcttctttgcaaagtagttcttcatgaggaagcaaaggcatcctactaagaagtgcatagcgttggctagcaacagcacccaagggaaaccagtcaccaattgcaaagttcactgcttcaccacaattgaagcctacagttccaaaaggaaaagtacaatttcttcaaataattacagtaaatatatatatatagatcaagagagagagagagagagaaattagcaacataaatgatgtttaccatgattaaatccagcatgatatgctctagggaacgtaactatgaactctccaggtttttgcactgccttgtacacagggacgtcatgatccaatagaatattaggaggaaataaagttgttttccccaataaaacatcaaaagccccatcctccccatcagcagaaaggatgtcatgagtatacacatgttccctaacaaccttttcaaactgcaaagcagcatgacctggaatgccataccaagtttttgaagcaccgcagtgatgataattaatactgcagtaagagtgagaaaattaatagagtggggagaatttgaataacacaaaattagcatgaaagttttatggcttcactgcctctcaagaataacatatacctgtatagataatgatcctcgacatgccaagcaaacatgctaaatagcattccaatatagagcataggatcagttactccctgtgaaaaaaaataaacattagtaatttaagaaagcacatcatatatagatgatgaaaagattgtagataagttagtattgttacaggaattgctctttctagaagacgcaatactgactttgatagccttgacagtctctgaaacaaaataaaaaagaattgaaattgataagtttataaacatgcagagcgatacataaaaaatgtgaataaattaatctatacaaataaacaccaaaaaacacccttagatctagacaattttgtgttgagtaattataatttaccaaaaactaggaaaaatagtgcctataacataagagcctgaactaaataggggaagaccaaaaacaaaaacatagctacaaagaaatagaatgagattcgataacaccatacagcttctttctgtccttgcattgaacagaaaacgtatttttaaattatattcatctaaatgctataatgagatacaaatctgtagtcattatctcctctcattcttcacatcacaaaacatttatttattattaacataactcatatagagaaaagagaacgcatctcaaacagcagaatcattcaaaagaaatcattataatgattacattacggcagaacaagtgcagctagttttaactttgttgctttgcaattatcctcaaaaaaattattatccttcaactaaccaatataaatacgtacatacatatccaagtaaagattacggacagaaatcaaaatacagatcacaaacaagaataaaaaggagcattaagtagaattaacaccttcaagttccatttgctattcccaagctgatcactgggagaagctgagaaagcgctaccatcaacatcacaagcatactccaccgtttcggtctttccacaagcaatctcatgccaaaattccttttccagatatgaggcaggaagactaccagagctataatatcgtctagaaaaaaccttgtttgccattttctcaaaatcacggaatgtataatttctgaaaacatccatccatattcaaaaaatgagaaaaaactaaaataaatcaaatcaagtatcatccttctatttagtatacacacttaccttccgctcatgaaaaatgtaactttatcatcagtgtcccactcagctagacggagaggttgcactcgggttgtgaacttaaatcccggttgctccttcatcaatacaaccccagctggaacagaagcagtcacaggtgaaacaatcttgatcatacctgaaattatcaagcaaaaaaaatcatagaactaacatgttagtattcattttgaatagacagctgcaacttaaggtttatgtattggaaccaataataataacaaagtgggacatgatgtgtgcgtgtatgtatgtgtgtgcgaaaaataaaacgggaaacaaataaatgaataaaataaaaattaaaagaaaaaggtgttaacaatacactatttttctttttaaatcaggatagagaaaggacagttccaagcttcgtaacaagctacaacagtagactgtcagaaagtgaatcaaacctgaagtacagatatgaacacttcctcttagcctaactttagaaatgtagctctttttataaagataaagaaattatggtctagacagcacaaagagaatacatattgcaagctaaccatatgatgaagcctaatttcttgtaataaaggttaaattaataatataaggtgtggccacattcatcataaaatactcaaaactccactactgctatattataaataagctaacttgatcaaaaacaatgtaaataatatctcaatgaccaactgatatgtgatggtgcctcaggacaatgtggaaaactaggatcttaacttcattattgctgtgctagtttagatagcatttgctttttctggtcataaaacaatgaagatgcaatgcattttacattttacatctgtcaaattaaaataatgtaatttaagaaaaatgtattagacaaacaaactgcaaaaataacaagagacacttcaaagaataataccatatccagaagcttctggagcgatcttctgcaaataaatcagagggtcctcaaactcctcttttgttggataatatataggacactcaggaattttttcagtccattccagatcacttgtgtcaaacttatccaccttccgcttcgaaataacatccttcccacttaaaggaccatttgatccagaaaaaacatctgcattgccatgtaatctaacaccgcaagatgctgaagatcttaatgcatctcctccacttcgagccattgtactgctgatagtttctgccttcatacgctgaagccttttgcgcttcagaaactccaacccatttctgacctcctttgataaacaaacccttccctccacctgtcaaacaagtttacatgggacaatcagtacataaagataaaaacatgaaacattatacacatgcaaatctaacattgagtataaatagacaaataagaaatgatattacaaaacccttacctatttcaaaatattacttaattcaaaaaattaataataataaatcaagaaaccaatgacatgaatagacccatctccatgccaattgaaaaaaaaaagagcgtaagaaggcttctataaaaataaacagattgcagttatatctaagcgcttccttttccgcttgaagaccttagatccttaggctgtctttgtacgggaataattttcaccttctactttataagatgaattacgaaggccacgcacaattacaaatgtcaatggtgaaagaagataatctaccataaacaaccacagcaccaccaacacaaatcaaatcataccaaagctccctagagcaaacgggacccaaaaaaatttataattgcggggtaaaataatttacaacagaaagtgtcggttttatccaattttcaataaagcatagagaaataaatcttcggcttaaaaaataaacatataataatcataaaaataagggaaaaaattaatcaagctcagccgtattcaacaaaaacatgcagtcaaacttaaaaggagtcttcgtcaaaattattactactcaaattccgaggctacaatagcctggatttattaaagcgaagatcaggaaataaaattagttactttgtttggatccttttcacatataatcaaatattaaacttatccaaataaattcaaatatttcctaaacagaaccaattgcaggcctaaacctataaacattttctaaaatgcatatttcagaagttcctcccgatagtaatcacggattcccagaatgaaatcgacgaaataataagacgataatatattagacctatcaaaatagggaacaaccggatatgtacgcatacacaagtatatgtgtgcg

SEQ ID NO: 391 - DNA, 2526 bp, Cannabis sativa
atgaaggcagaaactatcagcagtacaatggctcgaagtggaggagatgcattaagatcttcagcatcttgcggtgttagattacatggcaatgcagatgttttttctggatcaaatggtcctttaagtgggaaggatgttatttcgaagcggaaggtggataagtttgacacaagtgatctggaatggactgaaaaaattcctgagtgtcctatatattatccaacaaaagaggagtttgaggaccctctgatttatttgcagaagatcgctccagaagcttctggatatggtattggtatgatcaagattgtttcacctgtgactgcttctgttccagctggggttgtattgatgaaggagcaaccgggatttaagttcacaacccgagtgcaacctctccgtctagctgagtgggacactgatgataaagttacatttttcatgagcggaaggagaaattatacattccgtgattttgagaaaatggcaaacaaggttttttctagacgatattatagctctggtagtcttcctgcctcatatctggaaaaggaattttggcatgagattgcttgtggaaagaccgaaacggtggagtatgcttgtgatgttgatggtagcgctttctcagcttctcccagtgatcagcttgggaatagcaaatggaacttgaaggtgttaattctacttaatgctccttttggagtaactgatcctatgctctatattggaatgctatttagcatgtttgcttggcatgtcgaggatcattatctatacagtattaattatcatcactgcggtgcttcaaaaacttggtatggcattccaggtcatgctgctttgcagtttgaaaaggttgttagggaacatgtgtatactcatgacatcctttctgctgatggggaggatggggcttttgatgttttattggggaaaacaactttatttcctcctaatattctattggatcatgacgtccctgtgtacaaggcagtgcaaaaacctggagagttcatagttacgttccctagagcatatcatgctggatttaatcatggtttcaattgtggtgaagcagtgaactttgcaattggtgactggtttcccttgggtgctgttgctagccaacgctatgcacttcttagtaggatgcctttgcttcctcatgaagaactactttgcaaagaagcaatgcttctctattcgagtttagagcttgaagatttggatcattcttctgctgacttggcctctcattgttccattaaaacttcttttgtgaagttgatccgtttcttgcatcgagctcgctggttgctaatgaaatcagggatatgtactagtgtgtctgcaaatcggcatggaactatcctgtgcagcttatgtaaacgtgactgttatgttgcttttgtcaactgtagctgttacatgcatcctgtctgccttcgtcatgatatgacgtctttaaatttttcctgtggaagacactgtacgctctgtttaagggaggatatatcagaaatggaagatgcagctagaaagtttgaaatggaggatagagtaccagaaaagaagattaatcagcaatctaatagggttcatgacttgtattcagatccattagcaaacatgtcacataaagttaagcaggaggtatacattccttactgtccgatcaaatttgactcgatcccaaaacttagtgatactactcagtatcaatcaaaagagcaagatcatgtttcgtgtagtgcacaggtgtcaagtagtggaagtttaaattctgaaatgttaggaacatctatgtcctgtaccacatccactctacattctcttcctgtagaccctacggaagccgcaatcactcccagcaatgtagggaacactagctttgatgtgaggagtcttgactctagaaagttttcagaagacttgtcacggagtgcacttgaatctactctgtcttccccgtcatgtgatgaacacttgactgtaaatcaagattattctcatgaaaagctggttaggcctactctagatgaagaaagtgatagttctgattcagagatattcagggttaaacgtcgttcaacacagaagatggaaaagaagaacatgaatgataacatattatcgaagcattctgaccaccaggtacttagtttacacgtaaactttagatccatggacaggggttcagacgacttaagaaatttcagcctgacagaagatctgggtcgtcaggttgcaccagaattgtgggatcaaattctaaaccactctttcaacgacagaaagatttgtcaaggaaggggtaccattccgatctctataaaattcaagaatgtggttaatgagggtagtagtaacagactgagagaacaacccaaaaaggataggtttcagttagagttgggtaagagcacgagggaaccacacccagtagatattggagcaaaacgccttaaagtcagaggcccttcatttctagggtcagagacaggtcacaggttagattga

SEQ ID NO: 392 - protein, 868 aa, Cannabis sativa
QVEGRVCLSKEVRNGLEFLKRKRLQRMKAETISSTMARSGGDALRSSASCGVRLHGNADVFSGSNGPLSGKDVISKRKVDKFDTSDLEWTEKIPECPIYYPTKEEFEDPLIYLQKIAPEASGYGIGMIKIVSPVTASVPAGVVLMKEQPGFKFTTRVQPLRLAEWDTDDKVTFFMSGRRNYTFRDFEKMANKVFSRRYYSSGSLPASYLEKEFWHEIACGKTETVEYACDVDGSAFSASPSDQLGNSKWNLKVLILLNAPFGVTDPMLYIGMLFSMFAWHVEDHYLYSINYHHCGASKTWYGIPGHAALQFEKVVREHVYTHDILSADGEDGAFDVLLGKTTLFPPNILLDHDVPVYKAVQKPGEFIVTFPRAYHAGFNHGFNCGEAVNFAIGDWFPLGAVASQRYALLSRMPLLPHEELLCKEAMLLYSSLELEDLDHSSADLASHCSIKTSFVKLIRFLHRARWLLMKSGICTSVSANRHGTILCSLCKRDCYVAFVNCSCYMHPVCLRHDMTSLNFSCGRHCTLCLREDISEMEDAARKFEMEDRVPEKKINQQSNRVHDLYSDPLANMSHKVKQEVYIPYCPIKFDSIPKLSDTTQYQSKEQDHVSCSAQVSSSGTSLNSEMLGTSMSCTTSTLHSLPVDPTEAAITPSNVGNTSFDVRSLDSRKFSEDLSRSALESTLSSPSCDEHLTVNQDYSHEKLVRPTLDEESDSSDSEIFRVKRRSTQKMEKKNMNDNILSKHSDHQVLSLHVNFRSMDRGSDDLRNFSLTEDLGRQVAPELWDQILNHSFNDRKICQGRGTIPISIKFKNVVNEGSSNRLREQPKKDRFQLELGKSTREPHPVDIGAKRLKVRGPSFLGSETGHRLD

SEQ ID NO: 393 - DNA, 20 bp, Cannabis sativa
gaaaatgaagttagcaagtc

SEQ ID NO: 394 - DNA, 20 bp, Cannabis sativa
caggctctagtttgatattg

SEQ ID NO: 395 - DNA, 20 bp, Cannabis sativa
atattgtggctccagagagc

SEQ ID NO: 396 - DNA, 20 bp, Cannabis sativa
ttttttattgacctgctctc

SEQ ID NO: 397 - DNA, 20 bp, Cannabis sativa
agagcaggtcaataaaaaaa

SEQ ID NO: 398 - DNA, 20 bp, Cannabis sativa
tgtgtaatctaataatgaaa

SEQ ID NO: 399 - DNA, 20 bp, Cannabis sativa
ctaataatgaaatggtgaaa

SEQ ID NO: 400 - DNA, 20 bp, Cannabis sativa
ggttagattgaaagaagaac

SEQ ID NO: 401 - DNA, 20 bp, Cannabis sativa
tagggtcagagacaggtcac

SEQ ID NO: 402 - DNA, 20 bp, Cannabis sativa
tcatttctagggtcagagac

SEQ ID NO: 403 - DNA, 20 bp, Cannabis sativa
tcagaggcccttcatttcta

SEQ ID NO: 404 - DNA, 20 bp, Cannabis sativa
gtcagaggcccttcatttct

SEQ ID NO: 405 - DNA, 20 bp, Cannabis sativa
gtctctgaccctagaaatga

SEQ ID NO: 406 - DNA, 20 bp, Cannabis sativa
tctctgaccctagaaatgaa

SEQ ID NO: 407 - DNA, 20 bp, Cannabis sativa
gcaaaacgccttaaagtcag

SEQ ID NO: 408 - DNA, 20 bp, Cannabis sativa
atgaagggcctctgacttta

SEQ ID NO: 409 - DNA, 20 bp, Cannabis sativa
gaaccacacccagtagatat

SEQ ID NO: 410 - DNA, 20 bp, Cannabis sativa
cgttttgctccaatatctac

SEQ ID NO: 411 - DNA, 20 bp, Cannabis sativa
gttttgctccaatatctact

SEQ ID NO: 412 - DNA, 20 bp, Cannabis sativa
gctccaatatctactgggtg

SEQ ID NO: 413 - DNA, 20 bp, Cannabis sativa
agagttgggtaagagcacga

SEQ ID NO: 414 - DNA, 20 bp, Cannabis sativa
tagagttgggtaagagcacg

SEQ ID NO: 415 - DNA, 20 bp, Cannabis sativa
gataggtttcagttagagtt

SEQ ID NO: 416 - DNA, 20 bp, Cannabis sativa
ggataggtttcagttagagt

SEQ ID NO: 417 - DNA, 20 bp, Cannabis sativa
gagaacaacccaaaaaggat

SEQ ID NO: 418 - DNA, 20 bp, Cannabis sativa
taactgaaacctatcctttt

SEQ ID NO: 419 - DNA, 20 bp, Cannabis sativa
aactgaaacctatccttttt

SEQ ID NO: 420 - DNA, 20 bp, Cannabis sativa
actgagagaacaacccaaaa

SEQ ID NO: 421 - DNA, 20 bp, Cannabis sativa
ttcaagaatgtggttaatga

SEQ ID NO: 422 - DNA, 20 bp, Cannabis sativa
attcaagaatgtggttaatg

SEQ ID NO: 423 - DNA, 20 bp, Cannabis sativa
ctctataaaattcaagaatg

SEQ ID NO: 424 - DNA, 20 bp, Cannabis sativa
ttcttgaattttatagagat

SEQ ID NO: 425 - DNA, 20 bp, Cannabis sativa
gaattttatagagatcggaa

SEQ ID NO: 426 - DNA, 20 bp, Cannabis sativa
agaaagatttgtcaaggaag

SEQ ID NO: 427 - DNA, 20 bp, Cannabis sativa
cagaaagatttgtcaaggaa

SEQ ID NO: 428 - DNA, 20 bp, Cannabis sativa
acagaaagatttgtcaagga

SEQ ID NO: 429 - DNA, 20 bp, Cannabis sativa
aacgacagaaagatttgtca

SEQ ID NO: 430 - DNA, 20 bp, Cannabis sativa
ctatgattagtgagcgtagt

SEQ ID NO: 431 - DNA, 20 bp, Cannabis sativa
tcaggttgcaccagaattgt

SEQ ID NO: 432 - DNA, 20 bp, Cannabis sativa
agaatttgatcccacaattc

SEQ ID NO: 433 - DNA, 20 bp, Cannabis sativa
gtcaggttgcaccagaattg

SEQ ID NO: 434 - DNA, 20 bp, Cannabis sativa
gacagaagatctgggtcgtc

SEQ ID NO: 435 - DNA, 20 bp, Cannabis sativa
ttcagcctgacagaagatct

SEQ ID NO: 436 - DNA, 20 bp, Cannabis sativa
tttcagcctgacagaagatc

SEQ ID NO: 437 - DNA, 20 bp, Cannabis sativa
gacgacccagatcttctgtc

SEQ ID NO: 438 - DNA, 20 bp, Cannabis sativa
ctgaaatttttaatggacag

SEQ ID NO: 439 - DNA, 20 bp, Cannabis sativa
gctgaaatttttaatggaca

SEQ ID NO: 440 - DNA, 20 bp, Cannabis sativa
tgctgaaatttttaatggac

SEQ ID NO: 441 - DNA, 20 bp, Cannabis sativa
ctggttgctgaaatttttaa

SEQ ID NO: 442 - DNA, 20 bp, Cannabis sativa
caattgatgattaacttttc

SEQ ID NO: 443 - DNA, 20 bp, Cannabis sativa
aaagttaatcatcaattgtt

SEQ ID NO: 444 - DNA, 20 bp, Cannabis sativa
caattgttaggacacaatac

SEQ ID NO: 445 - DNA, 20 bp, Cannabis sativa
caatactggtttatgaaact

SEQ ID NO: 446 - DNA, 20 bp, Cannabis sativa
ttacgtgtaaactaagtacc

SEQ ID NO: 447 - DNA, 20 bp, Cannabis sativa
cgtgtaaactaagtacctgg

SEQ ID NO: 448 - DNA, 20 bp, Cannabis sativa
atcgaagcattctgaccacc

SEQ ID NO: 449 - DNA, 20 bp, Cannabis sativa
acgtcgttcaacacagaaga

SEQ ID NO: 450 - DNA, 20 bp, Cannabis sativa
ttctgattcagagatattca

SEQ ID NO: 451 - DNA, 20 bp, Cannabis sativa
gttctgattcagagatattc

SEQ ID NO: 452 - DNA, 20 bp, Cannabis sativa
tcactttcttcatctagagt

SEQ ID NO: 453 - DNA, 20 bp, Cannabis sativa
attctcatgaaaagctggtt

SEQ ID NO: 454 - DNA, 20 bp, Cannabis sativa
agattattctcatgaaaagc

SEQ ID NO: 455 - DNA, 20 bp, Cannabis sativa
gtcaagtgttcatcacatga

SEQ ID NO: 456 - DNA, 20 bp, Cannabis sativa
tcaagtgttcatcacatgac

SEQ ID NO: 457 - DNA, 20 bp, Cannabis sativa
caagtgttcatcacatgacg

SEQ ID NO: 458 - DNA, 20 bp, Cannabis sativa
agttttcagaagacttgtca

SEQ ID NO: 459 - DNA, 20 bp, Cannabis sativa
ggaacactagctttgatgtg

SEQ ID NO: 460 - DNA, 20 bp, Cannabis sativa
tttattttctccaggtacat

SEQ ID NO: 461 - DNA, 20 bp, Cannabis sativa
agctagtgttcccatgtacc

SEQ ID NO: 462 - DNA, 20 bp, Cannabis sativa
atttattttctccaggtaca

SEQ ID NO: 463 - DNA, 20 bp, Cannabis sativa
tgcatctatttattttctcc

SEQ ID NO: 464 - DNA, 20 bp, Cannabis sativa
gcactctaaataattctgaa

SEQ ID NO: 465 - DNA, 20 bp, Cannabis sativa
atacagaagtagccaactaa

SEQ ID NO: 466 - DNA, 20 bp, Cannabis sativa
agatacagatctcctttagt

SEQ ID NO: 467 - DNA, 20 bp, Cannabis sativa
agatctgtatcttacattgc

SEQ ID NO: 468 - DNA, 20 bp, Cannabis sativa
gatctgtatcttacattgct

SEQ ID NO: 469 - DNA, 20 bp, Cannabis sativa
tacattgctgggagtgattg

SEQ ID NO: 470 - DNA, 20 bp, Cannabis sativa
ggagtgattgcggcttccgt

SEQ ID NO: 471 - DNA, 20 bp, Cannabis sativa
gagtgattgcggcttccgta

SEQ ID NO: 472 - DNA, 20 bp, Cannabis sativa
ttctcttcctgtagacccta

SEQ ID NO: 473 - DNA, 20 bp, Cannabis sativa
gcggcttccgtagggtctac

SEQ ID NO: 474 - DNA, 20 bp, Cannabis sativa
tacaggaagagaatgtagag

SEQ ID NO: 475 - DNA, 20 bp, Cannabis sativa
aagagaatgtagagtggatg

SEQ ID NO: 476 - DNA, 20 bp, Cannabis sativa
atgtagagtggatgtggtac

SEQ ID NO: 477 - DNA, 20 bp, Cannabis sativa
agtttaaattctgaaatgtt

SEQ ID NO: 478 - DNA, 20 bp, Cannabis sativa
agtgcacaggtgtcaagtag

SEQ ID NO: 479 - DNA, 20 bp, Cannabis sativa
tcatgtttcgtgtagtgcac

SEQ ID NO: 480 - DNA, 20 bp, Cannabis sativa
tgagtagtatcactaagttt

SEQ ID NO: 481 - DNA, 20 bp, Cannabis sativa
gagtagtatcactaagtttt

SEQ ID NO: 482 - DNA, 20 bp, Cannabis sativa
gggatcgagtcaaatttgat

SEQ ID NO: 483 - DNA, 20 bp, Cannabis sativa
tcaaatttgatcggacagta

SEQ ID NO: 484 - DNA, 20 bp, Cannabis sativa
gtcacataaagttaagcagg

SEQ ID NO: 485 - DNA, 20 bp, Cannabis sativa
catgtcacataaagttaagc

SEQ ID NO: 486 - DNA, 20 bp, Cannabis sativa
ttatgtgacatgtttgctaa

SEQ ID NO: 487 - DNA, 20 bp, Cannabis sativa
gattaatcagcaatctaata

SEQ ID NO: 488 - DNA, 20 bp, Cannabis sativa
agattaatcagcaatctaat

SEQ ID NO: 489 - DNA, 20 bp, Cannabis sativa
tgctgattaatcttcttttc

SEQ ID NO: 490 - DNA, 20 bp, Cannabis sativa
agctagaaagtttgaaatgg

SEQ ID NO: 491 - DNA, 20 bp, Cannabis sativa
tgcagctagaaagtttgaaa

SEQ ID NO: 492 - DNA, 20 bp, Cannabis sativa
aagggaggatatatcagaaa

SEQ ID NO: 493 - DNA, 20 bp, Cannabis sativa
ctgtacgctctgtttaaggg

SEQ ID NO: 494 - DNA, 20 bp, Cannabis sativa
acactgtacgctctgtttaa

SEQ ID NO: 495 - DNA, 20 bp, Cannabis sativa
gacactgtacgctctgttta

SEQ ID NO: 496 - DNA, 20 bp, Cannabis sativa
gagcgtacagtgtcttccac

SEQ ID NO: 497 - DNA, 20 bp, Cannabis sativa
acgtctttaaatttttcctg

SEQ ID NO: 498 - DNA, 20 bp, Cannabis sativa
taagagtgtgtaagaatcca

SEQ ID NO: 499 - DNA, 20 bp, Cannabis sativa
aatgctatatgtttctgcct

SEQ ID NO: 500 - DNA, 20 bp, Cannabis sativa
aaggcagaaacatatagcat

SEQ ID NO: 501 - DNA, 20 bp, Cannabis sativa
gcattggtaaaatccccctg

SEQ ID NO: 502 - DNA, 20 bp, Cannabis sativa
cattggtaaaatccccctgt

SEQ ID NO: 503 - DNA, 20 bp, Cannabis sativa
acttaacagcagcccacagg

SEQ ID NO: 504 - DNA, 20 bp, Cannabis sativa
tacttaacagcagcccacag

SEQ ID NO: 505 - DNA, 20 bp, Cannabis sativa
ctacttaacagcagcccaca

SEQ ID NO: 506 - DNA, 20 bp, Cannabis sativa
cctacttaacagcagcccac

SEQ ID NO: 507 - DNA, 20 bp, Cannabis sativa
cctgtgggctgctgttaagt

SEQ ID NO: 508 - DNA, 20 bp, Cannabis sativa
gggctgctgttaagtaggaa

SEQ ID NO: 509 - DNA, 20 bp, Cannabis sativa
ccaatgggacttttgatact

SEQ ID NO: 510 - DNA, 20 bp, Cannabis sativa
cccaatgggacttttgatac

SEQ ID NO: 511 - DNA, 20 bp, Cannabis sativa
cccagtatcaaaagtcccat

SEQ ID NO: 512 - DNA, 20 bp, Cannabis sativa
ccagtatcaaaagtcccatt

SEQ ID NO: 513 - DNA, 20 bp, Cannabis sativa
aattatgacctctacccaat

SEQ ID NO: 514 - DNA, 20 bp, Cannabis sativa
gaattatgacctctacccaa

SEQ ID NO: 515 - DNA, 20 bp, Cannabis sativa
tcaaaagtcccattgggtag

SEQ ID NO: 516 - DNA, 20 bp, Cannabis sativa
gtattcatcaccattttaat

SEQ ID NO: 517 - DNA, 20 bp, Cannabis sativa
aagtttatatcccattaaaa

SEQ ID NO: 518 - DNA, 20 bp, Cannabis sativa
tgtattcatcaccattttaa

SEQ ID NO: 519 - DNA, 20 bp, Cannabis sativa
cagcctagtgtttgcgtgtt

SEQ ID NO: 520 - DNA, 20 bp, Cannabis sativa
aatccaaacacgcaaacact

SEQ ID NO: 521 - DNA, 20 bp, Cannabis sativa
caaacacgcaaacactaggc

SEQ ID NO: 522 - DNA, 20 bp, Cannabis sativa
atagaagcataccatgacga

SEQ ID NO: 523 - DNA, 20 bp, Cannabis sativa
catcctgtctgccttcgtca

SEQ ID NO: 524 - DNA, 20 bp, Cannabis sativa
ataccatgacgaaggcagac

SEQ ID NO: 525 - DNA, 20 bp, Cannabis sativa
cacgtttacataagctgcac

SEQ ID NO: 526 - DNA, 20 bp, Cannabis sativa
agtgtgtctgcaaatcggca

SEQ ID NO: 527 - DNA, 20 bp, Cannabis sativa
gtactagtgtgtctgcaaat

SEQ ID NO: 528 - DNA, 20 bp, Cannabis sativa
gctggttgctaatgaaatca

SEQ ID NO: 529 - DNA, 20 bp, Cannabis sativa
cgctggttgctaatgaaatc

SEQ ID NO: 530 - DNA, 20 bp, Cannabis sativa
gtttcttgcatcgagctcgc

SEQ ID NO: 531 - DNA, 20 bp, Cannabis sativa
agcgagctcgatgcaagaaa

SEQ ID NO: 532 - DNA, 20 bp, Cannabis sativa
cttcacaaaagaagttttaa

SEQ ID NO: 533 - DNA, 20 bp, Cannabis sativa
agttttaatggaacaatgag

SEQ ID NO: 534 - DNA, 20 bp, Cannabis sativa
ggatcattcttctgctgact

SEQ ID NO: 535 - DNA, 20 bp, Cannabis sativa
gagtttagagcttgaagatt

SEQ ID NO: 536 - DNA, 20 bp, Cannabis sativa
ttgcaaagtagttcttcatg

SEQ ID NO: 537 - DNA, 20 bp, Cannabis sativa
agttcttcatgaggaagcaa

SEQ ID NO: 538 - DNA, 20 bp, Cannabis sativa
aacgctatgcacttcttagt

SEQ ID NO: 539 - DNA, 20 bp, Cannabis sativa
tactaagaagtgcatagcgt

SEQ ID NO: 540 - DNA, 20 bp, Cannabis sativa
ttggctagcaacagcaccca

SEQ ID NO: 541 - DNA, 20 bp, Cannabis sativa
tggctagcaacagcacccaa

SEQ ID NO: 542 - DNA, 20 bp, Cannabis sativa
attggtgactggtttccctt

SEQ ID NO: 543 - DNA, 20 bp, Cannabis sativa
aattggtgactggtttccct

SEQ ID NO: 544 - DNA, 20 bp, Cannabis sativa
tgaactttgcaattggtgac

SEQ ID NO: 545 - DNA, 20 bp, Cannabis sativa
gaagcagtgaactttgcaat

SEQ ID NO: 546 - DNA, 20 bp, Cannabis sativa
ggaactgtaggcttcaattg

SEQ ID NO: 547 - DNA, 20 bp, Cannabis sativa
acttttccttttggaactgt

SEQ ID NO: 548 - DNA, 20 bp, Cannabis sativa
ttgaagcctacagttccaaa

SEQ ID NO: 549 - DNA, 20 bp, Cannabis sativa
agaaattgtacttttccttt

SEQ ID NO: 550 - DNA, 20 bp, Cannabis sativa
tatcatgctggatttaatca

SEQ ID NO: 551 - DNA, 20 bp, Cannabis sativa
ttccctagagcatatcatgc

SEQ ID NO: 552 - DNA, 20 bp, Cannabis sativa
aatccagcatgatatgctct

SEQ ID NO: 553 - DNA, 20 bp, Cannabis sativa
atccagcatgatatgctcta

SEQ ID NO: 554 - DNA, 20 bp, Cannabis sativa
aacgtaactatgaactctcc

SEQ ID NO: 555 - DNA, 20 bp, Cannabis sativa
tacaaggcagtgcaaaaacc

SEQ ID NO: 556 - DNA, 20 bp, Cannabis sativa
tcatgacgtccctgtgtaca

SEQ ID NO: 557 - DNA, 20 bp, Cannabis sativa
ttttgcactgccttgtacac

SEQ ID NO: 558 - DNA, 20 bp, Cannabis sativa
tttgcactgccttgtacaca

SEQ ID NO: 559 - DNA, 20 bp, Cannabis sativa
atttcctcctaatattctat

SEQ ID NO: 560 - DNA, 20 bp, Cannabis sativa
tcatgatccaatagaatatt

SEQ ID NO: 561 - DNA, 20 bp, Cannabis sativa
tgatccaatagaatattagg

SEQ ID NO: 562 - DNA, 20 bp, Cannabis sativa
gggcttttgatgttttattg

SEQ ID NO: 563 - DNA, 20 bp, Cannabis sativa
ggggcttttgatgttttatt

SEQ ID NO: 564 - DNA, 20 bp, Cannabis sativa
tggggcttttgatgttttat

SEQ ID NO: 565 - DNA, 20 bp, Cannabis sativa
ttctgctgatggggaggatg

SEQ ID NO: 566 - DNA, 20 bp, Cannabis sativa
tttctgctgatggggaggat

SEQ ID NO: 567 - DNA, 20 bp, Cannabis sativa
ctttctgctgatggggagga

SEQ ID NO: 568 - DNA, 20 bp, Cannabis sativa
catcctttctgctgatgggg

SEQ ID NO: 569 - DNA, 20 bp, Cannabis sativa
tgacatcctttctgctgatg

SEQ ID NO: 570 - DNA, 20 bp, Cannabis sativa
atgacatcctttctgctgat

SEQ ID NO: 571 - DNA, 20 bp, Cannabis sativa
catgacatcctttctgctga

SEQ ID NO: 572 - DNA, 20 bp, Cannabis sativa
catcctccccatcagcagaa

SEQ ID NO: 573 - DNA, 20 bp, Cannabis sativa
gcagtttgaaaaggttgtta

SEQ ID NO: 574 - DNA, 20 bp, Cannabis sativa
tgcagtttgaaaaggttgtt

SEQ ID NO: 575 - DNA, 20 bp, Cannabis sativa
tgctgctttgcagtttgaaa

SEQ ID NO: 576 - DNA, 20 bp, Cannabis sativa
aactgcaaagcagcatgacc

SEQ ID NO: 577 - DNA, 20 bp, Cannabis sativa
aaaacttggtatggcattcc

SEQ ID NO: 578 - DNA, 20 bp, Cannabis sativa
ggtgcttcaaaaacttggta

SEQ ID NO: 579 - DNA, 20 bp, Cannabis sativa
actgcggtgcttcaaaaact

SEQ ID NO: 580 - DNA, 20 bp, Cannabis sativa
agtattaattatcatcactg

SEQ ID NO: 581 - DNA, 20 bp, Cannabis sativa
gagtgagaaaattaatagag

SEQ ID NO: 582 - DNA, 20 bp, Cannabis sativa
agtgagaaaattaatagagt

SEQ ID NO: 583 - DNA, 20 bp, Cannabis sativa
gtgagaaaattaatagagtg

SEQ ID NO: 584 - DNA, 20 bp, Cannabis sativa
aaattagcatgaaagtttta

SEQ ID NO: 585 - DNA, 20 bp, Cannabis sativa
aggtatatgttattcttgag

SEQ ID NO: 586 - DNA, 20 bp, Cannabis sativa
tcgaggatcattatctatac

SEQ ID NO: 587 - DNA, 20 bp, Cannabis sativa
catgtttgcttggcatgtcg

SEQ ID NO: 588 - DNA, 20 bp, Cannabis sativa
tgctatttagcatgtttgct

SEQ ID NO: 589 - DNA, 20 bp, Cannabis sativa
actgatcctatgctctatat

SEQ ID NO: 590 - DNA, 20 bp, Cannabis sativa
agcattccaatatagagcat

SEQ ID NO: 591 - DNA, 20 bp, Cannabis sativa
aatgtttattttttttcaca

SEQ ID NO: 592 - DNA, 20 bp, Cannabis sativa
taatgtttattttttttcac

SEQ ID NO: 593 - DNA, 20 bp, Cannabis sativa
agataagttagtattgttac

SEQ ID NO: 594 - DNA, 20 bp, Cannabis sativa
ttttgtttcagagactgtca

SEQ ID NO: 595 - DNA, 20 bp, Cannabis sativa
ctagatctaagggtgttttt

SEQ ID NO: 596 - DNA, 20 bp, Cannabis sativa
acaaaattgtctagatctaa

SEQ ID NO: 597 - DNA, 20 bp, Cannabis sativa
cacaaaattgtctagatcta

SEQ ID NO: 598 - DNA, 20 bp, Cannabis sativa
attataatttaccaaaaact

SEQ ID NO: 599 - DNA, 20 bp, Cannabis sativa
gcactatttttcctagtttt

SEQ ID NO: 600 - DNA, 20 bp, Cannabis sativa
agttcaggctcttatgttat

SEQ ID NO: 601 - DNA, 20 bp, Cannabis sativa
ggtcttcccctatttagttc

SEQ ID NO: 602 - DNA, 20 bp, Cannabis sativa
cataagagcctgaactaaat

SEQ ID NO: 603 - DNA, 20 bp, Cannabis sativa
ataagagcctgaactaaata

SEQ ID NO: 604 - DNA, 20 bp, Cannabis sativa
taagagcctgaactaaatag

SEQ ID NO: 605 - DNA, 20 bp, Cannabis sativa
tgtagctatgtttttgtttt

SEQ ID NO: 606 - DNA, 20 bp, Cannabis sativa
aaggacagaaagaagctgta

SEQ ID NO: 607 - DNA, 20 bp, Cannabis sativa
tacgttttctgttcaatgca

SEQ ID NO: 608 - DNA, 20 bp, Cannabis sativa
tcattataatgattacatta

SEQ ID NO: 609 - DNA, 20 bp, Cannabis sativa
gaaggataataatttttttg

SEQ ID NO: 610 - DNA, 20 bp, Cannabis sativa
tatttatattggttagttga

SEQ ID NO: 611 - DNA, 20 bp, Cannabis sativa
atgtatgtacgtatttatat

SEQ ID NO: 612 - DNA, 20 bp, Cannabis sativa
ttctgtccgtaatctttact

SEQ ID NO: 613 - DNA, 20 bp, Cannabis sativa
acatatccaagtaaagatta

SEQ ID NO: 614 - DNA, 20 bp, Cannabis sativa
gatcacaaacaagaataaaa

SEQ ID NO: 615 - DNA, 20 bp, Cannabis sativa
gaatagcaaatggaacttga

SEQ ID NO: 616 - DNA, 20 bp, Cannabis sativa
atcagcttgggaatagcaaa

SEQ ID NO: 617 - DNA, 20 bp, Cannabis sativa
cttctcccagtgatcagctt

SEQ ID NO: 618 - DNA, 20 bp, Cannabis sativa
gcttctcccagtgatcagct

SEQ ID NO: 619 - DNA, 20 bp, Cannabis sativa
gctattcccaagctgatcac

SEQ ID NO: 620 - DNA, 20 bp, Cannabis sativa
ctattcccaagctgatcact

SEQ ID NO: 621 - DNA, 20 bp, Cannabis sativa
gagtatgcttgtgatgttga

SEQ ID NO: 622 - DNA, 20 bp, Cannabis sativa
acaagcatactccaccgttt

SEQ ID NO: 623 - DNA, 20 bp, Cannabis sativa
ttgtggaaagaccgaaacgg

SEQ ID NO: 624 - DNA, 20 bp, Cannabis sativa
tgcttgtggaaagaccgaaa

SEQ ID NO: 625 - DNA, 20 bp, Cannabis sativa
ttttggcatgagattgcttg

SEQ ID NO: 626 - DNA, 20 bp, Cannabis sativa
catatctggaaaaggaattt

SEQ ID NO: 627 - DNA, 20 bp, Cannabis sativa
tcctgcctcatatctggaaa

SEQ ID NO: 628 - DNA, 20 bp, Cannabis sativa
aaattccttttccagatatg

SEQ ID NO: 629 - DNA, 20 bp, Cannabis sativa
tagtcttcctgcctcatatc

SEQ ID NO: 630 - DNA, 20 bp, Cannabis sativa
tccttttccagatatgaggc

SEQ ID NO: 631 - DNA, 20 bp, Cannabis sativa
tctagacgatattatagctc

SEQ ID NO: 632 - DNA, 20 bp, Cannabis sativa
ttttgagaaaatggcaaaca

SEQ ID NO: 633 - DNA, 20 bp, Cannabis sativa
attccgtgattttgagaaaa

SEQ ID NO: 634 - DNA, 20 bp, Cannabis sativa
ttgccattttctcaaaatca

SEQ ID NO: 635 - DNA, 20 bp, Cannabis sativa
tctcattttttgaatatgga

SEQ ID NO: 636 - DNA, 20 bp, Cannabis sativa
tttttctcattttttgaata

SEQ ID NO: 637 - DNA, 20 bp, Cannabis sativa
aagtgtgtatactaaataga

SEQ ID NO: 638 - DNA, 20 bp, Cannabis sativa
ttacatttttcatgagcgga

SEQ ID NO: 639 - DNA, 20 bp, Cannabis sativa
aaagttacatttttcatgag

SEQ ID NO: 640 - DNA, 20 bp, Cannabis sativa
acctctccgtctagctgagt

SEQ ID NO: 641 - DNA, 20 bp, Cannabis sativa
aacctctccgtctagctgag

SEQ ID NO: 642 - DNA, 20 bp, Cannabis sativa
cagtgtcccactcagctaga

SEQ ID NO: 643 - DNA, 20 bp, Cannabis sativa
tcccactcagctagacggag

SEQ ID NO: 644 - DNA, 20 bp, Cannabis sativa
ctagacggagaggttgcact

SEQ ID NO: 645 - DNA, 20 bp, Cannabis sativa
tagacggagaggttgcactc

SEQ ID NO: 646 - DNA, 20 bp, Cannabis sativa
cgggttgtgaacttaaatcc

SEQ ID NO: 647 - DNA, 20 bp, Cannabis sativa
gtattgatgaaggagcaacc

SEQ ID NO: 648 - DNA, 20 bp, Cannabis sativa
tgtattgatgaaggagcaac

SEQ ID NO: 649 - DNA, 20 bp, Cannabis sativa
agctggggttgtattgatga

SEQ ID NO: 650 - DNA, 20 bp, Cannabis sativa
ttcatcaatacaaccccagc

SEQ ID NO: 651 - DNA, 20 bp, Cannabis sativa
gactgcttctgttccagctg

SEQ ID NO: 652 - DNA, 20 bp, Cannabis sativa
tgactgcttctgttccagct

SEQ ID NO: 653 - DNA, 20 bp, Cannabis sativa
gtgactgcttctgttccagc

SEQ ID NO: 654 - DNA, 20 bp, Cannabis sativa
gctggaacagaagcagtcac

SEQ ID NO: 655 - DNA, 20 bp, Cannabis sativa
tttttttgcttgataatttc

SEQ ID NO: 656 - DNA, 20 bp, Cannabis sativa
gaatagacagctgcaactta

SEQ ID NO: 657 - DNA, 20 bp, Cannabis sativa
gcaacttaaggtttatgtat

SEQ ID NO: 658 - DNA, 20 bp, Cannabis sativa
tcccactttgttattattat

SEQ ID NO: 659 - DNA, 20 bp, Cannabis sativa
aaccaataataataacaaag

SEQ ID NO: 660 - DNA, 20 bp, Cannabis sativa
accaataataataacaaagt

SEQ ID NO: 661 - DNA, 20 bp, Cannabis sativa
tgtgtgtgcgaaaaataaaa

SEQ ID NO: 662 - DNA, 20 bp, Cannabis sativa
gtgtgtgcgaaaaataaaac

SEQ ID NO: 663 - DNA, 20 bp, Cannabis sativa
aaataaaaattaaaagaaaa

SEQ ID NO: 664 - DNA, 20 bp, Cannabis sativa
actatttttctttttaaatc

SEQ ID NO: 665 - DNA, 20 bp, Cannabis sativa
ttttaaatcaggatagagaa

SEQ ID NO: 666 - DNA, 20 bp, Cannabis sativa
ttgtagcttgttacgaagct

SEQ ID NO: 667 - DNA, 20 bp, Cannabis sativa
agtgttcatatctgtacttc

SEQ ID NO: 668 - DNA, 20 bp, Cannabis sativa
atttctaaagttaggctaag

SEQ ID NO: 669 - DNA, 20 bp, Cannabis sativa
agagctacatttctaaagtt

SEQ ID NO: 670 - DNA, 20 bp, Cannabis sativa
ttataaagataaagaaatta

SEQ ID NO: 671 - DNA, 20 bp, Cannabis sativa
agaaattaggcttcatcata

SEQ ID NO: 672 - DNA, 20 bp, Cannabis sativa
aacctttattacaagaaatt

SEQ ID NO: 673 - DNA, 20 bp, Cannabis sativa
agcctaatttcttgtaataa

SEQ ID NO: 674 - DNA, 20 bp, Cannabis sativa
aaggttaaattaataatata

SEQ ID NO: 675 - DNA, 20 bp, Cannabis sativa
taaattaataatataaggtg

SEQ ID NO: 676 - DNA, 20 bp, Cannabis sativa
tgagtattttatgatgaatg

SEQ ID NO: 677 - DNA, 20 bp, Cannabis sativa
tatttataatatagcagtag

SEQ ID NO: 678 - DNA, 20 bp, Cannabis sativa
aggcaccatcacatatcagt

SEQ ID NO: 679 - DNA, 20 bp, Cannabis sativa
aatgaccaactgatatgtga

SEQ ID NO: 680 - DNA, 20 bp, Cannabis sativa
ctgatatgtgatggtgcctc

SEQ ID NO: 681 - DNA, 20 bp, Cannabis sativa
tagttttccacattgtcctg

SEQ ID NO: 682 - DNA, 20 bp, Cannabis sativa
gatggtgcctcaggacaatg

SEQ ID NO: 683 - DNA, 20 bp, Cannabis sativa
tcaggacaatgtggaaaact

SEQ ID NO: 684 - DNA, 20 bp, Cannabis sativa
tagatagcatttgctttttc

SEQ ID NO: 685 - DNA, 20 bp, Cannabis sativa
gctccagaagcttctggata

SEQ ID NO: 686 - DNA, 20 bp, Cannabis sativa
aagatcgctccagaagcttc

SEQ ID NO: 687 - DNA, 20 bp, Cannabis sativa
ataccatatccagaagcttc

SEQ ID NO: 688 - DNA, 20 bp, Cannabis sativa
atcttctgcaaataaatcag

SEQ ID NO: 689 - DNA, 20 bp, Cannabis sativa
tcttctgcaaataaatcaga

SEQ ID NO: 690 - DNA, 20 bp, Cannabis sativa
tccaacaaaagaggagtttg

SEQ ID NO: 691 - DNA, 20 bp, Cannabis sativa
tatatattatccaacaaaag

SEQ ID NO: 692 - DNA, 20 bp, Cannabis sativa
tcctcaaactcctcttttgt

SEQ ID NO: 693 - DNA, 20 bp, Cannabis sativa
tcttttgttggataatatat

SEQ ID NO: 694 - DNA, 20 bp, Cannabis sativa
ggataatatataggacactc

SEQ ID NO: 695 - DNA, 20 bp, Cannabis sativa
ttgacacaagtgatctggaa

SEQ ID NO: 696 - DNA, 20 bp, Cannabis sativa
taagtttgacacaagtgatc

SEQ ID NO: 697 - DNA, 20 bp, Cannabis sativa
tgttatttcgaagcggaagg

SEQ ID NO: 698 - DNA, 20 bp, Cannabis sativa
ggatgttatttcgaagcgga

SEQ ID NO: 699 - DNA, 20 bp, Cannabis sativa
ggaaggatgttatttcgaag

SEQ ID NO: 700 - DNA, 20 bp, Cannabis sativa
aaatggtcctttaagtggga

SEQ ID NO: 701 - DNA, 20 bp, Cannabis sativa
ataacatccttcccacttaa

SEQ ID NO: 702 - DNA, 20 bp, Cannabis sativa
gatcaaatggtcctttaagt

SEQ ID NO: 703 - DNA, 20 bp, Cannabis sativa
ggatcaaatggtcctttaag

SEQ ID NO: 704 - DNA, 20 bp, Cannabis sativa
gatgttttttctggatcaaa

SEQ ID NO: 705 - DNA, 20 bp, Cannabis sativa
ggcaatgcagatgttttttc

SEQ ID NO: 706 - DNA, 20 bp, Cannabis sativa
tcttgcggtgttagattaca

SEQ ID NO: 707 - DNA, 20 bp, Cannabis sativa
ttaagatcttcagcatcttg

SEQ ID NO: 708 - DNA, 20 bp, Cannabis sativa
agtacaatggctcgaagtgg

SEQ ID NO: 709 - DNA, 20 bp, Cannabis sativa
agcagtacaatggctcgaag

SEQ ID NO: 710 - DNA, 20 bp, Cannabis sativa
agaaactatcagcagtacaa

SEQ ID NO: 711 - DNA, 20 bp, Cannabis sativa
caaaaggcttcagcgtatga

SEQ ID NO: 712 - DNA, 20 bp, Cannabis sativa
tggagtttctgaagcgcaaa

SEQ ID NO: 713 - DNA, 20 bp, Cannabis sativa
aaaggaggtcagaaatgggt

SEQ ID NO: 714 - DNA, 20 bp, Cannabis sativa
tatcaaaggaggtcagaaat

SEQ ID NO: 715 - DNA, 20 bp, Cannabis sativa
ttatcaaaggaggtcagaaa

SEQ ID NO: 716 - DNA, 20 bp, Cannabis sativa
aagggtttgtttatcaaagg

SEQ ID NO: 717 - DNA, 20 bp, Cannabis sativa
gggaagggtttgtttatcaa

SEQ ID NO: 718 - DNA, 20 bp, Cannabis sativa
tgtttgacaggtggagggaa

SEQ ID NO: 719 - DNA, 20 bp, Cannabis sativa
ttgtttgacaggtggaggga

SEQ ID NO: 720 - DNA, 20 bp, Cannabis sativa
aaacttgtttgacaggtgga

SEQ ID NO: 721 - DNA, 20 bp, Cannabis sativa
taaacttgtttgacaggtgg

SEQ ID NO: 722 - DNA, 20 bp, Cannabis sativa
atgtaaacttgtttgacagg

SEQ ID NO: 723 - DNA, 20 bp, Cannabis sativa
cccatgtaaacttgtttgac

SEQ ID NO: 724 - DNA, 20 bp, Cannabis sativa
acctgtcaaacaagtttaca

SEQ ID NO: 725 - DNA, 20 bp, Cannabis sativa
cctgtcaaacaagtttacat

SEQ ID NO: 726 - DNA, 2917 bp, Cannabis sativa
gccgagagagagatcatatatagatagatatagatagagtactacttcttctactactactatattctatactatactaacactcccactacttcttatccttttctctaattcactttttatttattttgctttcctcttttgtttatcattttcacaggcgattcctgttgcgggttttgtctatttgatataaaaataataatgtattgtttcgggaggtgtgaattataagagaaagtagtactactatatagctaggactgtaaatttttatttaattattttattatatatttatttatatttcaaaataaaaatatgaacacatcagggtttaggtgaggaggctataagaagtgatgttgtgtctggctagaggatccaatactaccaaaaacaagagcaacaaaacggctttgcatcattgtttctcacttggctggcgactactataactatatttatatttacatatagaaagagaactcagttctccaaagctggttcatataagcaatcattatggcttcatcaaaccgacactggcctagtatgttcaagtccaagccttgcaatagccaccaccaatggcagcatgacatcaacccatcgctcatgactagtggataccacagacctccgtacgcttcaggtatatatatatagatatattttgacaagattatctttttcgttctttcaatatcctattttttgaaaagttgtcaaacaaactttctaataattttatgaccaagaagtacatgcaatattcccatgtgttgcattttataattaaaacctttctatactacagttggtgggtgtgatgagaggagtccggagccgaagcccagatggaacccgaaaccggagcagattcgaatccttgaagcgatctttaactctggcatggtgaatcccccgagagacgagattagaaggatcagagcccaattgcaagagtatggacaagttggtgatgccaatgtgttttattggtttcaaaacagaaaatctaggagcaaacacaagctccgccacctccaaaactccaaacaacaaacccttcaaaatccaactccaactcctccttcctcagccaccgctccttcctcttcgtcttcttcgtcgtctgagaaaacgtctcccaaatcccgatcgagaggcttctcatcatcattgggcttctcaaacctagccagtactactaatcatcatcatgattatcaccatcaaactagtccaacttcttcggtcaataataatcagaccatctttcaaaccaacaacattaataataatactcatcacgagttcttaccaaccgatcaacactatttcttcccggccgtgcctaatcatcaccccagttctactcctgttcctgctaataccagtcaagggttttgcttccctcctcaaaatcatgagctctctcatggggtagtactacctcatcacatggagatacagaatatagggccttgtacaagccttttacttagtgagatcatgaaccaaaacggtgacttaaagaaagaccatcacgaggtgaataattatcataagaatttgatgatgaagatgagctctacggatgacaatgatgaatctcagttgaattacacttgtctgatgacgccgactaggactccggccactactccgtctaacgtcgtcgtttcatctcctcaccaccacgctactactgcaacgactactgtatctgctccatctgtccccctcaatcaaatccaaggtaaaataaaataaaatgttaccacatggaactgtaactgaaaatcgaaattttgtttggatgtgcataaaataaaaatatagctaaattaattaaatgttatataaaaattttaacacgggagaggtgcaaatttactgcagttaattgaatttacaaattattatacattggtaaaagatcaggacaatattgtctaatgggtccagcgatctgagtgtagatggacgtgtgcatggatttggtggtgttggtccttaggtcgactgtttcatattgatctttgtattgggtaaatatgtttagaataataaatatttagaatataattttatttatttatgacgaaagtaatagggtactactgggtttatttaattaataaattattaggattagaaagtttgtgactactatcctcctttataactttgctttttctgataagttgtacgtataccttattcgatcgatcgtggagaataataatattattacatatatttatgagaacaggtgttgtaggagaagatgaacagggagtggtgggtccaggtggagatggtgtggcaaataaaacgatggtgttcataaacgacgtggccttcgaggtggccgctgggccattcaacgtgcgcgaggcttttggtgaagacgctgtacttattcactccaatggtcaacttgttcccaccgacgactggggcgtcactcctcactccctccagcacggcgctttttactatcttatctggtaataattcaccttttcacaccattatttacgtaaatctttttatttgattataatatttgtggcctctataattgcagatttaggagctcatatatatacgagtaagagattttgaagcaatatatttatatatatatgtagcttttggattagggttttcaatcattgcttatcatattaattctcattttaatgatcgatgtctccgcaatttacatcttggacgacgacttatctgtactcatttggtttttgaacctcttttgttgctgttgttcttctttttatttctatatatatttataaaaatctaggtggttttaatgagtatggatatgtttagtcttatgtttatctttctgtttggacgatggcatataagtagtgagtttaat

SEQ ID NO: 727 - DNA, 1173 bp, Artificial Sequence
  CsMultiflora CDS
atggcttcatcaaaccgacactggcctagtatgttcaagtccaagccttgcaatagccaccaccaatggcagcatgacatcaacccatcgctcatgactagtggataccacagacctccgtacgggtgtgatgagaggagtccggagccgaagcccagatggaacccgaaaccggagcagattcgaatccttgaagcgatctttaactctggcatggtgaatcccccgagagacgagattagaaggatcagagcccaattgcaagagtatggacaagttggtgatgccaatgtgttttattggtttcaaaacagaaaatctaggagcaaacacaagctccgccacctccaaaactccaaacaacaaacccttcaaaatccaactccaactcctccttcctcagccaccgctccttcctcttcgtcttcttcgtcgtctgagaaaacgtctcccaaatcccgatcgagaggcttctcatcatcattgggcttctcaaacctagccagtactactaatcatcatcatgattatcaccatcaaactagtccaacttcttcggtcaataataatcagaccatctttcaaaccaacaacattaataataatactcatcacgagttcttaccaaccgatcaacactatttcttcccggccgtgcctaatcatcaccccagttctactcctgttcctgctaataccagtcaagggttttgcttccctcctcaaaatcatgagctctctcatggggtagtactacctcatcacatggagatacagaatatagggccttgtacaagccttttacttagtgagatcatgaaccaaaacggtgacttaaagaaagaccatcacgaggtgaataattatcataagaatttgatgatgaagatgagctctacggatgacaatgatgaatctcagttgaattacacttgtctgatgacgccgactaggactggagtggtgggtccaggtggagatggtgtggcaaataaaacgatggtgttcataaacgacgtggccttcgaggtggccgctgggccattcaacgtgcgcgaggcttttggtgaagacgctgtacttattcactccaatggtcaacttgttcccaccgacgactggggcgtcactcctcactccctccagcacggcgctttttactatcttatctgg

SEQ ID NO: 728 - protein, 391 aa, Artificial Sequence
  CsMultiflora AA
MASSNRHWPSMFKSKPCNSHHQWQHDINPSLMTSGYHRPPYGCDERSPEPKPRWNPKPEQIRILEAIFNSGMVNPPRDEIRRIRAQLQEYGQVGDANVFYWFQNRKSRSKHKLRHLQNSKQQTLQNPTPTPPSSATAPSSSSSSSSEKTSPKSRSRGFSSSLGFSNLASTTNHHHDYHHQTSPTSSVNNNQTIFQTNNINNNTHHEFLPTDQHYFFPAVPNHHPSSTPVPANTSQGFCFPPQNHELSHGVVLPHHMEIQNIGPCTSLLLSEIMNQNGDLKKDHHEVNNYHKNLMMKMSSTDDNDESQLNYTCLMTPTRTGVVGPGGDGVANKTMVFINDVAFEVAAGPFNVREAFGEDAVLIHSNGQLVPTDDWGVTPHSLQHGAFYYLIW

SEQ ID NO: 729 - DNA, 20 bp, Cannabis sativa
ataagaagtgatgttgtgtc

SEQ ID NO: 730 - DNA, 20 bp, Cannabis sativa
gtgatgttgtgtctggctag

SEQ ID NO: 731 - DNA, 20 bp, Cannabis sativa
gctcttgtttttggtagtat

SEQ ID NO: 732 - DNA, 20 bp, Cannabis sativa
cgttttgttgctcttgtttt

SEQ ID NO: 733 - DNA, 20 bp, Cannabis sativa
caaaaacaagagcaacaaaa

SEQ ID NO: 734 - DNA, 20 bp, Cannabis sativa
ttgcatcattgtttctcact

SEQ ID NO: 735 - DNA, 20 bp, Cannabis sativa
atcattgtttctcacttggc

SEQ ID NO: 736 - DNA, 20 bp, Cannabis sativa
agaactcagttctccaaagc

SEQ ID NO: 737 - DNA, 20 bp, Cannabis sativa
ttgcttatatgaaccagctt

SEQ ID NO: 738 - DNA, 20 bp, Cannabis sativa
gttcatataagcaatcatta

SEQ ID NO: 739 - DNA, 20 bp, Cannabis sativa
tggcttcatcaaaccgacac

SEQ ID NO: 740 - DNA, 20 bp, Cannabis sativa
tgaacatactaggccagtgt

SEQ ID NO: 741 - DNA, 20 bp, Cannabis sativa
ggcttggacttgaacatact

SEQ ID NO: 742 - DNA, 20 bp, Cannabis sativa
gtggtggctattgcaaggct

SEQ ID NO: 743 - DNA, 20 bp, Cannabis sativa
cattggtggtggctattgca

SEQ ID NO: 744 - DNA, 20 bp, Cannabis sativa
tgtcatgctgccattggtgg

SEQ ID NO: 745 - DNA, 20 bp, Cannabis sativa
cttgcaatagccaccaccaa

SEQ ID NO: 746 - DNA, 20 bp, Cannabis sativa
tgatgtcatgctgccattgg

SEQ ID NO: 747 - DNA, 20 bp, Cannabis sativa
ggttgatgtcatgctgccat

SEQ ID NO: 748 - DNA, 20 bp, Cannabis sativa
atccactagtcatgagcgat

SEQ ID NO: 749 - DNA, 20 bp, Cannabis sativa
tatccactagtcatgagcga

SEQ ID NO: 750 - DNA, 20 bp, Cannabis sativa
aacccatcgctcatgactag

SEQ ID NO: 751 - DNA, 20 bp, Cannabis sativa
ctgaagcgtacggaggtctg

SEQ ID NO: 752 - DNA, 20 bp, Cannabis sativa
tatatacctgaagcgtacgg

SEQ ID NO: 753 - DNA, 20 bp, Cannabis sativa
atatatatacctgaagcgta

SEQ ID NO: 754 - DNA, 20 bp, Cannabis sativa
cacagacctccgtacgcttc

SEQ ID NO: 755 - DNA, 20 bp, Cannabis sativa
ttgacaacttttcaaaaaat

SEQ ID NO: 756 - DNA, 20 bp, Cannabis sativa
ggaatattgcatgtacttct

SEQ ID NO: 757 - DNA, 20 bp, Cannabis sativa
aattataaaatgcaacacat

SEQ ID NO: 758 - DNA, 20 bp, Cannabis sativa
taattataaaatgcaacaca

SEQ ID NO: 759 - DNA, 20 bp, Cannabis sativa
ccaccaactgtagtatagaa

SEQ ID NO: 760 - DNA, 20 bp, Cannabis sativa
aaacctttctatactacagt

SEQ ID NO: 761 - DNA, 20 bp, Cannabis sativa
cctttctatactacagttgg

SEQ ID NO: 762 - DNA, 20 bp, Cannabis sativa
ctttctatactacagttggt

SEQ ID NO: 763 - DNA, 20 bp, Cannabis sativa
cagttggtgggtgtgatgag

SEQ ID NO: 764 - DNA, 20 bp, Cannabis sativa
tgggtgtgatgagaggagtc

SEQ ID NO: 765 - DNA, 20 bp, Cannabis sativa
ttccatctgggcttcggctc

SEQ ID NO: 766 - DNA, 20 bp, Cannabis sativa
ttcgggttccatctgggctt

SEQ ID NO: 767 - DNA, 20 bp, Cannabis sativa
gtccggagccgaagcccaga

SEQ ID NO: 768 - DNA, 20 bp, Cannabis sativa
tccggtttcgggttccatct

SEQ ID NO: 769 - DNA, 20 bp, Cannabis sativa
ctccggtttcgggttccatc

SEQ ID NO: 770 - DNA, 20 bp, Cannabis sativa
gcccagatggaacccgaaac

SEQ ID NO: 771 - DNA, 20 bp, Cannabis sativa
ttcgaatctgctccggtttc

SEQ ID NO: 772 - DNA, 20 bp, Cannabis sativa
attcgaatctgctccggttt

SEQ ID NO: 773 - DNA, 20 bp, Cannabis sativa
tcaaggattcgaatctgctc

SEQ ID NO: 774 - DNA, 20 bp, Cannabis sativa
cagagttaaagatcgcttca

SEQ ID NO: 775 - DNA, 20 bp, Cannabis sativa
cttgaagcgatctttaactc

SEQ ID NO: 776 - DNA, 20 bp, Cannabis sativa
agcgatctttaactctggca

SEQ ID NO: 777 - DNA, 20 bp, Cannabis sativa
cttctaatctcgtctctcgg

SEQ ID NO: 778 - DNA, 20 bp, Cannabis sativa
ccttctaatctcgtctctcg

SEQ ID NO: 779 - DNA, 20 bp, Cannabis sativa
tccttctaatctcgtctctc

SEQ ID NO: 780 - DNA, 20 bp, Cannabis sativa
atccttctaatctcgtctct

SEQ ID NO: 781 - DNA, 20 bp, Cannabis sativa
ccccgagagacgagattaga

SEQ ID NO: 782 - DNA, 20 bp, Cannabis sativa
ttgtccatactcttgcaatt

SEQ ID NO: 783 - DNA, 20 bp, Cannabis sativa
cttgtccatactcttgcaat

SEQ ID NO: 784 - DNA, 20 bp, Cannabis sativa
agagcccaattgcaagagta

SEQ ID NO: 785 - DNA, 20 bp, Cannabis sativa
ttgcaagagtatggacaagt

SEQ ID NO: 786 - DNA, 20 bp, Cannabis sativa
ttgaaaccaataaaacacat

SEQ ID NO: 787 - DNA, 20 bp, Cannabis sativa
gtgatgccaatgtgttttat

SEQ ID NO: 788 - DNA, 20 bp, Cannabis sativa
ggtttcaaaacagaaaatct

SEQ ID NO: 789 - DNA, 20 bp, Cannabis sativa
gtttggagttttggaggtgg

SEQ ID NO: 790 - DNA, 20 bp, Cannabis sativa
gttgtttggagttttggagg

SEQ ID NO: 791 - DNA, 20 bp, Cannabis sativa
tttgttgtttggagttttgg

SEQ ID NO: 792 - DNA, 20 bp, Cannabis sativa
gggtttgttgtttggagttt

SEQ ID NO: 793 - DNA, 20 bp, Cannabis sativa
attttgaagggtttgttgtt

SEQ ID NO: 794 - DNA, 20 bp, Cannabis sativa
agttggagttggattttgaa

SEQ ID NO: 795 - DNA, 20 bp, Cannabis sativa
gagttggagttggattttga

SEQ ID NO: 796 - DNA, 20 bp, Cannabis sativa
gaggaaggaggagttggagt

SEQ ID NO: 797 - DNA, 20 bp, Cannabis sativa
gtggctgaggaaggaggagt

SEQ ID NO: 798 - DNA, 20 bp, Cannabis sativa
ggagcggtggctgaggaagg

SEQ ID NO: 799 - DNA, 20 bp, Cannabis sativa
gaaggagcggtggctgagga

SEQ ID NO: 800 - DNA, 20 bp, Cannabis sativa
agaggaaggagcggtggctg

SEQ ID NO: 801 - DNA, 20 bp, Cannabis sativa
agacgaagaggaaggagcgg

SEQ ID NO: 802 - DNA, 20 bp, Cannabis sativa
agaagacgaagaggaaggag

SEQ ID NO: 803 - DNA, 20 bp, Cannabis sativa
gacgaagaagacgaagagga

SEQ ID NO: 804 - DNA, 20 bp, Cannabis sativa
agacgacgaagaagacgaag

SEQ ID NO: 805 - DNA, 20 bp, Cannabis sativa
aagcctctcgatcgggattt

SEQ ID NO: 806 - DNA, 20 bp, Cannabis sativa
gaagcctctcgatcgggatt

SEQ ID NO: 807 - DNA, 20 bp, Cannabis sativa
tgatgagaagcctctcgatc

SEQ ID NO: 808 - DNA, 20 bp, Cannabis sativa
tctcccaaatcccgatcgag

SEQ ID NO: 809 - DNA, 20 bp, Cannabis sativa
atgatgagaagcctctcgat

SEQ ID NO: 810 - DNA, 20 bp, Cannabis sativa
gagaggcttctcatcatcat

SEQ ID NO: 811 - DNA, 20 bp, Cannabis sativa
agaggcttctcatcatcatt

SEQ ID NO: 812 - DNA, 20 bp, Cannabis sativa
gatgattagtagtactggct

SEQ ID NO: 813 - DNA, 20 bp, Cannabis sativa
atgatgatgattagtagtac

SEQ ID NO: 814 - DNA, 20 bp, Cannabis sativa
aagaagttggactagtttga

SEQ ID NO: 815 - DNA, 20 bp, Cannabis sativa
ttattattgaccgaagaagt

SEQ ID NO: 816 - DNA, 20 bp, Cannabis sativa
tcaaactagtccaacttctt

SEQ ID NO: 817 - DNA, 20 bp, Cannabis sativa
aatgttgttggtttgaaaga

SEQ ID NO: 818 - DNA, 20 bp, Cannabis sativa
agtattattattaatgttgt

SEQ ID NO: 819 - DNA, 20 bp, Cannabis sativa
aagaaatagtgttgatcggt

SEQ ID NO: 820 - DNA, 20 bp, Cannabis sativa
cgggaagaaatagtgttgat

SEQ ID NO: 821 - DNA, 20 bp, Cannabis sativa
cgatcaacactatttcttcc

SEQ ID NO: 822 - DNA, 20 bp, Cannabis sativa
ggtgatgattaggcacggcc

SEQ ID NO: 823 - DNA, 20 bp, Cannabis sativa
gggtgatgattaggcacggc

SEQ ID NO: 824 - DNA, 20 bp, Cannabis sativa
actggggtgatgattaggca

SEQ ID NO: 825 - DNA, 20 bp, Cannabis sativa
gtagaactggggtgatgatt

SEQ ID NO: 826 - DNA, 20 bp, Cannabis sativa
caggaacaggagtagaactg

SEQ ID NO: 827 - DNA, 20 bp, Cannabis sativa
gcaggaacaggagtagaact

SEQ ID NO: 828 - DNA, 20 bp, Cannabis sativa
agcaggaacaggagtagaac

SEQ ID NO: 829 - DNA, 20 bp, Cannabis sativa
tgactggtattagcaggaac

SEQ ID NO: 830 - DNA, 20 bp, Cannabis sativa
aacccttgactggtattagc

SEQ ID NO: 831 - DNA, 20 bp, Cannabis sativa
gttcctgctaataccagtca

SEQ ID NO: 832 - DNA, 20 bp, Cannabis sativa
ttcctgctaataccagtcaa

SEQ ID NO: 833 - DNA, 20 bp, Cannabis sativa
agggaagcaaaacccttgac

SEQ ID NO: 834 - DNA, 20 bp, Cannabis sativa
agagctcatgattttgagga

SEQ ID NO: 835 - DNA, 20 bp, Cannabis sativa
gagagctcatgattttgagg

SEQ ID NO: 836 - DNA, 20 bp, Cannabis sativa
tgagagagctcatgattttg

SEQ ID NO: 837 - DNA, 20 bp, Cannabis sativa
caaaatcatgagctctctca

SEQ ID NO: 838 - DNA, 20 bp, Cannabis sativa
aaaatcatgagctctctcat

SEQ ID NO: 839 - DNA, 20 bp, Cannabis sativa
aaatcatgagctctctcatg

SEQ ID NO: 840 - DNA, 20 bp, Cannabis sativa
ttctgtatctccatgtgatg

SEQ ID NO: 841 - DNA, 20 bp, Cannabis sativa
ggtagtactacctcatcaca

SEQ ID NO: 842 - DNA, 20 bp, Cannabis sativa
cacatggagatacagaatat

SEQ ID NO: 843 - DNA, 20 bp, Cannabis sativa
acatggagatacagaatata

SEQ ID NO: 844 - DNA, 20 bp, Cannabis sativa
ctaagtaaaaggcttgtaca

SEQ ID NO: 845 - DNA, 20 bp, Cannabis sativa
tcatgatctcactaagtaaa

SEQ ID NO: 846 - DNA, 20 bp, Cannabis sativa
agtgagatcatgaaccaaaa

SEQ ID NO: 847 - DNA, 20 bp, Cannabis sativa
ctttctttaagtcaccgttt

SEQ ID NO: 848 - DNA, 20 bp, Cannabis sativa
cttaaagaaagaccatcacg

SEQ ID NO: 849 - DNA, 20 bp, Cannabis sativa
gataattattcacctcgtga

SEQ ID NO: 850 - DNA, 20 bp, Cannabis sativa
gatgatgaagatgagctcta

SEQ ID NO: 851 - DNA, 20 bp, Cannabis sativa
cttgtctgatgacgccgact

SEQ ID NO: 852 - DNA, 20 bp, Cannabis sativa
gtagtggccggagtcctagt

SEQ ID NO: 853 - DNA, 20 bp, Cannabis sativa
gatgacgccgactaggactc

SEQ ID NO: 854 - DNA, 20 bp, Cannabis sativa
acgttagacggagtagtggc

SEQ ID NO: 855 - DNA, 20 bp, Cannabis sativa
gacgacgttagacggagtag

SEQ ID NO: 856 - DNA, 20 bp, Cannabis sativa
gatgaaacgacgacgttaga

SEQ ID NO: 857 - DNA, 20 bp, Cannabis sativa
gcagtagtagcgtggtggtg

SEQ ID NO: 858 - DNA, 20 bp, Cannabis sativa
tcgttgcagtagtagcgtgg

SEQ ID NO: 859 - DNA, 20 bp, Cannabis sativa
tagtcgttgcagtagtagcg

SEQ ID NO: 860 - DNA, 20 bp, Cannabis sativa
atttgattgagggggacaga

SEQ ID NO: 861 - DNA, 20 bp, Cannabis sativa
taccttggatttgattgagg

SEQ ID NO: 862 - DNA, 20 bp, Cannabis sativa
ttaccttggatttgattgag

SEQ ID NO: 863 - DNA, 20 bp, Cannabis sativa
tttaccttggatttgattga

SEQ ID NO: 864 - DNA, 20 bp, Cannabis sativa
ttttaccttggatttgattg

SEQ ID NO: 865 - DNA, 20 bp, Cannabis sativa
gtccccctcaatcaaatcca

SEQ ID NO: 866 - DNA, 20 bp, Cannabis sativa
cattttattttattttacct

SEQ ID NO: 867 - DNA, 20 bp, Cannabis sativa
taaaataaaatgttaccaca

SEQ ID NO: 868 - DNA, 20 bp, Cannabis sativa
ttttcagttacagttccatg

SEQ ID NO: 869 - DNA, 20 bp, Cannabis sativa
ctgaaaatcgaaattttgtt

SEQ ID NO: 870 - DNA, 20 bp, Cannabis sativa
gttatataaaaattttaaca

SEQ ID NO: 871 - DNA, 20 bp, Cannabis sativa
ttatataaaaattttaacac

SEQ ID NO: 872 - DNA, 20 bp, Cannabis sativa
taaaaattttaacacgggag

SEQ ID NO: 873 - DNA, 20 bp, Cannabis sativa
atttacaaattattatacat

SEQ ID NO: 874 - DNA, 20 bp, Cannabis sativa
ttatacattggtaaaagatc

SEQ ID NO: 875 - DNA, 20 bp, Cannabis sativa
atcaggacaatattgtctaa

SEQ ID NO: 876 - DNA, 20 bp, Cannabis sativa
tcaggacaatattgtctaat

SEQ ID NO: 877 - DNA, 20 bp, Cannabis sativa
tccatctacactcagatcgc

SEQ ID NO: 878 - DNA, 20 bp, Cannabis sativa
tccagcgatctgagtgtaga

SEQ ID NO: 879 - DNA, 20 bp, Cannabis sativa
agtgtagatggacgtgtgca

SEQ ID NO: 880 - DNA, 20 bp, Cannabis sativa
gatggacgtgtgcatggatt

SEQ ID NO: 881 - DNA, 20 bp, Cannabis sativa
ggacgtgtgcatggatttgg

SEQ ID NO: 882 - DNA, 20 bp, Cannabis sativa
gtgcatggatttggtggtgt

SEQ ID NO: 883 - DNA, 20 bp, Cannabis sativa
atttggtggtgttggtcctt

SEQ ID NO: 884 - DNA, 20 bp, Cannabis sativa
aatatgaaacagtcgaccta

SEQ ID NO: 885 - DNA, 20 bp, Cannabis sativa
tttcatattgatctttgtat

SEQ ID NO: 886 - DNA, 20 bp, Cannabis sativa
ttcatattgatctttgtatt

SEQ ID NO: 887 - DNA, 20 bp, Cannabis sativa
ttatttatgacgaaagtaat

SEQ ID NO: 888 - DNA, 20 bp, Cannabis sativa
tatttatgacgaaagtaata

SEQ ID NO: 889 - DNA, 20 bp, Cannabis sativa
cgaaagtaatagggtactac

SEQ ID NO: 890 - DNA, 20 bp, Cannabis sativa
gaaagtaatagggtactact

SEQ ID NO: 891 - DNA, 20 bp, Cannabis sativa
tatttaattaataaattatt

SEQ ID NO: 892 - DNA, 20 bp, Cannabis sativa
aaaaagcaaagttataaagg

SEQ ID NO: 893 - DNA, 20 bp, Cannabis sativa
cagaaaaagcaaagttataa

SEQ ID NO: 894 - DNA, 20 bp, Cannabis sativa
tctccacgatcgatcgaata

SEQ ID NO: 895 - DNA, 20 bp, Cannabis sativa
ataccttattcgatcgatcg

SEQ ID NO: 896 - DNA, 20 bp, Cannabis sativa
ttacatatatttatgagaac

SEQ ID NO: 897 - DNA, 20 bp, Cannabis sativa
tttatgagaacaggtgttgt

SEQ ID NO: 898 - DNA, 20 bp, Cannabis sativa
tgttgtaggagaagatgaac

SEQ ID NO: 899 - DNA, 20 bp, Cannabis sativa
gttgtaggagaagatgaaca

SEQ ID NO: 900 - DNA, 20 bp, Cannabis sativa
aggagaagatgaacagggag

SEQ ID NO: 901 - DNA, 20 bp, Cannabis sativa
agaagatgaacagggagtgg

SEQ ID NO: 902 - DNA, 20 bp, Cannabis sativa
gaagatgaacagggagtggt

SEQ ID NO: 903 - DNA, 20 bp, Cannabis sativa
gaacagggagtggtgggtcc

SEQ ID NO: 904 - DNA, 20 bp, Cannabis sativa
cagggagtggtgggtccagg

SEQ ID NO: 905 - DNA, 20 bp, Cannabis sativa
tttgccacaccatctccacc

SEQ ID NO: 906 - DNA, 20 bp, Cannabis sativa
gtggtgggtccaggtggaga

SEQ ID NO: 907 - DNA, 20 bp, Cannabis sativa
gggtccaggtggagatggtg

SEQ ID NO: 908 - DNA, 20 bp, Cannabis sativa
tggtgtggcaaataaaacga

SEQ ID NO: 909 - DNA, 20 bp, Cannabis sativa
gatggtgttcataaacgacg

SEQ ID NO: 910 - DNA, 20 bp, Cannabis sativa
cataaacgacgtggccttcg

SEQ ID NO: 911 - DNA, 20 bp, Cannabis sativa
aaacgacgtggccttcgagg

SEQ ID NO: 912 - DNA, 20 bp, Cannabis sativa
tggcccagcggccacctcga

SEQ ID NO: 913 - DNA, 20 bp, Cannabis sativa
gtggccttcgaggtggccgc

SEQ ID NO: 914 - DNA, 20 bp, Cannabis sativa
tggccttcgaggtggccgct

SEQ ID NO: 915 - DNA, 20 bp, Cannabis sativa
gcgcacgttgaatggcccag

SEQ ID NO: 916 - DNA, 20 bp, Cannabis sativa
aaagcctcgcgcacgttgaa

SEQ ID NO: 917 - DNA, 20 bp, Cannabis sativa
tgggccattcaacgtgcgcg

SEQ ID NO: 918 - DNA, 20 bp, Cannabis sativa
ttcaacgtgcgcgaggcttt

SEQ ID NO: 919 - DNA, 20 bp, Cannabis sativa
gctgtacttattcactccaa

SEQ ID NO: 920 - DNA, 20 bp, Cannabis sativa
ggtgggaacaagttgaccat

SEQ ID NO: 921 - DNA, 20 bp, Cannabis sativa
gtgacgccccagtcgtcggt

SEQ ID NO: 922 - DNA, 20 bp, Cannabis sativa
agtgacgccccagtcgtcgg

SEQ ID NO: 923 - DNA, 20 bp, Cannabis sativa
aacttgttcccaccgacgac

SEQ ID NO: 924 - DNA, 20 bp, Cannabis sativa
acttgttcccaccgacgact

SEQ ID NO: 925 - DNA, 20 bp, Cannabis sativa
aggagtgacgccccagtcgt

SEQ ID NO: 926 - DNA, 20 bp, Cannabis sativa
cttgttcccaccgacgactg

SEQ ID NO: 927 - DNA, 20 bp, Cannabis sativa
gcgccgtgctggagggagtg

SEQ ID NO: 928 - DNA, 20 bp, Cannabis sativa
gtaaaaagcgccgtgctgga

SEQ ID NO: 929 - DNA, 20 bp, Cannabis sativa
actcctcactccctccagca

SEQ ID NO: 930 - DNA, 20 bp, Cannabis sativa
agtaaaaagcgccgtgctgg

SEQ ID NO: 931 - DNA, 20 bp, Cannabis sativa
gatagtaaaaagcgccgtgc

SEQ ID NO: 932 - DNA, 20 bp, Cannabis sativa
gcgctttttactatcttatc

SEQ ID NO: 933 - DNA, 20 bp, Cannabis sativa
acgtaaataatggtgtgaaa

SEQ ID NO: 934 - DNA, 20 bp, Cannabis sativa
aaaaagatttacgtaaataa

SEQ ID NO: 935 - DNA, 20 bp, Cannabis sativa
ttatttgattataatatttg

SEQ ID NO: 936 - DNA, 4912 bp, Cannabis sativa
taccattttagaaaatacaagatccattttgtcatttaacaaaacataaggtccaatcgataacttttgcaaaacacaaggtccaaaatatataatatttatccgtaaaatcttttattattttatttttattatcaatttttttaagaaaaaaacatcttcagatttattttgaatatacacgatagttgttgagttgggtgcgtgatcaatgtaatgattcttatggtaaagtgcatgaagattataattaaacaaataatttttatatatttaagggtaaatattattgtgaaatctatatttagtaaaaattattgactatataaattttcatatatgtttttcaaaatgaaacatatataaatatatatatttatattatataatattaagctcagttttattaaatgcaatgactcgaaatgtaaccttatgggataaacatgtatatattataaacaaaattatagatagaatgtcaaaagagatgtttgtatcagattaattttctcattttaaaagagaaaaaatcagttatatgccgatcgaatttgatcgagcaatacttacccatttgtaattgaacttttgtagctccaagtttgtaaagtgtttaattgatagtgcatacaattttatttgattacaaaaaagattaataaaatccttgaggtggtataacgaggctagttatatcttattattattattatttttattttttttttttgactaaaattgagattatgtattattttgtgttagaatttgaggaataagatgaatgaagttccatcccaaaatcaattggctatggaggagtagcctttttatcttatatatcataatttatttccacattagcaatgtgagactaactcttaatattttgagaaatacaaaatctgattttatttaacaaaaatatgaaatttgatcgataacttttgtaaacacatataggatccaaaaatgacatttaactatattttatttagtgaaaaatcttccaagaaacgtactctcaaattaattaatgtttaacatcactacaccaacgtgaatgacttggctctctatctaggatatatagataatttttttgttccaaaatatattaaacgtgggagatgagtaaattttttatgatatgttagcaattttttttaaaaaaaaaaaattatttggaacacattaattattttattttgaatattacaaacattattttattaacttttatatgatttattatttaacatagaaacaacataatatgtcattcatgtttctatttgtttatcgagctaattattaagagaggagataaagaaagaaaaatgaagatatataacaatattatatatataaggatatatgttatatatatatatgagaagatgaaaagttgacaacgttggacagaaaaataaggatgatcatcaaatgaatcttaaaaaaaaaaaaaattggtccacacaactcccaataactttacagtagtcggccattgctataagaattatataaataaataaatgtttttactaattaagagaatccacaatatatattagaaccttttgatgttacaaaagctaaaagagagaatataagttataactataaatagagccataaattgttgaatgtcagccaccaaattatataaccaaaactcatcaaaccctcttctgatctctctctacatatatttatatatagttgttatatatataaatggctaatagggatcctcttgtggttgggagagtgataagtgatgtgttggaccctttcacaaaaactgtgtctcttaaagtatcatatagtgggaatagggctattaacaatggtactgaccttagaccttctcaagttgctaactcacctagggttgagattggtggagatgatctcaggactttctacactttggtatatatatatatcttcactactctatcttcataataaatcattgataaattaatgtataattaatactgttaatgttgcaggttatggttgatcctgatgctcctaaccctagcgaacctaatcttaaagaatatttgcattggtatgtacttagtaatatatcacttcgagtatcatataatttaatttgataacttttagatatattacttaggaatcacaaggcaccatcaataagtgtgtgccactaataattattaatttttatttttcttaatttagtttaaatttaattaaagtgtataattttgtcaggttggtgactgatattcccgcaactacgggaacaggctttggtacatatttatacactcattttttttgttaatatttatgcacaaaacacaattataatttattttacagaaaataacaccaataagttaatggtaagttaacaacgttatttaaaaaaaatgttaaattaagaattatcattaaataattacttttaatatataaagatatagtgtgtgtgatgatttaatgatgggcgtacgcatatatgtaggacaagaagtgatgtgctacgagagcccaagaccgacggtgggaattcatcgctttgtgtttgttctgtttcgacagttaggaaggcagacagtgtatgcacccgggtggcgacataacttcaacacaagagattttgctgaaatctacaaccttggattgcctgttgctgccgtttacttcaactgccaaaaggaacttggctgtggtggaaggaggtcatcaacatcataaatattaattatataatatactaatcatattaatcataatctatccatctatctatctatatatgtcatattattatatattgaataatgcttgagacacaacaactacaactattaattttatatattctataagttattgatatatctatatatagttgtgatttctcttataataataaaacagtagtattataattaagctagctaacactattgtactacagatatataatatgaaattgagttgtaaatgtttcacgtactacgcatctagcaatatttaatatttttataaatattaaaattactagatgttaagttgtaaatgtttatgatgtatcatcaataatatatatggatgcttcttgttacttattaccatcttcattatatattattattaattagttttgtttttcaatttgtatatacttaaccaataaaaatattataagaaaaaaaattaaaaaactatagtacacgtgattttttttctttttctttttttttttttgaaaattaatgttggagattattttaccgtgatctttactactatgatgttatttaacatcctaaatatgaatttctaaatgactctttccccactcagatctctaacctttgtatcctttctctagcagagtatcaccaagatctgagcccgattctctttcaattgattggattattcacagtcttacacactatgattgaatatcaacttgctatatgcgtgagcatgtactctatcactataggatttgaaaatttagaagagagaaagagagagaaggattcgaattttgagaagatacactactacaaacaaaggcctttaatcccagtttttaaggccttttatcctggtttttcgctaaaataaaaaccgggatatatgccagtgggataaaaagtctttgaaaaactgggataaaaactttatcccggttatttcataaaaatcgggatagaaggggactttttatcccagtttttatgaaataaccgggattaaaagtaaaaaatataataagaaaataaacccaaaaatattatatcagttctttgaaataatttttacaattagattccaaaaatttatatcaaaatatacaaattaatacaccatcagggttcacaaaaaaaaaaactcatcaaggaccaccaatgacgaagcttcgtgcaccacaaaacgccatgaacctacaagcactgtgaccgacgaatggccacagatcagctctatttacctaagaaaataaagaaggagatagatccagaatgatgaccgaagaatcgggagtttattagcaaaaaaaataaaataagaacaagataaaataatacctggaggttttggagggtatgaggctcagatttgccctccgttgggtcgtccgtggctggagaaaggcggttggacaagctggtcttcatgcagcagaagagaaacatgagagagagagagagagagagagagagagagagagagagagagagagagagagagagagagagagagagaaggaagagagagcatggaaaggagagagagcagtagataaagaacatggagtgagagagaggggcttacacgctgagagagcggcggaagagacaacatataaatgaaaccctaaacctaaatgttctttatatatgtgcagctataaaaaagaaaattgggtgggactttttatcccgatttttataaaaagtcccacttaaaatgctaaggttgcgtttgacaaatgcataaaaatttattttccaacacctttttttgtgcgattgccaaacaaggcccttgctcctatcttttatttaattaaagttccaaatgtaaaagtgggatcaaaagtttttattccactttttattttttcaaatgtaaaaagtgggataaaaagtcccttttgttgtagtgatagaagactcaagttttgactgaaggctagtgtgacttaggctatcatcaactatcagttttgagtccatcactatctatttataggaatccttctaggtttaggtttaaattatttggcattaaaaacttgataaataaatggtaaaatcatgcaatagtggccgaccatagaatgtgggcccaccatttgcaattttgccatttttcaactatttatcctattctttca

SEQ ID NO: 937 - DNA, 537 bp, Cannabis sativa
atggctaatagggatcctcttgtggttgggagagtgataagtgatgtgttggaccctttcacaaaaactgtgtctcttaaagtatcatatagtgggaatagggctattaacaatggtactgaccttagaccttctcaagttgctaactcacctagggttgagattggtggagatgatctcaggactttctacactttggtagttatggttgatcctgatgctcctaaccctagcgaacctaatcttaaagaatatttgcattggtatgtgactgatattcccgcaactacgggaacaggctttggacaagaagtgatgtgctacgagagcccaagaccgacggtgggaattcatcgctttgtgtttgttctgtttcgacagttaggaaggcagacagtgtatgcacccgggtggcgacataacttcaacacaagagattttgctgaaatctacaaccttggattgcctgttgctgccgtttacttcaactgccaaaaggaacttggctgtggtggaaggaggtcatcaacatcataa

SEQ ID NO: 938 - protein, 178 aa, Artificial Sequence
  CsSFT1 AA
MANRDPLVVGRVISDVLDPFTKTVSLKVSYSGNRAINNGTDLRPSQVANSPRVEIGGDDLRTFYTLVVMVDPDAPNPSEPNLKEYLHWYVTDIPATTGTGFGQEVMCYESPRPTVGIHRFVFVLFRQLGRQTVYAPGWRHNFNTRDFAEIYNLGLPVAAVYFNCQKELGCGGRRSSTS

SEQ ID NO: 939 - DNA, 20 bp, Cannabis sativa
tgtagagagagatcagaaga

SEQ ID NO: 940 - DNA, 20 bp, Cannabis sativa
atgtagagagagatcagaag

SEQ ID NO: 941 - DNA, 20 bp, Cannabis sativa
atagttgttatatatataaa

SEQ ID NO: 942 - DNA, 20 bp, Cannabis sativa
tatatatataaatggctaat

SEQ ID NO: 943 - DNA, 20 bp, Cannabis sativa
atatatataaatggctaata

SEQ ID NO: 944 - DNA, 20 bp, Cannabis sativa
ggctaatagggatcctcttg

SEQ ID NO: 945 - DNA, 20 bp, Cannabis sativa
atcactctcccaaccacaag

SEQ ID NO: 946 - DNA, 20 bp, Cannabis sativa
aatagggatcctcttgtggt

SEQ ID NO: 947 - DNA, 20 bp, Cannabis sativa
atagggatcctcttgtggtt

SEQ ID NO: 948 - DNA, 20 bp, Cannabis sativa
gagagtgataagtgatgtgt

SEQ ID NO: 949 - DNA, 20 bp, Cannabis sativa
gagacacagtttttgtgaaa

SEQ ID NO: 950 - DNA, 20 bp, Cannabis sativa
agagacacagtttttgtgaa

SEQ ID NO: 951 - DNA, 20 bp, Cannabis sativa
tctcttaaagtatcatatag

SEQ ID NO: 952 - DNA, 20 bp, Cannabis sativa
ctcttaaagtatcatatagt

SEQ ID NO: 953 - DNA, 20 bp, Cannabis sativa
aagtatcatatagtgggaat

SEQ ID NO: 954 - DNA, 20 bp, Cannabis sativa
agtatcatatagtgggaata

SEQ ID NO: 955 - DNA, 20 bp, Cannabis sativa
gggaatagggctattaacaa

SEQ ID NO: 956 - DNA, 20 bp, Cannabis sativa
tagcaacttgagaaggtcta

SEQ ID NO: 957 - DNA, 20 bp, Cannabis sativa
ggtgagttagcaacttgaga

SEQ ID NO: 958 - DNA, 20 bp, Cannabis sativa
ctcaagttgctaactcacct

SEQ ID NO: 959 - DNA, 20 bp, Cannabis sativa
tcaagttgctaactcaccta

SEQ ID NO: 960 - DNA, 20 bp, Cannabis sativa
tctccaccaatctcaaccct

SEQ ID NO: 961 - DNA, 20 bp, Cannabis sativa
aactcacctagggttgagat

SEQ ID NO: 962 - DNA, 20 bp, Cannabis sativa
tcacctagggttgagattgg

SEQ ID NO: 963 - DNA, 20 bp, Cannabis sativa
agattggtggagatgatctc

SEQ ID NO: 964 - DNA, 20 bp, Cannabis sativa
tctcaggactttctacactt

SEQ ID NO: 965 - DNA, 20 bp, Cannabis sativa
attaatactgttaatgttgc

SEQ ID NO: 966 - DNA, 20 bp, Cannabis sativa
actgttaatgttgcaggtta

SEQ ID NO: 967 - DNA, 20 bp, Cannabis sativa
tcgctagggttaggagcatc

SEQ ID NO: 968 - DNA, 20 bp, Cannabis sativa
agattaggttcgctagggtt

SEQ ID NO: 969 - DNA, 20 bp, Cannabis sativa
ctttaagattaggttcgcta

SEQ ID NO: 970 - DNA, 20 bp, Cannabis sativa
tctttaagattaggttcgct

SEQ ID NO: 971 - DNA, 20 bp, Cannabis sativa
tgcaaatattctttaagatt

SEQ ID NO: 972 - DNA, 20 bp, Cannabis sativa
atcttaaagaatatttgcat

SEQ ID NO: 973 - DNA, 20 bp, Cannabis sativa
aacttttagatatattactt

SEQ ID NO: 974 - DNA, 20 bp, Cannabis sativa
tatattacttaggaatcaca

SEQ ID NO: 975 - DNA, 20 bp, Cannabis sativa
tagtggcacacacttattga

SEQ ID NO: 976 - DNA, 20 bp, Cannabis sativa
taaaaattaataattattag

SEQ ID NO: 977 - DNA, 20 bp, Cannabis sativa
ttaaagtgtataattttgtc

SEQ ID NO: 978 - DNA, 20 bp, Cannabis sativa
agtgtataattttgtcaggt

SEQ ID NO: 979 - DNA, 20 bp, Cannabis sativa
aagcctgttcccgtagttgc

SEQ ID NO: 980 - DNA, 20 bp, Cannabis sativa
gactgatattcccgcaacta

SEQ ID NO: 981 - DNA, 20 bp, Cannabis sativa
aaagcctgttcccgtagttg

SEQ ID NO: 982 - DNA, 20 bp, Cannabis sativa
actgatattcccgcaactac

SEQ ID NO: 983 - DNA, 20 bp, Cannabis sativa
attcccgcaactacgggaac

SEQ ID NO: 984 - DNA, 20 bp, Cannabis sativa
gcaactacgggaacaggctt

SEQ ID NO: 985 - DNA, 20 bp, Cannabis sativa
ttaacttaccattaacttat

SEQ ID NO: 986 - DNA, 20 bp, Cannabis sativa
aaataacaccaataagttaa

SEQ ID NO: 987 - DNA, 20 bp, Cannabis sativa
tgtgtgtgatgatttaatga

SEQ ID NO: 988 - DNA, 20 bp, Cannabis sativa
gtgtgtgatgatttaatgat

SEQ ID NO: 989 - DNA, 20 bp, Cannabis sativa
atgggcgtacgcatatatgt

SEQ ID NO: 990 - DNA, 20 bp, Cannabis sativa
gaattcccaccgtcggtctt

SEQ ID NO: 991 - DNA, 20 bp, Cannabis sativa
tgaattcccaccgtcggtct

SEQ ID NO: 992 - DNA, 20 bp, Cannabis sativa
ctacgagagcccaagaccga

SEQ ID NO: 993 - DNA, 20 bp, Cannabis sativa
cgagagcccaagaccgacgg

SEQ ID NO: 994 - DNA, 20 bp, Cannabis sativa
gagagcccaagaccgacggt

SEQ ID NO: 995 - DNA, 20 bp, Cannabis sativa
aagcgatgaattcccaccgt

SEQ ID NO: 996 - DNA, 20 bp, Cannabis sativa
tttgttctgtttcgacagtt

SEQ ID NO: 997 - DNA, 20 bp, Cannabis sativa
ttctgtttcgacagttagga

SEQ ID NO: 998 - DNA, 20 bp, Cannabis sativa
aggcagacagtgtatgcacc

SEQ ID NO: 999 - DNA, 20 bp, Cannabis sativa
ggcagacagtgtatgcaccc

SEQ ID NO: 1000 - DNA, 20 bp, Cannabis sativa
agacagtgtatgcacccggg

SEQ ID NO: 1001 - DNA, 20 bp, Cannabis sativa
ttgaagttatgtcgccaccc

SEQ ID NO: 1002 - DNA, 20 bp, Cannabis sativa
gttgaagttatgtcgccacc

SEQ ID NO: 1003 - DNA, 20 bp, Cannabis sativa
tttgctgaaatctacaacct

SEQ ID NO: 1004 - DNA, 20 bp, Cannabis sativa
cggcagcaacaggcaatcca

SEQ ID NO: 1005 - DNA, 20 bp, Cannabis sativa
ttgaagtaaacggcagcaac

SEQ ID NO: 1006 - DNA, 20 bp, Cannabis sativa
cttttggcagttgaagtaaa

SEQ ID NO: 1007 - DNA, 20 bp, Cannabis sativa
cgtttacttcaactgccaaa

SEQ ID NO: 1008 - DNA, 20 bp, Cannabis sativa
caccacagccaagttccttt

SEQ ID NO: 1009 - DNA, 20 bp, Cannabis sativa
ttcaactgccaaaaggaact

SEQ ID NO: 1010 - DNA, 20 bp, Cannabis sativa
tgccaaaaggaacttggctg

SEQ ID NO: 1011 - DNA, 20 bp, Cannabis sativa
caaaaggaacttggctgtgg

SEQ ID NO: 1012 - DNA, 20 bp, Cannabis sativa
aggaacttggctgtggtgga

SEQ ID NO: 1013 - DNA, 20 bp, Cannabis sativa
aacttggctgtggtggaagg

SEQ ID NO: 1014 - DNA, 20 bp, Cannabis sativa
gacatatatagatagataga

SEQ ID NO: 1015 - DNA, 3388 bp, Cannabis sativa
tttaaagactttttatcccactggcatatattccgatttttattttagcgaaaaaccgggataaaaggccttaaaaaccgggattaaaggcctttgtttgtagtagtgtatcttctcaaaatttgaatccttctctgtctttctctcttctaaattttcaaatcctatagtgatagagtacatgcccacgcatagcaagttgatattcaatcatagtgtgtaagactgtgaataatccaatcaactgaaagagaatcaggctcagatcttggtgatactctgctagagaaaggatacaatggatagatctgagtggggaaagagtcatttaattctgttgtaaccaatgtaaggtttcttgaactttatatgtgtttaaattctattgttttagaaatttatatttaggatgttaaataacatcatagtagtaaagattatggtaaaataatctccaatgttaatttttttttaaaaaaaaaaaaatcacgtgtaccatatttttttaattttttttcttataatattttgattggttaagtatatacaaattgaaaaacaaaactaattaataataatatataatgaagagggtaataagtaacaagaagcatccatatatatattattgatgatacatcataaacatttacaacttaacatctagtaattttaatatttataaaaatattaaatattgctagatgcgtagtacgtgaaacatttacaactcaatttcatatattatatatctgtagtacaatagtgttagctagcttaattatactactactattttattattacaagagaaatcacaactatataatatcaattacttaaaatatatataaatattaatagttgtagttgttgtgtctcaagcattattcaatataataatatgacatatggatagatagatagatggatatattatgattataattatatataattatatttatgaagttgatgacctccttccaccacagccaagttccttttggcagttgaagtaaacgacagcaacaggcaatccaaggttgtagatctcagcaaaatctcttgtgttgaagttatgtcgccacccaggtgcatacactgtctgccttcctaactgtcgaaacagaacaaacacatagcgatgtattcccaccgtcggtctagggctctcgtagcacatcacttcttgtcctacatatatgcgtatatacacacatcattaaatcatcacacataatatatatatatctttatatatcaaaagtaattatcgaatgataattcttgattaaaattttttttaacttcttaaaataacattgttaacttatcgttaacttattggtgttattagttctgtaaaatatatataattgtgttttgtgcataaatattaacaaaaaaatgagtgtattaatatgtaccaaaggctgttcccgtagttgcgggaatatcagtcaccaacctgaaaaagttatacacttaaattaaatttaaacttaagaaaaataaaattaataattattagtggcacacactattgatggtgccttgtcattcgttagtaatattctctaaaaattatcaaattaaattataccgaaatgatattactaagtacataccaatgcaaatattctttaagattaggttcgctagggttaggagcatcagatcaaccataacctgcaacattaacagtattaattatacgttaatttataaatgatttattatgaagatagagttaagatatatatatatatatactagttagatattacgtgcgatgcacgtatacttaattttatgctaggtgtttttttactttatttacaaacaatacaataaataattaaaaaaaaaaaaaacatattatttagagaagaataatagagaaaaatagttaacgatgattcgattacagaatttgtgtatttgtgcaattataagttaggccttattgttgtgggtctttgacatgtgattagtatcaaatatactaattaacagtgttaattttaaacggttaaacagaaaattcacaacaaatctgttaaacagaacaaatatcagttcaaatattaatgtgattcgttttatttttattttattgtatataaataatattttattattttattaaatataaaaaaaagtcacccactaatttttcataaaccaagaatttagttatataggcacactttatatagaatagatatagatataaatactgaccaaagtgtagaaagtcctgagatcatctccaccaatctcaaccctaggtgagttagcaacttgagaaggtctaaggtcagtaccattgttaatagccctaatcccactatatgatactttaagagagacagtttttgtgaaagggtccaacacatcacttatcactctcccaaccacaagaggatccctattagccatttatatatataacaactatatacagtataaatatgtagagagatagagagaagaagagggtttgatgagtttttggttgtataatttggtggctgacattcaacaatttatggctctatttatagttatcacttatattctctctgttagcttttgtaacatcaaaaggttctaatatatattgtggattctcttaattagtaaaaacatttatttatttatttatatattcttgtaacaatggccgactactgtaaagttattgggagttgtgtggaccaattttttttttttttttaaagattcatttgatgatcatccttatttttctgtccaacgttgtcaacttttcatcttctcatatatatatataacatatattcgtatatatattgttatatatcatcctttttcttctttctatatctcctatcttaataattagctcgataaacaatagaaacatgtatgacgtcatattatgttgtttctatgttaaaataatacatcatataaaagttaataaaataatgtttgtaatattcaaaataaaataattaatgtgtgttccaaataaaattatttttttttaaaaaaaaaaattgctaacatatcataaaaaattaactcatctcccacatttaatatattttggaacaaaaaaatgatctatatattctagatagagagccaagtcattcacgttggtgtagtcactacaagaaatgttacttttgctggcacatttttgtgctggcaaaagttggtattgcgctgataaaatagaatttacctatgtgttggcaaaagagggttggcaaatcaatgttggtatttgaacttttgccagcataaaatgaaaagcgttggcaaaaaagactttttccagcgcgtaaatgtgctagtaaaagttagattttagccactgcaaatgtacgctggt

SEQ ID NO: 1016 - DNA, 489 bp, Cannabis sativa
atggctaatagggatcctcttgtggttgggagagtgataagtgatgtgttggaccctttcacaaaaactgtctctcttaaagtatcatatagtgggattagggctattaacaatggtactgaccttagaccttctcaagttgctaactcacctagggttgagattggtggagatgatctcaggactttctacactttggtcgatgctcctaaccctagcgaacctaatcttaaagaatatttgcattggtatgtaggacaagaagtgatgtgctacgagagccctagaccgacggtgggaatacatcgctatgtgtttgttctgtttcgacagttaggaaggcagacagtgtatgcacctgggtggcgacataacttcaacacaagagattttgctgagatctacaaccttggattgcctgttgctgtcgtttacttcaactgccaaaaggaacttggctgtggtggaaggaggtcatcaacttcataa

SEQ ID NO: 1017 - protein, 162 aa, Artificial Sequence
  CsSFT2 AA
MANRDPLVVGRVISDVLDPFTKTVSLKVSYSGIRAINNGTDLRPSQVANSPRVEIGGDDLRTFYTLVDAPNPSEPNLKEYLHWYVGQEVMCYESPRPTVGIHRYVFVLFRQLGRQTVYAPGWRHNFNTRDFAEIYNLGLPVAVVYFNCQKELGCGGRRSSTS

SEQ ID NO: 1018 - DNA, 20 bp, Cannabis sativa
caatataataatatgacata

SEQ ID NO: 1019 - DNA, 20 bp, Cannabis sativa
catatggatagatagataga

SEQ ID NO: 1020 - DNA, 20 bp, Cannabis sativa
aacttggctgtggtggaagg

SEQ ID NO: 1021 - DNA, 20 bp, Cannabis sativa
aggaacttggctgtggtgga

SEQ ID NO: 1022 - DNA, 20 bp, Cannabis sativa
caaaaggaacttggctgtgg

SEQ ID NO: 1023 - DNA, 20 bp, Cannabis sativa
tgccaaaaggaacttggctg

SEQ ID NO: 1024 - DNA, 20 bp, Cannabis sativa
ttcaactgccaaaaggaact

SEQ ID NO: 1025 - DNA, 20 bp, Cannabis sativa
caccacagccaagttccttt

SEQ ID NO: 1026 - DNA, 20 bp, Cannabis sativa
cgtttacttcaactgccaaa

SEQ ID NO: 1027 - DNA, 20 bp, Cannabis sativa
ttgaagtaaacgacagcaac

SEQ ID NO: 1028 - DNA, 20 bp, Cannabis sativa
cgacagcaacaggcaatcca

SEQ ID NO: 1029 - DNA, 20 bp, Cannabis sativa
tttgctgagatctacaacct

SEQ ID NO: 1030 - DNA, 20 bp, Cannabis sativa
ttgaagttatgtcgccaccc

SEQ ID NO: 1031 - DNA, 20 bp, Cannabis sativa
agacagtgtatgcacctggg

SEQ ID NO: 1032 - DNA, 20 bp, Cannabis sativa
ggcagacagtgtatgcacct

SEQ ID NO: 1033 - DNA, 20 bp, Cannabis sativa
aggcagacagtgtatgcacc

SEQ ID NO: 1034 - DNA, 20 bp, Cannabis sativa
ttctgtttcgacagttagga

SEQ ID NO: 1035 - DNA, 20 bp, Cannabis sativa
tttgttctgtttcgacagtt

SEQ ID NO: 1036 - DNA, 20 bp, Cannabis sativa
tagcgatgtattcccaccgt

SEQ ID NO: 1037 - DNA, 20 bp, Cannabis sativa
gagagccctagaccgacggt

SEQ ID NO: 1038 - DNA, 20 bp, Cannabis sativa
cgagagccctagaccgacgg

SEQ ID NO: 1039 - DNA, 20 bp, Cannabis sativa
ctacgagagccctagaccga

SEQ ID NO: 1040 - DNA, 20 bp, Cannabis sativa
tgtattcccaccgtcggtct

SEQ ID NO: 1041 - DNA, 20 bp, Cannabis sativa
gtattcccaccgtcggtcta

SEQ ID NO: 1042 - DNA, 20 bp, Cannabis sativa
gtgtatatacgcatatatgt

SEQ ID NO: 1043 - DNA, 20 bp, Cannabis sativa
ttaacttatcgttaacttat

SEQ ID NO: 1044 - DNA, 20 bp, Cannabis sativa
agtgtattaatatgtaccaa

SEQ ID NO: 1045 - DNA, 20 bp, Cannabis sativa
gcaactacgggaacagcctt

SEQ ID NO: 1046 - DNA, 20 bp, Cannabis sativa
actgatattcccgcaactac

SEQ ID NO: 1047 - DNA, 20 bp, Cannabis sativa
aaaggctgttcccgtagttg

SEQ ID NO: 1048 - DNA, 20 bp, Cannabis sativa
gactgatattcccgcaacta

SEQ ID NO: 1049 - DNA, 20 bp, Cannabis sativa
aaggctgttcccgtagttgc

SEQ ID NO: 1050 - DNA, 20 bp, Cannabis sativa
agtgtataactttttcaggt

SEQ ID NO: 1051 - DNA, 20 bp, Cannabis sativa
tttaagtgtataactttttc

SEQ ID NO: 1052 - DNA, 20 bp, Cannabis sativa
ataaaattaataattattag

SEQ ID NO: 1053 - DNA, 20 bp, Cannabis sativa
ttagtggcacacactattga

SEQ ID NO: 1054 - DNA, 20 bp, Cannabis sativa
aatattactaacgaatgaca

SEQ ID NO: 1055 - DNA, 20 bp, Cannabis sativa
tgtacttagtaatatcattt

SEQ ID NO: 1056 - DNA, 20 bp, Cannabis sativa
atcttaaagaatatttgcat

SEQ ID NO: 1057 - DNA, 20 bp, Cannabis sativa
tgcaaatattctttaagatt

SEQ ID NO: 1058 - DNA, 20 bp, Cannabis sativa
tctttaagattaggttcgct

SEQ ID NO: 1059 - DNA, 20 bp, Cannabis sativa
ctttaagattaggttcgcta

SEQ ID NO: 1060 - DNA, 20 bp, Cannabis sativa
agattaggttcgctagggtt

SEQ ID NO: 1061 - DNA, 20 bp, Cannabis sativa
actgttaatgttgcaggtta

SEQ ID NO: 1062 - DNA, 20 bp, Cannabis sativa
attaatactgttaatgttgc

SEQ ID NO: 1063 - DNA, 20 bp, Cannabis sativa
cgtatacttaattttatgct

SEQ ID NO: 1064 - DNA, 20 bp, Cannabis sativa
tatttgtgcaattataagtt

SEQ ID NO: 1065 - DNA, 20 bp, Cannabis sativa
gtcaaagacccacaacaata

SEQ ID NO: 1066 - DNA, 20 bp, Cannabis sativa
taagttaggccttattgttg

SEQ ID NO: 1067 - DNA, 20 bp, Cannabis sativa
aagttaggccttattgttgt

SEQ ID NO: 1068 - DNA, 20 bp, Cannabis sativa
ttaacagtgttaattttaaa

SEQ ID NO: 1069 - DNA, 20 bp, Cannabis sativa
ttggtttatgaaaaattagt

SEQ ID NO: 1070 - DNA, 20 bp, Cannabis sativa
cttggtttatgaaaaattag

SEQ ID NO: 1071 - DNA, 20 bp, Cannabis sativa
tgcctatataactaaattct

SEQ ID NO: 1072 - DNA, 20 bp, Cannabis sativa
aaccaagaatttagttatat

SEQ ID NO: 1073 - DNA, 20 bp, Cannabis sativa
tctcaggactttctacactt

SEQ ID NO: 1074 - DNA, 20 bp, Cannabis sativa
agattggtggagatgatctc

SEQ ID NO: 1075 - DNA, 20 bp, Cannabis sativa
tcacctagggttgagattgg

SEQ ID NO: 1076 - DNA, 20 bp, Cannabis sativa
aactcacctagggttgagat

SEQ ID NO: 1077 - DNA, 20 bp, Cannabis sativa
tctccaccaatctcaaccct

SEQ ID NO: 1078 - DNA, 20 bp, Cannabis sativa
tcaagttgctaactcaccta

SEQ ID NO: 1079 - DNA, 20 bp, Cannabis sativa
ctcaagttgctaactcacct

SEQ ID NO: 1080 - DNA, 20 bp, Cannabis sativa
ggtgagttagcaacttgaga

SEQ ID NO: 1081 - DNA, 20 bp, Cannabis sativa
tagcaacttgagaaggtcta

SEQ ID NO: 1082 - DNA, 20 bp, Cannabis sativa
gggattagggctattaacaa

SEQ ID NO: 1083 - DNA, 20 bp, Cannabis sativa
agtatcatatagtgggatta

SEQ ID NO: 1084 - DNA, 20 bp, Cannabis sativa
aagtatcatatagtgggatt

SEQ ID NO: 1085 - DNA, 20 bp, Cannabis sativa
ctcttaaagtatcatatagt

SEQ ID NO: 1086 - DNA, 20 bp, Cannabis sativa
tctcttaaagtatcatatag

SEQ ID NO: 1087 - DNA, 20 bp, Cannabis sativa
agagagacagtttttgtgaa

SEQ ID NO: 1088 - DNA, 20 bp, Cannabis sativa
gagagacagtttttgtgaaa

SEQ ID NO: 1089 - DNA, 20 bp, Cannabis sativa
gagagtgataagtgatgtgt

SEQ ID NO: 1090 - DNA, 20 bp, Cannabis sativa
atagggatcctcttgtggtt

SEQ ID NO: 1091 - DNA, 20 bp, Cannabis sativa
aatagggatcctcttgtggt

SEQ ID NO: 1092 - DNA, 20 bp, Cannabis sativa
atcactctcccaaccacaag

SEQ ID NO: 1093 - DNA, 20 bp, Cannabis sativa
ggctaatagggatcctcttg

SEQ ID NO: 1094 - DNA, 20 bp, Cannabis sativa
atatatataaatggctaata

SEQ ID NO: 1095 - DNA, 20 bp, Cannabis sativa
tatatatataaatggctaat

SEQ ID NO: 1096 - DNA, 20 bp, Cannabis sativa
atagttgttatatatataaa

SEQ ID NO: 1097 - DNA, 20 bp, Cannabis sativa
agagagatagagagaagaag

SEQ ID NO: 1098 - DNA, 20 bp, Cannabis sativa
gagagatagagagaagaaga

SEQ ID NO: 1099 - DNA, 20 bp, Cannabis sativa
aagagggtttgatgagtttt

SEQ ID NO: 1100 - DNA, 20 bp, Cannabis sativa
gagtttttggttgtataatt

SEQ ID NO: 1101 - DNA, 20 bp, Cannabis sativa
tttttggttgtataatttgg

SEQ ID NO: 1102 - DNA, 20 bp, Cannabis sativa
ggctgacattcaacaattta

SEQ ID NO: 1103 - DNA, 20 bp, Cannabis sativa
ttagcttttgtaacatcaaa

SEQ ID NO: 1104 - DNA, 20 bp, Cannabis sativa
aaaggttctaatatatattg

SEQ ID NO: 1105 - DNA, 20 bp, Cannabis sativa
tttatatattcttgtaacaa

SEQ ID NO: 1106 - DNA, 3781 bp, Cannabis sativa
atatatatcacacaaaataaaataaataaaaattctactaagaatatcttcaatgaaaaataaaaaagaaaaaagagcagaatctcgtagcgatgttaacgtgaggaaaggaagataaattggtaaaaaataaaaaataataaaaataaaaataagaaaagtgtgtaacaataataatataaatagagatcgtcccctcttgaatgtgaaccacgaaaaacttggattcttctctctatattaatatataacaagtttggagtagtagagtatagcaatatagtgctgttgttgttctttttttatttaattaataataataataattaattaagaatatatggctagagatagggaccctcttgtggttggtagagtaataggagatgttttggatccttttacaaggtcagtctctcttagagtgagttatggtaatagagaggtcaacaatggttatgagcttaaaccttcccaaattgttaaccaacctcgtgttgatattggtggtgatgacctaaggaccttctacactttggtaaattaattaattaattaattacctcaatattcataatgatttcatctataaatatatatatatataaatatgtatattatgatcaatgtcatatattgaaaattattacaggtcatggtggatccggatgctcctagccctagcaaccccaatctaagggagtatttgcattggtaattaattaattaactaattaattaattgattatatattattattaaatagatgagggtttcatatcatgataagtagtataaaataattaattaaaattaataactttttttgagtaaatgatgatgagaaattattattattactcaaaagtatatgcttatatttttaattttgtataaaactttggaacgaatacaaaataaattatatattatatatgtttaaattatgtttttaattagtacaattaactatttattcttttttttagagaacactttttctaaattatttccaacatgtgttgtgtgcatgaaattgtttaattatgacacttatttgctaattcactacaccaaaatatgtctttactttttggtcattaattatgcattttataaagatgttagttgggcgtctgttgaaaaaatattgacgctaatattagttactgatatgttgagtataaagtgaaacgatgtgttttcgtgtaaatatataatgtgtcagttttgaaatcgataatgtcaataattgacgcttagactcgatagtgtcgattatataattaataaagtacttttagtgtcagttttgaattggaacttaccctaataattacagaattttgtaaaactataaccctaaagcttgttttaatttgttaaagtgactaggttattataatcatatttctagctaatatattaagtacccttaaattctttttacatgtttattaagccacaaattccatatatgctagctatataaatataaatatggaaaggatatatgggaatatacaatatatatgaatatataaaaagctgatgagaaagaatgtggtttggggatgaattttgaatgatgaagggatgaggctgtgtgtgtggaatcagttttaccctgctagctatagcatgtccccttttgtcccaccattaaaatggccacctcacactaagaaggttttcacaaattaatgtgtcatactctaagatcaacacatgtcaatatatatacatacacatgtatagggcattattagtaatttttatacattacttcagtaaattacacatatttcaactttgacccgaatttgaattcgaacatagactcagatttggaccctaaattcgaccctgaaccctgaaacacgaacttagactcagacctggacctggaccttgacccaaaccccgacccgaaccccgactcgaaccccgaaactagactggacctggactctggcctaggacctagctcgggtctagagttcaggtccagtccggggtccgaggtcaggatctgggttcgtgttctggggttcaggttgggttgggtctggagtctgggtctagggtccagattcaggatcgggttcaggttaaggtttgagtctgagtccaggtataggttctggtcgggatctgagttcgagagtttgaattcaaggtccaatttggagttggatgagttcatgtcatggtaaaatattatttttttagttaaaaaaaaattaaaataatcttaaaaaactgagccaaacactattaaaattttaaaataataaaaaataattttcaattctcacatttaaaacacaattttaaaaacaaaaaatgaaaaacacagtcaaatactcattattcataatttttaaattagagggaatgccctataagtaagataaaaataatatttcttatcataattttgtcttatatatactttttaactattttaatattaaattattagtttatgtagttatattcgttgtagttaccgtgaatattcggtacagtaaattaaggatggtaaaatagtgttctaaatttgttattatgattagataatagtactactttttcaatgaatttgttattaccacaatttatatttttatatatattattaatctagataatggtaatctaagagtcatttttgaactaataattgagctttgacctataatatatagtataattataataattatacatgtttggcaatttcaatttaggttggtgactgatattcctggaactaccgggccaagcttcggtgagccgttcatacttcataataatactcttatttactattttttctattatattaaattaagaacgctgtaaacaaaatagtgcggattaataattttttttaaaataatattaaataattataatatataatgttgtattacaaagtatataatttgttttttaattaattgtttattatgtgtaggacaagaggtggtgtgctacgagaacccgcggccgacggtggggattcatcggtatgtatttgtgttgttccggcaattgggaaggcaaacggtgttcgcgccggggtggcgtcagaacttcaacaccaaagactttgctgagctttataaccttggtttgcctgttgctgccctttattacaactgccaaagggagacaggatctgggggaaggaggtcctccacatagacatatatatatatatatatatcatcatcatatatcatatagatactaatatatattatatgatgattcttaatgatgatatcatggttttattatcaatgccattattaatataataagctttatatatatatatatatatagaataagatgaagaagaagaaaacaggactgcatgtcatgtacgtgtatgtatactgtatatgtattaatattaatgtaattgtatcttcattattgtacttaattcagctggtatgtacttaccttgctaatattatattatgtaataatgagatacacacatatatatggaagttagaatatatttatagaaaaatttaaagtgaatttcaaaattagcctacatgaggaatttattgtagttattatattaccaattacaaatagtattgaatgtactataatatttttaaggtgatcaattattctatatattatttttaattttttttaaaaaaaatttacggtttgggttcctaaattagttgcagcgctagttacaataggaattttaatatgattttttgttgtaatttaggttgc

SEQ ID NO: 1107 - DNA, 498 bp, Cannabis sativa
atggctagagatagggaccctcttgtggttggtagagtaataggagatgttttggatccttttacaaggtcagtctctcttagagtgagttatggtaatagagaggtcaacaatggttatgagcttaaaccttcccaaattgttaaccaacctcgtgttgatattggtggtgatgacctaaggaccttctacactttggtaaatgtcatggtggatccggatgctcctagccctagcaaccccaatctaagggagtatttgcattggggacaagaggtggtgtgctacgagaacccgcggccgacggtggggattcatcggtatgtatttgtgttgttccggcaattgggaaggcaaacggtgttcgcgccggggtggcgtcagaacttcaacaccaaagactttgctgagctttataaccttggtttgcctgttgctgccctttattacaactgccaaagggagacaggatctgggggaaggaggtcctccacatag

SEQ ID NO: 1108 - protein, 165 aa, Artificial Sequence
  CsSFT3 AA
MARDRDPLVVGRVIGDVLDPFTRSVSLRVSYGNREVNNGYELKPSQIVNQPRVDIGGDDLRTFYTLVNVMVDPDAPSPSNPNLREYLHWGQEVVCYENPRPTVGIHRYVFVLFRQLGRQTVFAPGWRQNFNTKDFAELYNLGLPVAALYYNCQRETGSGGRRSST

SEQ ID NO: 1109 - DNA, 20 bp, Cannabis sativa
ttactctaccaaccacaaga

SEQ ID NO: 1110 - DNA, 20 bp, Cannabis sativa
attactctaccaaccacaag

SEQ ID NO: 1111 - DNA, 20 bp, Cannabis sativa
gatagggaccctcttgtggt

SEQ ID NO: 1112 - DNA, 20 bp, Cannabis sativa
cttgtggttggtagagtaat

SEQ ID NO: 1113 - DNA, 20 bp, Cannabis sativa
tagagtaataggagatgttt

SEQ ID NO: 1114 - DNA, 20 bp, Cannabis sativa
atgttttggatccttttaca

SEQ ID NO: 1115 - DNA, 20 bp, Cannabis sativa
agagagactgaccttgtaaa

SEQ ID NO: 1116 - DNA, 20 bp, Cannabis sativa
gtctctcttagagtgagtta

SEQ ID NO: 1117 - DNA, 20 bp, Cannabis sativa
agtgagttatggtaatagag

SEQ ID NO: 1118 - DNA, 20 bp, Cannabis sativa
ggtaatagagaggtcaacaa

SEQ ID NO: 1119 - DNA, 20 bp, Cannabis sativa
ggttggttaacaatttggga

SEQ ID NO: 1120 - DNA, 20 bp, Cannabis sativa
acgaggttggttaacaattt

SEQ ID NO: 1121 - DNA, 20 bp, Cannabis sativa
cacgaggttggttaacaatt

SEQ ID NO: 1122 - DNA, 20 bp, Cannabis sativa
caccaatatcaacacgaggt

SEQ ID NO: 1123 - DNA, 20 bp, Cannabis sativa
tcaccaccaatatcaacacg

SEQ ID NO: 1124 - DNA, 20 bp, Cannabis sativa
aaccaacctcgtgttgatat

SEQ ID NO: 1125 - DNA, 20 bp, Cannabis sativa
caacctcgtgttgatattgg

SEQ ID NO: 1126 - DNA, 20 bp, Cannabis sativa
atattggtggtgatgaccta

SEQ ID NO: 1127 - DNA, 20 bp, Cannabis sativa
ccaaagtgtagaaggtcctt

SEQ ID NO: 1128 - DNA, 20 bp, Cannabis sativa
ttaatttaccaaagtgtaga

SEQ ID NO: 1129 - DNA, 20 bp, Cannabis sativa
cctaaggaccttctacactt

SEQ ID NO: 1130 - DNA, 20 bp, Cannabis sativa
tgaaatcattatgaatattg

SEQ ID NO: 1131 - DNA, 20 bp, Cannabis sativa
catatattgaaaattattac

SEQ ID NO: 1132 - DNA, 20 bp, Cannabis sativa
ttgaaaattattacaggtca

SEQ ID NO: 1133 - DNA, 20 bp, Cannabis sativa
aaaattattacaggtcatgg

SEQ ID NO: 1134 - DNA, 20 bp, Cannabis sativa
attacaggtcatggtggatc

SEQ ID NO: 1135 - DNA, 20 bp, Cannabis sativa
ttgctagggctaggagcatc

SEQ ID NO: 1136 - DNA, 20 bp, Cannabis sativa
agattggggttgctagggct

SEQ ID NO: 1137 - DNA, 20 bp, Cannabis sativa
cccttagattggggttgcta

SEQ ID NO: 1138 - DNA, 20 bp, Cannabis sativa
tcccttagattggggttgct

SEQ ID NO: 1139 - DNA, 20 bp, Cannabis sativa
gcaaatactcccttagattg

SEQ ID NO: 1140 - DNA, 20 bp, Cannabis sativa
gccctagcaaccccaatcta

SEQ ID NO: 1141 - DNA, 20 bp, Cannabis sativa
tgcaaatactcccttagatt

SEQ ID NO: 1142 - DNA, 20 bp, Cannabis sativa
ccctagcaaccccaatctaa

SEQ ID NO: 1143 - DNA, 20 bp, Cannabis sativa
atgcaaatactcccttagat

SEQ ID NO: 1144 - DNA, 20 bp, Cannabis sativa
atctaagggagtatttgcat

SEQ ID NO: 1145 - DNA, 20 bp, Cannabis sativa
atattattattaaatagatg

SEQ ID NO: 1146 - DNA, 20 bp, Cannabis sativa
tattattattaaatagatga

SEQ ID NO: 1147 - DNA, 20 bp, Cannabis sativa
tttaattttgtataaaactt

SEQ ID NO: 1148 - DNA, 20 bp, Cannabis sativa
ttcatgcacacaacacatgt

SEQ ID NO: 1149 - DNA, 20 bp, Cannabis sativa
caaaaagtaaagacatattt

SEQ ID NO: 1150 - DNA, 20 bp, Cannabis sativa
caaaatatgtctttactttt

SEQ ID NO: 1151 - DNA, 20 bp, Cannabis sativa
cattttataaagatgttagt

SEQ ID NO: 1152 - DNA, 20 bp, Cannabis sativa
attttataaagatgttagtt

SEQ ID NO: 1153 - DNA, 20 bp, Cannabis sativa
ttttagtgtcagttttgaat

SEQ ID NO: 1154 - DNA, 20 bp, Cannabis sativa
acaaaattctgtaattatta

SEQ ID NO: 1155 - DNA, 20 bp, Cannabis sativa
tacaaaattctgtaattatt

SEQ ID NO: 1156 - DNA, 20 bp, Cannabis sativa
acaaattaaaacaagcttta

SEQ ID NO: 1157 - DNA, 20 bp, Cannabis sativa
aacaaattaaaacaagcttt

SEQ ID NO: 1158 - DNA, 20 bp, Cannabis sativa
tttaatttgttaaagtgact

SEQ ID NO: 1159 - DNA, 20 bp, Cannabis sativa
aacatgtaaaaagaatttaa

SEQ ID NO: 1160 - DNA, 20 bp, Cannabis sativa
aaacatgtaaaaagaattta

SEQ ID NO: 1161 - DNA, 20 bp, Cannabis sativa
gctagcatatatggaatttg

SEQ ID NO: 1162 - DNA, 20 bp, Cannabis sativa
atttatatagctagcatata

SEQ ID NO: 1163 - DNA, 20 bp, Cannabis sativa
tagctatataaatataaata

SEQ ID NO: 1164 - DNA, 20 bp, Cannabis sativa
atataaatataaatatggaa

SEQ ID NO: 1165 - DNA, 20 bp, Cannabis sativa
ataaatatggaaaggatata

SEQ ID NO: 1166 - DNA, 20 bp, Cannabis sativa
taaatatggaaaggatatat

SEQ ID NO: 1167 - DNA, 20 bp, Cannabis sativa
aaagctgatgagaaagaatg

SEQ ID NO: 1168 - DNA, 20 bp, Cannabis sativa
tgatgagaaagaatgtggtt

SEQ ID NO: 1169 - DNA, 20 bp, Cannabis sativa
gatgagaaagaatgtggttt

SEQ ID NO: 1170 - DNA, 20 bp, Cannabis sativa
atgagaaagaatgtggtttg

SEQ ID NO: 1171 - DNA, 20 bp, Cannabis sativa
ggatgaattttgaatgatga

SEQ ID NO: 1172 - DNA, 20 bp, Cannabis sativa
gatgaattttgaatgatgaa

SEQ ID NO: 1173 - DNA, 20 bp, Cannabis sativa
ttttgaatgatgaagggatg

SEQ ID NO: 1174 - DNA, 20 bp, Cannabis sativa
aagggatgaggctgtgtgtg

SEQ ID NO: 1175 - DNA, 20 bp, Cannabis sativa
gggacatgctatagctagca

SEQ ID NO: 1176 - DNA, 20 bp, Cannabis sativa
ggggacatgctatagctagc

SEQ ID NO: 1177 - DNA, 20 bp, Cannabis sativa
ttttaatggtgggacaaaag

SEQ ID NO: 1178 - DNA, 20 bp, Cannabis sativa
attttaatggtgggacaaaa

SEQ ID NO: 1179 - DNA, 20 bp, Cannabis sativa
cattttaatggtgggacaaa

SEQ ID NO: 1180 - DNA, 20 bp, Cannabis sativa
gaggtggccattttaatggt

SEQ ID NO: 1181 - DNA, 20 bp, Cannabis sativa
tgaggtggccattttaatgg

SEQ ID NO: 1182 - DNA, 20 bp, Cannabis sativa
cttttgtcccaccattaaaa

SEQ ID NO: 1183 - DNA, 20 bp, Cannabis sativa
gtgtgaggtggccattttaa

SEQ ID NO: 1184 - DNA, 20 bp, Cannabis sativa
aaaaccttcttagtgtgagg

SEQ ID NO: 1185 - DNA, 20 bp, Cannabis sativa
gtgaaaaccttcttagtgtg

SEQ ID NO: 1186 - DNA, 20 bp, Cannabis sativa
atggccacctcacactaaga

SEQ ID NO: 1187 - DNA, 20 bp, Cannabis sativa
atatatacatacacatgtat

SEQ ID NO: 1188 - DNA, 20 bp, Cannabis sativa
tatatacatacacatgtata

SEQ ID NO: 1189 - DNA, 20 bp, Cannabis sativa
ctatgttcgaattcaaattc

SEQ ID NO: 1190 - DNA, 20 bp, Cannabis sativa
tctatgttcgaattcaaatt

SEQ ID NO: 1191 - DNA, 20 bp, Cannabis sativa
ttcgaacatagactcagatt

SEQ ID NO: 1192 - DNA, 20 bp, Cannabis sativa
agggttcagggtcgaattta

SEQ ID NO: 1193 - DNA, 20 bp, Cannabis sativa
cagggttcagggtcgaattt

SEQ ID NO: 1194 - DNA, 20 bp, Cannabis sativa
agttcgtgtttcagggttca

SEQ ID NO: 1195 - DNA, 20 bp, Cannabis sativa
aagttcgtgtttcagggttc

SEQ ID NO: 1196 - DNA, 20 bp, Cannabis sativa
gagtctaagttcgtgtttca

SEQ ID NO: 1197 - DNA, 20 bp, Cannabis sativa
tgagtctaagttcgtgtttc

SEQ ID NO: 1198 - DNA, 20 bp, Cannabis sativa
cacgaacttagactcagacc

SEQ ID NO: 1199 - DNA, 20 bp, Cannabis sativa
cttagactcagacctggacc

SEQ ID NO: 1200 - DNA, 20 bp, Cannabis sativa
ttgggtcaaggtccaggtcc

SEQ ID NO: 1201 - DNA, 20 bp, Cannabis sativa
cggggtttgggtcaaggtcc

SEQ ID NO: 1202 - DNA, 20 bp, Cannabis sativa
tcgggtcggggtttgggtca

SEQ ID NO: 1203 - DNA, 20 bp, Cannabis sativa
cggggttcgggtcggggttt

SEQ ID NO: 1204 - DNA, 20 bp, Cannabis sativa
tcggggttcgggtcggggtt

SEQ ID NO: 1205 - DNA, 20 bp, Cannabis sativa
tcgagtcggggttcgggtcg

SEQ ID NO: 1206 - DNA, 20 bp, Cannabis sativa
ttcgagtcggggttcgggtc

SEQ ID NO: 1207 - DNA, 20 bp, Cannabis sativa
gttcgagtcggggttcgggt

SEQ ID NO: 1208 - DNA, 20 bp, Cannabis sativa
cggggttcgagtcggggttc

SEQ ID NO: 1209 - DNA, 20 bp, Cannabis sativa
tcggggttcgagtcggggtt

SEQ ID NO: 1210 - DNA, 20 bp, Cannabis sativa
tagtttcggggttcgagtcg

SEQ ID NO: 1211 - DNA, 20 bp, Cannabis sativa
ctagtttcggggttcgagtc

SEQ ID NO: 1212 - DNA, 20 bp, Cannabis sativa
tctagtttcggggttcgagt

SEQ ID NO: 1213 - DNA, 20 bp, Cannabis sativa
ccaggtccagtctagtttcg

SEQ ID NO: 1214 - DNA, 20 bp, Cannabis sativa
tccaggtccagtctagtttc

SEQ ID NO: 1215 - DNA, 20 bp, Cannabis sativa
gtccaggtccagtctagttt

SEQ ID NO: 1216 - DNA, 20 bp, Cannabis sativa
ctcgaaccccgaaactagac

SEQ ID NO: 1217 - DNA, 20 bp, Cannabis sativa
ccccgaaactagactggacc

SEQ ID NO: 1218 - DNA, 20 bp, Cannabis sativa
actagactggacctggactc

SEQ ID NO: 1219 - DNA, 20 bp, Cannabis sativa
taggtcctaggccagagtcc

SEQ ID NO: 1220 - DNA, 20 bp, Cannabis sativa
ctggacctggactctggcct

SEQ ID NO: 1221 - DNA, 20 bp, Cannabis sativa
ctagacccgagctaggtcct

SEQ ID NO: 1222 - DNA, 20 bp, Cannabis sativa
ctctggcctaggacctagct

SEQ ID NO: 1223 - DNA, 20 bp, Cannabis sativa
tctggcctaggacctagctc

SEQ ID NO: 1224 - DNA, 20 bp, Cannabis sativa
ctgaactctagacccgagct

SEQ ID NO: 1225 - DNA, 20 bp, Cannabis sativa
ctagctcgggtctagagttc

SEQ ID NO: 1226 - DNA, 20 bp, Cannabis sativa
tctagagttcaggtccagtc

SEQ ID NO: 1227 - DNA, 20 bp, Cannabis sativa
ctagagttcaggtccagtcc

SEQ ID NO: 1228 - DNA, 20 bp, Cannabis sativa
tagagttcaggtccagtccg

SEQ ID NO: 1229 - DNA, 20 bp, Cannabis sativa
cctgacctcggaccccggac

SEQ ID NO: 1230 - DNA, 20 bp, Cannabis sativa
cagatcctgacctcggaccc

SEQ ID NO: 1231 - DNA, 20 bp, Cannabis sativa
caggtccagtccggggtccg

SEQ ID NO: 1232 - DNA, 20 bp, Cannabis sativa
ccagtccggggtccgaggtc

SEQ ID NO: 1233 - DNA, 20 bp, Cannabis sativa
acgaacccagatcctgacct

SEQ ID NO: 1234 - DNA, 20 bp, Cannabis sativa
cggggtccgaggtcaggatc

SEQ ID NO: 1235 - DNA, 20 bp, Cannabis sativa
ggggtccgaggtcaggatct

SEQ ID NO: 1236 - DNA, 20 bp, Cannabis sativa
caggatctgggttcgtgttc

SEQ ID NO: 1237 - DNA, 20 bp, Cannabis sativa
aggatctgggttcgtgttct

SEQ ID NO: 1238 - DNA, 20 bp, Cannabis sativa
ggatctgggttcgtgttctg

SEQ ID NO: 1239 - DNA, 20 bp, Cannabis sativa
gggttcgtgttctggggttc

SEQ ID NO: 1240 - DNA, 20 bp, Cannabis sativa
tcgtgttctggggttcaggt

SEQ ID NO: 1241 - DNA, 20 bp, Cannabis sativa
cgtgttctggggttcaggtt

SEQ ID NO: 1242 - DNA, 20 bp, Cannabis sativa
ttctggggttcaggttgggt

SEQ ID NO: 1243 - DNA, 20 bp, Cannabis sativa
tctggggttcaggttgggtt

SEQ ID NO: 1244 - DNA, 20 bp, Cannabis sativa
ggttcaggttgggttgggtc

SEQ ID NO: 1245 - DNA, 20 bp, Cannabis sativa
gttgggttgggtctggagtc

SEQ ID NO: 1246 - DNA, 20 bp, Cannabis sativa
ttgggttgggtctggagtct

SEQ ID NO: 1247 - DNA, 20 bp, Cannabis sativa
tgggtctggagtctgggtct

SEQ ID NO: 1248 - DNA, 20 bp, Cannabis sativa
gggtctggagtctgggtcta

SEQ ID NO: 1249 - DNA, 20 bp, Cannabis sativa
tgggtctagggtccagattc

SEQ ID NO: 1250 - DNA, 20 bp, Cannabis sativa
cctgaacccgatcctgaatc

SEQ ID NO: 1251 - DNA, 20 bp, Cannabis sativa
ctagggtccagattcaggat

SEQ ID NO: 1252 - DNA, 20 bp, Cannabis sativa
tagggtccagattcaggatc

SEQ ID NO: 1253 - DNA, 20 bp, Cannabis sativa
ccagattcaggatcgggttc

SEQ ID NO: 1254 - DNA, 20 bp, Cannabis sativa
tcaggatcgggttcaggtta

SEQ ID NO: 1255 - DNA, 20 bp, Cannabis sativa
taaggtttgagtctgagtcc

SEQ ID NO: 1256 - DNA, 20 bp, Cannabis sativa
ttgagtctgagtccaggtat

SEQ ID NO: 1257 - DNA, 20 bp, Cannabis sativa
tcccgaccagaacctatacc

SEQ ID NO: 1258 - DNA, 20 bp, Cannabis sativa
ctgagtccaggtataggttc

SEQ ID NO: 1259 - DNA, 20 bp, Cannabis sativa
gtccaggtataggttctggt

SEQ ID NO: 1260 - DNA, 20 bp, Cannabis sativa
tccaggtataggttctggtc

SEQ ID NO: 1261 - DNA, 20 bp, Cannabis sativa
agttcgagagtttgaattca

SEQ ID NO: 1262 - DNA, 20 bp, Cannabis sativa
tttgaattcaaggtccaatt

SEQ ID NO: 1263 - DNA, 20 bp, Cannabis sativa
gaactcatccaactccaaat

SEQ ID NO: 1264 - DNA, 20 bp, Cannabis sativa
ttcaaggtccaatttggagt

SEQ ID NO: 1265 - DNA, 20 bp, Cannabis sativa
agttggatgagttcatgtca

SEQ ID NO: 1266 - DNA, 20 bp, Cannabis sativa
tttaaaattttaatagtgtt

SEQ ID NO: 1267 - DNA, 20 bp, Cannabis sativa
attcataatttttaaattag

SEQ ID NO: 1268 - DNA, 20 bp, Cannabis sativa
ttcataatttttaaattaga

SEQ ID NO: 1269 - DNA, 20 bp, Cannabis sativa
ttatttttatcttacttata

SEQ ID NO: 1270 - DNA, 20 bp, Cannabis sativa
attatttttatcttacttat

SEQ ID NO: 1271 - DNA, 20 bp, Cannabis sativa
tttactgtaccgaatattca

SEQ ID NO: 1272 - DNA, 20 bp, Cannabis sativa
ttgtagttaccgtgaatatt

SEQ ID NO: 1273 - DNA, 20 bp, Cannabis sativa
atattcggtacagtaaatta

SEQ ID NO: 1274 - DNA, 20 bp, Cannabis sativa
tcggtacagtaaattaagga

SEQ ID NO: 1275 - DNA, 20 bp, Cannabis sativa
atatataaaaatataaattg

SEQ ID NO: 1276 - DNA, 20 bp, Cannabis sativa
tatattattaatctagataa

SEQ ID NO: 1277 - DNA, 20 bp, Cannabis sativa
ataattatactatatattat

SEQ ID NO: 1278 - DNA, 20 bp, Cannabis sativa
ttataataattatacatgtt

SEQ ID NO: 1279 - DNA, 20 bp, Cannabis sativa
atgtttggcaatttcaattt

SEQ ID NO: 1280 - DNA, 20 bp, Cannabis sativa
ttggcaatttcaatttaggt

SEQ ID NO: 1281 - DNA, 20 bp, Cannabis sativa
aggttggtgactgatattcc

SEQ ID NO: 1282 - DNA, 20 bp, Cannabis sativa
aagcttggcccggtagttcc

SEQ ID NO: 1283 - DNA, 20 bp, Cannabis sativa
actgatattcctggaactac

SEQ ID NO: 1284 - DNA, 20 bp, Cannabis sativa
ctgatattcctggaactacc

SEQ ID NO: 1285 - DNA, 20 bp, Cannabis sativa
cggctcaccgaagcttggcc

SEQ ID NO: 1286 - DNA, 20 bp, Cannabis sativa
ggaactaccgggccaagctt

SEQ ID NO: 1287 - DNA, 20 bp, Cannabis sativa
atgaacggctcaccgaagct

SEQ ID NO: 1288 - DNA, 20 bp, Cannabis sativa
gtattattatgaagtatgaa

SEQ ID NO: 1289 - DNA, 20 bp, Cannabis sativa
acgctgtaaacaaaatagtg

SEQ ID NO: 1290 - DNA, 20 bp, Cannabis sativa
attaattgtttattatgtgt

SEQ ID NO: 1291 - DNA, 20 bp, Cannabis sativa
tttattatgtgtaggacaag

SEQ ID NO: 1292 - DNA, 20 bp, Cannabis sativa
attatgtgtaggacaagagg

SEQ ID NO: 1293 - DNA, 20 bp, Cannabis sativa
tggtgtgctacgagaacccg

SEQ ID NO: 1294 - DNA, 20 bp, Cannabis sativa
gaatccccaccgtcggccgc

SEQ ID NO: 1295 - DNA, 20 bp, Cannabis sativa
tgaatccccaccgtcggccg

SEQ ID NO: 1296 - DNA, 20 bp, Cannabis sativa
ctacgagaacccgcggccga

SEQ ID NO: 1297 - DNA, 20 bp, Cannabis sativa
cgagaacccgcggccgacgg

SEQ ID NO: 1298 - DNA, 20 bp, Cannabis sativa
gagaacccgcggccgacggt

SEQ ID NO: 1299 - DNA, 20 bp, Cannabis sativa
agaacccgcggccgacggtg

SEQ ID NO: 1300 - DNA, 20 bp, Cannabis sativa
taccgatgaatccccaccgt

SEQ ID NO: 1301 - DNA, 20 bp, Cannabis sativa
ggccgacggtggggattcat

SEQ ID NO: 1302 - DNA, 20 bp, Cannabis sativa
ggtatgtatttgtgttgttc

SEQ ID NO: 1303 - DNA, 20 bp, Cannabis sativa
atttgtgttgttccggcaat

SEQ ID NO: 1304 - DNA, 20 bp, Cannabis sativa
tttgtgttgttccggcaatt

SEQ ID NO: 1305 - DNA, 20 bp, Cannabis sativa
ccgtttgccttcccaattgc

SEQ ID NO: 1306 - DNA, 20 bp, Cannabis sativa
tgttgttccggcaattggga

SEQ ID NO: 1307 - DNA, 20 bp, Cannabis sativa
ccggcaattgggaaggcaaa

SEQ ID NO: 1308 - DNA, 20 bp, Cannabis sativa
aaggcaaacggtgttcgcgc

SEQ ID NO: 1309 - DNA, 20 bp, Cannabis sativa
aggcaaacggtgttcgcgcc

SEQ ID NO: 1310 - DNA, 20 bp, Cannabis sativa
ggcaaacggtgttcgcgccg

SEQ ID NO: 1311 - DNA, 20 bp, Cannabis sativa
aaacggtgttcgcgccgggg

SEQ ID NO: 1312 - DNA, 20 bp, Cannabis sativa
ttgaagttctgacgccaccc

SEQ ID NO: 1313 - DNA, 20 bp, Cannabis sativa
ataaagctcagcaaagtctt

SEQ ID NO: 1314 - DNA, 20 bp, Cannabis sativa
tttgctgagctttataacct

SEQ ID NO: 1315 - DNA, 20 bp, Cannabis sativa
gggcagcaacaggcaaacca

SEQ ID NO: 1316 - DNA, 20 bp, Cannabis sativa
ttgtaataaagggcagcaac

SEQ ID NO: 1317 - DNA, 20 bp, Cannabis sativa
cctttggcagttgtaataaa

SEQ ID NO: 1318 - DNA, 20 bp, Cannabis sativa
ccctttggcagttgtaataa

SEQ ID NO: 1319 - DNA, 20 bp, Cannabis sativa
ccctttattacaactgccaa

SEQ ID NO: 1320 - DNA, 20 bp, Cannabis sativa
cctttattacaactgccaaa

SEQ ID NO: 1321 - DNA, 20 bp, Cannabis sativa
ccccagatcctgtctccctt

SEQ ID NO: 1322 - DNA, 20 bp, Cannabis sativa
tacaactgccaaagggagac

SEQ ID NO: 1323 - DNA, 20 bp, Cannabis sativa
tgccaaagggagacaggatc

SEQ ID NO: 1324 - DNA, 20 bp, Cannabis sativa
gccaaagggagacaggatct

SEQ ID NO: 1325 - DNA, 20 bp, Cannabis sativa
ccaaagggagacaggatctg

SEQ ID NO: 1326 - DNA, 20 bp, Cannabis sativa
caaagggagacaggatctgg

SEQ ID NO: 1327 - DNA, 20 bp, Cannabis sativa
gggagacaggatctggggga

SEQ ID NO: 1328 - DNA, 20 bp, Cannabis sativa
agacaggatctgggggaagg

SEQ ID NO: 1329 - DNA, 20 bp, Cannabis sativa
atatatatatgtctatgtgg

SEQ ID NO: 1330 - DNA, 20 bp, Cannabis sativa
tatatatatatatgtctatg

SEQ ID NO: 1331 - DNA, 20 bp, Cannabis sativa
gattcttaatgatgatatca

SEQ ID NO: 1332 - DNA, 20 bp, Cannabis sativa
aaagcttattatattaataa

SEQ ID NO: 1333 - DNA, 20 bp, Cannabis sativa
aagatgaagaagaagaaaac

SEQ ID NO: 1334 - DNA, 20 bp, Artificial Sequence
  gRNA_CsSFT3_3465
attattgtacttaattcagc

SEQ ID NO: 1335 - DNA, 2476 bp, Artificial Sequence
  CsSPGB genomic CM011611.165513425-65515200
tgacacattttttcttttcattttcatatcatatattgtttctttactttaatttgttttttcattaaagtaaagacaatttataaccatatgatatttatacatataattggccacacccacggatttacagtagccacagaattagtttttgcatacagagagaaaagcaaacaaaaaaaaagagaaaaagaaaaagaaagtttttataaagtcaaacaaaacacatacaaacaaacaaaggaggcccaccaagttaagttccagcctcttaggaaataacaaggaaacactttgttttttgttacaacaaaagcattagtagtctctattctaaaccttaaaataccacttacatgtttcgatataaaaccttcctttccccaacaaaagcatacatatatatatataaatataaacattgataaaaaaaaaacccctcttcattagtcataacaactcaaaagagaccctctctcaagctaaggtagtgagatatatagtgttaggaaagtaaaggtattgagatatttatatatactcatattaaaatggagcagttaacgttcgttgaagactacgctttttgggaagtttatcagcagcagcagcagcctcttcattaatcttaaaaacaaataaaataaaacaaaattaccccaaaatagaagaaagcaaaataaataatagtaatgttattagtgtaaaatagttaatacaaactttacctggttttgccttcttctaagtctttcattttcttcactcaattgtgcaacaacaagctccagctcatttgtatatgcctgtaaataggtgacaaaaaaaaaaaagcccagatagataccctctaagaatattaatacaaagaaataataaatagttaaataccatgtacttttgcaaattaaactactaattaaaaatgctaactttgataagaaaaagttattaatataataataagttaataacccatttgaggaaagcacgtaaataacaagcttgcctgttttctagctcttgatcgagcagctgactctcgatttttgatcatcctcttgtttcgtctgtcccccgaagctgacccattttcagtttggaaccttttcttcccaaaagtgtttaacaaacaagaagaggaatctaaagtctcaaatgggcttgtgaaagatgaaacattggagatgtggttgtttggatgcaaggatggatcggtgaggcttccatcgagaaatggaaactcagggccagaattcaagctcaatgctgtaagaggctgaggcagaggagacccaaacagagtactgtccggagacggagctggaggagggaccatggtggtcggaggtcgatcagtgcaaaagggtcttgctaaaaagtcttggtagatcatgtttcggtagttggtgtggttgttattgatgttgttgagttgtggtggtggtggtggaacaacagtaactgtattagtagtctcagaaagagaagtcaagttcaaatctttccaaacttcttccatggcttttgttgtagaaggagcagttgaacttgttcttgaagatgttgaagaagaagaaaaagacttctttgaagaagaggaagagtatgaacctgtgttgtgtaagcttaagctattatttcttcttcttcttcttctggtttctgatgtactacttgtgttgcttatttcgatctcttcagcacctgatgacgacgacattagtataagactgatgggtttttatttactataatatatatatatattgagagagagagagagagagagagagagagagagagagatgagaagagtagagctggtgtatgtagctacagctatgtatgaaggtatagatagagagagagagagagaagaagaagaactggtgtatattgtattgagccacgcgcggaagagtactgtttttattgggaggattcagattcccaaattaaagagacttatagggcctggcctgtaaactgtatacttcagtccaatctcatctgctgttccacaacattatgagaatctaaaaggacttcgctcccaccactcctttttaacagtgccactaaccatccccctatatatgtacatgcttgtagtactctatcactgtgtacaatgtacatcaatactgttgtgaccatttacttagcattatgtcttattaatccatgaataagattaattgactaatttgcaactactctctcaaggtgggttgtgtattaattaggcctggtgtgaatgtggaatattggcatatatagtatggtgtctagtctagtctaagtgacactctagttttctttgctatatactgttttgctttaattctttttttactttgttttgagattttaaaagggtgtccatgaatgaaacaggcaaagtttgtctttgacaggattggtctgaattttggtgacagaaaaccatagaaaaagaaggtaggaaagggaa

SEQ ID NO: 1336 - DNA, 708 bp, Artificial Sequence
  CsSPGB CDS
atgtcgtcgtcatcaggtgctgaagagatcgaaataagcaacacaagtagtacatcagaaaccagaagaagaagaagaagaaataatagcttaagcttacacaacacaggttcatactcttcctcttcttcaaagaagtctttttcttcttcttcaacatcttcaagaacaagttcaactgctccttctacaacaaaagccatggaagaagtttggaaagatttgaacttgacttctctttctgagactactaatacagttactgttgttccaccaccaccaccacaactcaacaacatcaataacaaccacaccaactaccgaaacatgatctaccaagactttttagcaagacccttttgcactgatcgacctccgaccaccatggtccctcctccagctccgtctccggacagtactctgttgagcttgaattctggccctgagtttccatttctcgatggaagcctcaccgatccatccttgcatccaaacaaccacatctccaatgtttcatctttcgggaagaaaaggttccaaactgaaaatgggtcagcttcgggggacagacgaaacaagaggatgatcaaaaatcgagagtcagctgctcgatcaagagctagaaaacaggcatatacaaatgagctggagcttgttgttgcacaattgagtgaagaaaatgaaagacttagaagaaggcaaaaccag

SEQ ID NO: 1337 - protein, 236 aa, Artificial Sequence
  CsSPGB AA
MSSSSGAEEIEISNTSSTSETRRRRRRNNSLSLHNTGSYSSSSSKKSFSSSSTSSRTSSTAPSTTKAMEEVWKDLNLTSLSETTNTVTVVPPPPPQLNNINNNHTNYRNMIYQDFLARPFCTDRPPTTMVPPPAPSPDSTLLSLNSGPEFPFLDGSLTDPSLHPNNHISNVSSFGKKRFQTENGSASGDRRNKRMIKNRESAARSRARKQAYTNELELVVAQLSEENERLRRRQNQ

SEQ ID NO: 1338 - DNA, 20 bp, Cannabis sativa
ttatatgtataaatatcata

SEQ ID NO: 1339 - DNA, 20 bp, Cannabis sativa
atgatatttatacatataat

SEQ ID NO: 1340 - DNA, 20 bp, Cannabis sativa
catataattggccacaccca

SEQ ID NO: 1341 - DNA, 20 bp, Cannabis sativa
ctactgtaaatccgtgggtg

SEQ ID NO: 1342 - DNA, 20 bp, Cannabis sativa
tgtggctactgtaaatccgt

SEQ ID NO: 1343 - DNA, 20 bp, Cannabis sativa
ctgtggctactgtaaatccg

SEQ ID NO: 1344 - DNA, 20 bp, Cannabis sativa
gtatgcaaaaactaattctg

SEQ ID NO: 1345 - DNA, 20 bp, Cannabis sativa
aaacacatacaaacaaacaa

SEQ ID NO: 1346 - DNA, 20 bp, Cannabis sativa
cacatacaaacaaacaaagg

SEQ ID NO: 1347 - DNA, 20 bp, Cannabis sativa
aggctggaacttaacttggt

SEQ ID NO: 1348 - DNA, 20 bp, Cannabis sativa
gaggctggaacttaacttgg

SEQ ID NO: 1349 - DNA, 20 bp, Cannabis sativa
taagaggctggaacttaact

SEQ ID NO: 1350 - DNA, 20 bp, Cannabis sativa
cttgttatttcctaagaggc

SEQ ID NO: 1351 - DNA, 20 bp, Cannabis sativa
aagttaagttccagcctctt

SEQ ID NO: 1352 - DNA, 20 bp, Cannabis sativa
tttccttgttatttcctaag

SEQ ID NO: 1353 - DNA, 20 bp, Cannabis sativa
cagcctcttaggaaataaca

SEQ ID NO: 1354 - DNA, 20 bp, Cannabis sativa
aacatgtaagtggtatttta

SEQ ID NO: 1355 - DNA, 20 bp, Cannabis sativa
tttatatcgaaacatgtaag

SEQ ID NO: 1356 - DNA, 20 bp, Cannabis sativa
tgcttttgttggggaaagga

SEQ ID NO: 1357 - DNA, 20 bp, Cannabis sativa
tgtatgcttttgttggggaa

SEQ ID NO: 1358 - DNA, 20 bp, Cannabis sativa
atatatgtatgcttttgttg

SEQ ID NO: 1359 - DNA, 20 bp, Cannabis sativa
tatatatgtatgcttttgtt

SEQ ID NO: 1360 - DNA, 20 bp, Cannabis sativa
atatatatgtatgcttttgt

SEQ ID NO: 1361 - DNA, 20 bp, Cannabis sativa
ttgttatgactaatgaagag

SEQ ID NO: 1362 - DNA, 20 bp, Cannabis sativa
gttgttatgactaatgaaga

SEQ ID NO: 1363 - DNA, 20 bp, Cannabis sativa
agttgttatgactaatgaag

SEQ ID NO: 1364 - DNA, 20 bp, Cannabis sativa
cactaccttagcttgagaga

SEQ ID NO: 1365 - DNA, 20 bp, Cannabis sativa
tcactaccttagcttgagag

SEQ ID NO: 1366 - DNA, 20 bp, Cannabis sativa
agagaccctctctcaagcta

SEQ ID NO: 1367 - DNA, 20 bp, Cannabis sativa
gtagtgagatatatagtgtt

SEQ ID NO: 1368 - DNA, 20 bp, Cannabis sativa
atatagtgttaggaaagtaa

SEQ ID NO: 1369 - DNA, 20 bp, Cannabis sativa
tatatatactcatattaaaa

SEQ ID NO: 1370 - DNA, 20 bp, Cannabis sativa
tcgttgaagactacgctttt

SEQ ID NO: 1371 - DNA, 20 bp, Cannabis sativa
cgttgaagactacgcttttt

SEQ ID NO: 1372 - DNA, 20 bp, Cannabis sativa
tgtttttaagattaatgaag

SEQ ID NO: 1373 - DNA, 20 bp, Cannabis sativa
ttttgctttcttctattttg

SEQ ID NO: 1374 - DNA, 20 bp, Cannabis sativa
attttgctttcttctatttt

SEQ ID NO: 1375 - DNA, 20 bp, Cannabis sativa
tattttgctttcttctattt

SEQ ID NO: 1376 - DNA, 20 bp, Cannabis sativa
tagttaatacaaactttacc

SEQ ID NO: 1377 - DNA, 20 bp, Cannabis sativa
acttagaagaaggcaaaacc

SEQ ID NO: 1378 - DNA, 20 bp, Cannabis sativa
aaaatgaaagacttagaaga

SEQ ID NO: 1379 - DNA, 20 bp, Cannabis sativa
acaggcatatacaaatgagc

SEQ ID NO: 1380 - DNA, 20 bp, Cannabis sativa
atttgtatatgcctgtaaat

SEQ ID NO: 1381 - DNA, 20 bp, Cannabis sativa
tttttttgtcacctatttac

SEQ ID NO: 1382 - DNA, 20 bp, Cannabis sativa
ttcttagagggtatctatct

SEQ ID NO: 1383 - DNA, 20 bp, Cannabis sativa
attcttagagggtatctatc

SEQ ID NO: 1384 - DNA, 20 bp, Cannabis sativa
tttgtattaatattcttaga

SEQ ID NO: 1385 - DNA, 20 bp, Cannabis sativa
ctttgtattaatattcttag

SEQ ID NO: 1386 - DNA, 20 bp, Cannabis sativa
gtttaatttgcaaaagtaca

SEQ ID NO: 1387 - DNA, 20 bp, Cannabis sativa
ataagttaataacccatttg

SEQ ID NO: 1388 - DNA, 20 bp, Cannabis sativa
tttacgtgctttcctcaaat

SEQ ID NO: 1389 - DNA, 20 bp, Cannabis sativa
atttacgtgctttcctcaaa

SEQ ID NO: 1390 - DNA, 20 bp, Cannabis sativa
tcgatcaagagctagaaaac

SEQ ID NO: 1391 - DNA, 20 bp, Cannabis sativa
cgggggacagacgaaacaag

SEQ ID NO: 1392 - DNA, 20 bp, Cannabis sativa
tgaaaatgggtcagcttcgg

SEQ ID NO: 1393 - DNA, 20 bp, Cannabis sativa
ctgaaaatgggtcagcttcg

SEQ ID NO: 1394 - DNA, 20 bp, Cannabis sativa
actgaaaatgggtcagcttc

SEQ ID NO: 1395 - DNA, 20 bp, Cannabis sativa
aactgaaaatgggtcagctt

SEQ ID NO: 1396 - DNA, 20 bp, Cannabis sativa
aaaggttccaaactgaaaat

SEQ ID NO: 1397 - DNA, 20 bp, Cannabis sativa
aaaaggttccaaactgaaaa

SEQ ID NO: 1398 - DNA, 20 bp, Cannabis sativa
aagctgacccattttcagtt

SEQ ID NO: 1399 - DNA, 20 bp, Cannabis sativa
taaacacttttgggaagaaa

SEQ ID NO: 1400 - DNA, 20 bp, Cannabis sativa
cttgtttgttaaacactttt

SEQ ID NO: 1401 - DNA, 20 bp, Cannabis sativa
tcttgtttgttaaacacttt

SEQ ID NO: 1402 - DNA, 20 bp, Cannabis sativa
agtgtttaacaaacaagaag

SEQ ID NO: 1403 - DNA, 20 bp, Cannabis sativa
gaggaatctaaagtctcaaa

SEQ ID NO: 1404 - DNA, 20 bp, Cannabis sativa
aggaatctaaagtctcaaat

SEQ ID NO: 1405 - DNA, 20 bp, Cannabis sativa
gcttgtgaaagatgaaacat

SEQ ID NO: 1406 - DNA, 20 bp, Cannabis sativa
aagatgaaacattggagatg

SEQ ID NO: 1407 - DNA, 20 bp, Cannabis sativa
acattggagatgtggttgtt

SEQ ID NO: 1408 - DNA, 20 bp, Cannabis sativa
gatgtggttgtttggatgca

SEQ ID NO: 1409 - DNA, 20 bp, Cannabis sativa
tggttgtttggatgcaagga

SEQ ID NO: 1410 - DNA, 20 bp, Cannabis sativa
gtttggatgcaaggatggat

SEQ ID NO: 1411 - DNA, 20 bp, Cannabis sativa
gatgcaaggatggatcggtg

SEQ ID NO: 1412 - DNA, 20 bp, Cannabis sativa
cctgagtttccatttctcga

SEQ ID NO: 1413 - DNA, 20 bp, Cannabis sativa
gtgaggcttccatcgagaaa

SEQ ID NO: 1414 - DNA, 20 bp, Cannabis sativa
ccatcgagaaatggaaactc

SEQ ID NO: 1415 - DNA, 20 bp, Cannabis sativa
catcgagaaatggaaactca

SEQ ID NO: 1416 - DNA, 20 bp, Cannabis sativa
acagcattgagcttgaattc

SEQ ID NO: 1417 - DNA, 20 bp, Cannabis sativa
ttcaagctcaatgctgtaag

SEQ ID NO: 1418 - DNA, 20 bp, Cannabis sativa
ctcaatgctgtaagaggctg

SEQ ID NO: 1419 - DNA, 20 bp, Cannabis sativa
gctgtaagaggctgaggcag

SEQ ID NO: 1420 - DNA, 20 bp, Cannabis sativa
ctccggacagtactctgttt

SEQ ID NO: 1421 - DNA, 20 bp, Cannabis sativa
tctccggacagtactctgtt

SEQ ID NO: 1422 - DNA, 20 bp, Cannabis sativa
gacccaaacagagtactgtc

SEQ ID NO: 1423 - DNA, 20 bp, Cannabis sativa
aacagagtactgtccggaga

SEQ ID NO: 1424 - DNA, 20 bp, Cannabis sativa
ccctcctccagctccgtctc

SEQ ID NO: 1425 - DNA, 20 bp, Cannabis sativa
gtactgtccggagacggagc

SEQ ID NO: 1426 - DNA, 20 bp, Cannabis sativa
ctgtccggagacggagctgg

SEQ ID NO: 1427 - DNA, 20 bp, Cannabis sativa
tccggagacggagctggagg

SEQ ID NO: 1428 - DNA, 20 bp, Cannabis sativa
ccggagacggagctggagga

SEQ ID NO: 1429 - DNA, 20 bp, Cannabis sativa
cggagctggaggagggacca

SEQ ID NO: 1430 - DNA, 20 bp, Cannabis sativa
agctggaggagggaccatgg

SEQ ID NO: 1431 - DNA, 20 bp, Cannabis sativa
tgatcgacctccgaccacca

SEQ ID NO: 1432 - DNA, 20 bp, Cannabis sativa
ggaggagggaccatggtggt

SEQ ID NO: 1433 - DNA, 20 bp, Cannabis sativa
ggagggaccatggtggtcgg

SEQ ID NO: 1434 - DNA, 20 bp, Cannabis sativa
cggaggtcgatcagtgcaaa

SEQ ID NO: 1435 - DNA, 20 bp, Cannabis sativa
ggaggtcgatcagtgcaaaa

SEQ ID NO: 1436 - DNA, 20 bp, Cannabis sativa
agggtcttgctaaaaagtct

SEQ ID NO: 1437 - DNA, 20 bp, Cannabis sativa
agtcttggtagatcatgttt

SEQ ID NO: 1438 - DNA, 20 bp, Cannabis sativa
gtagatcatgtttcggtagt

SEQ ID NO: 1439 - DNA, 20 bp, Cannabis sativa
tcatgtttcggtagttggtg

SEQ ID NO: 1440 - DNA, 20 bp, Cannabis sativa
ttattgatgttgttgagttg

SEQ ID NO: 1441 - DNA, 20 bp, Cannabis sativa
ttgatgttgttgagttgtgg

SEQ ID NO: 1442 - DNA, 20 bp, Cannabis sativa
atgttgttgagttgtggtgg

SEQ ID NO: 1443 - DNA, 20 bp, Cannabis sativa
ttgttgagttgtggtggtgg

SEQ ID NO: 1444 - DNA, 20 bp, Cannabis sativa
ttgagttgtggtggtggtgg

SEQ ID NO: 1445 - DNA, 20 bp, Cannabis sativa
caaaagccatggaagaagtt

SEQ ID NO: 1446 - DNA, 20 bp, Cannabis sativa
atctttccaaacttcttcca

SEQ ID NO: 1447 - DNA, 20 bp, Cannabis sativa
tccttctacaacaaaagcca

SEQ ID NO: 1448 - DNA, 20 bp, Cannabis sativa
tccatggcttttgttgtaga

SEQ ID NO: 1449 - DNA, 20 bp, Cannabis sativa
aaaagacttctttgaagaag

SEQ ID NO: 1450 - DNA, 20 bp, Cannabis sativa
agcttaagcttacacaacac

SEQ ID NO: 1451 - DNA, 20 bp, Cannabis sativa
atttcttcttcttcttcttc

SEQ ID NO: 1452 - DNA, 20 bp, Cannabis sativa
atactaatgtcgtcgtcatc

SEQ ID NO: 1453 - DNA, 20 bp, Cannabis sativa
cgacattagtataagactga

SEQ ID NO: 1454 - DNA, 20 bp, Cannabis sativa
gacattagtataagactgat

SEQ ID NO: 1455 - DNA, 20 bp, Cannabis sativa
agagatgagaagagtagagc

SEQ ID NO: 1456 - DNA, 20 bp, Cannabis sativa
gtagctacagctatgtatga

SEQ ID NO: 1457 - DNA, 20 bp, Cannabis sativa
agagagagaagaagaagaac

SEQ ID NO: 1458 - DNA, 20 bp, Cannabis sativa
atattgtattgagccacgcg

SEQ ID NO: 1459 - DNA, 20 bp, Cannabis sativa
aaacagtactcttccgcgcg

SEQ ID NO: 1460 - DNA, 20 bp, Cannabis sativa
cggaagagtactgtttttat

SEQ ID NO: 1461 - DNA, 20 bp, Cannabis sativa
ggaagagtactgtttttatt

SEQ ID NO: 1462 - DNA, 20 bp, Cannabis sativa
agagtactgtttttattggg

SEQ ID NO: 1463 - DNA, 20 bp, Cannabis sativa
cctataagtctctttaattt

SEQ ID NO: 1464 - DNA, 20 bp, Cannabis sativa
ccctataagtctctttaatt

SEQ ID NO: 1465 - DNA, 20 bp, Cannabis sativa
cccaaattaaagagacttat

SEQ ID NO: 1466 - DNA, 20 bp, Cannabis sativa
ccaaattaaagagacttata

SEQ ID NO: 1467 - DNA, 20 bp, Cannabis sativa
ttaaagagacttatagggcc

SEQ ID NO: 1468 - DNA, 20 bp, Cannabis sativa
aagtatacagtttacaggcc

SEQ ID NO: 1469 - DNA, 20 bp, Cannabis sativa
gactgaagtatacagtttac

SEQ ID NO: 1470 - DNA, 20 bp, Cannabis sativa
tgtggaacagcagatgagat

SEQ ID NO: 1471 - DNA, 20 bp, Cannabis sativa
tttagattctcataatgttg

SEQ ID NO: 1472 - DNA, 20 bp, Cannabis sativa
caacattatgagaatctaaa

SEQ ID NO: 1473 - DNA, 20 bp, Cannabis sativa
cactgttaaaaaggagtggt

SEQ ID NO: 1474 - DNA, 20 bp, Cannabis sativa
gcactgttaaaaaggagtgg

SEQ ID NO: 1475 - DNA, 20 bp, Cannabis sativa
gtggcactgttaaaaaggag

SEQ ID NO: 1476 - DNA, 20 bp, Cannabis sativa
ggttagtggcactgttaaaa

SEQ ID NO: 1477 - DNA, 20 bp, Cannabis sativa
atatatagggggatggttag

SEQ ID NO: 1478 - DNA, 20 bp, Cannabis sativa
catgtacatatataggggga

SEQ ID NO: 1479 - DNA, 20 bp, Cannabis sativa
caagcatgtacatatatagg

SEQ ID NO: 1480 - DNA, 20 bp, Cannabis sativa
acaagcatgtacatatatag

SEQ ID NO: 1481 - DNA, 20 bp, Cannabis sativa
tacaagcatgtacatatata

SEQ ID NO: 1482 - DNA, 20 bp, Cannabis sativa
ctacaagcatgtacatatat

SEQ ID NO: 1483 - DNA, 20 bp, Cannabis sativa
aagacataatgctaagtaaa

SEQ ID NO: 1484 - DNA, 20 bp, Cannabis sativa
tagtcaattaatcttattca

SEQ ID NO: 1485 - DNA, 20 bp, Cannabis sativa
aatttgcaactactctctca

SEQ ID NO: 1486 - DNA, 20 bp, Cannabis sativa
ttgcaactactctctcaagg

SEQ ID NO: 1487 - DNA, 20 bp, Cannabis sativa
tgcaactactctctcaaggt

SEQ ID NO: 1488 - DNA, 20 bp, Cannabis sativa
aggtgggttgtgtattaatt

SEQ ID NO: 1489 - DNA, 20 bp, Cannabis sativa
ggttgtgtattaattaggcc

SEQ ID NO: 1490 - DNA, 20 bp, Cannabis sativa
caatattccacattcacacc

SEQ ID NO: 1491 - DNA, 20 bp, Cannabis sativa
aattaggcctggtgtgaatg

SEQ ID NO: 1492 - DNA, 20 bp, Cannabis sativa
ctggtgtgaatgtggaatat

SEQ ID NO: 1493 - DNA, 20 bp, Cannabis sativa
gaatattggcatatatagta

SEQ ID NO: 1494 - DNA, 20 bp, Cannabis sativa
ctttgttttgagattttaaa

SEQ ID NO: 1495 - DNA, 20 bp, Cannabis sativa
tttgttttgagattttaaaa

SEQ ID NO: 1496 - DNA, 20 bp, Cannabis sativa
aactttgcctgtttcattca

SEQ ID NO: 1497 - DNA, 20 bp, Cannabis sativa
agggtgtccatgaatgaaac

SEQ ID NO: 1498 - DNA, 20 bp, Cannabis sativa
aggcaaagtttgtctttgac

SEQ ID NO: 1499 - DNA, 20 bp, Cannabis sativa
aagtttgtctttgacaggat

SEQ ID NO: 1500 - DNA, 20 bp, Cannabis sativa
gacaggattggtctgaattt

SEQ ID NO: 1501 - DNA, 65 bp, Artificial Sequence
  mutated multiflora gene
ccaccaatggcagcatgacatcaacccatcgctcatgacatagtggataccacagacctccgtac

SEQ ID NO: 1502 - DNA, 65 bp, Artificial Sequence
  mutated multiflora gene
ccaccaatggcagcatgacatcaacccatcgctcatgacctagtggataccacagacctccgtac

SEQ ID NO: 1503 - DNA, 65 bp, Artificial Sequence
  mutated multiflora gene
ccaccaatggcagcatgacatcaacccatcgctcatgacttagtggataccacagacctccgtac